(19) United States
(12) Patent Application Publication
(10) Pub. No.: US 2013/0023532 A1
(43) Pub. Date: Jan. 24, 2013

(54) INDAZOLYL-PYRIMIDINES AS KINASE INHIBITORS

(71) Applicants: Linda N. Casillas, Collegeville, PA (US); Subhas J. Chakravorty, Collegeville, PA (US); Adam Kenneth Charnley, Collegeville, PA (US); Pamela A. Haile, King of Prussia, PA (US); Terry Vincent Hughes, Collegeville, PA (US); Jae U. Jeong, Collegeville, PA (US); Jianxing Kang, Collegeville, PA (US); Ami Lakdawala Shah, King of Prussia, PA (US); Lara Kathryn Leister, Collegeville, PA (US); Robert W. Marquis, JR., Collegeville, PA (US); Nathan Andrew Miller, Collegeville, PA (US); Daniel J. Price, Research Triangle Park, NC (US); Clark A. Sehon, King of Prussia, PA (US); Gren Z. Wang, Collegeville, PA (US); Daohua Zhang, Collegeville, PA (US); Patrick Eidam, Collegeville, PA (US)

(72) Inventors: Linda N. Casillas, Collegeville, PA (US); Subhas J. Chakravorty, Collegeville, PA (US); Adam Kenneth Charnley, Collegeville, PA (US); Pamela A. Haile, King of Prussia, PA (US); Terry Vincent Hughes, Collegeville, PA (US); Jae U. Jeong, Collegeville, PA (US); Jianxing Kang, Collegeville, PA (US); Ami Lakdawala Shah, King of Prussia, PA (US); Lara Kathryn Leister, Collegeville, PA (US); Robert W. Marquis, JR., Collegeville, PA (US); Nathan Andrew Miller, Collegeville, PA (US); Daniel J. Price, Research Triangle Park, NC (US); Clark A. Sehon, King of Prussia, PA (US); Gren Z. Wang, Collegeville, PA (US); Daohua Zhang, Collegeville, PA (US); Patrick Eidam, Collegeville, PA (US)

(21) Appl. No.: 13/637,419

(22) PCT Filed: Mar. 26, 2011
(86) PCT No.: PCT/US11/30103

§ 371 Date: Sep. 26, 2012

Related U.S. Application Data
(60) Provisional application No. 61/318,112, filed on Mar. 26, 2010.

(51) Int. Cl. A61K 31/5377; A61K 31/437; A61K 31/454; C07D 409/14; C07D 471/04; C07D 401/12; C07D 405/14; A61K 31/506; C07D 403/12

(52) U.S. Cl. 514/234.2; 514/275; 514/303; 514/338; 544/122; 544/323; 544/324; 546/119; 546/275.7

(57) ABSTRACT

Disclosed are compounds having the formula: or a salt thereof, wherein A, n, R¹, R^{1A}, and R² are as defined herein, and methods of making and using the same.

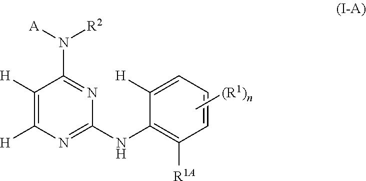

### FIELD OF THE INVENTION

The present invention relates to indazolyl-pyrimidinyl diamines that inhibit RIP2 kinase and methods of making and using the same. Specifically, the present invention relates to substituted indazoles as RIP2 kinase inhibitors.

### BACKGROUND OF THE INVENTION

Receptor interacting protein-2 (RIP2) kinase, which is also referred to as CARD3, RICK, CARDIAK, or RIPK2, is a TKL family serine/threonine protein kinase involved in innate immune signaling. RIP2 kinase is composed of an N-terminal kinase domain and a C-terminal caspase-recruitment domain (CARD) linked via an intermediate (IM) region ((1998) J. Biol. Chem. 273, 12296-12300; (1998) Current Biology 8, 885-889; and (1998) J. Biol. Chem. 273, 16968-16975). The CARD domain of RIP2 kinase mediates interaction with other CARD-containing proteins, such as NOD1 and NOD2 ((2000) J. Biol. Chem. 275, 27823-27831 and (2001) EMBO reports 2, 736-742). NOD1 and NOD2 are cytoplasmic receptors which play a key role in innate immune surveillance. They recognize both gram positive and gram negative bacterial pathogens and are activated by specific peptidoglycan motifs, diaminopimelic acid (i.e., DAP) and muramyl dipeptide (MDP), respectively ((2007) J Immunol 178, 2380-2386).

Following activation, RIP2 kinase associates with NOD1 or NOD2 and appears to function principally as a molecular scaffold to bring together other kinases (TAK1, IKKα/β/γ) involved in NF-κB and mitogen-activated protein kinase activation ((2006) Nature Reviews Immunology 6, 9-20). RIP2 kinase undergoes a K63-linked polyubiquitination on lysine-209 which facilitates TAK1 recruitment ((2008) EMBO Journal 27, 373-383). This post-translational modification is required for signaling as mutation of this residue prevents NOD1/2 mediated NF-kB activation. RIP2 kinase also undergoes autophosphorylation on serine-176, and possibly other residues ((2006) Cellular Signalling 18, 2223-2229). Studies using kinase dead mutants (K47A) and non-selective small molecule inhibitors have demonstrated that RIP2 kinase activity is important for regulating the stability of RIP2 kinase expression and signaling ((2007) Biochem J 404, 179-190 and (2009) J. Biol. Chem. 284, 19183-19188).

Dysregulation of RIP2-dependent signaling has been linked to autoinflammatory diseases. Gain-of-function mutations in the NACHT-domain of NOD2 cause Blau Syndrome/Early-onset Sarcoidosis, a pediatric granulomatous disease characterized by uveitis, dermatitis, and arthritis ((2001) Nature Genetics 29, 19-20; (2005) Journal of Rheumatology 32, 373-375; (2005) Current Rheumatology Reports 7, 427-433; (2005) Blood 105, 1195-1197; (2005) European Journal of Human Genetics 13, 742-747; (2006) American Journal of Ophthalmology 142, 1089-1092; (2006) Arthritis & Rheumatism 54, 3337-3344; (2009) Arthritis & Rheumatism 60, 1797-1803; and (2010) Rheumatology 49, 194-196). Mutations in the LRR-domain of NOD2 have been strongly linked to susceptibility to Crohn's Disease ((2002) Am. J. Hum. Genet. 70, 845-857; (2004) European Journal of Human Genetics 12, 206-212; (2008) Mucosal Immunology (2008) 1 (Suppl 1), S5-S9. 1, S5-S9; (2008) Inflammatory Bowel Diseases 14, 295-302; (2008) Experimental Dermatology 17, 1057-1058; (2008) British Medical Bulletin 87, 17-30; (2009) Inflammatory Bowel Diseases 15, 1145-1154 and (2009) Microbes and Infection 11, 912-918). Mutations in NOD1 have been associated with asthma ((2005) Hum. Mol. Genet. 14, 935-941) and early-onset and extra-intestinal inflammatory bowel disease ((2005) Hum. Mol. Genet. 14, 1245-1250). Genetic and functional studies have also suggested a role for RIP2-dependent signaling in a variety of other granulomateous disorders, such as sarcoidosis ((2009) Journal of Clinical Immunology 29, 78-89 and (2006) Sarcoidosis Vasculitis and Diffuse Lung Diseases 23, 23-29) and Wegner's Granulomatosis ((2009) Diagnostic Pathology 4, 23).

A potent, selective, small molecule inhibitor of RIP2 kinase activity would block RIP2-dependent pro-inflammatory signaling and thereby provide a therapeutic benefit in autoinflammatory diseases characterized in increased and/or dysregulated RIP2 kinase activity.

### SUMMARY OF THE INVENTION

The invention is directed to novel indazolyl-pyrimidinyl diamines. Specifically, the invention is directed to a compound according to Formula (I):

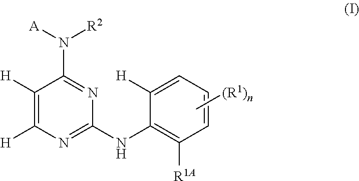

wherein:

R^{1A} is H, fluoro, methyl, methoxy or ethoxy;

n is 1, 2 or 3;

each R¹ is independently selected from halogen, hydroxy, (C₁-C₆)alkyl, cyano(C₁-C₆)alkyl, halo(C₁-C₆)alkyl, (C₁-C₄ alkyl)(C₁-C₄ alkyl)amino-halo(C₂-C₆)alkyl, (C₁-C₄ alkyl)(C₁-C₄ alkyl)amino-, —OR^{x}, —SO₂R^{x}, —NR^{z}SO₂R^{x}, (C₁-C₄ alkyl)(C₁-C₄ alkyl)amino-SO₂NR^{z}—, —CONR^{y}R^{z}, —SO₂NR^{y}R^{z}, —SO₂-heterocycloalkyl, heterocycloalkyl,

and wherein any of said heterocycloalkyl (that is, the heterocycloalkyl group and the heterocycloalkyl moiety of the —SO₂heterocycloalkyl group) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from halogen, (C₁-C₆)alkyl, halo(C₁-C₄)alkyl, —CO(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo, R^{x} is selected from (C₁-C₆)alkyl, halo(C₁-C₆)alkyl, hydroxy(C₂-C₆)alkyl-, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,

R^{y} is selected from H, (C₁-C₆)alkyl, (C₁-C₆)alkoxy, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and

R^{z} is H or (C₁-C₆)alkyl;

or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NR¹ⁿ, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NR¹ⁿ—, —O—, —S— and —SO₂—, where R¹ⁿ is H or —SO₂(C₁-C₄ alkyl);

R² is H or (C₁-C₄)alkyl;

A is

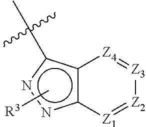

wherein:

R³ is H, (C₁-C₄)alkyl or an optionally substituted phenyl or 5-6 membered heteroaryl, where said phenyl or heteroaryl is optionally substituted by 1-3 groups independently selected from halogen, hydroxy, amino, C₁-C₃ alkyl, haloC₁-C₃ alkyl, C₁-C₃ alkoxy, haloC₁-C₃ alkoxy, (C₁-C₃)alkylamino- and ((C₁-C₃)alkyl)((C₁-C₃)alkyl)amino;

each Z₁, Z₂, Z₃, and Z₄ is independently selected from CH and CR⁴;

any one or two of Z₁, Z₂, Z₃, and Z₄ is N, and each of the remaining two or three of Z₁, Z₂, Z₃, and Z₄ is independently selected from CH and CR⁴, or

any one of Z₁, Z₂, Z₃, and Z₄ is NO, and each of the remaining three of Z₁, Z₂, Z₃, and Z₄ is independently selected from CH and CR⁴,

where each R⁴ is independently selected from halogen, (C₁-C₄)alkyl, halo(C₁-C₄)alkyl, (C₁-C₄)alkoxy, phenyl-oxy, and phenyl(C₁-C₄)alkoxy, wherein the phenyl moiety of said phenyl-oxy or phenyl(C₁-C₄)alkoxy is optionally substituted by 1-3 independently selected (C₁-C₃)alkyl groups;

provided that the compound is not:
N²-(3-{[2-(diethylamino)ethyl]oxy}phenyl)-N-(4-fluoro-2H-indazol-3-yl)-2,4-pyrimidinediamine; or
N²-[3-{[2-(diethylamino)ethyl]oxy}-4-(methyloxy)phenyl]-N⁴-(4-fluoro-2H-indazol-3-yl)-2,4-pyrimidinediamine;

or a salt, particularly a pharmaceutically acceptable salt, thereof.

The present invention is also directed to a method of inhibiting RIP2 kinase which comprises contacting the kinase with a compound or salt, thereof, according to Formula (I-A).

wherein:

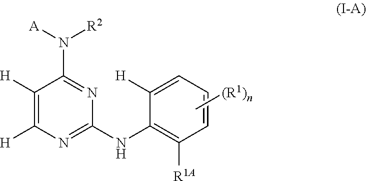

R^{1A} is H, fluoro, methyl, methoxy or ethoxy;

n is 1, 2 or 3;

each R¹ is independently selected from halogen, hydroxy, (C₁-C₆)alkyl, cyano(C₁-C₆)alkyl, halo(C₁-C₆)alkyl, (C₁-C₄ alkyl)(C₁-C₄ alkyl)amino-halo(C₂-C₆)alkyl, (C₁-C₄ alkyl)(C₁-C₄ alkyl)amino-, —OR^{x}, —SO₂R^{x}, —NR^{z}SO₂R^{x}, (C₁-C₄ alkyl)(C₁-C₄ alkyl)amino-SO₂NR^{z}—, —CONR^{y}R^{z}, —SO₂NR^{y}R^{z}, —SO₂-heterocycloalkyl, heterocycloalkyl,

and wherein any of said heterocycloalkyl (that is, the heterocycloalkyl group and the heterocycloalkyl moiety of the —SO₂ heterocycloalkyl group) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from halogen, (C₁-C₆)alkyl, halo(C₁-C₄)alkyl, —CO(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,

R^{x} is selected from (C₁-C₆)alkyl, halo(C₁-C₆)alkyl, hydroxy(C₂-C₆)alkyl-, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,

R^{y} is selected from H, (C₁-C₆)alkyl, (C₁-C₆)alkoxy, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and

R^{z} is H or (C₁-C₆)alkyl;

or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NR¹ⁿ—, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NR¹ⁿ—, —O—, —S— and —SO₂—, where R¹ⁿ is H or —SO₂(C₁-C₄ alkyl);

R² is H or (C₁-C₄)alkyl;

A is:

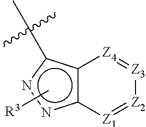

wherein:

R³ is H, (C₁-C₄)alkyl or an optionally substituted phenyl or 5-6 membered heteroaryl, where said phenyl or heteroaryl is optionally substituted by 1-3 groups independently selected from halogen, hydroxy, amino, C₁-C₃ alkyl, haloC₁-C₃ alkyl, C₁-C₃ alkoxy, haloC₁-C₃ alkoxy, (C₁-C₃)alkylamino- and ((C₁-C₃)alkyl)((C₁-C₃)alkyl)amino;

each Z₁, Z₂, Z₃, and Z₄ is independently selected from CH and CR⁴;

any one or two of Z₁, Z₂, Z₃, and Z₄ is N, and each of the remaining two or three of Z₁, Z₂, Z₃, and Z₄ is independently selected from CH and CR⁴, or

any one of Z₁, Z₂, Z₃, and Z₄ is NO(N-oxide), and each of the remaining three of Z₁, Z₂, Z₃, and Z₄ is independently selected from CH and CR⁴,

where each R⁴ is independently selected from halogen, (C₁-C₄)alkyl, halo(C₁-C₄)alkyl, (C₁-C₄)alkoxy, phenyl-oxy, and phenyl(C₁-C₄)alkoxy, wherein the phenyl moiety of said phenyl-oxy or phenyl(C₁-C₄)alkoxy is optionally substituted by 1-3 independently selected (C₁-C₃)alkyl groups;

or a salt, particularly a pharmaceutically acceptable salt, thereof.

The compounds of the invention (that is the compounds of Formula (I) and (I-A), and salts thereof), are inhibitors of RIP2 kinase and can be useful for the treatment of RIP2-mediated diseases and disorders, particularly uveitis, dermatitis, arthritis Crohn's disease, asthma, early-onset and extra-intestinal inflammatory bowel disease, and granulomateous disorders, such as adult sarcoidosis, Blau syndrome, early-onset sarcoidosis, and Wegner's Granulomatosis. Accordingly, the invention is further directed to pharmaceutical compositions comprising a compound of the invention.

The invention is still further directed to methods of inhibiting RIP2 kinase and treatment of conditions associated therewith using a compound of the invention or a pharmaceutical composition comprising a compound of the invention.

### DETAILED DESCRIPTION OF THE INVENTION

It will be appreciated by those skilled in the art that the compounds of this invention, represented by generic Formula (II):

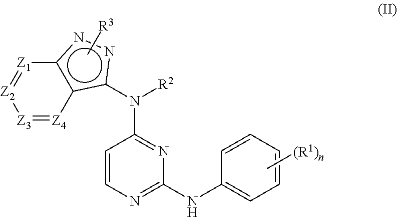

exist as tautomers (where R³ is H) or regioisomers (where R³ is alkyl or (het)aryl) of Formula (II)-A and Formula (II)-B, as follows:

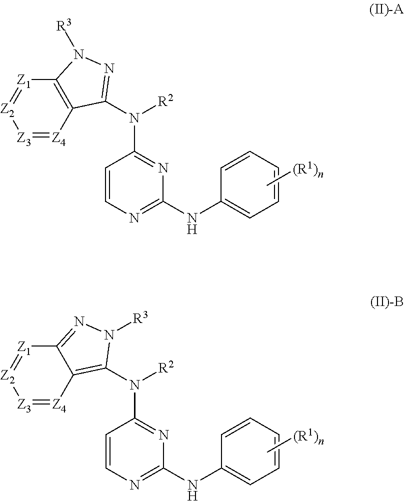

wherein the group A is represented as formula A-a and A-b, respectively:

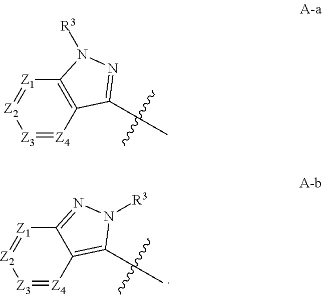

In addition, it will be appreciated by those skilled in the art that the compounds of this invention may exist in several other tautomeric forms. All tautomeric forms of the compounds described herein are intended to be encompassed within the scope of the present invention. It is to be understood that any reference to a named compound of this invention is intended to encompass all tautomers of the named compound and any mixtures of tautomers of the named compound.

The alternative definitions for the various groups and substituent groups of Formula I provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

In one embodiment of the compounds of this invention, R^{1A} is H. In another embodiment, R^{1A} is fluoro. In a further embodiment, R^{1A} is methyl. In yet another embodiment, R^{1A} is methoxy or ethoxy.

In a further embodiment, each R¹ is independently selected from halogen, hydroxy, (C₁-C₄)alkyl, cyano(C₁-C₄)alkyl, halo(C₁-C₄)alkyl, (C₁-C₄ alkyl)(C₁-C₄ alkyl)amino-halo(C₂-C₄)alkyl, (C₁-C₄ alkyl)(C₁-C₄ alkyl)amino-, —OR^{x}, —SO₂R^{x}, —NR^{z}SO₂R^{x}, (C₁-C₄ alkyl)(C₁-C₄ alkyl)amino-SO₂NR^{z}—, —CONR^{y}R^{z}, —SO₂NR^{y}R^{z}, —SO₂-heterocycloalkyl, heterocycloalkyl,

and wherein any of said heterocycloalkyl is a 5-6 membered non-aromatic ring containing one heteroatom selected from N, O and S, or containing one nitrogen atom and one additional heteroatom selected from N, O and S; which heterocycloalkyl is optionally substituted by 1-3 substituents independently selected from halogen, (C₁-C₄)alkyl, halo(C₁-C₄)alkyl, —CO(C₁-C₄)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,

R^{x} is selected from (C₁-C₄)alkyl, halo(C₁-C₄)alkyl, hydroxy(C₂-C₄)alkyl-, (C₃-C₇)cycloalkyl, amino(C₂-C₄)alkyl-, ((C₁-C₄)alkyl)amino(C₂-C₄)alkyl-, and ((C₁-C₄)alkyl)((C₁-C₄)alkyl)amino(C₂-C₄)alkyl-,

R^{y} is selected from H, (C₁-C₄)alkyl, (C₁-C₄)alkoxy, (C₃-C₇)cycloalkyl, amino(C₂-C₄)alkyl-, ((C₁-C₄)alkyl)amino(C₂-C₄)alkyl-, and ((C₁-C₄)alkyl)((C₁-C₄)alkyl)amino(C₂-C₄)alkyl-, and

R^{z} is H or (C₁-C₄)alkyl;

or one of R^{1A}, taken together with an adjacent R¹ group and the carbon atoms connecting the R^{1A} and R¹ groups or two adjacent R¹ groups taken together with the carbon atoms connecting the two R¹ groups form a 6 membered, non-aromatic heterocyclic ring containing two —O— ring moieties or a 5 membered, aromatic or non-aromatic heterocyclic ring containing one —NR¹ⁿ—, —O—, —S— or —SO₂— ring moiety, where R¹ⁿ is H or —SO₂(C₁-C₄ alkyl).

In a still further embodiment, each R¹ is independently selected from halogen, hydroxy, (C₁-C₄)alkyl, cyano(C₁-C₄)alkyl, halo(C₁-C₄)alkyl, (C₁-C₄ alkyl)(C₁-C₄ alkyl)amino-halo(C₂-C₄)alkyl, (C₁-C₄ alkyl)(C₁-C₄ alkyl)amino-, —OR^{x}, —SO₂R^{x}, —NR^{z}SO₂R^{x}, (C₁-C₄ alkyl)(C₁-C₄ alkyl)amino-SO₂NR^{z}—, —CONR^{y}R^{z}, —SO₂NR^{y}R^{z}, —SO₂-heterocycloalkyl, heterocycloalkyl,

and wherein any of said heterocycloalkyl is a 5-6 membered non-aromatic ring containing one heteroatom selected from N, O and S, or containing one nitrogen atom and one additional heteroatom selected from N, O and S; which heterocycloalkyl is optionally substituted by 1-3 independently selected (C₁-C₄)alkyl substituents,

R^{x} is selected from (C₁-C₄)alkyl, halo(C₁-C₄)alkyl, hydroxy(C₂-C₄)alkyl-, and ((C₁-C₄)alkyl)((C₁-C₄)alkyl)amino(C₂-C₄)alkyl-,

R^{y} is selected from H, (C₁-C₄)alkyl, (C₁-C₄)alkoxy, and ((C₁-C₄)alkyl)((C₁-C₄)alkyl)amino(C₂-C₄)alkyl-, and

R^{z} is H or (C₁-C₄)alkyl;

or one of R^{1A}, taken together with an adjacent R¹ group and the carbon atoms connecting the R^{1A} and R¹ groups or two adjacent R¹ groups taken together with the carbon atoms connecting the two R¹ groups form a 6 membered, non-aromatic heterocyclic ring containing two —O— ring moieties or a 5 membered, aromatic or non-aromatic heterocyclic ring containing one —NR¹ⁿ—, —O—, —S— or —SO₂— ring moiety, where R¹ⁿ is H or —SO₂(C₁-C₄ alkyl).

In another embodiment of this invention, each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring containing one heteroatom selected from N, O and S, or containing one nitrogen atom and optionally containing 1 additional heteroatom selected from N, O and S, where said heterocyclic ring is optionally substituted one or two times, independently, by (C₁-C₆)alkyl,

wherein R^{y} and R^{z} are each independently selected from H and (C₁-C₄ alkyl) or R^{y} is H, (C₁-C₂ alkyl), or (C₁-C₂ alkyl)(C₁-C₂ alkyl)amino(C₂-C₃ alkyl)- and R^{z} is H or (C₁-C₂ alkyl), or R^{y} and R^{z}, taken together are —CH₂CH₂CH₂CH₂—.

In yet another embodiment of this invention, each R¹ is independently selected from hydroxy, chloro, fluoro, —OCH₃, —OCH₂CH₃, —OCHF₂, —CH₃, —CF₃, —CH(CF₃)N(CH₃)₂, —N(CH₃)₂, —C(CN)(CH₃)₂, —CONH₂, —SO₂CH₃, —SO₂CF₃, —SO₂CH₂CH₃, —SO₂CH(CH₃)₂, —SO₂C(CH₃)₃, —SO₂C(CH₃)₂CH₂OH, —SO₂NH₂, —SO₂N(CH₃)₂, —NHSO₂CH₃, —N(CH₃)SO₂CH₃, —NHSO₂N(CH₃)₂, —SO₂NHOCH₃, —SO₂N(CH₃)OCH₃, —SO₂N(CH₃)(CH₂CH₂N(CH₃)₂), —SO₂-pyrrolidin-1-yl, —SO₂-morpholin-4-yl, —SO₂-tetrahydropyran-4-yl, -pyrrolidin-1-yl, 3,3-difluoro-pyrrolidin-1-yl, 1,1-dioxido-isothiazolidin-2-yl, morpholin-4-yl, and 4-methyl-piperazin-1-yl.

In another embodiment of this invention, R¹ is —SO₂NH₂, and R^{1A} taken together with an adjacent R¹ group form a —OCH₂CH₂— moiety. In a further embodiment, R^{1A} taken together with an adjacent R¹ group form a —CH═CH₂S— or a —CH═CH₂NH— moiety or two adjacent R¹ groups form a —SO₂CH═CH₂—, —OCH₂CH₂O—, —CH₂CH₂O—, —CH═CH₂NH— or a —CH═CH₂N(SO₂CH₃)— moiety.

In a further embodiment of this invention, each R¹ is independently selected from chloro, fluoro, methoxy, —SO₂(CH₃), —SO₂ pyrrolidin-1-yl, —SO₂NH₂, —SO₂N(CH₃)₂, —SO₂N(CH₃)(CH₂CH₂N(CH₃)₂), and 4-methyl-piperazin-1-yl.

In one embodiment, n is 2 or 3 and each R¹ is independently selected from (C₁-C₄)alkoxy.

In another embodiment, one R¹ is —SO₂R^{x}, —SO₂NR^{y}R^{z}, —SO₂-heterocycloalkyl or heterocycloalkyl, wherein

R^{x} is (C₁-C₄)alkyl, trifluoromethyl, or hydroxy(C₂-C₄)alkyl-;

R^{y} is H, (C₁-C₂)alkyl, (C₁-C₂)alkoxy, or (C₁-C₂ alkyl)(C₁-C₂ alkyl)amino(C₂-C₃ alkyl)-,

R^{z} is H or (C₁-C₂ alkyl),

any of said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,

and, when n is 2 or 3, each other R¹ is independently selected from halogen, (C₁-C₂)alkyl, halo(C₁-C₂)alkyl, (C₁-C₂)alkoxy, halo(C₁-C₂)alkoxy, and —SO₂(C₁-C₄)alkyl.

In yet another embodiment, n is 1, 2 or 3, one R¹ is —SO₂R^{x}, wherein R^{x} is (C₁-C₄)alkyl, trifluoromethyl, or hydroxy(C₂-C₄)alkyl-, and

when n is 2 or 3, each other R¹ is independently selected from halogen, (C₁-C₂)alkyl, halo(C₁-C₂)alkyl, hydroxy, (C₁-C₂)alkoxy, halo(C₁-C₂)alkoxy, —SO₂(C₁-C₄)alkyl, —CO₂(C₁-C₄)alkyl and an optionally substituted 5 or 6-membered non-aromatic heterocyclic ring, wherein the 5-6 membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents.

In yet another embodiment, n is 1, 2 or 3, one R¹ is —SO₂NR^{y}R^{z}, wherein R^{y} is H, (C₁-C₂)alkyl, (C₁-C₂)alkoxy, or (C₁-C₂ alkyl)(C₁-C₂ alkyl)amino(C₂-C₃ alkyl)-,

and when n is 2 or 3, each other R¹ is independently selected from halogen, (C₁-C₂)alkyl, halo(C₁-C₂)alkyl, and (C₁-C₂)alkoxy.

In yet another embodiment, n is 1, 2 or 3, one R¹ is —SO₂-heterocycloalkyl, wherein said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,

and when n is 2 or 3, each other R¹ is independently selected from halogen, (C₁-C₂)alkyl, halo(C₁-C₂)alkyl, and (C₁-C₂)alkoxy.

In a further embodiment, n is 1 or 2 and one R¹ is heterocycloalkyl, wherein said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents.

and when n is 2, the other R¹ is independently selected from halogen and (C₁-C₂)alkyl.

In specific embodiments of this invention, R² is H or methyl. In another specific embodiment, R² is ethyl.

In another embodiment of this invention, R³ is H or (C₁-C₄)alkyl.

In a further embodiment, R³ is an optionally substituted phenyl or 5-6 membered heteroaryl, where said phenyl or heteroaryl is optionally substituted by 1-2 groups independently selected from halogen, hydroxy, amino, C₁-C₃ alkyl, C₁-C₃ alkoxy, (C₁-C₃)alkylamino- and ((C₁-C₃)alkyl)((C₁-C₃)alkyl)amino, and

where said optionally substituted 5-membered heteroaryl contains one heteroatom selected from N, O and S and optionally contains 1, 2 or 3 additional nitrogen atoms, and

said optionally substituted 6-membered heteroaryl contains 1, 2 or 3 nitrogen atoms.

In specific embodiments of this invention, R³ is H or methyl. In more specific embodiments of this invention, R³ is H.

In further embodiments, each of Z₁, Z₂, Z₃, and Z₄ are CH.

In another embodiment, one of Z₁, Z₂, Z₃, and Z₄ is CR⁴ and the remaining three of Z₁, Z₂, Z₃, and Z₄ are CH.

In yet another embodiment, two of Z₁, Z₂, Z₃, and Z₄ are CR⁴ and the remaining two of Z₁, Z₂, Z₃, and Z₄ are CH.

In a further embodiment, three of Z₁, Z₂, Z₃, and Z₄ are CR⁴ and the remaining Z₁, Z₂, Z₃, and Z₄ is CH.

In other embodiments, one of Z₁, Z₂, Z₃, and Z₄ is N or NO and each of the remaining three of Z₁, Z₂, Z₃, and Z₄ are CH. Specifically, Z₁ is N or NO and Z₂, Z₃, and Z₄ are CH.

In additional embodiments, one of Z₁, Z₂, Z₃, and Z₄ is N or NO, another one of Z₁, Z₂, Z₃, and Z₄ is CR⁴, and the remaining two of Z₁, Z₂, Z₃, and Z₄ are CH. Specifically, Z₁ is N or NO, any one of Z₂, Z₃, or Z₄ is CR⁴, and the other two of Z₂, Z₃, and Z₄ are CH. More specifically, Z₁ is N or NO, Z₃ is CR⁴, and Z₂ and Z₄ are CH or Z₁ is N, Z₂ is CR⁴, and Z₃ and Z₄ are CH.

In other embodiments, one of Z₁, Z₂, Z₃, and Z₄ is N or NO, two of the remaining Z₁, Z₂, Z₃, and Z₄ are CR⁴, and the remaining Z₁, Z₂, Z₃, or Z₄ is CH. Specifically, Z₁ is N or NO, any two of Z₂, Z₃, and Z₄ are CR⁴, and the other one of Z₂, Z₃, or Z₄ is CH. More specifically, Z₁ is N or NO, Z₂ and Z₃ are CR⁴, and Z₄ is CH.

In a further embodiment, each R⁴ is independently selected from halogen, (C₁-C₂)alkyl, halo(C₁-C₂)alkyl, (C₁-C₄)alkoxy, phenyl-oxy, and phenyl(C₁-C₄)alkoxy, wherein the phenyl moiety of said phenyl-oxy or phenyl(C₁-C₄)alkoxy is optionally substituted by 1 or 2 independently selected (C₁-C₂)alkyl groups.

In other embodiments, each R⁴ is independently selected from halogen, C₁-C₃ alkyl, C₁-C₃ alkoxy, and phenylC₁-C₂ alkoxy. In specific embodiments, each R⁴ is independently selected from chloro, fluoro, methyl, trifluoromethyl, methoxy, n-propyloxy, 3-methylphenoxy- and benzyloxy. In other specific embodiments, each R⁴ is independently selected from chloro, fluoro, methyl and methoxy. In other embodiments, each R⁴ is independently selected from halogen, C₁-C₃ alkyl and C₁-C₃ alkoxy, specifically, each R⁴ is independently selected from chloro, fluoro, methyl, and methoxy.

In specific embodiments where one of Z₁, Z₂, Z₃, and Z₄ is CR⁴, the remaining three of Z₁, Z₂, Z₃, and Z₄ are CH, R⁴ is chloro, fluoro, methyl, methoxy, or benzyloxy. In other specific embodiments where one of Z₁, Z₂, Z₃, and Z₄ is CR⁴, the remaining three of Z₁, Z₂, Z₃, and Z₄ are CH, R⁴ is chloro, fluoro, methyl, trifluoromethyl, methoxy, or n-propoxy.

In embodiments where two of Z₁, Z₂, Z₃, and Z₄ are CR⁴, the other two of Z₁, Z₂, Z₃, and Z₄ are CH, each R⁴ is independently selected from halogen, C₁-C₄ alkyl, and C₁-C₄ alkoxy; specifically, each R⁴ is independently selected from chloro, fluoro, methyl, and methoxy. In specific embodiments where two of Z₁, Z₂, Z₃, and Z₄ are CR⁴, each R⁴ is independently a halogen, specifically each R⁴ is independently selected from chloro and fluoro or both R⁴ are fluoro.

In other specific embodiments where two of Z₁, Z₂, Z₃, and Z₄ are CR⁴, one R⁴ is a halogen and the other R⁴ is C₁-C₄ alkyl, specifically one R⁴ is chloro or fluoro and the other R⁴ is methyl.

In specific embodiments of this invention, A is 1H-indazol-3-yl, 4-(methyloxy)-1H-indazol-3-yl, 5-(methyloxy)-1H-indazol-3-yl, 6-(methyloxy)-1H-indazol-3-yl, 6-(n-propyloxy)-1H-indazol-3-yl, 5-methyl-1H-indazol-3-yl, 6-methyl-1H-indazol-3-yl, 5-trifluoromethyl-1H-indazol-3-yl, 6-trifluoromethyl-1H-indazol-3-yl, 7-trifluoromethyl-1H-indazol-3-yl, 4-chloro-1H-indazol-3-yl, 5-chloro-1H-indazol-3-yl, 6-chloro-1H-indazol-3-yl, 7-chloro-1H-indazol-3-yl, 4,5-dichloro-1H-indazol-3-yl, 5-methyl-6-fluoro-indazol-3-yl, 6-methyl-7-fluoro-indazol-3-yl, 5-fluoro-6-methyl-indazol-3-yl, 5-methyloxy-1-methyl-indazol-3-yl, 5-fluoro-1,6-dimethyl-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,6-difluoro-1H-indazol-3-yl, 5,7-difluoro-1H-indazol-3-yl, 6,7-difluoro-1H-indazol-3-yl, or 1H-pyrazolo[3,4-b]pyridin-3-yl.

In other specific embodiments of this invention, A is 6-benzyloxy-1H-indazol-3-yl or 4-[(3-methylphenyl)oxy]-1H-indazol-3-yl.

In another specific embodiments of this invention, A is 1H-pyrazolo[3,4-b]pyridin-3-yl, 5-methyl-1H-pyrazolo[3,4-b]pyridin-3-yl, 6-methyl-1H-pyrazolo[3,4-b]pyridin-3-yl, 5-fluoro-6-methyl-1H-pyrazolo[3,4-b]pyridin-3-yl, 5-fluoro-1H-pyrazolo[3,4-b]pyridin-3-yl), 5-fluoro-7-oxido-1H-pyrazolo[3,4-b]pyridin-3-yl, or 5-fluoro-6-methyl-7-oxido-1H-pyrazolo[3,4-b]pyridin-3-yl.

In other specific embodiments, A is 1H-indazol-3-yl, 4-(methyloxy)-1H-indazol-3-yl, 5-(methyloxy)-1H-indazol-3-yl, 6-(methyloxy)-1H-indazol-3-yl, 5-methyl-1H-indazol-3-yl, 6-methyl-1H-indazol-3-yl, 5-chloro-1H-indazol-3-yl, 6-chloro-1H-indazol-3-yl, 4,5-dichloro-1H-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,7-difluoro-1H-indazol-3-yl, 6,7-difluoro-1H-indazol-3-yl, 1H-pyrazolo[3,4-b]pyridin-3-yl, or 6-benzyloxy-1H-indazol-3-yl.

The invention is directed to novel pyrazolyl-pyrimidinyl diamines. Specifically, the invention is directed to a compound according to Formula (I-B):

wherein:

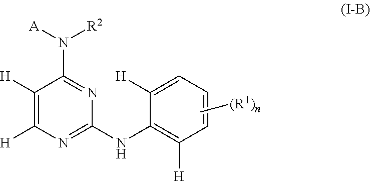

n is 1, 2 or 3;

R¹ is halogen, (C₁-C₆)haloalkoxy, —OR^{x}—SO₂R^{x}, —SO₂NR^{y}R^{z} or heterocycloalkyl,

wherein said heterocycloalkyl is a 5-6 membered non-aromatic ring containing one heteroatom selected from N, O and S, or containing one nitrogen atom and optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, aminoC₁-C₄ alkyl-, (C₁-C₄ alkyl)aminoC₁-C₄ alkyl-, (C₁-C₄ alkyl)(C₁-C₄ alkyl)aminoC₁-C₄ alkyl-, and oxo; and

wherein R^{x} is H, (C₁-C₆ alkyl), (C₃-C₇)cycloalkyl, amino(C₂-C₆ alkyl)-, (C₁-C₄ alkyl)amino(C₂-C₆ alkyl)-, or (C₁-C₄ alkyl)(C₁-C₄ alkyl)amino(C₂-C₆ alkyl)-, or

R^{y} is H, (C₁-C₆ alkyl), (C₃-C₇)cycloalkyl, amino(C₂-C₆ alkyl)-, (C₁-C₄ alkyl)amino(C₂-C₆ alkyl)-, or (C₁-C₄ alkyl)(C₁-C₄ alkyl)amino(C₂-C₆ alkyl)-, and

R^{z} is H or (C₁-C₆ alkyl), or

R^{y} and R^{z} taken together with the nitrogen atom to which they are attached form a 4-7 membered non-aromatic heterocyclic ring optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, amino(C₁-C₄ alkyl)-, (C₁-C₄ alkyl)amino(C₁-C₄ alkyl)-, (C₁-C₄ alkyl)(C₁-C₄ alkyl)amino(C₁-C₄ alkyl)-, and oxo;

R² is H or (C₁-C₄)alkyl;

A is:

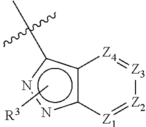

wherein:

R³ is H, (C₁-C₄)alkyl or an optionally substituted phenyl or 5-6 membered heteroaryl, where said phenyl or heteroaryl is optionally substituted by 1-3 groups independently selected from halogen, hydroxy, amino, C₁-C₃ alkyl, haloC₁-C₃ alkyl, C₁-C₃ alkoxy, haloC₁-C₃ alkoxy, (C₁-C₃)alkylamino- and ((C₁-C₃)alkyl)((C₁-C₃)alkyl)amino;

each Z₁, Z₂, Z₃, and Z₄ is independently selected from CH and CR⁴;

or any one or two of Z₁, Z₂, Z₃, and Z₄ is N, and each of the remaining two or three of Z₁, Z₂, Z₃, and Z₄ is independently selected from CH and CR⁴,

where each R⁴ is independently selected from halogen, C₁-C₄ alkyl, C₁-C₄ alkoxy, and phenylC₁-C₄ alkoxy;

provided that the compound is not:
N-(3-{[2-(diethylamino)ethyl]oxy}phenyl)-N⁴-(4-fluoro-2H-indazol-3-yl)-2,4-pyrimidinediamine; or
N²-[3-{[2-(diethylamino)ethyl]oxy}-4-(methyloxy)phenyl]-N⁴-(4-fluoro-2H-indazol-3-yl)-2,4-pyrimidinediamine;

or a salt, particularly a pharmaceutically acceptable salt, thereof.

The invention is further directed to a compound according to Formula (I) or Formula (I-A), wherein R^{1A} is H or a compound according to Formula (I-B) wherein:

each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring containing one heteroatom selected from N, O and S, or containing one nitrogen atom and optionally containing 1 additional heteroatom selected from N, O and S, where said heterocyclic ring is optionally substituted one or two times, independently, by (C₁-C₆)alkyl,

wherein R^{y} H, (C₁-C₂ alkyl), or (C₁-C₂ alkyl)(C₁-C₂ alkyl)amino(C₂-C₃ alkyl)- and R^{z} is H or (C₁-C₂ alkyl), or R^{y} and R^{z}, taken together are —CH₂CH₂CH₂CH₂—;

R² is H or methyl;

R³ is H or methyl;

one of Z₁, Z₂, Z₃, and Z₄ is CR⁴, and R⁴ is chloro, fluoro, methyl, methoxy, or benzyloxy; or two of Z₁, Z₂, Z₃, and Z₄ are CR⁴, each R⁴ is independently chloro or fluoro;

or a salt, particularly a pharmaceutically acceptable salt, thereof.

The invention is further directed to a compound according to Formula (I) or Formula (I-A), wherein R^{1A} is H or a compound according to Formula (I-B) wherein:

n is 1, 2 or 3;

R¹ is methoxy or —SO₂CH₃;

R² is H or methyl;

R³ is H or methyl;

each of Z₁, Z₂, Z₃, and Z₄ are CH, or

one of Z₁, Z₂, Z₃, and Z₄ is CR⁴ and the remaining three of Z₁, Z₂, Z₃, and Z₄ are CH, or

two of Z₁, Z₂, Z₃, and Z₄ are CR⁴ and the remaining two of Z₁, Z₂, Z₃, and Z₄ are CH, or
one of Z₁, Z₂, Z₃, and Z₄ is N and each of the remaining three of Z₁, Z₂, Z₃, and Z₄ are CH, or

one of Z₁, Z₂, Z₃, and Z₄ is N, another one of Z₁, Z₂, Z₃, and Z₄ is CR⁴, and the remaining two of Z₁, Z₂, Z₃, and Z₄ are CH;

where each R⁴ is independently selected from chloro, fluoro, methyl, and methoxy;

or a salt, particularly a pharmaceutically acceptable salt, thereof.

The invention is further directed to a compound according to Formula (I) or Formula (I-A), wherein:

R^{1A} is H;

n is 1, 2 or 3;

one R¹ is —SO₂R^{x}, —SO₂NR^{y}R^{z}, —SO₂-heterocycloalkyl or heterocycloalkyl, wherein

R^{x} is (C₁-C₄)alkyl, trifluoromethyl, or hydroxy(C₂-C₄)alkyl-;

R^{y} is H, (C₁-C₂)alkyl, (C₁-C₂)alkoxy, or (C₁-C₂ alkyl)(C₁-C₂ alkyl)amino(C₂-C₃ alkyl)-,

R^{z} is H or (C₁-C₂ alkyl),

any of said heterocycloalkyl is an optionally substituted 5-6 membered non-aromatic heterocyclic ring, wherein the 5 or 6-membered non-aromatic heterocyclic ring contains one heteroatom selected from N and O, or contains one nitrogen atom and one additional heteroatom selected from N and O, and is optionally substituted by 1-3 independently selected (C₁-C₂)alkyl substituents,

and, when n is 2 or 3, each other R¹ is independently selected from halogen, (C₁-C₂)alkyl, halo(C₁-C₂)alkyl, (C₁-C₂)alkoxy, halo(C₁-C₂)alkoxy, and —SO₂(C₁-C₄)alkyl;

R² is H or methyl;

R³ is H or methyl;

one of Z₁, Z₂, Z₃, and Z₄ is CR⁴, the remaining three of Z₁, Z₂, Z₃, and Z₄ are CH, and R⁴ is chloro, fluoro, methyl, trifluoromethyl, methoxy, or n-propoxy, or

two of Z₁, Z₂, Z₃, and Z₄ are CR⁴, and the other two of Z₁, Z₂, Z₃, and Z₄ are CH, where each R⁴ is independently selected from chloro and fluoro, or one R⁴ is a is chloro or fluoro and the other R⁴ is C₁-C₄ alkyl, specifically one R⁴ is chloro or fluoro and the other R⁴ is methyl.

The invention is further directed to a compound according to Formula (I) or Formula (I-A), wherein:

R^{1A} is H, fluoro, methyl, methoxy or ethoxy;

n is 1, 2 or 3;

each R¹ is independently selected from hydroxy, chloro, fluoro, —OCH₃, —OCH₂CH₃, —OCHF₂, —CH₃, —CF₃, —CH(CF₃)N(CH₃)₂, —N(CH₃)₂, —C(CN)(CH₃)₂, —CONH₂, —SO₂CH₃, —SO₂CF₃, —SO₂CH₂CH₃, —SO₂CH(CH₃)₂, —SO₂C(CH₃)₃, —SO₂C(CH₃)₂CH₂OH, —SO₂NH₂, —SO₂N(CH₃)₂, —NHSO₂CH₃, —N(CH₃)SO₂CH₃, —NHSO₂N(CH₃)₂, —SO₂NHOCH₃, —SO₂N(CH₃)OCH₃, —SO₂N(CH₃)(CH₂CH₂N(CH₃)₂), —SO₂-pyrrolidin-1-yl, —SO₂-morpholin-4-yl, —SO₂-tetrahydropyran-4-yl, -pyrrolidin-1-yl, 3,3-difluoro-pyrrolidin-1-yl, 1,1-dioxido-isothiazolidin-2-yl, morpholin-4-yl, and 4-methyl-piperazin-1-yl,

or R¹ is —SO₂NH₂, and R^{1A} taken together with an adjacent R¹ group form a —OCH₂CH₂— moiety, or

R^{1A} taken together with an adjacent R¹ group form a —CH═CH₂S— or a —CH═CH₂NH-moiety,

or R^{1A} is H and two adjacent R¹ groups form a —SO₂CH═CH₂—, —OCH₂CH₂O—, —CH₂CH₂O—, —CH═CH₂NH— or a —CH═CH₂N(SO₂CH₃)— moiety;

R² is H or methyl;

A is 1H-indazol-3-yl, 4-(methyloxy)-1H-indazol-3-yl, 5-(methyloxy)-1H-indazol-3-yl, 6-(methyloxy)-1H-indazol-3-yl, 6-(n-propyloxy)-1H-indazol-3-yl, 5-methyl-1H-indazol-3-yl, 6-methyl-1H-indazol-3-yl, 5-trifluoromethyl-1H-indazol-3-yl, 6-trifluoromethyl-1H-indazol-3-yl, 7-trifluoromethyl-1H-indazol-3-yl, 4-chloro-1H-indazol-3-yl, 5-chloro-1H-indazol-3-yl, 6-chloro-1H-indazol-3-yl, 7-chloro-1H-indazol-3-yl, 4,5-dichloro-1H-indazol-3-yl, 5-methyl-6-fluoro-indazol-3-yl, 6-methyl-7-fluoro-indazol-3-yl, 5-fluoro-6-methyl-indazol-3-yl, 5-methyloxy-1-methyl-indazol-3-yl, 5-fluoro-1,6-dimethyl-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,6-difluoro-1H-indazol-3-yl, 5,7-difluoro-1H-indazol-3-yl, 6,7-difluoro-1H-indazol-3-yl, or 1H-pyrazolo[3,4-b]pyridin-3-yl, or

A is 1H-pyrazolo[3,4-b]pyridin-3-yl, 5-methyl-1H-pyrazolo[3,4-b]pyridin-3-yl, 6-methyl-1H-pyrazolo[3,4-b]pyridin-3-yl, 5-fluoro-6-methyl-1H-pyrazolo[3,4-b]pyridin-3-yl, 5-fluoro-1H-pyrazolo[3,4-b]pyridin-3-yl), 5-fluoro-7-oxido-1H-pyrazolo[3,4-b]pyridin-3-yl, or 5-fluoro-6-methyl-7-oxido-1H-pyrazolo[3,4-b]pyridin-3-yl;

or a salt, particularly a pharmaceutically acceptable salt, thereof.

In another embodiment according to Formula (I) or Formula (I-A), wherein R^{1A} is H or a compound according to Formula (I-B) wherein n is 1, 2 or 3; each R¹ is independently selected from chloro, fluoro, methoxy, —SO₂(CH₃), —SO₂ pyrrolidin-1-yl, —SO₂NH₂, —SO₂N(CH₃)₂, —SO₂N(CH₃)(CH₂CH₂N(CH₃)₂), and 4-methyl-piperazin-1-yl; R² is H or methyl; R³ is H or methyl; A is 1H-indazol-3-yl, 4-(methyloxy)-1H-indazol-3-yl, 5-(methyloxy)-1-methyl-indazol-3-yl, 5-(methyloxy)-1H-indazol-3-yl, 6-(methyloxy)-1H-indazol-3-yl, 5-methyl-1H-indazol-3-yl, 6-methyl-1H-indazol-3-yl, 5-chloro-1H-indazol-3-yl, 6-chloro-1H-indazol-3-yl, 4,5-dichloro-1H-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,7-difluoro-1H-indazol-3-yl, 6,7-difluoro-1H-indazol-3-yl, 1H-pyrazolo[3,4-b]pyridin-3-yl, 6-benzyloxy-1H-indazol-3-yl; or a salt, particularly a pharmaceutically acceptable salt, thereof.

As used herein, the term “alkyl” represents a saturated, straight or branched hydrocarbon moiety, which may be unsubstituted or substituted by one, or more of the substituents defined herein. Exemplary alkyls include, but are not limited to methyl (Me), ethyl (Et), propyl, isopropyl, butyl, isobutyl, t-butyl and pentyl. The term “C₁-C₄” refers to an alkyl containing from 1 to 4 carbon atoms.

When the term “alkyl” is used in combination with other substituent groups, such as “haloalkyl” or “hydroxyalkyl” or “arylalkyl”, the term “alkyl” is intended to encompass a divalent straight or branched-chain hydrocarbon radical. For example, “arylalkyl” is intended to mean the radical -alkylaryl, wherein the alkyl moiety thereof is a divalent straight or branched-chain carbon radical and the aryl moiety thereof is as defined herein, and is represented by the bonding arrangement present in a benzyl group (—CH₂-phenyl).

As used herein, the term “alkenyl” refers to a straight or branched hydrocarbon moiety containing at least 1 and up to 3 carbon-carbon double bonds. Examples include ethenyl and propenyl.

As used herein, the term “alkynyl” refers to a straight or branched hydrocarbon moiety containing at least 1 and up to 3 carbon-carbon triple bonds. Examples include ethynyl and propynyl.

As used herein, the term “cycloalkyl” refers to a non-aromatic, saturated, cyclic hydrocarbon ring. The term “(C₃-C₈)cycloalkyl” refers to a non-aromatic cyclic hydrocarbon ring having from three to eight ring carbon atoms. Exemplary “(C₃-C₈)cycloalkyl” groups useful in the present invention include cyclopropyl, cyclobutyl, cyclopentyl, cyclohexyl, cycloheptyl, and cyclooctyl.

“Alkoxy” refers to a group containing an alkyl radical attached through an oxygen linking atom. The term “(C₁-C₄)alkoxy” refers to a straight- or branched-chain hydrocarbon radical having at least 1 and up to 4 carbon atoms attached through an oxygen linking atom. Exemplary “(C₁-C₄)alkoxy” groups useful in the present invention include, but are not limited to, methoxy, ethoxy, n-propoxy, isopropoxy, n-butoxy, s-butoxy, and t-butoxy.

“Alkylthio-” refers to a group containing an alkyl radical attached through a sulfur linking atom. The term “(C₁-C₄)alkylthio-” refers to a straight- or branched-chain hydrocarbon radical having at least 1 and up to 4 carbon atoms attached through a sulfur linking atom. Exemplary “(C₁-C₄)alkylthio-” groups useful in the present invention include, but are not limited to, methylthio-, ethylthio-, n-propylthio-, isopropylthio-, n-butylthio-, s-butylthio-, and t-butylthio-.

“Cycloalkyloxy” and “cycloalkylthio” refers to a group containing a saturated carbocyclic ring atoms attached through an oxygen or sulfur linking atom, respectively. Examples of “cycloalkyloxy” moieties include, but are not limited to, cyclopropoxy, cyclobutoxy, cyclopentyloxy, cyclohexyloxy, and the like.

“Aryl” represents a group or moiety comprising an aromatic, monovalent monocyclic or bicyclic hydrocarbon radical containing from 6 to 10 carbon ring atoms, which may be unsubstituted or substituted by one or more of the substituents defined herein, and to which may be fused one or more cycloalkyl rings, which may be unsubstituted or substituted by one or more substituents defined herein.

Generally, in the compounds of this invention, aryl is phenyl.

Heterocyclic groups may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms selected from nitrogen, oxygen and sulfur, and which may be unsubstituted or substituted by one or more of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl.

Generally, in the compounds of this invention, heterocycloalkyl groups are 5-membered and/or 6-membered heterocycloalkyl groups, such as pyrrolidyl (or pyrrolidinyl), tetrahydrofuryl (or tetrahydrofuranyl), tetrahydrothienyl, dihydrofuryl, oxazolinyl, thiazolinyl or pyrazolinyl, piperidyl (or piperidinyl), piperazinyl, morpholinyl, tetrahydropyranyl, dihydropyranyl, 1,3-dioxanyl, tetrahydro-2H-1,4-thiazinyl, 1,4-dioxanyl, 1,3-oxathianyl, and 1,3-dithianyl.

“Heteroaryl” represents a group or moiety comprising an aromatic monovalent monocyclic or bicyclic radical, containing 5 to 10 ring atoms, including 1 to 4 heteroatoms selected from nitrogen, oxygen and sulfur, which may be unsubstituted or substituted by one or more of the substituents defined herein. This term also encompasses bicyclic heterocyclic-aryl compounds containing an aryl ring moiety fused to a heterocycloalkyl ring moiety, containing 5 to 10 ring atoms, including 1 to 4 heteroatoms selected from nitrogen, oxygen and sulfur, which may be unsubstituted or substituted by one or more of the substituents defined herein. Illustrative examples of heteroaryls include, but are not limited to, thienyl, pyrrolyl, imidazolyl, pyrazolyl, furyl (or furanyl), isothiazolyl, furazanyl, isoxazolyl, oxazolyl, oxadiazolyl, thiazolyl, pyridyl (or pyridinyl), pyrazinyl, pyrimidinyl, pyridazinyl, triazinyl, tetrazinyl, triazolyl, tetrazolyl, benzo[b]thienyl, isobenzofuryl, 2,3-dihydrobenzofuryl, chromenyl, chromanyl, indolizinyl, isoindolyl, indolyl, indazolyl, purinyl, isoquinolyl, quinolyl, phthalazinyl, naphthridinyl, quinzolinyl, benzothiazolyl, benzimidazolyl, tetrahydroquinolinyl, cinnolinyl, pteridinyl, isothiazolyl.

Generally, the heteroaryl groups present in the compounds of this invention are 5-membered and/or 6-membered monocyclic heteroaryl groups. Selected 5-membered heteroaryl groups contain one nitrogen, oxygen or sulfur ring heteroatom, and optionally contain 1, 2 or 3 additional nitrogen ring atoms. Selected 6-membered heteroaryl groups contain 1, 2, 3 or 4 nitrogen ring heteroatoms. Selected 5- or 6-membered heteroaryl groups include thienyl, pyrrolyl, imidazolyl, pyrazolyl, furyl, isothiazolyl, furazanyl, isoxazolyl, oxazolyl, oxadiazolyl, thiazolyl, triazolyl, and tetrazolyl or pyridyl, pyrazinyl, pyrimidinyl, pyridazinyl, and triazinyl.

It is to be understood that the terms heterocycle, heterocyclic, heteroaryl, heterocycloalkyl, are intended to encompass stable heterocyclic groups where a ring nitrogen heteroatom is optionally oxidized (e.g., heterocyclic groups containing an N-oxide, such as pyridine-N-oxide) or where a ring sulfur heteroatom is optionally oxidized (e.g., heterocyclic groups containing sulfones or sulfoxide moieties, such as tetrahydrothienyl-1-oxide (a tetramethylene sulfoxide) or tetrahydrothienyl-1,1-dioxide (a tetramethylene sulfone)).

“Oxo” represents a double-bonded oxygen moiety; for example, if attached directly to a carbon atom forms a carbonyl moiety (C═O). The terms “halogen” and “halo” represent chloro, fluoro, bromo or iodo substituents. “Hydroxy” or “hydroxyl” is intended to mean the radical —OH.

As used herein, the term “compound(s) of the invention” means a compound of Formula (I), (I-A) or (I-B) (as defined above) in any form, i.e., any salt or non-salt form (e.g., as a free acid or base form, or as a pharmaceutically acceptable salt thereof) and any physical form thereof (e.g., including non-solid forms (e.g., liquid or semi-solid forms), and solid forms (e.g., amorphous or crystalline forms, specific polymorphic forms, solvates, including hydrates (e.g., mono-, di- and hemi-hydrates)), and mixtures of various forms.

As used herein, the term “optionally substituted” means unsubstituted groups or rings (e.g., cycloalkyl, heterocycle, and heteroaryl rings) and groups or rings substituted with one or more specified substituents.

Specific compounds of this invention are: N⁴-(5-fluoro-1H-indazol-3-yl)-N⁴-methyl-N²-[3,4,5-tris(methyloxy)phenyl]-2,4-pyrimidinediamine,
N²-[3,4-bis(methyloxy)phenyl]-N⁴-1H-pyrazolo[3,4-b]pyridin-3-yl-2,4-pyrimidinediamine,
3-({4-[(5-fluoro-1H-indazol-3-yl)amino]-2-pyrimidinyl}amino)-N,N-dimethyl-benzenesulfonamide,
N⁴-(5-fluoro-1H-indazol-3-yl)-N²-[3-(methoxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-1H-pyrazolo[3,4-b]pyridin-3-yl)-N⁴-methyl-N²-[3-(methyloxy)-5-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N²-(1,1-dioxido-1-benzothien-4-yl)-N⁴-(5-fluoro-1H-indazol-3-yl)-2,4-pyrimidinediamine,
3-((4-((5-fluoro-6-methyl-1H-indazol-3-yl)(methyl)amino)pyrimidin-2-yl)amino)-5-methylbenzenesulfonamide,
N²-[2,3-dimethyl-5-(methylsulfonyl)phenyl]-N⁴-(5-fluoro-1H-indazol-3-yl)-2,4-pyrimidinediamine,
2-{[3-({4-[(5-fluoro-1H-indazol-3-yl)amino]-2-pyrimidinyl}amino)phenyl]sulfinyl}ethanol,
N²-[3,4-bis(methyloxy)phenyl]-N⁴-[4-(methyloxy)-1H-indazol-3-yl]-2,4-pyrimidinediamine,
N²-[3,4-bis(methyloxy)phenyl]-N⁴-(6-methyl-1H-indazol-3-yl)-2,4-pyrimidinediamine,
N²-[3,4-bis(methyloxy)phenyl]-N⁴-{6-[(phenylmethyl)oxy]-1H-indazol-3-yl}-2,4-pyrimidinediamine,
N²-[3,4-bis(methyloxy)phenyl]-N⁴-1H-indazol-3-yl-2,4-pyrimidinediamine,
N⁴-[5-(methyloxy)-1H-indazol-3-yl]-N²-[3,4,5-tris(methyloxy)phenyl]-2,4-pyrimidinediamine,
N²-[3,4-bis(methyloxy)phenyl]-N⁴-(6-chloro-1H-indazol-3-yl)-2,4-pyrimidinediamine,
N²-[3,4-bis(methyloxy)phenyl]-N⁴-(5-chloro-1H-indazol-3-yl)-2,4-pyrimidinediamine,
N²-[3,4-bis(methyloxy)phenyl]-N⁴-[5-(methyloxy)-1H-indazol-3-yl]-2,4-pyrimidinediamine,
N²-[3,4-bis(methyloxy)phenyl]-N⁴-(7-fluoro-1H-indazol-3-yl)-2,4-pyrimidinediamine,
N⁴-1H-indazol-3-yl-N²-[4-(4-methyl-1-piperazinyl)phenyl]-2,4-pyrimidinediamine,
N²-[3,4-bis(methyloxy)phenyl]-N⁴-(4-fluoro-1H-indazol-3-yl)-2,4-pyrimidinediamine,
N²-[3,4-bis(methyloxy)phenyl]-N⁴-[6-(methyloxy)-1H-indazol-3-yl]-2,4-pyrimidinediamine,
N²-[3,4-bis(methyloxy)phenyl]-N⁴-(4,5-dichloro-1H-indazol-3-yl)-2,4-pyrimidinediamine,
N²-[3,4-bis(methyloxy)phenyl]-N⁴-(6-chloro-1-methyl-1H-indazol-3-yl)-2,4-pyrimidinediamine,
3-[(4-{[5-(methyloxy)-1H-indazol-3-yl]amino}-2-pyrimidinyl)amino]benzenesulfonamide,
N²-[3,4-bis(methyloxy)phenyl]-N⁴-(5-fluoro-1H-indazol-3-yl)-2,4-pyrimidinediamine,
N²-[3,4-bis(methyloxy)phenyl]-N⁴-(5,7-difluoro-1H-indazol-3-yl)-2,4-pyrimidinediamine,
N²-[3,4-bis(methyloxy)phenyl]-N⁴-(6,7-difluoro-1H-indazol-3-yl)-2,4-pyrimidinediamine,
N⁴-(5-fluoro-1H-indazol-3-yl)-N²-[3-(1-pyrrolidinylsulfonyl)phenyl]-2,4-pyrimidinediamine,
N²-[4-fluoro-3-(methylsulfonyl)phenyl]-N⁴-[5-(methyloxy)-1H-indazol-3-yl]-2,4-pyrimidinediamine,
N⁴-(5-fluoro-1H-indazol-3-yl)-N²-[4-fluoro-3-(methylsulfonyl)phenyl]2,4-pyrimidinediamine,
N⁴-(5-fluoro-1H-indazol-3-yl)-N⁴-methyl-N²-[3,4,5-tris(methyloxy)phenyl]-2,4-pyrimidinediamine,
N⁴-(5-fluoro-1H-indazol-3-yl)-N⁴-methyl-N²-[3-(methylsulfonyl)phenyl]-2,4-pyrimidinediamine,
N²-[3,4-bis(methyloxy)phenyl]-N⁴-[1-methyl-5-(methyloxy)-1H-indazol-3-yl]-2,4-pyrimidinediamine,
N⁴-[5-(methyloxy)-1H-indazol-3-yl]-N²-[3,4,5-tris(methyloxy)phenyl]-2,4-pyrimidinediamine,
N²-[4-fluoro-3-(methyloxy)phenyl]-N⁴-[5-(methyloxy)-1H-indazol-3-yl]-2,4-pyrimidinediamine,
N-[2-(dimethylamino)ethyl]-N-methyl-3-[(4-{[5-(methyloxy)-1H-indazol-3-yl]amino}-2-pyrimidinyl)amino]benzenesulfonamide,
N,N-dimethyl-3-[(4-{[5-(methyloxy)-1H-indazol-3-yl]amino}-2-pyrimidinyl)amino]benzenesulfonamide,
N⁴-[5-(methyloxy)-1H-indazol-3-yl]-N²-[3-(1-pyrrolidinylsulfonyl)phenyl]-2,4-pyrimidinediamine,
N²-{3-[1-(dimethylamino)-2,2,2-trifluoroethyl]phenyl}-N⁴-(5-fluoro-1H-indazol-3-yl)-2,4-pyrimidinediamine,
2-[3-({4-[(5-fluoro-1H-indazol-3-yl)amino]-2-pyrimidinyl}amino)phenyl]-2-methylpropanenitrile,
N⁴-(5-fluoro-1H-indazol-3-yl)-N²-[3-(methylsulfonyl)phenyl]-2,4-pyrimidinediamine,
N²-(2,3-dihydro-1,4-benzodioxin-6-yl)-N⁴-(5-fluoro-1H-indazol-3-yl)-2,4-pyrimidinediamine,
N⁴-(5-fluoro-1H-indazol-3-yl)-N²-[4-methyl-3-(methylsulfonyl)phenyl]-2,4-pyrimidinediamine,
2-{[3-({4-[(5-fluoro-1H-indazol-3-yl)amino]-2-pyrimidinyl}amino)phenyl]sulfonyl}-2-methyl-1-propanol,
4-({4-[(5-fluoro-1H-indazol-3-yl)amino]-2-pyrimidinyl}amino)-2,6-bis(methyloxy)phenol,
N⁴-(5-fluoro-1H-indazol-3-yl)-N²-1H-indol-6-yl-2,4-pyrimidinediamine,
N⁴-(5-fluoro-1H-indazol-3-yl)-N²-1H-indol-4-yl-2,4-pyrimidinediamine,
N⁴-(5-fluoro-1H-indazol-3-yl)-N²-[1-(methylsulfonyl)-1H-indol-6-yl]-2,4-pyrimidinediamine,
N⁴-(5-fluoro-1H-indazol-3-yl)-N²-[2-methyl-3-(methylsulfonyl)phenyl]-2,4-pyrimidinediamine,
N⁴-(5-fluoro-1H-indazol-3-yl)-N²-[1-(methylsulfonyl)-1H-indol-5-yl]-2,4-pyrimidinediamine,
N⁴-(7-chloro-1H-indazol-3-yl)-N²-[4-methyl-3-(methylsulfonyl)phenyl]-2,4-pyrimidinediamine,
N²-[4-methyl-3-(methylsulfonyl)phenyl]-N⁴-[7-(trifluoromethyl)-1H-indazol-3-yl]-2,4-pyrimidinediamine,
N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[4-(methylsulfonyl)phenyl]-2,4-pyrimidinediamine,
N⁴-(5-fluoro-1H-indazol-3-yl)-N²-[3-(4-morpholinylsulfonyl)phenyl]-2,4-pyrimidinediamine,
3-({4-[(5-fluoro-1H-indazol-3-yl)amino]-2-pyrimidinyl}amino)-N-(methyloxy)benzenesulfonamide,
3-({4-[(5-fluoro-1H-indazol-3-yl)amino]-2-pyrimidinyl}amino)-N-methyl-N-(methyloxy)benzenesulfonamide,
5-({4-[(5-fluoro-1H-indazol-3-yl)amino]-2-pyrimidinyl}amino)-N,2-DImethyl-N-(methyloxy)benzenesulfonamide,
N²-[3-(ethyloxy)-5-(methylsulfonyl)phenyl]-N⁴-(5-fluoro-1H-indazol-3-yl)-2,4-pyrimidinediamine,
N⁴-(5-fluoro-1H-indazol-3-yl)-N²-{3-[(1-methylethyl)sulfonyl]phenyl}-2,4-pyrimidinediamine,
N²-(3,5-dimethylphenyl)-N⁴-(5-fluoro-1H-indazol-3-yl)-2,4-pyrimidinediamine,
N⁴-(5-fluoro-1H-indazol-3-yl)-N²-{4-methyl-3-[(1-methylethyl)sulfonyl]phenyl}-2,4-pyrimidinediamine,
N⁴-(5-fluoro-1H-indazol-3-yl)-N²-[4-methyl-3-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N⁴-(5-fluoro-1H-indazol-3-yl)-N²-[3-(methyloxy)-5-(methylsulfonyl)phenyl]-2,4-pyrimidinediamine,
N²-[3-(ethylsulfonyl)-5-(methyloxy)phenyl]-N⁴-(5-fluoro-1H-indazol-3-yl)-2,4-pyrimidinediamine,
N²-{4-(ethyloxy)-3-[(1-methylethyl)sulfonyl]phenyl}-N⁴-(5-fluoro-1H-indazol-3-yl)-2,4-pyrimidinediamine,
N⁴-(5-fluoro-1H-indazol-3-yl)-N⁴-methyl-N²-[4-methyl-3-(methylsulfonyl)phenyl]-2,4-pyrimidinediamine,
N⁴-(5-fluoro-1H-indazol-3-yl)-N⁴-methyl-N²-[3-methyl-5-(methylsulfonyl)phenyl]-2,4-pyrimidinediamine,
N⁴-ethyl-N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[3-(methylsulfonyl)-5-(4-morpholinyl)phenyl]-2,4-pyrimidinediamine,
N⁴-(5-fluoro-1H-indazol-3-yl)-N²-(1-methyl-1H-imidazol-2-yl)-2,4-pyrimidinediamine,
N⁴-(5-fluoro-1H-indazol-3-yl)-N²-[3-(methyloxy)-5-(methylsulfonyl)phenyl]-2,4-pyrimidinediamine,
N⁴-(5-fluoro-1H-indazol-3-yl)-N²-[3-fluoro-5-(methylsulfonyl)phenyl]-2,4-pyrimidinediamine,
2-{[5-({4-[(5-fluoro-1H-indazol-3-yl)amino]-2-pyrimidinyl}amino)-2-methylphenyl]sulfonyl}-2-methyl-1-propanol,
N⁴-(5-fluoro-1H-indazol-3-yl)-N²-{4-fluoro-3-[(1-methylethyl)sulfonyl]phenyl}-2,4-pyrimidinediamine,
N⁴-(5-fluoro-1H-indazol-3-yl)-N⁴-methyl-N²-[3-(methyloxy)-5-(methylsulfonyl)phenyl]-2,4-pyrimidinediamine,
N⁴-(5-fluoro-1H-indazol-3-yl)-N²-[3-fluoro-4-methyl-5-(methylsulfonyl)phenyl]-2,4-pyrimidinediamine,
N-[3-({4-[(5-fluoro-1H-indazol-3-yl)amino]-2-pyrimidinyl}amino)phenyl]-N-methylmethanesulfonamide,
N²-[3-(dimethylamino)-5-(methylsulfonyl)phenyl]-N⁴-(5-fluoro-1H-indazol-3-yl)-2,4-pyrimidinediamine,
3-({4-[(5-fluoro-1H-indazol-3-yl)amino]-2-pyrimidinyl}amino)benzamide,
N⁴-(5-fluoro-1H-indazol-3-yl)-N²-[2-fluoro-4-(methylsulfonyl)phenyl]-2,4-pyrimidinediamine,
N⁴-(5-fluoro-1H-indazol-3-yl)-N²-[3-(methylsulfonyl)-5-(1-pyrrolidinyl)phenyl]-2,4-pyrimidinediamine,
N²-[3-(ethyloxy)-5-(ethylsulfonyl)phenyl]-N⁴-(5-fluoro-1H-indazol-3-yl)-2,4-pyrimidinediamine,
N⁴-(5-fluoro-1H-indazol-3-yl)-N²-[4-(methylsulfonyl)-2,3-dihydro-1-benzofuran-6-yl]-2,4-pyrimidinediamine,
N⁴-(5-fluoro-1H-indazol-3-yl)-N²-[4-fluoro-3-(methylsulfonyl)phenyl]-N⁴-methyl-2,4-pyrimidinediamine,
N⁴-(5-fluoro-1H-indazol-3-yl)-N⁴-methyl-N²-[2-methyl-3-(methylsulfonyl)phenyl]-2,4-pyrimidinediamine,
N²-[3,4-DImethyl-5-(methylsulfonyl)phenyl]-N⁴-(5-fluoro-1H-indazol-3-yl)-N⁴-methyl-2,4-pyrimidinediamine,
N⁴-(5-fluoro-1H-indazol-3-yl)-N²-[2-(methyloxy)-5-(methylsulfonyl)phenyl]-2,4-pyrimidinediamine,
3-({4-[(5-fluoro-1H-indazol-3-yl)amino]-2-pyrimidinyl}amino)-4-(methyloxy)benzenesulfonamide,
N⁴-(5-fluoro-1H-indazol-3-yl)-N²-{3-[(trifluoromethyl)sulfonyl]phenyl}-2,4-pyrimidinediamine,
N²-1-benzothien-4-yl-N⁴-(5-fluoro-1H-indazol-3-yl)-2,4-pyrimidinediamine,
N²-[2,4-dimethyl-5-(methylsulfonyl)phenyl]-N⁴-(5-fluoro-1H-indazol-3-yl)-2,4-pyrimidinediamine,
7-({4-[(5-fluoro-1H-indazol-3-yl)amino]-2-pyrimidinyl}amino)-2,3-dihydro-1-benzofuran-5-sulfonamide,
N⁴-(5-fluoro-1H-indazol-3-yl)-N²-[2-fluoro-3-(methylsulfonyl)phenyl]-2,4-pyrimidinediamine,
N²-[4-methyl-3-(methylsulfonyl)phenyl]-N⁴-[6-(trifluoromethyl)-1H-indazol-3-yl]-2,4-pyrimidinediamine,
N⁴-(6,7-DIfluoro-1H-indazol-3-yl)-N²-[4-methyl-3-(methylsulfonyl)phenyl]-2,4-pyrimidinediamine,
N⁴-(6-methyl-1H-indazol-3-yl)-N²-[4-methyl-3-(methylsulfonyl)phenyl]-2,4-pyrimidinediamine,
N⁴-(5-chloro-1H-indazol-3-yl)-N²-4-methyl-3-(methylsulfonyl)phenyl]-2,4-pyrimidinediamine,
N⁴-(4-chloro-1H-indazol-3-yl)-N²-[4-methyl-3-(methylsulfonyl)phenyl]-2,4-pyrimidinediamine,
N⁴-(7-fluoro-1H-indazol-3-yl)-N²-[4-methyl-3-(methylsulfonyl)phenyl]-2,4-pyrimidinediamine,
N⁴-(5,7-DIfluoro-1H-indazol-3-yl)-N²-[4-methyl-3-(methylsulfonyl)phenyl]-2,4-pyrimidinediamine,
N⁴-(5,6-difluoro-1H-indazol-3-yl)-N²-[4-methyl-3-(methylsulfonyl)phenyl]-2,4-pyrimidinediamine,
N⁴-(5-fluoro-1H-indazol-3-yl)-N²-[2-methyl-5-(methylsulfonyl)phenyl]-2,4-pyrimidinediamine,
N⁴-(5-fluoro-1H-indazol-3-yl)-N²-[3,4,5-tris(methyloxy)phenyl]-2,4-pyrimidinediamine,
N²-[4-methyl-3-(methylsulfonyl)phenyl]-N⁴-[5-(methyloxy)-1H-indazol-3-yl]-2,4-pyrimidinediamine,
N′-[3-({4-[(5-fluoro-1H-indazol-3-yl)amino]-2-pyrimidinyl}amino)phenyl]-N,N-dimethylsulfamide,
3-({4-[(5-fluoro-1H-indazol-3-yl)amino]-2-pyrimidinyl}amino)benzenesulfonamide,
5-({4-[(5-fluoro-1H-indazol-3-yl)amino]-2-pyrimidinyl}amino)-2-methylbenzenesulfonamide,
N⁴-(6,7-difluoro-1H-indazol-3-yl)-N⁴-methyl-N²-[4-methyl-3-(methylsulfonyl)phenyl]-2,4-pyrimidinediamine,
N⁴-(6,7-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3,4,5-tris(methyloxy)phenyl]-2,4-pyrimidinediamine,
N⁴-(7-fluoro-1H-indazol-3-yl)-N⁴-methyl-N²-[4-methyl-3-(methylsulfonyl)phenyl]-2,4-pyrimidinediamine,
N⁴-(7-fluoro-1H-indazol-3-yl)-N⁴-methyl-N²-[3-methyl-5-(methylsulfonyl)phenyl]-2,4-pyrimidinediamine,
N⁴-(7-fluoro-1H-indazol-3-yl)-N⁴-methyl-N²-[3-(methyloxy)-5-(methylsulfonyl)phenyl]-2,4-pyrimidinediamine,
N⁴-(7-fluoro-1H-indazol-3-yl)-N⁴-methyl-N²-[3,4,5-tris(methyloxy)phenyl]-2,4-pyrimidinediamine,
N⁴-(5,6-difluoro-1H-indazol-3-yl)-N⁴-methyl-N²-[3,4,5-tris(methyloxy)phenyl]2,4-pyrimidinediamine,
N⁴-(5,6-difluoro-1H-indazol-3-yl)-N⁴-methyl-N²-[3-(methyloxy)-5-(methylsulfonyl)phenyl]-2,4-pyrimidinediamine,
N⁴-(5,6-difluoro-1H-indazol-3-yl)-N⁴-methyl-N²-4-methyl-3-(methylsulfonyl)phenyl]-2,4-pyrimidinediamine,
N⁴-(5,6-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3-(methyloxy)-5-(methylsulfonyl)phenyl]-2,4-pyrimidinediamine,
N⁴-(1H-indazol-3-yl)-N²-(3-methyl-5-(methylsulfonyl)phenyl)pyrimidine-2,4-diamine,
N⁴-(1H-indazol-3-yl)-N²-(4-methyl-3-(methylsulfonyl)phenyl)pyrimidine-2,4-diamine,
N⁴-(7-fluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(4-methyl-3-(methylsulfonyl)phenyl)pyrimidine-2,4-diamine,
N⁴-(6,7-DIfluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(3-methyl-5-(methylsulfonyl)phenyl)pyrimidine-2,4-diamine,
N⁴-(5-fluoro-6-methyl-1H-indazol-3-yl)-N⁴-methyl-N²-[3-methyl-5-(methylsulfonyl)phenyl]-2,4-pyrimidinediamine,
N⁴-(7-fluoro-6-methyl-1H-indazol-3-yl)-N⁴-methyl-N²-[3-methyl-5-(methylsulfonyl)phenyl]-2,4-pyrimidinediamine,
N⁴-(5,6-difluoro-1H-indazol-3-yl)-N²-[3-methyl-5-(methylsulfonyl)phenyl]-2,4-pyrimidinediamine,
N⁴-(5,6-difluoro-1H-indazol-3-yl)-N²-[3-(methyloxy)-5-(methylsulfonyl)phenyl]-2,4-pyrimidinediamine,
N⁴-(5,6-difluoro-1H-indazol-3-yl)-N²-[3,4,5-tris(methyloxy)phenyl]-2,4-pyrimidinediamine,
N⁴-(6-fluoro-5-methyl-1H-indazol-3-yl)-N⁴-methyl-N²-[3-methyl-5-(methylsulfonyl)phenyl]-2,4-pyrimidinediamine,
2-chloro-5-({4-[(5-fluoro-1H-indazol-3-yl)amino]-2-pyrimidinyl}amino)benzenesulfonamide,
N⁴-(5-fluoro-6-methyl-1H-indazol-3-yl)-N⁴-methyl-N²-[3-methyl-5-(methylsulfonyl)phenyl]-2,4-pyrimidinediamine,
3-({4-[(7-fluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-(methylsulfonyl)-5-(pyrrolidin-1-yl)phenyl)pyrimidine-2,4-diamine,
3-({4-[(6,7-difluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-methyl-5-(pyrrolidin-1-yl)phenyl)pyrimidine-2,4-diamine,
N⁴-(7-fluoro-1H-indazol-3-yl)-N⁴-methyl-N²-(3-methyl-5-morpholinophenyl)pyrimidine-2,4-diamine,
N⁴-(7-fluoro-6-methyl-1H-indazol-3-yl)-N⁴-methyl-N²-[3-(methyloxy)-5-(methylsulfonyl)phenyl]-2,4-pyrimidinediamine,
N⁴-(5-fluoro-6-methyl-1H-indazol-3-yl)-N⁴-methyl-N²-[3-(methyloxy)-5-(methylsulfonyl)phenyl]-2,4-pyrimidinediamine,
N⁴-(6,7-difluoro-1H-indazol-3-yl)-N²-(1,1-dioxido-1-benzothien-6-yl)-N⁴-methyl-2,4-pyrimidinediamine,
N⁴-methyl-N⁴-(5-methyl-1H-indazol-3-yl)-N²-[3-(methyloxy)-5-(methylsulfonyl)phenyl]-2,4-pyrimidinediamine,
N⁴-methyl-N⁴-(5-methyl-1H-indazol-3-yl)-N²-[3-methyl-5-(methylsulfonyl)phenyl]-2,4-pyrimidinediamine,
3-({4-[(7-fluoro-6-methyl-1H-indazol-3-yl)(methyl)amino]-2-pyrimidinyl}amino)-5-methylbenzenesulfonamide,
N⁴-(7-fluoro-1H-indazol-3-yl)-N²-(3-methoxy-5-(methylsulfonyl)phenyl)pyrimidine-2,4-diamine,
5-((4-((7-fluoro-1H-indazol-3-yl)(methyl)amino)pyrimidin-2-yl)amino)-2-methoxybenzenesulfonamide,
N⁴-(6-methyl-1H-indazol-3-yl)-N²-[3-methyl-5-(methylsulfonyl)phenyl]-2,4-pyrimidinediamine,
N⁴-(6,7-difluoro-1H-indazol-3-yl)-N²-[3-(methyloxy)-5-(methylsulfonyl)phenyl]-2,4-pyrimidinediamine,
3-((4-((5,6-difluoro-1H-indazol-3-yl)(methyl)amino)pyrimidin-2-yl)amino)-5-methylbenzenesulfonamide,
N⁴-(5,6-difluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N⁴-methyl-N²-(3-methyl-5-((tetrahydro-2H-pyran-4-yl)sulfonyl)phenyl)pyrimidine-2,4-diamine,
3-({4-[(6,7-difluoro-1H-indazol-3-yl)(methyl)amino]-2-pyrimidinyl}amino)-5-(methyloxy)benzenesulfonamide,
3-((4-((5,6-difluoro-1H-indazol-3-yl)(methyl)amino)pyrimidin-2-yl)amino)-5-methoxybenzenesulfonamide,
3-({4-[(5-fluoro-6-methyl-1H-indazol-3-yl)(methyl)amino]-2-pyrimidinyl}amino)-5-(methyloxy)benzenesulfonamide,
3-({4-[(7-fluoro-6-methyl-1H-indazol-3-yl)(methyl)amino]-2-pyrimidinyl}amino)-5-(methyloxy)benzenesulfonamide,
3-({4-[(6,7-difluoro-1H-indazol-3-yl)amino]-2-pyrimidinyl}amino)-5-methylbenzenesulfonamide,
3-((4-((7-fluoro-1H-indazol-3-yl)amino)pyrimidin-2-yl)amino)-5-methoxybenzenesulfonamide,
3-((4-((7-fluoro-1H-indazol-3-yl)(methyl)amino)pyrimidin-2-yl)amino)-5-methoxybenzenesulfonamide,
N²-[4-(ethyloxy)-3-(methylsulfonyl)phenyl]-N⁴-(7-fluoro-1H-indazol-3-yl)-N⁴-methyl-2,4-pyrimidinediamine,
3-methoxy-5-((4-(methyl(7-(trifluoromethyl)-1H-indazol-3-yl)amino)pyrimidin-2-yl)amino)benzenesulfonamide,
3-methyl-5-[(4-{methyl[6-(trifluoromethyl)-1H-indazol-3-yl]amino}-2-pyrimidinyl)amino]benzenesulfonamide,
3-(methyloxy)-5-[(4-{methyl[6-(trifluoromethyl)-1H-indazol-3-yl]amino}-2-pyrimidinyl)amino]benzenesulfonamide,
3-methyl-5-((4-(methyl(7-(trifluoromethyl)-1H-indazol-3-yl)amino)pyrimidin-2-yl)amino)benzenesulfonamide,
N,N-dimethyl-3-{[4-(1H-pyrazolo[3,4-b]pyridin-3-ylamino)-2-pyrimidinyl]amino}benzenesulfonamide,
N⁴-(5-methyl-1H-pyrazolo[3,4-b]pyridin-3-yl)-N²-[3-(methylsulfonyl)phenyl]-2,4-pyrimidinediamine,
N²-[4-methyl-3-(methylsulfonyl)phenyl]-N⁴-(5-methyl-1H-pyrazolo[3,4-b]pyridin-3-yl)-2,4-pyrimidinediamine,
N²-[4-fluoro-3-(methylsulfonyl)phenyl]-N⁴-(5-methyl-1H-pyrazolo[3,4-b]pyridin-3-yl)-2,4-pyrimidinediamine,
N⁴-(5-fluoro-1H-pyrazolo[3,4-b]pyridin-3-yl)-N²-[3-(methylsulfonyl)phenyl]-2,4-pyrimidinediamine,
N²-[4-fluoro-3-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N⁴-methyl-N²-[3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N²-[3-(methyloxy)-5-(methylsulfonyl)phenyl]-2,4-pyrimidinediamine,
N⁴-(5-fluoro-1H-pyrazolo[3,4-b]pyridin-3-yl)-N⁴-methyl-N²-[3-methyl-5-(methylsulfonyl)phenyl]-2,4-pyrimidinediamine,
N⁴-(5-fluoro-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-1H-pyrazolo[3,4-b]pyridin-3-yl)-N²-[3-(methylsulfonyl)-5-(4-morpholinyl)phenyl]-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N²-[3,4-DImethyl-5-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N⁴-methyl-N²-[4-methyl-3-(methylsulfonyl)-5-(1-pyrrolidinyl)phenyl]-2,4-pyrimidinediamine,
N⁴-(6-methyl-1H-pyrazolo[3,4-b]pyridin-3-yl)-N²-[3-(methylsulfonyl)phenyl]-2,4-pyrimidinediamine,
N²-[3-[(difluoromethyl)oxy]-5-(methylsulfonyl)phenyl]-N⁴-(5-fluoro-1H-pyrazolo[3,4-b]pyridin-3-yl)-N⁴-methyl-2,4-pyrimidinediamine,
N²-[3-(3,3-difluoro-1-pyrrolidinyl)-5-(methylsulfonyl)phenyl]-N⁴-(5-fluoro-1H-pyrazolo[3,4-b]pyridin-3-yl)-N⁴-methyl-2,4-pyrimidinediamine,
N⁴-(5-fluoro-1H-pyrazolo[3,4-b]pyridin-3-yl)-N⁴-methyl-N²-[4-(methylsulfonyl)-2,3-dihydro-1-benzofuran-6-yl]-2,4-pyrimidinediamine,
N⁴-(5-fluoro-1H-pyrazolo[3,4-b]pyridin-3-yl)-N²-[3,4,5-tris(methyloxy)phenyl]-2,4-pyrimidinediamine,
N⁴-(5-fluoro-1H-pyrazolo[3,4-b]pyridin-3-yl)-N⁴-methyl-N²-[3,4,5-tris(methyloxy)phenyl]-2,4-pyrimidinediamine,
N²-[4-methyl-3-(methylsulfonyl)phenyl]-N⁴-(6-methyl-1H-pyrazolo[3,4-b]pyridin-3-yl)-2,4-pyrimidinediamine,
N²-{3-[(1,1-DImethylethyl)sulfonyl]-5-methylphenyl}-N⁴-(5-fluoro-1H-pyrazolo[3,4-b]pyridin-3-yl)-2,4-pyrimidinediamine,
N²-[3-[(1,1-DImethylethyl)sulfonyl]-5-(trifluoromethyl)phenyl]-N⁴-(5-fluoro-1H-pyrazolo[3,4-b]pyridin-3-yl)-2,4-pyrimidinediamine,
N⁴-(5-fluoro-6-methyl-1H-pyrazolo[3,4-b]pyridin-3-yl)-N⁴-methyl-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-6-methyl-1H-pyrazolo[3,4-b]pyridin-3-yl)-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)-5-(1-pyrrolidinyl)phenyl]-2,4-pyrimidinediamine,
N²-[3-[(difluoromethyl)oxy]-5-(methylsulfonyl)phenyl]-N⁴-(5-fluoro-6-methyl-1H-pyrazolo[3,4-b]pyridin-3-yl)-N⁴-methyl-2,4-pyrimidinediamine,
N⁴-(5-fluoro-6-methyl-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3,4,5-tris(methyloxy)phenyl]-2,4-pyrimidinediamine,
N²-{3-[(1,1-dimethylethyl)sulfonyl]-5-methylphenyl}-N⁴-(5-fluoro-6-methyl H-pyrazolo[3,4-b]pyridin-3-yl)-N⁴-methyl-2,4-pyrimidinediamine,
N⁴-(5-fluoro-1,6-dimethyl-1H-pyrazolo[3,4-b]pyridin-3-yl)-N⁴-methyl-N²-[3-methyl-5-(methylsulfonyl)phenyl]-2,4-pyrimidinediamine,
3-({4-[(5-fluoro-6-methyl-1H-pyrazolo[3,4-b]pyridin-3-yl)(methyl)amino]-2-pyrimidinyl}amino)benzenesulfonamide,
3-({4-[(5-fluoro-1H-pyrazolo[3,4-b]pyridin-3-yl)amino]-2-pyrimidinyl}amino)benzenesulfonamide,
N⁴-(5-fluoro-6-methyl-1H-pyrazolo[3,4-b]pyridin-3-yl)-N²-[3-fluoro-5-(methylsulfonyl)phenyl]-N⁴-methyl-2,4-pyrimidinediamine,
3-({4-[(5-fluoro-1H-pyrazolo[3,4-b]pyridin-3-yl)(methyl)amino]-2-pyrimidinyl}amino)benzenesulfonamide,
2-chloro-5-({4-[(5-fluoro-1H-pyrazolo[3,4-b]pyridin-3-yl)amino]-2-pyrimidinyl}amino)benzenesulfonamide,
2-chloro-5-({4-[(5-fluoro-1H-pyrazolo[3,4-b]pyridin-3-yl)(methyl)amino]-2-pyrimidinyl}amino)benzenesulfonamide,
5-({4-[(5-fluoro-1H-pyrazolo[3,4-b]pyridin-3-yl)(methyl)amino]-2-pyrimidinyl}amino)-2-methylbenzenesulfonamide,
N⁴-(5-fluoro-7-oxido-1H-pyrazolo[3,4-b]pyridin-3-yl)-N⁴-methyl-N²-[3-methyl-5-(methylsulfonyl)phenyl]-2,4-pyrimidinediamine,
5-fluoro-6-methyl-3-(methyl(2-((3-methyl-5-(methylsulfonyl)phenyl)amino)pyrimidin-4-yl)amino)-1H-pyrazolo[3,4-b]pyridine 7-oxide, or
N⁴-(5-fluoro-6-methyl-1H-pyrazolo[3,4-b]pyridin-3-yl)-N⁴-methyl-N²-(3-methyl-5-((tetrahydro-2H-pyran-4-yl)sulfonyl)phenyl)pyrimidine-2,4-diamine,

and a salt, particularly a pharmaceutically acceptable salt, thereof.

Representative compounds of this invention include the compounds of Examples 1-210.

Compound names were generated using the software naming program ACD/Name Pro V6.02 available from Advanced Chemistry Development, Inc., 110 Yonge Street, 14^{th} Floor, Toronto, Ontario, Canada, M5C 1T4 (http://www.acdlabs.com/). It will be appreciated by those skilled in the art that many of the compounds of this invention, as well as compounds used in the preparation of the compounds of Formula (I), (I-A) or (I-B) may exist in tautomeric forms. The program used to name the compounds of this invention will only name one of such tautomeric forms at a time. It is to be understood that any reference to a named compound or a structurally depicted compound is intended to encompass all tautomers of such compounds and any mixtures of tautomers thereof. For example, the tautomer of the compound of Example 13, N⁴-(5-methoxy-2H-indazol-3-yl-N²-(3,4,5-trimethoxyphenyl)pyrimidine-2,4-diamine, is intended to be encompassed by the depicted structure of Example 13 and the name provided for that structure by the naming program: N⁴-[5-(methyloxy)-1H-indazol-3-yl]-N²-[3,4,5-tris(methyloxy)phenyl]-2,4-pyrimidinediamine.

The compounds according to Formula (I), (I-A) or (I-B) may contain one or more asymmetric center (also referred to as a chiral center) and may, therefore, exist as individual enantiomers, diastereomers, or other stereoisomeric forms, or as mixtures thereof. Chiral centers, such as chiral carbon atoms, may also be present in a substituent such as an alkyl group. Where the stereochemistry of a chiral center present in a compound of this invention, or in any chemical structure illustrated herein, is not specified the structure is intended to encompass all individual stereoisomers and all mixtures thereof. Thus, compounds according to Formula (I), (I-A) or (I-B) containing one or more chiral center may be used as racemic mixtures, enantiomerically enriched mixtures, or as enantiomerically pure individual stereoisomers.

Individual stereoisomers of a compound according to according to Formula (I), (I-A) or (I-B) which contain one or more asymmetric center may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

Because of their potential use in medicine, the salts of the compounds of according to Formula (I), (I-A) or (I-B) are preferably pharmaceutically acceptable salts. Suitable pharmaceutically acceptable salts include those described by Berge, Bighley and Monkhouse J. Pharm. Sci (1977) 66, pp 1-19. Salts encompassed within the term “pharmaceutically acceptable salts” refer to non-toxic salts of the compounds of this invention.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, and the like, or with a pyranosidyl acid, such as glucuronic acid or galacturonic acid, or with an alpha-hydroxy acid, such as citric acid or tartaric acid, or with an amino acid, such as aspartic acid or glutamic acid, or with an aromatic acid, such as benzoic acid or cinnamic acid, or with a sulfonic acid, such as p-toluenesulfonic acid, methanesulfonic acid, ethanesulfonic acid or the like.

Suitable addition salts are formed from acids which form non-toxic salts and examples include acetate, p-aminobenzoate, ascorbate, aspartate, benzenesulfonate, benzoate, bicarbonate, bismethylenesalicylate, bisulfate, bitartrate, borate, calcium edetate, camsylate, carbonate, clavulanate, citrate, cyclohexylsulfamate, edetate, edisylate, estolate, esylate, ethanedisulfonate, ethanesulfonate, formate, fumarate, gluceptate, gluconate, glutamate, glycollate, glycollylarsanilate, hexylresorcinate, hydrabamine, hydrobromide, hydrochloride, dihydrochloride, hydrofumarate, hydrogen phosphate, hydroiodide, hydromaleate, hydrosuccinate, hydroxynaphthoate, isethionate, itaconate, lactate, lactobionate, laurate, malate, maleate, mandelate, mesylate, methylsulfate, monopotassium maleate, mucate, napsylate, nitrate, N-methylglucamine, oxalate, oxaloacetate, pamoate (embonate), palmate, palmitate, pantothenate, phosphate/diphosphate, pyruvate, polygalacturonate, propionate, saccharate, salicylate, stearate, subacetate, succinate, sulfate, tannate, tartrate, teoclate, tosylate, triethiodide, trifluoroacetate and valerate.

Other exemplary acid addition salts include pyrosulfate, sulfite, bisulfite, decanoate, caprylate, acrylate, isobutyrate, caproate, heptanoate, propiolate, oxalate, malonate, suberate, sebacate, butyne-1,4-dioate, hexyne-1,6-dioate, chlorobenzoate, methylbenzoate, dinitrobenzoate, hydroxybenzoate, methoxybenzoate, phthalate, phenylacetate, phenylpropionate, phenylbutrate, lactate, γ-hydroxybutyrate, mandelate, and sulfonates, such as xylenesulfonate, propanesulfonate, naphthalene-1-sulfonate and naphthalene-2-sulfonate.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as N-methyl-D-glucamine, diethylamine, isopropylamine, trimethylamine, ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

Compounds of the invention having both a basic and acidic moiety may be in the form of zwitterions, acid-addition salt of the basic moiety or base salts of the acidic moiety.

This invention also provides for the conversion of one pharmaceutically acceptable salt of a compound of this invention, e.g., a hydrochloride salt, into another pharmaceutically acceptable salt of a compound of this invention, e.g., a sodium salt.

For solvates of the compounds of the invention, or salts thereof that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The subject invention also includes isotopically-labeled compounds which are identical to those recited in according to Formula (I), (I-A) or (I-B) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, i.e., ³H, and carbon-14, i.e., ¹⁴C, isotopes are particularly preferred for their ease of preparation and detectability. ¹¹C and ¹⁸F isotopes are particularly useful in PET (positron emission tomography).

Because the compounds of according to Formula (I), (I-A) or (I-B) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### General Synthetic Methods

The compounds of according to Formula (I), (I-A) or (I-B) may be obtained by using synthetic procedures illustrated in the Schemes below or by drawing on the knowledge of a skilled organic chemist. The synthesis provided in these Schemes are applicable for producing compounds of the invention having a variety of different R¹ and R² groups employing appropriate precursors, which are suitably protected if needed, to achieve compatibility with the reactions outlined herein. Subsequent deprotection, where needed, affords compounds of the nature generally disclosed. While the Schemes are shown with compounds only of according to Formula (I), (I-A) or (I-B), they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

### Scheme 1

Nucleophilic aromatic substitution of 4-fluoronitroanilines with 4-methylpiperazine followed by hydrogenation of the nitro group afforded the piperazinoanilines

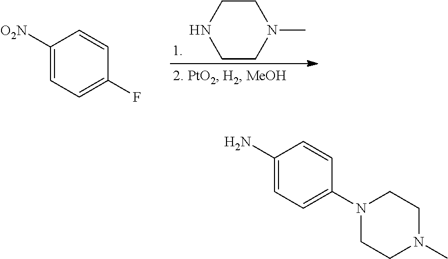

The requisite 2-anilinopyrimidines were prepared via displacement of the thioether moiety of 2-(Methylthio)-4(1H)-pyrimidinone using solvent free conditions.

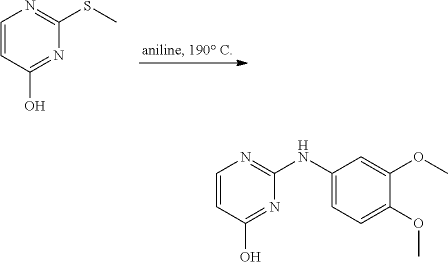

### Scheme 2

Conversion of the 2-anilinopyrimidinones was accomplished using POCl₃ and heat.

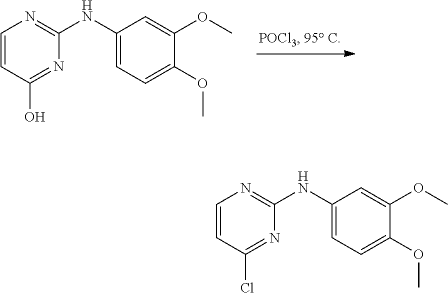 [SMILES: COc1ccc(Nc2nccc(O)n2)cc1OC ; COc1ccc(Nc2nccc(Cl)n2)cc1OC]

### Scheme 3

Non-commercial indazoles can be prepared from the corresponding 2-fluorobenzonitriles upon heating with a source of hydrazine in an alcohol solvent.

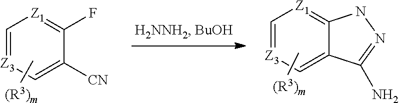

### Scheme 4

Introduction of the R⁵ methyl substituent can be accomplished via methylation of the corresponding indazole.

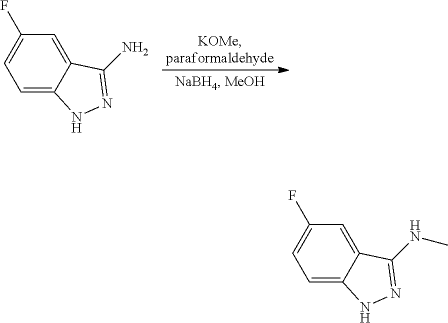

### Scheme 5

Substitution of the dichloropyrimidines with aminoindazoles can be accomplished using a variety of methods including heating in water or an acceptable solvent via thermal conditions. Substitution of the indazolochloropyrimidines with anilines can be accomplished using a variety of methods including heating in NMP or an acceptable solvent via thermal condition or μw irradiation conditions.

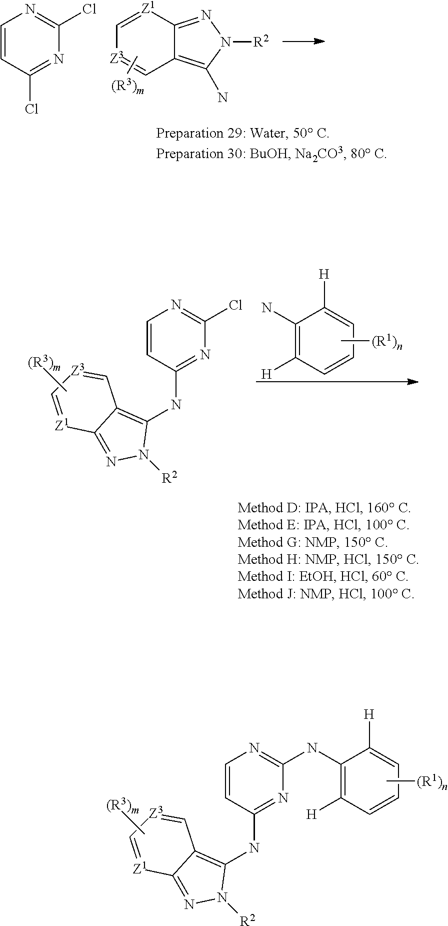

### Scheme 6

Substitution of the 4-chloropyrimidines with aminoindazoles can be accomplished using a variety of methods including heating in NMP or an acceptable solvent via thermal or μw irradiation. The addition of acid may be required for unreactive substrates. A palladium mediated cross coupling reaction can also be utilized via heating of the reactants in dioxane in the presence of Pd(OAc)₂, binap, and CsCO₃.

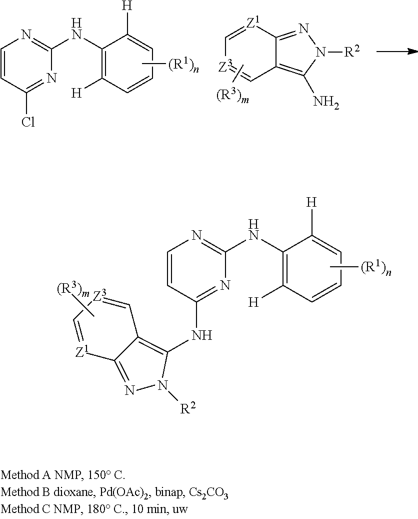

The present invention is also directed to a method of inhibiting RIP2 kinase which comprises contacting the kinase with a compound according to Formula (I), (I-A) or (I-B), or a salt, particularly a pharmaceutically acceptable salt, thereof. This invention is also directed to a method of treatment of a RIP2-mediated disease or disorder comprising administering a therapeutically effective amount of a compound of according to Formula (I), (I-A) or (I-B), or a salt thereof, particularly a pharmaceutically acceptable salt thereof, to a patient, specifically a human, in need thereof. As used herein, “patient” refers to a human or other mammal.

The compounds of this invention may be particularly useful for treatment of RIP2-mediated diseases or disorders, particularly, uveitis, interleukin-1 converting enzyme (ICE, also known as Caspase-1) associated fever syndrome, dermatitis, type 2 diabetes mellitus, acute lung injury, arthritis (specifically rheumatoid arthritis), inflammatory bowel disorders (such as ulcerative colitis and Crohn's disease), prevention of ischemia reperfusion injury in solid organ transplant, liver diseases (non-alcohol steatohepatitis, alcohol steatohepatitis, autoimmune hepatitis), allergic diseases (such as asthma), autoimmune diseases (such as systemic lupus erythematosus and Multiple Sclerosis), transplant reactions (such as graft versus host disease) and granulomateous disorders, such as adult sarcoidosis, Blau syndrome, early-onset sarcoidosis, cutaneous sarcoidosis, Wegner's granulomatosis, and interstitial pulmonary disease. The compounds of this invention may be particularly useful in the treatment of uveitis, ICE fever, Blau Syndrome/early-onset sarcoidosis, ulcerative colitis, Crohn's disease, Wegener's granulamatosis and sarcoidosis.

Treatment of RIP2-mediated disease conditions, or more broadly, treatment of immune mediated disease, such as, but not limited to, allergic diseases, autoimmune diseases, prevention of transplant rejection and the like, may be achieved using a compound of this invention of as a monotherapy, or in dual or multiple combination therapy, particularly for the treatment of refractory cases, such as in combination with other anti-inflammatory and/or anti-TNF agents, which may be administered in therapeutically effective amounts as is known in the art. For example, the compounds of this invention may be administered in combination with corticosteroids and/or anti-TNF agents to treat Blau syndrome/early-onset sarcoidosis; or in combination with anti-TNF biologics or other anti-inflammatory biologics to treat Crohn's Disease; or in combination with low-dose corticosteroids and/or methotrexate to treat Wegener's granulamatosis or sarcoidosis or interstitial pulmonary disease; or in combination with a biologic (e.g. anti-TNF, anti-IL-6, etc.) to treat rheumatoid arthritis; or in combination with anti-IL6 and or methotrexate to treat ICE fever.

Examples of suitable anti-inflammatory agents include corticosteroids, particularly low-dose corticosteroids (such as Deltasone® (prednisone)) and anti-inflammatory biologics (such as Acterma® (anti-IL6R mAb) and Rituximab® (anti-CD20 mAb)). Examples of suitable anti-TNF agents include anti-TNF biologics (such as Enbrel® (etanecerpt)), Humira® (adalimumab), Remicade® (infliximab) and Simponi® (golimumab)).

This invention also provides a compound of according to Formula (I), (I-A) or (I-B), or a salt thereof, particularly a pharmaceutically acceptable salt thereof, for use in the treatment or prophylaxis of RIP2-mediated diseases or disorders, for example those diseases and disorders mentioned hereinabove.

The invention also provides the use of a compound of according to Formula (I), (I-A) or (I-B), or a salt thereof, particularly a pharmaceutically acceptable salt thereof, in the manufacture of a medicament for the treatment or prophylaxis of RIP2-mediated diseases or disorders, for example those diseases and disorders mentioned hereinabove.

A therapeutically “effective amount” is intended to mean that amount of a compound that, when administered to a patient in need of such treatment, is sufficient to effect treatment, as defined herein. Thus, e.g., a therapeutically effective amount of a compound of according to Formula (I), (I-A) or (I-B), or a pharmaceutically acceptable salt thereof, is a quantity of an inventive agent that, when administered to a human in need thereof, is sufficient to modulate or inhibit the activity of RIP2 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmaceutical characteristics), disease or condition and its severity and the specific composition and method being used, but can nevertheless be determined by one of skill in the art.

“Treating” or “treatment” is intended to mean at least the mitigation of a disease condition in a patient. The methods of treatment for mitigation of a disease condition include the use of the compounds in this invention in any conventionally acceptable manner, for example for prevention, retardation, prophylaxis, therapy or cure of a mediated disease. Specific diseases and conditions that may be particularly susceptible to treatment using a compound of this invention are described herein.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

For use in therapy, the compounds of the invention will be normally, but not necessarily, formulated into a pharmaceutical composition prior to administration to a patient. Accordingly, the invention is also directed to pharmaceutical compositions comprising a compound of the invention and a pharmaceutically-acceptable excipient.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. For oral application, for example, one or more tablets or capsules may be administered. A dose of the pharmaceutical composition contains at least a therapeutically effective amount of a compound of this invention (i.e., a compound of according to Formula (I), (I-A) or (I-B) or a salt, particularly a pharmaceutically acceptable salt, thereof). When prepared in unit dosage form, the pharmaceutical compositions may contain from 1 mg to 1000 mg of a compound of this invention.

The pharmaceutical compositions of the invention typically contain one compound of the invention. However, in certain embodiments, the pharmaceutical compositions of the invention contain more than one compound of the invention. In addition, the pharmaceutical compositions of the invention may optionally further comprise one or more additional pharmaceutically active compounds.

As used herein, “pharmaceutically-acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically-acceptable are avoided. In addition, each excipient must of course be of sufficiently high purity to render it pharmaceutically-acceptable.

The compounds of the invention and the pharmaceutically-acceptable excipient or excipients will typically be formulated into a dosage form adapted for administration to the patient by the desired route of administration. Conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

Names for the intermediate and final compounds described herein were generated using a software naming program. It will be appreciated by those skilled in the art that in certain instances this program will name a structurally depicted compound as a tautomer of that compound. It is to be understood that any reference to a named compound or a structurally depicted compound is intended to encompass all tautomers of such compounds and any mixtures of tautomers thereof. the following experimental descriptions, the following abbreviations may be used:

In the following experimental descriptions, the following abbreviations may be used:

| Abbreviation | Meaning |
|---|---|
| aq | aqueous |
| brine | saturated aqueous NaCl |
| CH₂Cl₂, DCM | methylene chloride |
| CH₃CN or MeCN | acetonitrile |
| CH₃SNa | sodium methyl mercaptide |
| d | day |
| DIEA | diisopropylethylamine |
| DMF | N,N-dimethylformamide |
| DMSO | Dimethylsulfoxide |
| equiv | equivalents |
| Et | ethyl |
| Et₃N | triethylamine |
| Et₂O | diethyl ether |
| EtOAc | ethyl acetate |
| h, hr | hour |
| HATU | 2-(1H-7-azabenzotriazol-1-yl)-1,1,3,3- |
| | tetramethyluronium hexafluorophosphate |
| HCl | hydrochloric acid |
| i-Pr₂NEt | N′,N′-diisopropylethylamine |
| KOMe | potassium methoxide |
| LCMS | liquid chromatography-mass spectroscopy |
| Me | methyl |
| MeI | methyl iodide |
| MeOH or CH₃OH | methanol |
| MgSO₄ | magnesium sulfate |
| min | minute |
| MS | mass spectrum |
| μw | microwave |
| NMP | N-methyl-2-pyrrolidine |
| NaH | sodium hydride |
| NaHCO₃ | sodium bicarbonate |
| Na₂SO₄ | sodium sulfate |
| NH₄Cl | ammonium chloride |
| Pd/C | palladium on carbon |
| Ph | phenyl |
| rt | room temperature |
| satd | saturated |
| SPE | solid phase extraction |
| TFA | trifluoroacetic acid |
| THF | tetrahydrofuran |
| t_{R} | retention time |
| ptfe | polytetrafluoroethylene |
| binap | 2,2′-bis(diphenylphosphino)-1,1′-binaphthalene |

### Preparation 1

### 2-(Dimethylamino)ethyl(4-nitrophenyl)amine

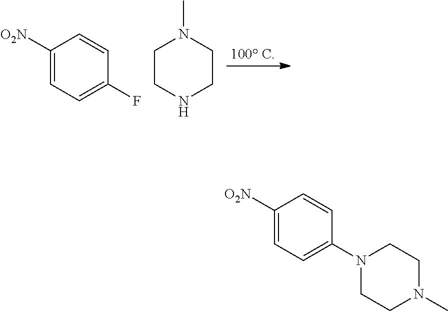

A mixture of 1-methylpiperazine (35.5 g, 354 mmol) and 1-fluoro-4-nitrobenzene (10 g, 70.9 mmol) was heated in a sealed tube at 100° C. for 20 hours. The reaction was cooled to rt. Cold water was added and the resulting solid was collected via filtration, washing with water, then air dried to give the title compound as an orange solid 15.4 g.

### (4-Aminophenyl)[2-(dimethylamino)ethyl]ethylamine

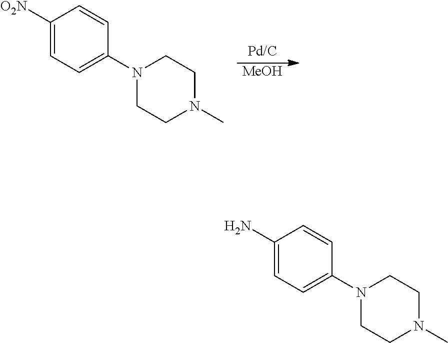 [SMILES: "MeOH" ; CN1CCN(c2ccc([N+](=O)[O-])cc2)CC1 ; CN1CCN(c2ccc(N)cc2)CC1]

1-Methyl-4-(4-nitrophenyl)piperazine (7.7 g, 34.8 mmol) was dissolved in MeOH (200 mL) and Pd/C (0.185 g, 1.740 mmol) was added. The reaction mixture was placed under an atmosphere of H₂ for 24 hour. The reaction was filtered through a pad of celite, washed with MeOH, concentrated and dried on high vacuum to give the product as a brown solid 6.6 g.

### Preparation 2

### 3-amino-N-[(1E)-(dimethylamino)methylidene]-5-methylbenzenesulfonamide

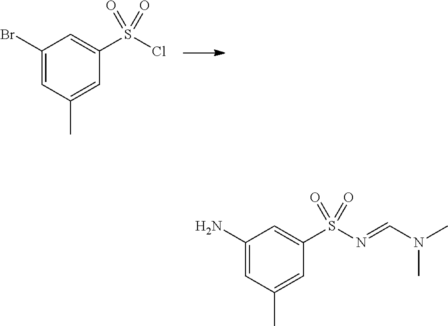 [SMILES: Cc1cc(Br)cc(S(=O)(=O)Cl)c1 ; Cc1cc(N)cc(S(=O)(=O)/N=C/N(C)C)c1]

3-bromo-5-methylbenzenesulfonyl chloride (3 g, 11.13 mmol) was dissolved in MeOH (20 mL) and ammonia in MeOH (9.54 mL, 66.8 mmol) was added dropwise and an exotherm was noted. The reaction was stirred for one hour and LCMS analysis (A1) showed the reaction was complete. The solvent was removed and the resulting residue dissolved in MeOH, concentrated onto silica, and purified via flash chromatography using a 100 g SNAP cartridge eluting with 0-100% EtOAc/hexanes to give the desired product as a white solid (2.1 g, 74%). The product was then added to a solution of N,N-dimethylformamide dimethyl acetal (5.54 ml, 41.4 mmol) in N,N-Dimethylformamide (DMF) (6.28 ml) and the reaction was stirred at rt for one hour. Water (2 mL) was added and the mixture was stirred for several minutes then filtered washing with water. The resulting solid was dried on the vacuum funnel (2.4 g, 95%). The solid was placed in a flask along with PdOAc2 (0.088 g, 0.393 mmol), xantphos (0.455 g, 0.786 mmol), and cesium carbonate (7.69 g, 23.59 mmol). To the mixture was added 1,4-Dioxane (24.23 ml) followed by benzophenone imine (1.979 ml, 11.80 mmol). The reaction was degassed by bubbling through N₂ for several minutes, then was heated to 95° C. for 5 hours. The reaction was filtered through celite washing with MeOH then concentrated onto silica and purified via flash chromatography eluting with 30-50% EtOAc/hexanes for 30 min then 100% EtOAc for 20 min. The product was recrystallized from EtOAc to give the title compound as a yellow solid (1.34 g). The solid was a 1:1 mixture of product and benzophenone imine byproduct. ¹H NMR (DMSO-d₆) δ: 8.05 (s, 1H), 7.63-7.69 (m, 2H), 7.52-7.58 (m, 1H), 7.45-7.51 (m, 2H), 7.30-7.37 (m, 3H), 7.10-7.18 (m, 3H), 6.80 (s, 1H), 6.76 (s, 1H), 3.14 (s, 3H), 2.86 (s, 3H), 2.24 (s, 3H); MS (m/z) 242 (M+H⁺).

### Preparation 3

### 2-fluoro-3-(methylsulfonyl)aniline

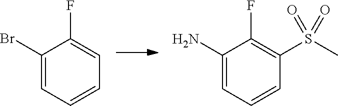

A solution of 1-bromo-2-fluorobenzene (310 μl, 2.86 mmol) in Tetrahydrofuran (THF) (1.21E+04 μl) was cooled to −78° C. and treated with LDA (1571 μl, 3.14 mmol) and stirred for 20 min. Then dimethyldisulfide (330 μl, 3.71 mmol) was added and the reaction was allowed to slowly warmed to rt overnight. LCMS showed a peak at 1.01 min, but the mass didn't match the product. The crude mixture was quenched with 10% H2SO4 and the layers were separated and the aqueous layer extracted with EtOAc. The combined extracts were washed with satd aq NaHCO3, dried over MgSO4, filtered, and concentrated onto silica. The crude product was purified via flash chromatography using a 25 g column eluting with 0-100% EtOAc/hexanes. The product was dissolved in Dichloromethane (DCM) (1.07E+04 μl) and treated with MCPBA (779 mg, 4.51 mmol). The reaction was stirred for one hour at rt then 1 mL of DMF was added and the reaction was stirred over the weekend at rt. The reaction was concentrated onto silica and purified via flash chromatography using a 25 g column eluting with 0-100% EtOAc/hexanes. The product eluted was a mixture of 3-chlorobenzoic acid and product based on NMR analysis. The sample was dissolved in EtOAc and washed with 2× satd aq NaHCO3 and the organic solution was dried over MgSO4, filtered, and concentrated to an orange oil (422 mg, 78%). The oil was then combined with Pd2(dba)3 (79 mg, 0.086 mmol), xantphos (100 mg, 0.173 mmol), cesium carbonate (844 mg, 2.59 mmol), and tert-butyl carbamate (303 mg, 2.59 mmol). 1,4-Dioxane (5756 μl) was added and the solution was degassed with N2. The reaction was then heated to 100° C. for 20 hours. The reaction was filtered then concentrated and the resulting residue was dissolved in DCM. The solution was filtered then treated with TFA (2.5 mL) and the reaction was stirred at rt for 2 hours. The reaction was then concentrated onto silica and the crude material was purified via flash chromatography using a 40 g column eluting with 0-100% EtOAc/hexanes to give the title compound as a brown oil (132 mg, 40%). ¹H NMR (DMSO-d₆) δ: 7.05-7.16 (m, 2H), 6.93 (dt, J=6.0, 2.9 Hz, 1H), 3.26 (s, 3H); MS (m/z) 190 (M+H⁺).

### Preparation 4

### Ethyl 2-[(3-aminophenyl)thio]-2-methylpropanoate

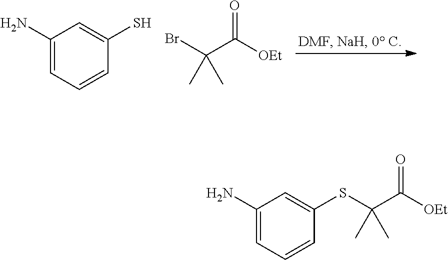

A solution of 3-aminothiophenol (97 mg, 0.775 mmol) in N,N-Dimethylformamide (DMF) (1823 μl) at 0° C. was treated with sodium hydride (30.9 mg, 1.286 mmol) and stirred for 20 min. Ethyl 2-bromo-2-methylpropanoate (114 μl, 0.775 mmol) was added and the reaction was warmed to rt over 16 hours. The reaction was diluted with DCM washing with water and brine and the organic solution was dried over Na₂SO₄, filtered, and concentrated onto silica. The crude material was purified via flash chromatography eluting with 0-50% EtOAc/hexanes on a 40 g column. ¹H NMR (DMSO-d₆) δ: 6.99 (t, J=7.8 Hz, 1H), 6.62-6.68 (m, 1H), 6.59 (dd, J=8.1, 2.0 Hz, 1H), 6.53 (d, J=8.1 Hz, 1H), 5.22 (s, 2H), 4.03 (q, J=7.1 Hz, 2H), 1.40 (s, 6H), 1.13 (t, J=7.1 Hz, 3H); MS (m/z) 239 (M⁺).

The following intermediate, used for the preparation of named example compounds, was synthesized using methods analogous to the ones described above.

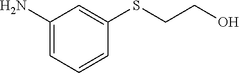 [SMILES: Nc1cccc(SCCO)c1]

### Preparation 5

### 2-[(3-aminophenyl)thio]-2-methyl-1-propanol

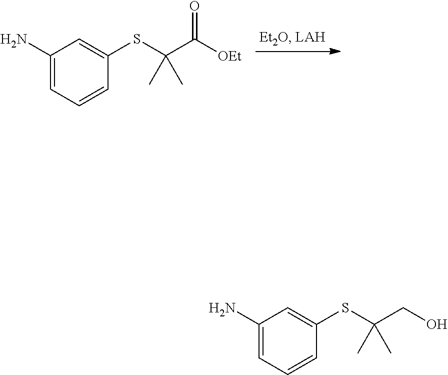 [SMILES: CCOC(=O)C(C)(C)Sc1cccc(N)c1 ; CC(C)(CO)Sc1cccc(N)c1]

A solution of ethyl 2-[(3-aminophenyl)thio]-2-methylpropanoate (85 mg, 0.355 mmol) in Diethyl ether (1421 μl) was treated with LAH (355 μl, 0.355 mmol) and stirred for 1 hour. There was no more starting material based on LCMS analysis and two new product peaks, one with the desired mass and the other unidentified. The reaction was diluted with EtOAc and satd aq NH4Cl and stirred vigorously overnight. The biphasic mixture was filtered through celite to remove the aluminum salts and the aqueous layer was extracted with DCM. The combined extracts were dried over Na2SO4, filtered, and concentrated to give the desired product. ¹H NMR (DMSO-d₆) δ: 6.98 (t, J=7.7 Hz, 1H), 6.72 (t, J=2.0 Hz, 1H), 6.62 (s, 1H), 6.54-6.59 (m, 1H), 5.17 (s, 2H), 4.82 (t, J=5.9 Hz, 1H), 3.28 (d, J=5.8 Hz, 2H), 1.13 (s, 6H); MS (m/z) 197 (M⁺).

### Preparation 6

### 2-[(3-aminophenyl)sulfonyl]-2-methyl-1-propanol

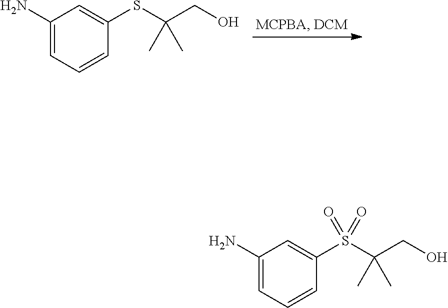

A solution of 2-[(3-aminophenyl)thio]-2-methyl-1-propanol (33.0 mg, 0.167 mmol) in Dichloromethane (DCM) (836 μl) was treated with MCPBA (57.7 mg, 0.335 mmol) and the reaction was stirred for 1 hour at rt. The mixture was diluted with DCM to dissolve the precipitate then the solution was concentrated onto silica and the crude product purified via flash chromatography using a 12 g column eluting with 0-10% MeOH/DCM. The fractions containing product were concentrated and the resulting residue was dissolved in MeOH and loaded onto a 0.5 g SCX cartridge. The cartridge was flushed with four volumes of MeOH and then the product was extracted with 2N NH3 in MeOH using three volumes. The extracts were concentrated to give the desired product as a white solid (23 mg, 60%). ¹H NMR (DMSO-d₆) δ: 7.25 (t, J=7.8 Hz, 1H), 6.97-7.01 (m, 1H), 6.83-6.90 (m, 2H), 5.65 (s, 2H), 5.02 (t, J=6.1 Hz, 1H), 3.47 (d, J=6.1 Hz, 2H), 1.17 (s, 6H); MS (m/z) 229 (M⁺).

### Preparation 7

### 3,5-dinitrophenyl methyl sulfone

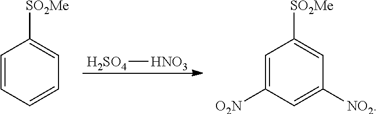

To an ice-bath cooled solution of nitric acid (90%) (20 mL) and fuming sulfuric acid (40 mL) was slowly added (methylsulfonyl)benzene (10.0 g, 64.0 mmol) in 4 portions. The ice-bath was removed then the reaction mixture was slowly heated to 140 deg C. in an oil bath for 16 h. The reaction mixture was cooled to rt then slowly poured over solid ice while swirling. The solid was collected by filtration then washed with water (300 ml), ethanol (80 mL) and ethylether (100 mL). The solid was suspended and stirred in DMSO (40 mL) for 5-10 min then filtered. The solid was washed successively with water, ethanol, then ethyl ether to give the title compound as a white solid (4.04 g, 25.6%). ¹H NMR (DMSO-d₆) δ: 9.10 (t, 1H), 9.02 (d, J=2.0 Hz, 2H), 3.51 (s, 3H); MS (m/z) 247.1 (M+H⁺).

The following intermediates, used for the preparation of named example compounds, were synthesized using methods analogous to the ones described above.

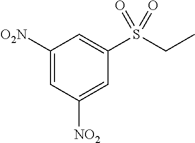 [SMILES: CCS(=O)(=O)c1cc([N+](=O)[O-])cc([N+](=O)[O-])c1]

### Preparation 8

### 1-(methyloxy)-3-(methylsulfonyl)-5-nitrobenzene

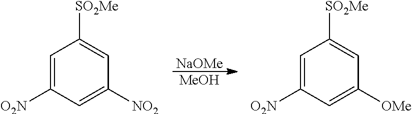 [SMILES: "MeOH" ; CS(=O)(=O)c1cc([N+](=O)[O-])cc([N+](=O)[O-])c1 ; COc1cc([N+](=O)[O-])cc(S(C)(=O)=O)c1 ; "NaOMe"]

To a suspension of 3,5-dinitrophenyl methyl sulfone (1.5 g, 6.09 mmol) in methanol (15.0 mL) was added sodium methoxide (1.672 g, 7.74 mmol) as a solution in methanol (25% w/w). The reaction mixture was heated to 70° C. for 2 h then the reaction mixture was poured onto ice. The solid was collected by filtration and washed with water, ethanol, and ethylether to yield the title compound as a light brown solid (1.16 g, 82%). ¹H NMR (DMSO-d₆) δ: 8.21-8.24 (m, 1H), 8.05-8.07 (m, 1H), 7.89-7.91 (m, 1H), 4.00 (s, 3H), 3.38 (s, 3H).

The following intermediates, used for the preparation of named example compounds, were synthesized using methods analogous to the ones described above.

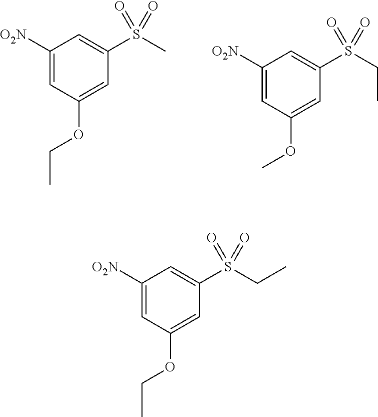 [SMILES: CCOc1cc([N+](=O)[O-])cc(S(C)(=O)=O)c1 ; CCS(=O)(=O)c1cc(OC)cc([N+](=O)[O-])c1 ; CCOc1cc([N+](=O)[O-])cc(S(=O)(=O)CC)c1]

### Preparation 9

### 1-(methyloxy)-3-(methylsulfonyl)-5-nitrobenzene

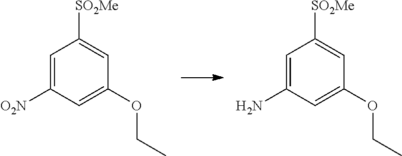 [SMILES: CCOc1cc([N+](=O)[O-])cc(S(C)(=O)=O)c1 ; CCOc1cc(N)cc(S(C)(=O)=O)c1]

To a magnetically stirred solution of 1-(ethyloxy)-3-(methylsulfonyl)-5-nitrobenzene (277 mg, 1.129 mmol) in Acetic Acid (3 mL) at room temperature was added zinc (378 mg, 5.78 mmol) in one portion. The reaction stirred at room temperature for 1 hour. The mixture was diluted with EtOAc and then filtered through Celite. The solution was diluted with water and neutralized through the addition of solid K₂CO₃. The layers were separated and the organic layer was concentrated under reduced pressure to give [3-(ethyloxy)-5-(methylsulfonyl)phenyl]amine (111 mg, 0.516 mmol, 45.7% yield) which was carried on without further purification. MS (m/z) 216 (M+H⁺).

The following intermediates, used for the preparation of named example compounds, were synthesized using methods analogous to the ones described above.

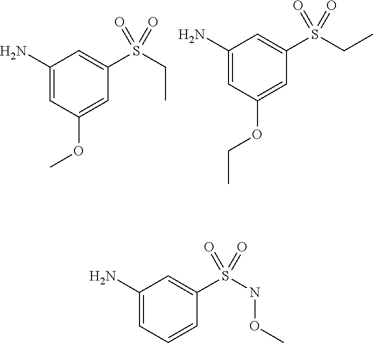

### Preparation 10

### 4-(ethyloxy)-3-[(1-methylethyl)sulfonyl]aniline

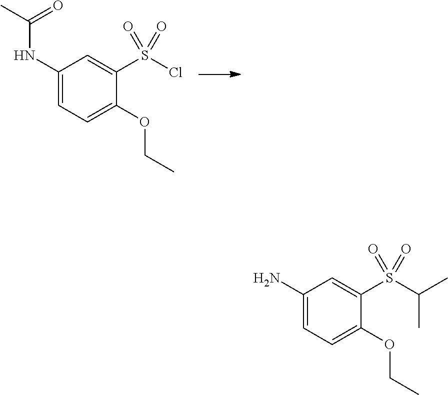 [SMILES: CCOc1ccc(NC(C)=O)cc1S(=O)(=O)Cl ; CCOc1ccc(N)cc1S(=O)(=O)C(C)C]

To a mixture of sodium bicarbonate (318 mg, 3.78 mmol) and sodium sulfite (454 mg, 3.60 mmol) in Water (33 mL) was added a solution of 5-(acetylamino)-2-(ethyloxy)benzenesulfonyl chloride (500 mg, 1.800 mmol) in Ethanol (0.65 μl). The reaction was heated to 50° C. for one hour then concentrated. The resulting residue was dissolved in N,N-Dimethylformamide (DMF) (4901 μl) and 2-iodopropane (180 μl, 1.800 mmol) was added and the reaction stirred at rt for 16 hours. The solution was diluted with EtOAc and washed three times with satd aq NH4Cl, dried over Na2SO4, filtered, and concentrated. The resulting residue was dissolved in a solution of 4 N hydrochloric acid in 1,4-dioxane (202 μl, 0.806 mmol) and was heated to 100° C. for 6 hours. Water was added to the reaction and it was heated to 100° C. for 3 hours then stirred at rt for 16 hours. The reaction was diluted with DCM and neutralized with satd aq NaHCO3. The organic solution was passed through a phase separator and concentrated. The material was dissolved in MeOH and passed through an SCX cartridge washing with MeOH. The product was eluted with a solution of 2 N NH3 in MeOH to give the title compound as a yellow film (60 mg, 31%). ¹H NMR (DMSO-d₆) δ: 7.04-7.09 (m, 1H), 7.00 (d, J=8.6 Hz, 1H), 6.81-6.88 (m, 1H), 5.28 (br. s., 2H), 4.03 (q, J=7.0 Hz, 2H), 3.60-3.74 (m, 1H), 1.27-1.33 (m, 3H), 1.15 (d, J=6.8 Hz, 6H).

### Preparation 11

### 2-methyl-1-(methylsulfonyl)-3-nitrobenzene

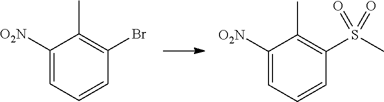

A mixture of 2-methyl-1-bromo-3-nitrobenzene (200 mg, 0.926 mmol), sodium thiomethoxide (68.1 mg, 0.972 mmol), xantphos (107 mg, 0.185 mmol), and Pd2(dba)3 (85 mg, 0.093 mmol) was treated with Toluene (46 mL) and nitrogen was bubbled through the solution for several minutes. The reaction was then heated to 100° C. overnight. The reaction was then filtered through celite washing with MeOH and concentrated onto silica. The crude material was purified via flash chromatography using a 40 g column eluting with 0-20% EtOAc/hexanes for 20 min then 20-50% for 10 min. The sample was dissolved in DCM (34 mL) and treated with MCPBA (294 mg, 1.706 mmol) at rt and the reaction was stirred for 1 hour. The mixture was diluted with DCM and concentrated onto silica. The crude material was purified via flash chromatography on a 12 g column eluting with 0-100% EtOAc/hexanes to give the title compound (47 mg, 24%) as a white solid, ¹H NMR (DMSO-d₆) δ: 7.66-7.71 (m, 1H), 7.32 (d, J=8.1 Hz, 1H), 6.91 (t, J=8.1 Hz, 1H), 6.59 (s, 1H), 2.16 (s, 3H).

### Preparation 12

### 3-(methylsulfonyl)-5-nitrophenol

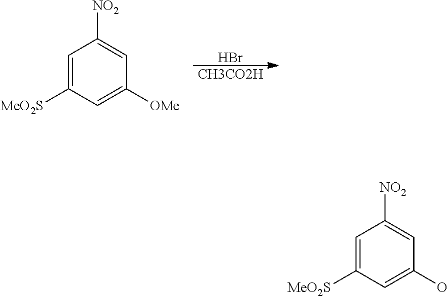

A solution of 1-(methyloxy)-3-(methylsulfonyl)-5-nitrobenzene (435.0 mg, 1.881 mmol) in 6.0 mL of a solution of HBr in Acetic acid (40% w/w) was heated at 90.0° C. in an oil bath. The reaction mixture was poured onto solid ice then the aqueous suspension was extracted with EtOAc. The organic layer was washed with sat.aq NaHCO₃, dried over sodium sulfate, filtered then concentrated. The residue was purified by FCC using a Biotage unit [EtOAc-Hex: 10 to 40%] to yield the title compound (191.0 mg, 46.7%). MS (m/z): 218.0 (M+H⁺).

### Preparation 13

### 1-[(difluoromethyl)oxy]-3-(methylsulfonyl)-5-nitrobenzene

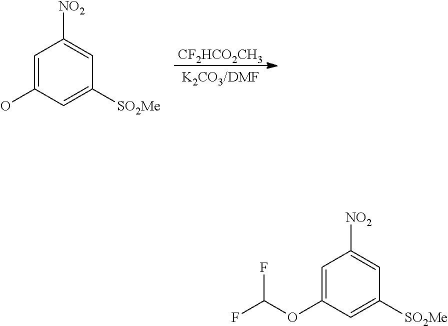 [SMILES: CS(=O)(=O)c1cc(O)cc([N+](=O)[O-])c1 ; "CF2HCO2CH3" ; CS(=O)(=O)c1cc(OC(F)F)cc([N+](=O)[O-])c1]

To a solution of 3-(methylsulfonyl)-5-nitrophenol (66.0 mg, 0.304 mmol) in DMF (2.5 mL) was added potassium carbonate (147 mg, 1.064 mmol). The mixture was stirred for 20 min at rt then methyl chloro(difluoro)acetate (0.081 mL, 0.760 mmol) was added and the rm was heated to 90° C.

After 1 h the rm was cooled to rt then diluted with EtOAc and washed with water. The organic layer was dried over sodium sulfate, filtered then concentrated. The residue was purified by FCC using a Biotage unit [EtOAc-Hex: 10 to 50%]. ¹H NMR (DMSO-d₆) δ: 8.50-8.55 (m, 1H), 8.36-8.40 (m, 1H), 8.17-8.20 (m, 1H), 7.38-7.78 (m, 1H), 3.43 (s, 3H); MS (m/z): 268.3 (M+H⁺).

### Preparation 14

### [3-[(difluoromethyl)oxy]-5-(methylsulfonyl)phenyl]amine

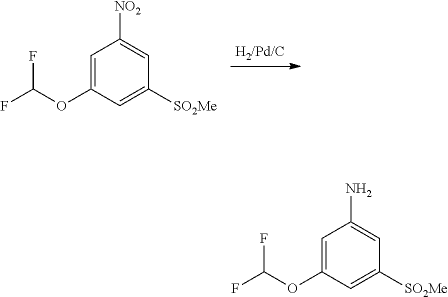 [SMILES: CS(=O)(=O)c1cc(OC(F)F)cc([N+](=O)[O-])c1 ; CS(=O)(=O)c1cc(N)cc(OC(F)F)c1]

To a solution of 1-[(difluoromethyl)oxy]-3-(methylsulfonyl)-5-nitrobenzene (110.0 mg, 0.412 mmol) in 4 mL ethyl acetate-ethanol (3:1) was added Pd/C (10% w/w) (43.8 mg, 0.041 mmol). The mixture was stirred under hydrogen gas which was supplied from a balloon. After 4 h the rm was filtered then concentrated in-vacuo to yield the title compound. MS (m/z): 238.1 (M+H⁺).

### Preparation 15

### 2,2,2-trifluoro-1-(3-nitrophenyl)ethanamine

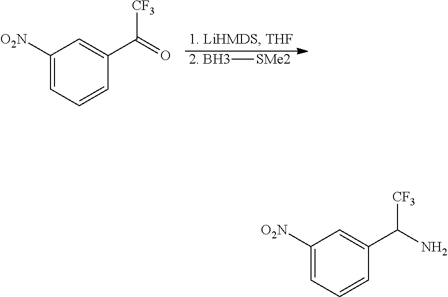

2,2,2-trifluoro-1-(3-nitrophenyl)ethanone (5.00 g, 22.82 mmol) was dissolved in toluene (30 mL) at room temperature. A solution of 1M LiHMDS in THF (25.6 mL, 25.6 mmol) was added into the reaction solution slowly over 10 min period of time. The mixture was stirred at room temperature for 15 min, then BH₃.DMS (4.40 mL, 46.3 mmol) was added. The reaction mixture was stirred at room temperature for 2 h. The reaction mixture was then quenched with Ice-water mixture. The quenched reaction mixture was partitioned between water and dichloromethane. The organic layer was washed by brine, dried over MgSO₄, filtered, and concentrated to about 10 ml of the toluene solution. A solution of 3 ml 4N HCl in dioxane was added dropwise. The resulting white precipitate was collected by filtration, and then was dried under high vacuum for 16 h to give the desired product as a white solid (5.3 g, 91% yield). ¹H NMR (400 MHz, DMSO-d₆) δ: 8.40 (dd, J=8.2, 1.4 Hz, 1H), 8.17 (d, J=7.8 Hz, 1H), 7.86 (t, J=8.1 Hz, 1H), 7.05 (m, 1H), 5.87 (m, 1H), 3.89 (s, 2H); MS (m/z) 221 (M+H⁺).

### Preparation 16

### 2,2,2-trifluoro-N,N-dimethyl-1-(3-nitrophenyl)ethanamine

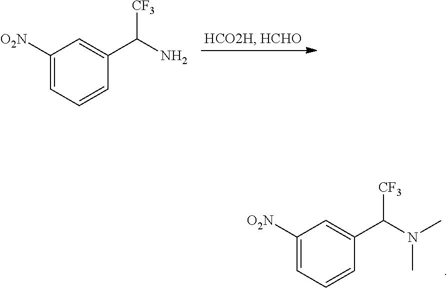 [SMILES: NC(c1cccc([N+](=O)[O-])c1)C(F)(F)F ; CN(C)C(c1cccc([N+](=O)[O-])c1)C(F)(F)F]

2,2,2-trifluoro-1-(3-nitrophenyl)ethanamine (1 g, 4.54 mmol) was dissolved in formic acid (3484 μl, 91 mmol) at room temperature. Paraformaldehyde (546 mg, 18.17 mmol) was added to the reaction mixture, and then the mixture was stirred at 100° C. for 3 h. The reaction mixture was added into 150 ml of sat. Na₂CO₃(aq) slowly, and then was extracted by dichloromethane. The organic layer was washed by brine, dried over MgSO₄ and concentrated to a brown oil. The crude oil was purified by Isco Combiflash (5%-20% EtOAc/Hexane; 40 g column). Collected fractions were combined and concentrated to give the desired product as a colorless oil (600 mg, 53% yield). MS (m/z) 221 (M+H⁺).

### Preparation 17

### 1-bromo-3-[(1,1-dimethylethyl)sulfonyl]-5-methylbenzene

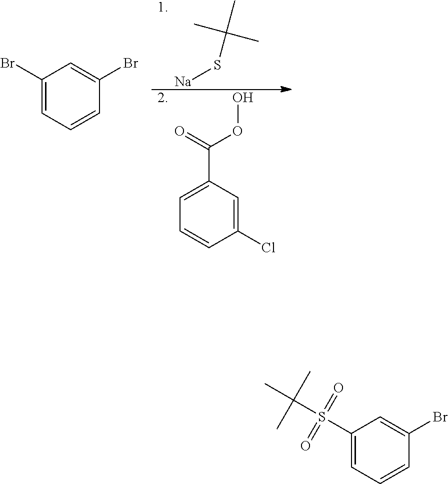

1,3-dibromobenzene (1.023 mL, 8.48 mmol) was dissolved in N-Methyl-2-pyrrolidone (NMP) (20 mL) and t-butylthiol sodium salt (3.17 g, 25.4 mmol) was slowly added at room temperature. Reaction was slightly exothermic and turned medium pink/red color. Reaction was heated at 80° C. for 5 days. Reaction was cooled to room temperature and 0.5 mL 6M NaOH (aq) and 20 mL water were added and mixture was stirred for 10 minutes. Hexanes were added, layers were separated and hexanes were washed again with brine. Organics, were concentrated to give 3.0 g light yellow liquid, a mixture of −2:1 desired product:bis-alkylated product. MS (m/z), did not ionize. Crude product was used as-is in next reaction. 1-bromo-3-[(1,1-dimethylethyl)thio]benzene (3.00 g, 12.24 mmol) was dissolved in Dichloromethane (DCM) (50 mL) and cooled in an ice/water bath, MCPBA (6.86 g, 30.6 mmol) was added in portions and reaction was stirred at room temperature for 120 minutes. Reaction was diluted with satd. NaHCO₃, layers were separated and organics were washed with 2M NaOH and brine. Organics were concentrated and purified by column chromatography to give the title compound as a white solid (1.90 g, 90% yield for oxidation, based on 62% purity of starting material). ¹H NMR (400 MHz, CHLOROFORM-d) δ 8.05 (t, J=1.77 Hz, 1H), 7.82 (m, 2H), 7.46 (t, J=7.83 Hz, 1H), 1.38 (s, 9H); MS (m/z) 277/279.

The following intermediates, used for the preparation of named example compounds, were synthesized using methods analogous to the ones described above.

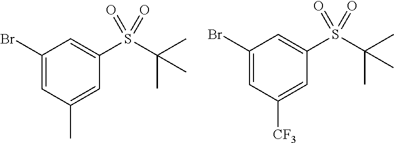

### Preparation 18

### 3-bromo-N,N,5-trimethylbenzenesulfonamide

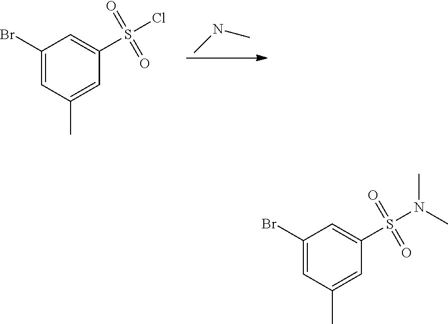

3-bromo-5-methylbenzenesulfonyl chloride (500 mg, 1.855 mmol) was dissolved in Dichloromethane (DCM) (15 mL) and cooled in an ice/water bath. Then dimethylamine (2.78 mL, 5.56 mmol) was added and reaction was stirred at room temperature for 20 minutes. Reaction was diluted with satd. NaHCO₃, layers were separated and organics were dried over sodium sulfate. Organics were concentrated and dried to give the title compound as a light orange solid (513 mg, 96%). ¹H NMR (400 MHz, CHLOROFORM-d) δ 7.74 (s, 1H), 7.58 (s, 1H), 7.53 (s, 1H), 2.75 (s, 6H), 2.45 (s, 3H); MS (m/z) 278/280 (M+H⁺).

The following intermediate, used for the preparation of named example compounds, was synthesized using methods analogous to the ones described above.

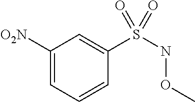

### Preparation 19

### 1-bromo-3-methyl-5-(methylsulfonyl)benzene

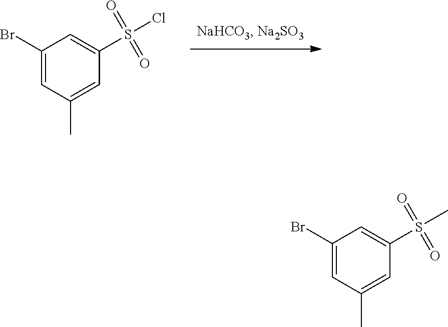

To a solution of sodium sulfite (1.870 g, 14.84 mmol) and sodium bicarbonate (1.309 g, 15.58 mmol) in Water (10 mL) was added 3-bromo-5-methylbenzenesulfonyl chloride (2.00 g, 7.42 mmol) and Ethanol (5.00 mL). Mixture was heated at 50° C. for 45 minutes and concentrated to dryness. Crude was suspended in N,N-Dimethylformamide (DMF) (15 mL), iodomethane (2.315 mL, 37.1 mmol) was added and mixture was stirred at room temperature for 15 minutes. Reaction was diluted with diethyl ether and satd. NaHCO₃ and layers were separated. Organics were washed with water, dried over sodium sulfate, filtered, concentrated and dried to give title compound as a light yellow solid (1.64 g, 84%). ¹H NMR (400 MHz, CHLOROFORM-d) δ 7.91 (s, 1H), 7.71 (s, 1H), 7.63 (s, 1H), 3.08 (s, 3H), 2.46 (s, 3H); MS (m/z) 249/251 (M+H⁺).

The following intermediates, used for the preparation of named example compounds, were synthesized using methods analogous to the ones described above.

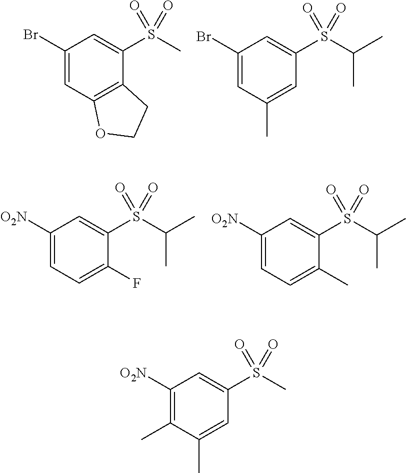

### Preparation 20

### 3-[(diphenylmethylidene)amino]-N,N,5-trimethylbenzenesulfonamide

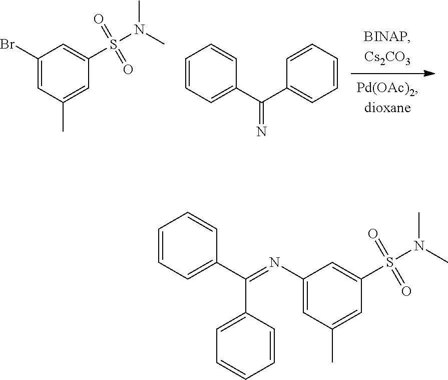

3-bromo-N,N,5-trimethylbenzenesulfonamide (510 mg, 1.833 mmol), benzophenone imine (0.369 mL, 2.200 mmol), cesium carbonate (836 mg, 2.57 mmol) and BINAP (114 mg, 0.183 mmol) and palladium(II)acetate (41.2 mg, 0.183 mmol) were mixed in 1,4-Dioxane (8 mL) and nitrogen was bubbled through for 5 minutes. The reaction was microwaved at 130° C. for 30 minutes, diluted with water and DCM and layers were separated. Organics were concentrated and purified by chromatography (25 g silica column; 3-20% E/H, 25 min.) to give the title compound as a yellow foam (532 mg, 73%). ¹H NMR (400 MHz, CHLOROFORM-d) δ 7.71-7.77 (m, 2H), 7.49-7.55 (m, 1H), 7.41-7.47 (m, 3H), 7.29-7.32 (m, 2H), 7.11-7.18 (m, 3H), 6.97 (s, 1H), 6.78 (s, 1H), 2.43 (s, 6H), 2.36 (s, 3H); MS (m/z) 379.2 (M+H⁺).

### 3-amino-N,N,5-trimethylbenzenesulfonamide

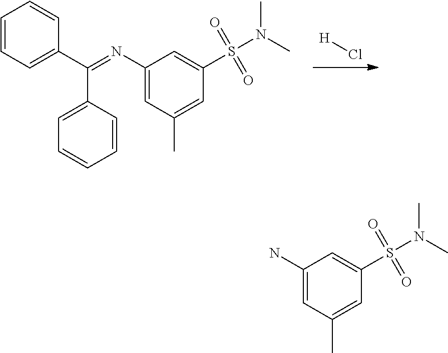

3-[(diphenylmethylidene)amino]-N,N,5-trimethylbenzenesulfonamide (529 mg, 1.258 mmol) was dissolved in Tetrahydrofuran (THF) (7 mL) and HCl (0.839 mL, 5.03 mmol, 6M aqueous) was added. Reaction was stirred at room temperature for 60 minutes and then concentrated. Crude was partitioned between ethyl acetate and satd. NaHCO₃ and layers were separated. Organics were concentrated and triturated in diethyl ether, filtered and dried to give the title compound as a pale yellow solid (220 mg, 81%). ¹H NMR (400 MHz, CHLOROFORM-d) δ 6.96 (s, 1H), 6.88 (s, 1H), 6.70 (s, 1H), 3.87 (br. s., 2H), 2.72 (s, 6H), 2.34 (s, 3H); MS (m/z) 215.0 (M+H⁺).

The following intermediates, used for the preparation of named example compounds, were synthesized using methods analogous to the ones described above.

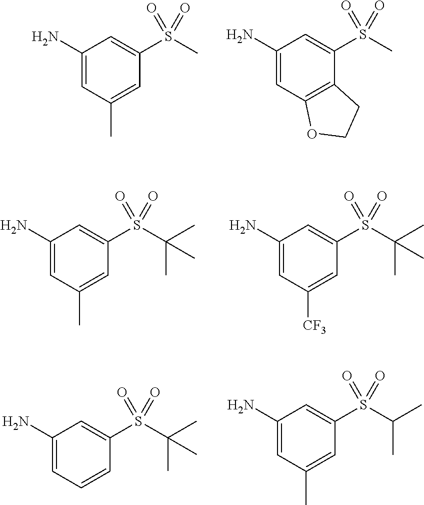 [SMILES: Cc1cc(N)cc(S(C)(=O)=O)c1 ; CS(=O)(=O)c1cc(N)cc2c1CCO2 ; Cc1cc(N)cc(S(=O)(=O)C(C)(C)C)c1 ; CC(C)(C)S(=O)(=O)c1cc(N)cc(C(F)(F)F)c1 ; CC(C)(C)S(=O)(=O)c1cccc(N)c1 ; Cc1cc(N)cc(S(=O)(=O)C(C)C)c1]

### Preparation 21

### 1-methoxy-2-(methylsulfonyl)-4-nitrobenzene

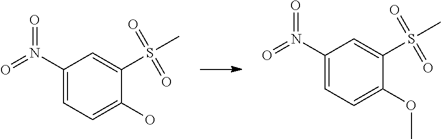

To a solution of 2-(methylsulfonyl)-4-nitrophenol (200 mg, 0.921 mmol) in N,N-Dimethylformamide (DMF) (4 mL) were added potassium carbonate (191 mg, 1.381 mmol) and iodomethane (0.115 mL, 1.842 mmol) at rt. After overnight, the reaction was quenched with water followed by extraction with ethyl acetate (×2). The combined organic solution was washed with 1N HCl solution, sat'd aq NaHCO3 solution, and brine. After drying over MgSO4, filtration, and concentration in vacuo, the brownish solid was collected and washed with 1:1 Ethyl ether and hexane to provide 1-methoxy-2-(methylsulfonyl)-4-nitrobenzene (163 mg, 0.677 mmol, 73.5% yield); ¹H NMR (DMSO-d₆) δ: 8.56-8.60 (m, 1H), 8.54 (d, J=2.8 Hz, 1H), 7.55 (d, J=9.3 Hz, 1H), 4.12 (s, 3H), 3.34 (s, 3H); LCMS (m/z) 232.0 (M+H⁺).

The following intermediates, used for the preparation of named example compounds, were synthesized using methods analogous to the ones described above.

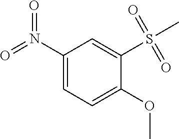

### Preparation 22

### 1-iodo-2,3-dimethyl-5-nitrobenzene

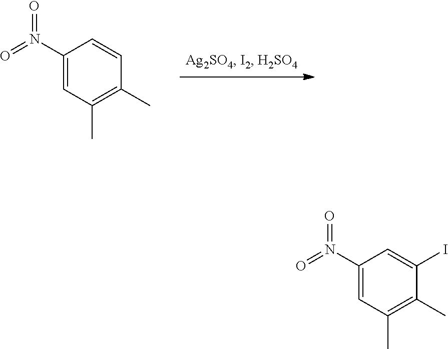

1,2-dimethyl-4-nitrobenzene (1.00 g, 6.62 mmol) and silver sulfate (1.238 g, 3.97 mmol) were mixed in sulfuric acid (5 mL) and iodine (1.763 g, 6.95 mmol) was added. Mixture was stirred at room temperature overnight and then added dropwise to a solution of sodium hydrogen sulfite (aq). A precipitate formed and mixture was stirred for 5 minutes. Precipitate was filtered, partitioned between DCM and water and any solid that did not dissolve was filtered. Organics were concentrated and dried to give the title compound as a light yellow solid (1.20 g, 61%) in 93% purity. 7% starting material remained. ¹H NMR (400 MHz, CHLOROFORM-d) δ 8.55 (d, J=2.27 Hz, 1H), 8.02 (d, J=2.27 Hz, 1H), 2.53 (s, 3H), 2.48 (s, 3H).

### Preparation 23

### 1,2-dimethyl-3-(methylsulfonyl)-5-nitrobenzene 2,3-dimethyl-5-nitrophenyl methyl sulfone

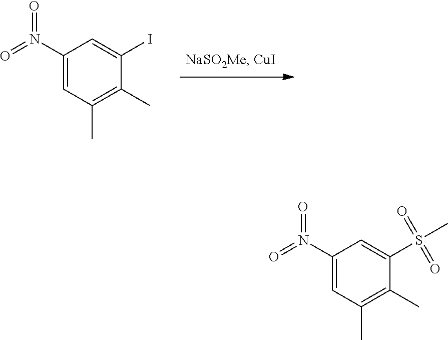

A mixture of 1-iodo-2,3-dimethyl-5-nitrobenzene (1.20 g, 4.03 mmol), sodium methanesulfinate (0.581 g, 4.83 mmol) and copper(I) iodide (1.151 g, 6.04 mmol) in N,N-Dimethylformamide (DMF) (6 mL) was heated at 110° C. for 2 hours. Reaction turned orange and a precipitate formed when it got up to temperature, brown after 2 h. More sodium methanesulfinate (0.581 g, 4.83 mmol) was added and mixture was heated another 1 hour. Then more copper(I) iodide (1.151 g, 6.04 mmol) was added and mixture was heated another 4 hours. Reaction was diluted with water and ethyl acetate and filtered to remove insolubles. Filtrate layers were separated and organics were concentrated and purified by column chromatography using an ethyl acetate/hexanes gradient to give the title compound as a light yellow solid (262 mg, 28%). ¹H NMR (400 MHz, CHLOROFORM-d) δ 8.83 (m, 1H), 8.31 (m, 1H), 3.18 (s, 3H), 2.77 (s, 3H), 2.52 (s, 3H); MS (m/z) 230.1 (M+H⁺).

The following intermediates, used for the preparation of named example compounds, were synthesized using methods analogous to the ones described above.

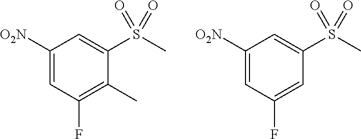

### Preparation 24

### 1-[2-methyl-3-(methylsulfonyl)-5-nitrophenyl]pyrrolidine

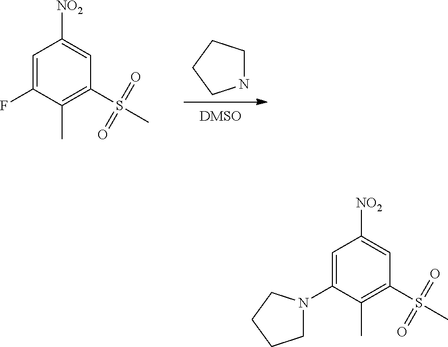 [SMILES: Cc1c(F)cc([N+](=O)[O-])cc1S(C)(=O)=O ; C1CCNC1 ; Cc1c(N2CCCC2)cc([N+](=O)[O-])cc1S(C)(=O)=O]

A mixture of 1-fluoro-2-methyl-3-(methylsulfonyl)-5-nitrobenzene (92.0 mg, 0.394 mmol), and pyrrolidine (140 mg, 1.972 mmol) were heated at 100.0° C. for 30 min. The rm was cooled to rt then quenched with cold water. The suspended solid was collected by filtration then purified by FCC using a Biotage unit [EtOAc-Hex: 10-35%] to yield the title compound (57.0 mg, 51.0%). MS (m/z) 285.2 (M+H⁺).

The following intermediates, used for the preparation of named example compounds, were synthesized using methods analogous to the ones described above.

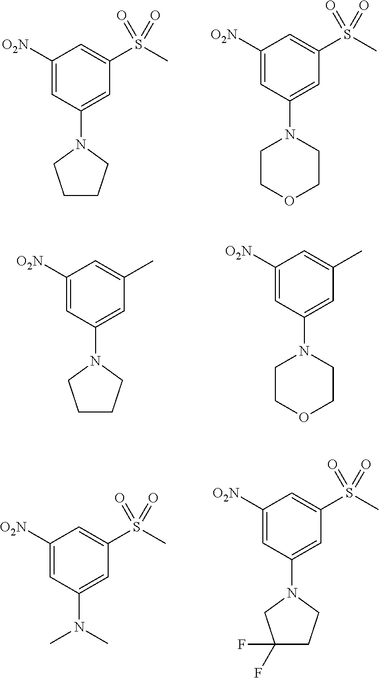 [SMILES: CS(=O)(=O)c1cc(N2CCCC2)cc([N+](=O)[O-])c1 ; CS(=O)(=O)c1cc(N2CCOCC2)cc([N+](=O)[O-])c1 ; Cc1cc(N2CCCC2)cc([N+](=O)[O-])c1 ; Cc1cc(N2CCOCC2)cc([N+](=O)[O-])c1 ; CN(C)c1cc([N+](=O)[O-])cc(S(C)(=O)=O)c1 ; CS(=O)(=O)c1cc(N2CCC(F)(F)C2)cc([N+](=O)[O-])c1]

### Preparation 25

### [3,4-dimethyl-5-(methylsulfonyl)phenyl]amine

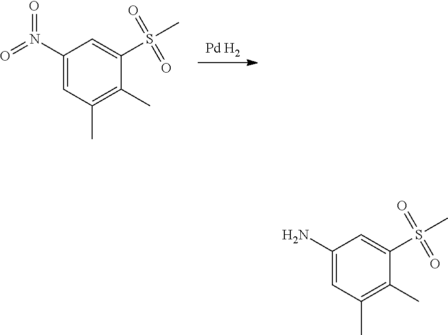

1,2-dimethyl-3-(methylsulfonyl)-5-nitrobenzene (255 mg, 1.112 mmol) was suspended in Ethanol (7 mL) and palladium on carbon (118 mg, 0.111 mmol) was added. Mixture was purged with nitrogen and then put under vacuum. Then vacuum was released with hydrogen (balloon) and reaction was stirred at room temperature overnight. Mixture was filtered through Celite, rinsing with methanol and filtrate was concentrated and dried to give the title compound as a dark green sticky oil (213 mg, 86%) in 90% purity. ¹H NMR (400 MHz, DMSO-d₆) δ 7.06 (d, J=2.27 Hz, 1H), 6.70 (m, 1H), 5.33 (s, 2H), 3.11 (s, 3H), 2.36 (s, 3H), 2.19 (s, 3H); MS (m/z) 200.1 (M+H⁺).

The following intermediates, used for the preparation of named example compounds, were synthesized using methods analogous to the ones described above.

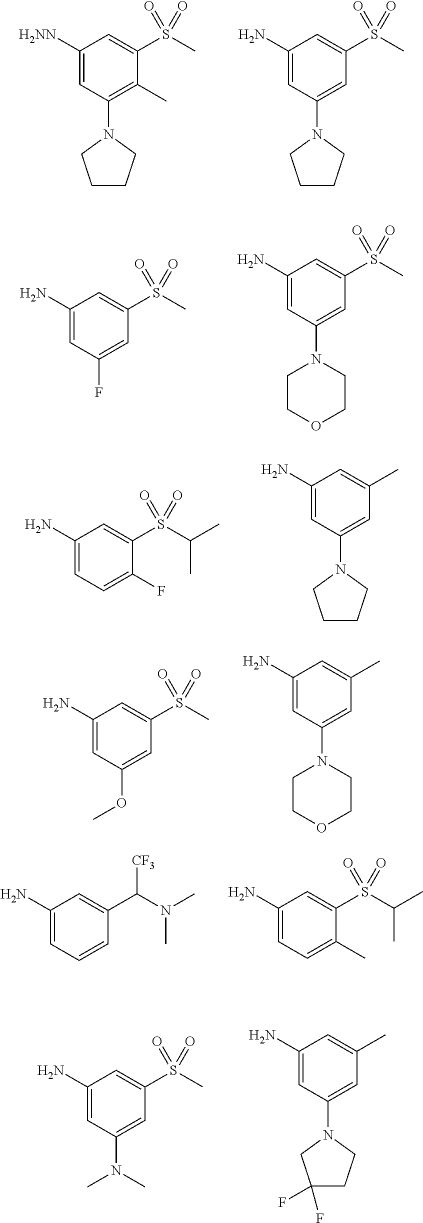

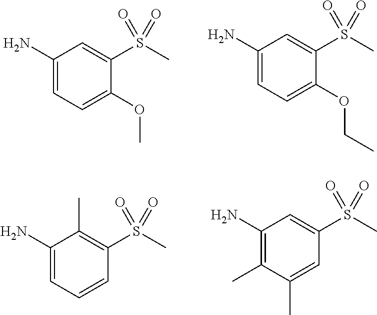

### Preparation 26

### N-[3,4-Bis(methyloxy)phenyl]-4-chloro-2-pyrimidinamine

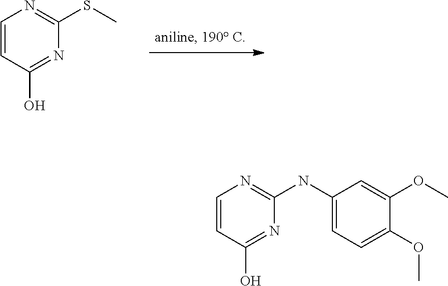

2-{[3,4-Bis(methyloxy)phenyl]amino}-4(1H)-pyrimidinone: 2-(Methylthio)-4(1H)-pyrimidinone (8.2 g, 57.7 mmol) and [[3,4-bis(methyloxy)phenyl]amine 3,4-bis(methyloxy)aniline (9.28 g, 60.6 mmol) were heated at 180° C. for 2 hours. The resulting residue was cooled to rt and triturated with EtOH (4 mL). The resulting solid was filtered, washed with EtOH, and air dried air to give the title compound (12.4 g 87%). ¹H NMR (400 MHz, DMSO-d₆) δ ppm 3.73 (s, 3H) 3.74 (s, 3H) 5.75 (d, J=6.02 Hz, 1H) 6.90 (d, J=8.78 Hz, 1H) 7.06 (br. s., 1H) 7.23 (br. s., 1H) 7.63-7.79 (m, 1H) 8.50-8.80 (m, 1H); MS (m/z) 248 (M+H⁺).

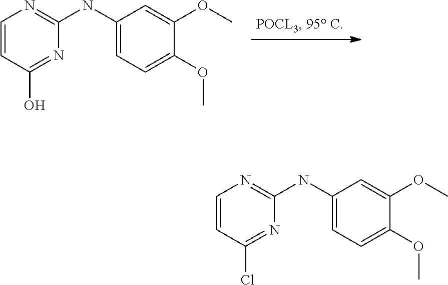 [SMILES: COc1ccc(Nc2nccc(O)n2)cc1OC ; COc1ccc(Nc2nccc(Cl)n2)cc1OC]

N-[3,4-Bis(methyloxy)phenyl]-4-chloro-2-pyrimidinamine: A flask was charged with 2-{[3,4-bis(methyloxy)phenyl]amino}-4(1H)-pyrimidinone (31 g, 125 mmol) followed by POCl₃ (180 mL, 1930 mmol). The reaction was heated to 95° C. for 4 hours then was cooled to rt and diluted with water. The solution was quenched with aq NaOH and the resulting precipitate collected via filtration (17 g). The filtrate was concentrated and more solid crashed out. The solid was isolated via filtration (4 g) and the filtrate was extracted with EtOAc. The combined extracts were concentrated onto silica and the crude material was purified via flash chromatography eluting with 20-60% THF/hexanes to give additional product (1.6 g). The three portions were combined to give the title compound. ¹H NMR (400 MHz, CHLOROFORM-d) δ ppm 3.89 (s, 3H) 3.92 (s, 3H) 6.73 (d, J=5.27 Hz, 1H) 6.87 (d, J=8.53 Hz, 1H) 7.01 (dd, J=8.53, 2.51 Hz, 1H) 7.21-7.28 (m, 1H) 7.32 (d, J=2.51 Hz, 1H) 8.27 (d, J=5.27 Hz, 1H); MS (m/z) 266 (M+H⁺).

The following intermediates, used for the preparation of named example compounds, were synthesized using methods analogous to the ones described above.

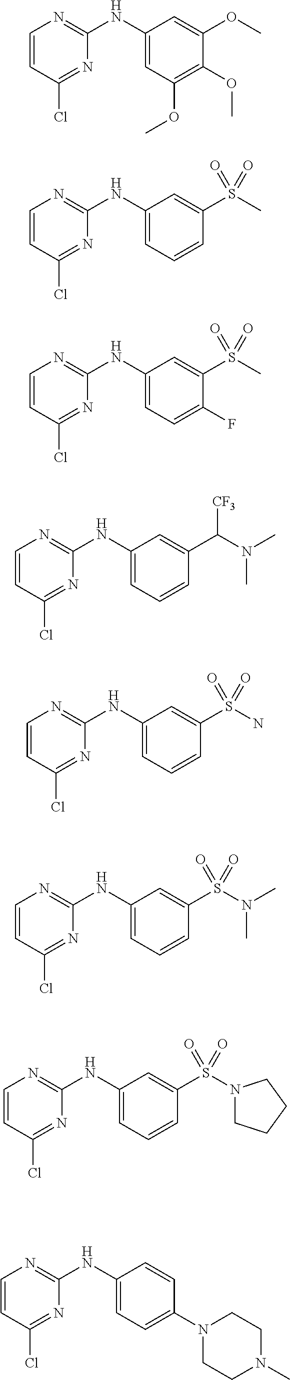

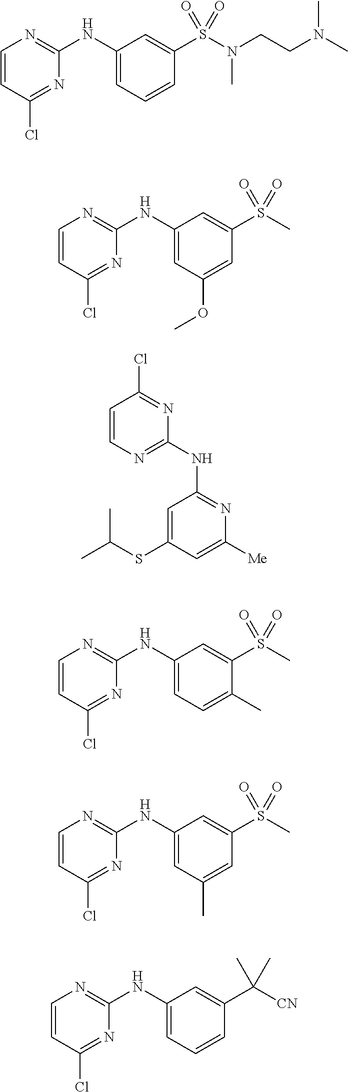

### Preparation 27

### 5-(Methyloxy)-1H-indazol-3-amine

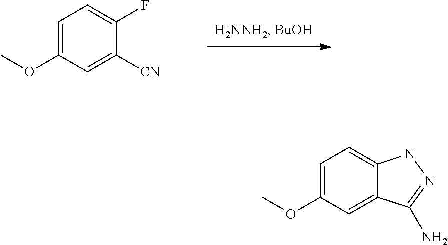

5-(Methyloxy)-1H-indazol-3-amine: 2-Fluoro-5-(methyloxy)benzonitrile (5 g, 33.1 mmol) was dissolved in 1-Butanol (50 mL), and hydrazine hydrate (20.61 mL, 662 mmol) was added. The reaction mixture was heated at 150° C. for 48 hours then it was cooled to rt, diluted with water, and extracted with EtOAc (2×100 mL). The organic layer was washed with brine, dried over Na₂SO₄ and concentrated under vacuum to give the title compound (2 g, 37%). ¹H NMR (400 MHz, DMSO-d₆) δ ppm 3.75 (s, 3H) 5.17 (s, 0H) 6.89 (dd, J=9.03, 2.51 Hz, 0H) 7.09-7.22 (m, 1H) 11.18 (br. s., 7H); MS (m/z) 164 (M+H⁺).

The following intermediates, used for the preparation of named example compounds, were synthesized using methods analogous to the ones described above.

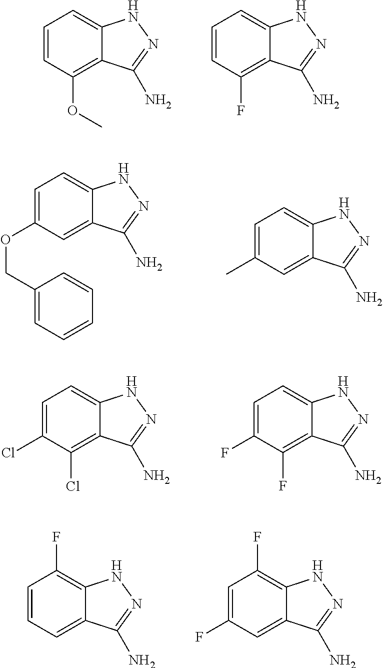

### Preparation 28

### 5-Fluoro-N-methyl-1H-indazol-3-amine

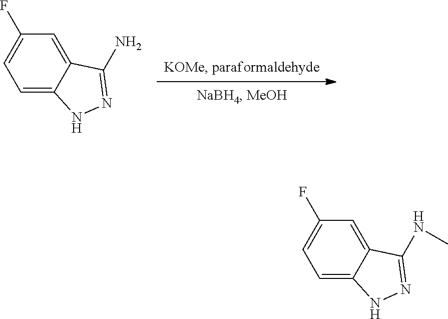

5-Fluoro-1H-indazol-3-amine (200 mg, 1.323 mmol) was dissolved in Methanol (4 mL) then paraformaldehyde (238 mg, 7.94 mmol) and potassium methoxide (1.172 mL, 3.97 mmol) were added. The resulting mixture was heated to 65° C. for 3.5 hours, then removed from heat. NaBH4 (125 mg, 3.31 mmol) was added and heating was continued overnight. The reaction was cooled to room temp and several drops of satd. NaHCO₃ were added. The MeOH was removed under vacuum and the resulting mixture diluted with satd. NaCl and DCM. The organic layer was isolated and concentrated and the resulting material purified via flash chromatography on a 10 g silica cartridge eluting with 0.5-5% MeOH/DCM to give desired product (73 mg, 90%) as a white solid. ¹H NMR (400 MHz, DMSO-d₆) δ ppm 11.47 (s, 1H), 7.41 (dd, J=2.53, 9.35 Hz, 1H), 7.25 (dd, J=4.29, 8.84 Hz, 1H), 7.11 (td, J=2.53, 9.09 Hz, 1H), 5.87 (d, J=5.05 Hz, 1H), 2.84 (d, J=5.31 Hz, 3H); MS (m/z) 166.0 (M+H⁺).

### Preparation 29

### N-(2-chloro-4-pyrimidinyl)-5-fluoro-N-methyl-1H-indazol-3-amine

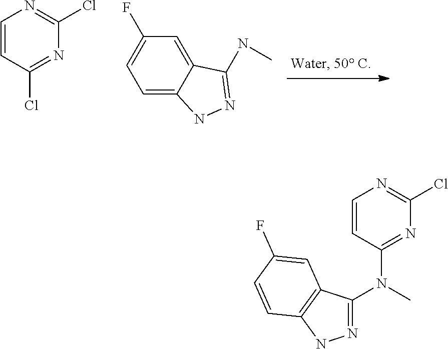

2,4-dichloropyrimidine (541 mg, 3.63 mmol) and 5-fluoro-N-methyl-1H-indazol-3-amine (500 mg, 3.03 mmol) were mixed in Water (30 mL) and heated at 50° C. for 20 hours and a thick precipitate was present. To the mixture was added 0.5 mL 2M HCl in diethyl ether and it was stirred for 2 hours. The reaction was cooled to room temperature and the solid was filtered, rinsing with water, and dried in vacuum oven to give the title compound as a light yellow solid (650 mg, 63% yield, 92% purity).

The following intermediates, used for the preparation of named example compounds, were synthesized using methods analogous to the one described above.

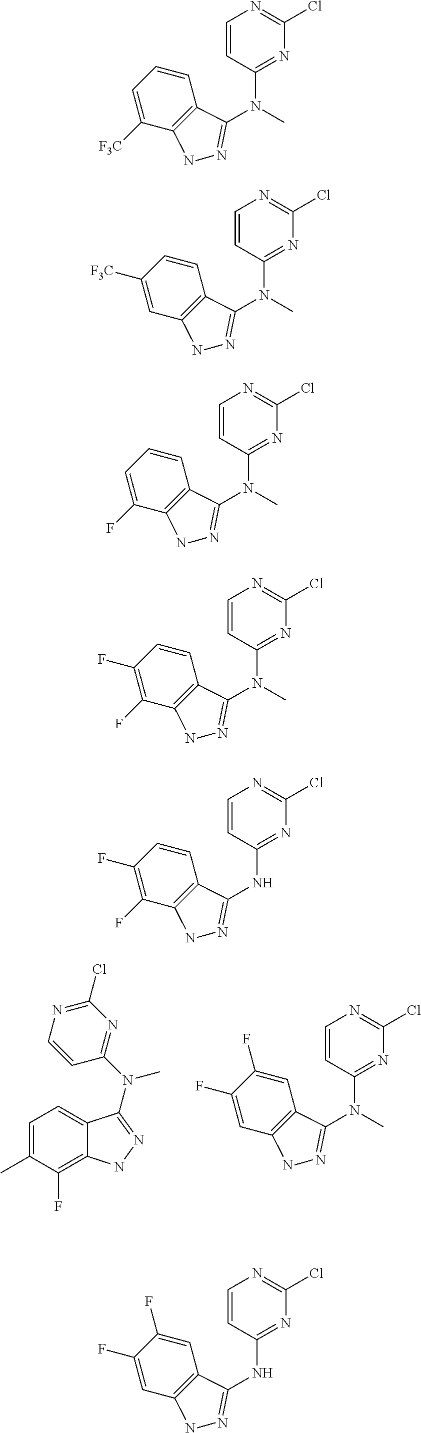

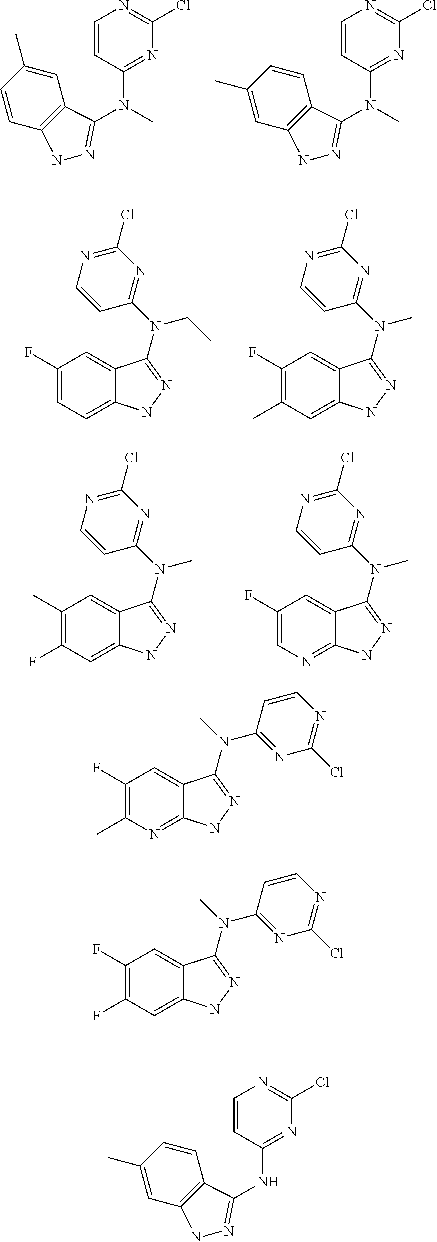

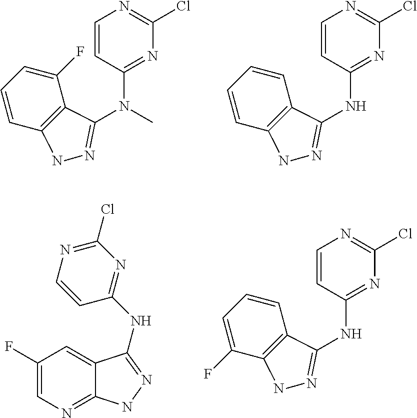 [SMILES: CN(c1ccnc(Cl)n1)c1n[nH]c2cccc(F)c12 ; Clc1nccc(Nc2n[nH]c3ccccc23)n1 ; Fc1cnc2[nH]nc(Nc3ccnc(Cl)n3)c2c1 ; Fc1cccc2c(Nc3ccnc(Cl)n3)n[nH]c12]

### Preparation 30

### N-(2-chloro-4-pyrimidinyl)-5-fluoro-1H-indazol-3-amine

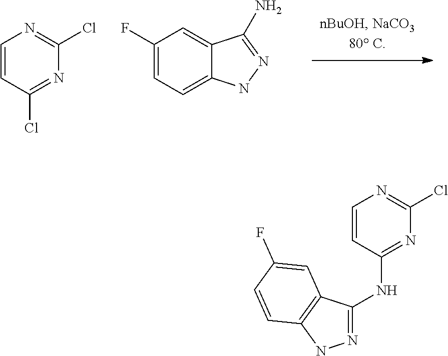

A mixture of 2,4-dichloropyrimidine (15.23 g, 102 mmol) and 5-fluoro-H-indazol-3-amine (15.45 g, 102 mmol) in 1-Butanol (511 ml) was treated with sodium carbonate (21.67 g, 102 mmol) and heated to 80° C. for 20 hours. Additional The 2,4-dichloropyrimidine (15.23 g, 204 mmol) was added and the reaction was stirred for 48 hours. Additional 2,4-dichloropyrimidine (7.6 g, 51 mmol) and the reaction was stirred for 72 hours. The reaction was filtered and the filtrate concentrated. The resulting solid was triturated with a solution of MeOH/EtOAc to give the title compound. ¹H NMR (DMSO-d₆) δ: 12.81 (br. s., 1H), 10.79 (br. s., 1H), 8.34 (d, J=5.8 Hz, 1H), 7.73-7.80 (m, 1H), 7.52 (dd, J=9.1, 4.3 Hz, 1H), 7.29 (td, 1H); MS (m/z) 263 (M⁺).

### Example 1

### N4-(5-Fluoro-1H-indazol-3-yl)-N4-methyl-N2-[3,4,5-tris(methyloxy)phenyl]-2,4-pyrimidinediamine

### Coupling Method A

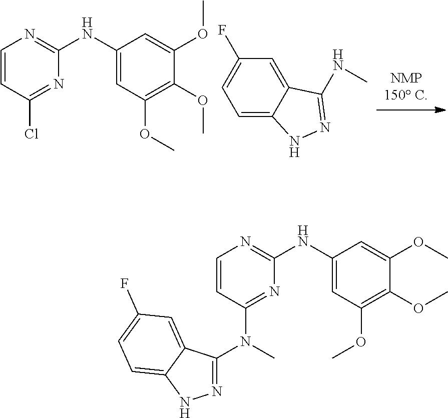

4-Chloro-N-[3,4,5-tris(methyloxy)phenyl]-2-pyrimidinamine (45 mg, 0.152 mmol) and 5-fluoro-N-methyl-1H-indazol-3-amine (27.9 mg, 0.152 mmol) were dissolved in N-Methyl-2-pyrrolidone (NMP) (2 mL) and the reaction was heated at 150° C. for 20 hours. The reaction was concentrated and purified via mass directed, prep HPLC using a Sunfire, 30×100 mm, C18 column eluting with 26-60% acetonitrile/water (with 0.1% TFA) over a 10.5 minute gradient. Fractions containing product were concentrated to give the title compound as the TFA salt (20.4 mg, 30%). ¹H NMR (500 MHz, DMSO-d₆) δ 7.90 (d, J=5.86 Hz, 1H), 7.61 (dd, J=3.78, 8.67 Hz, 1H), 7.27-7.35 (m, 2H), 7.21 (s, 2H), 5.80 (d, J=5.86 Hz, 1H), 3.72 (s, 6H), 3.61 (s, 3H), 3.56 (s, 3H); MS (m/z) 425.1 (M+H⁺).

### Example 2

N²-[3,4-Bis(methyloxy)phenyl]-N⁴-1H-pyrazolo[3,4-b]pyridin-3-yl-2,4-pyrimidinediamine

### Coupling Method B

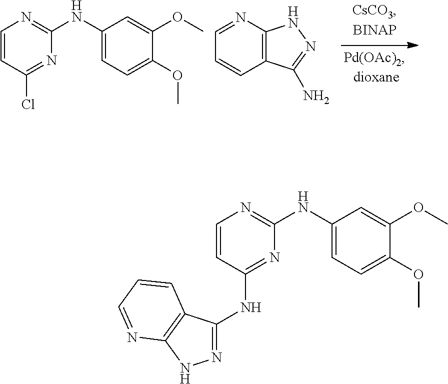

A vial was charged with N-[3,4-bis(methyloxy)phenyl]-4-chloro-2-pyrimidinamine (50.0 mg, 0.188 mmol) and 1H-pyrazolo[3,4-b]pyridin-3-amine (25.2 mg, 0.188 mmol). 1,4-Dioxane (941 μl) was added followed by cesium carbonate (184 mg, 0.565 mmol), binap (11.72 mg, 0.019 mmol), and finally palladium(II)acetate (4.22 mg, 0.019 mmol). The resulting mixture was heated to 90° C. overnight. The reaction was diluted with DMF and filtered through a 0.45 μm PTFE frit and purified via prep HPLC using a Sunfire 5 μm, 30×75 mm, C18 column eluting with 20-60% MeCN/water (with 0.1% TFA). The fractions containing product were concentrated to give the product as the TFA salt (22.2 mg, 25%). ¹H NMR (400 MHz, DMSO-d₆) δ ppm 3.30-3.66 (m, 3H) 3.73 (s, 3H) 6.58-7.18 (m, 5 H) 7.94-8.06 (m, 1H) 8.10-8.31 (m, 1H) 8.52 (d, J=3.02 Hz, 1H) 10.26 (br. s., 1H) 11.28 (br. s., 1H) 13.55 (br. s., 1H); MS (m/z) 364 (M+H⁺).

### Example 3

### 3-({4-[(5-Fluoro-1H-indazol-3-yl)amino]-2-pyrimidinyl}amino)-N,N-dimethyl-benzenesulfonamide

### Coupling Method C

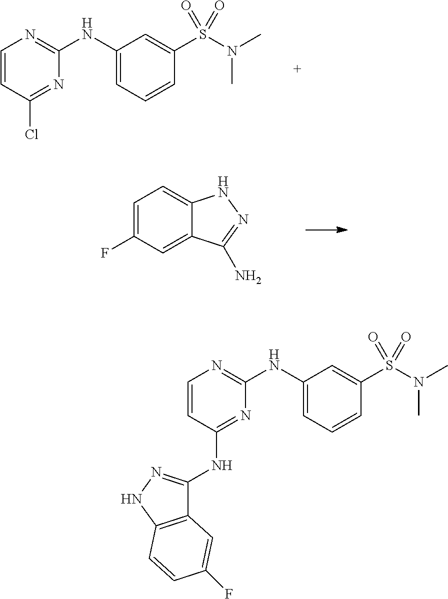 [SMILES: CN(C)S(=O)(=O)c1cccc(Nc2nccc(Cl)n2)c1 ; Nc1n[nH]c2ccc(F)cc12 ; CN(C)S(=O)(=O)c1cccc(Nc2nccc(Nc3n[nH]c4ccc(F)cc34)n2)c1]

A microwave vial was charged with 3-[(4-chloro-2-pyrimidinyl)amino]-N,N-dimethylbenzenesulfonamide (100 mg, 0.32 mmol), 5-fluoro-1H-indazol-3-amine (48.3 mg, 0.32 mmol), and N-Methyl-2-pyrrolidone (2 ml). The reaction vial was put in an Emrys Optimizer (150 W, absorption normal, 180° C., 20 min). The crude mixture was loaded onto a Strata SCX column (55 um, 70 A, 5 g/20 ml Giga Tubes). The column was first flushed with 20 ml of MeOH, followed by 20 ml of 1N NH3 in MeOH. The collected 1N NH3 in MeOH fraction was concentrated and the crude residue was purified via prep HPLC using a Sunfire (5 μm, 30×150 mm, C18 column) eluting with 10-40% MeCN/water (with 0.1% TFA). The fractions containing the product were combined and concentrated to afford the titled compound as the TFA salt (77.4 mg, 45%). ¹H NMR (500 MHz, DMSO-d₆) δ ppm 2.57 (s, 6H), 6.80 (br. s., 1H), 7.22-7.35 (m, 3H), 7.47-7.57 (m, 2H), 7.80 (br. s., 1 H), 8.12 (d, J=6.3 Hz, 2H), 9.98 (br. s., 1H), 10.40 (br. s., 1H), 12.92 (br. s., 1H); MS (m/z) 428 (M+H⁺).

### Example 4

### N4-(5-Fluoro-1H-indazol-3-yl)-N2-[3-(methoxy)-5-(methylsulfonyl)phenyl]-2,4-pyrimidinediamine

### Coupling Method D

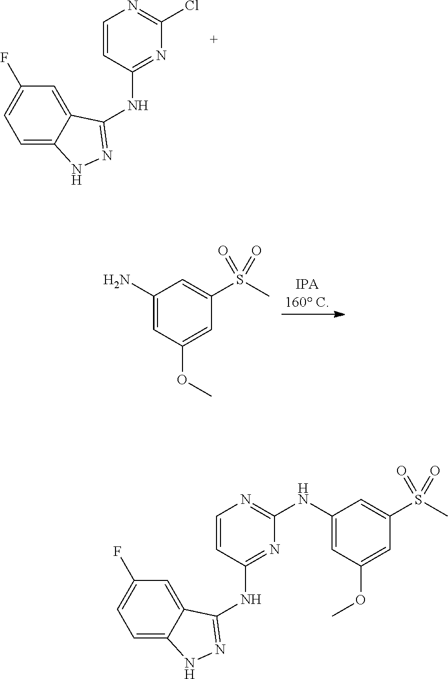 [SMILES: Fc1ccc2[nH]nc(Nc3ccnc(Cl)n3)c2c1 ; COc1cc(N)cc(S(C)(=O)=O)c1 ; COc1cc(Nc2nccc(Nc3n[nH]c4ccc(F)cc34)n2)cc(S(C)(=O)=O)c1]

N-(2-chloro-4-pyrimidinyl)-5-fluoro-1H-indazol-3-amine (50 mg, 0.190 mmol) and [3-(methyloxy)-5-(methylsulfonyl)phenyl]amine (38.2 mg, 0.190 mmol) were taken up in Isopropanol (3 mL) and HCl (4N in 1,4-dioxane; 1 drop) was added. The mixture was heated to 160° C. in the microwave for 20 minutes. The mixture was concentrated under reduced pressure, taken up in DMSO and purified by HPLC using a Sunfire (5 μm, 30×150 mm, C18 column) eluting with 10-50% MeCN/water (with 0.1% TFA). The fractions containing the product were combined and concentrated to afford the titled compound as the TFA salt (6.1 mg, 0.011 mmol, 5.93% yield). ¹H NMR (500 MHz, DMSO-d₆) δ ppm 3.14 (s, 3H) 3.68 (br. s., 3H) 6.79 (br. s., 1H) 6.99 (s, 1H) 7.26 (td, J=9.09, 2.32 Hz, 1 H) 7.44-7.50 (m, 1H) 7.53 (dd, J=9.03, 4.15 Hz, 1H) 7.65 (m, 2H) 8.12 (d, J=6.10 Hz, 1 H) 9.86 (br. s., 1H) 10.31 (br. s., 1H) 12.86 (br. s., 1H); MS (m/z) 429.1 (M+H⁺).

### Example 5

### N4-(5-fluoro-6-methyl-1H-pyrazolo[3,4-b]pyridin-3-yl)-N4-methyl-N2-[3-methyl-5-(methylsulfonyl)phenyl]pyrimidine-2,4-diamine

### Coupling Method E

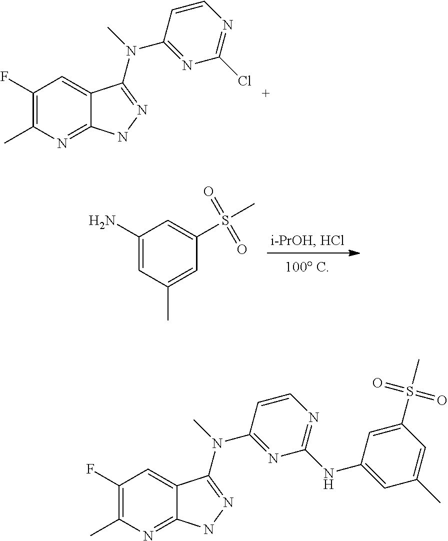 [SMILES: Cc1nc2[nH]nc(N(C)c3ccnc(Cl)n3)c2cc1F ; Cc1cc(N)cc(S(C)(=O)=O)c1 ; Cc1cc(Nc2nccc(N(C)c3n[nH]c4nc(C)c(F)cc34)n2)cc(S(C)(=O)=O)c1]

A mixture of N-(2-chloro-4-pyrimidinyl)-5-fluoro-6-methyl-1H-pyrazolo[3,4-b]pyridin-3-amine (1.0 g, 3.42 mmol) in isopropanol (50 ml) were added [3-methyl-5-methylsulfonyl]aniline (0.73 g, 3.93 mmol) at rt. HCl (4.0M in dioxane) (0.32 ml, 1.264 mmol) was added and the mixture were stirred at 100° C. for 12 hours. LC/MS indicated the reaction was completed. The desired product was precipitated out from the reaction mixture as the HCl salt. The solid was collected by filtration then dissolved in 900 ml of THF-ethyl acetate (10%) mix solvents. This solution was basified with 2M aq. sodium carbonate. The organic phase was separated and dried with sodium sulfate, filtered and the solvent was removed under reduced pressure. The white solid was washed with ether and hexane before was dried under vacuum to provide the title compound. (1.3 g, 86%). ¹HNMR (600 MHz, DMSO-d₆) δ ppm 2.25 (br.s, 3H) 2.53-2.61 (m, 3H) 3.16 (s, 3H) 3.59 (s, 3H) 6.19 (br.s., 1H) 7.35 (br. S., 1H) 7.62 (br. S., 1H) 7.90 (d, J=8.69 Hz, 1H) 8.03 (d, J=6.04 Hz, 1H) 8.23 (br, s., 1H) 10.14 (br. S., 1H) 13.71 (br.s., 1H); MS (m/z) 442 (M+H⁺).

### Example 6

### N4-(5-fluoro-1H-pyrazolo[3,4-b]pyridin-3-yl)-N4-methyl-N2-[3-(methyloxy)-5-(methylsulfonyl)phenyl]-2,4-pyrimidinediamine

### Coupling Method F

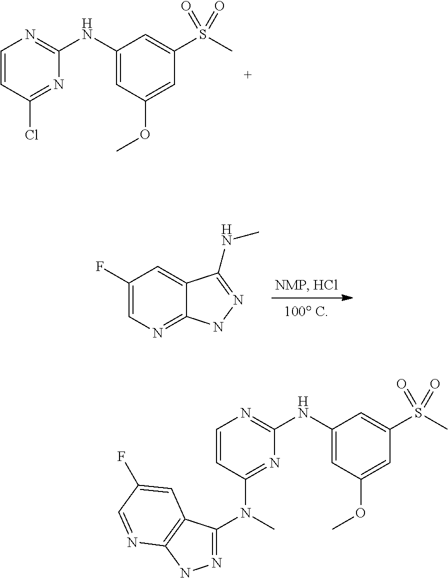 [SMILES: COc1cc(Nc2nccc(Cl)n2)cc(S(C)(=O)=O)c1 ; CNc1n[nH]c2ncc(F)cc12 ; COc1cc(Nc2nccc(N(C)c3n[nH]c4ncc(F)cc34)n2)cc(S(C)(=O)=O)c1]

A solution of 4-chloro-N-[3-(methyloxy)-5-(methylsulfonyl)phenyl]-2-pyrimidinamine (28.3 mg, 0.090 mmol) and 5-fluoro-N-methyl-1H-pyrazolo[3,4-b]pyridin-3-amine (15.00 mg, 0.090 mmol) in N-Methyl-2-pyrrolidone (NMP) (451 μl) was treated with 2 drops of 2N HCl then heated to 100° C. for 20 hours. The reaction mixture was diluted with MeOH filtering through a 0.2 μm ptfe frit. The crude solution was purified via prep HPLC using a 0.5 μm, 30×150 mm, C18 column eluting with 20-60% MeCN/water (with 0.1% TFA). The fractions containing product were combined and neutralized with satd aq NaHCO3, then extracted with DCM and the combined extracts were passed through a hydrophobic frit and concentrated to provide the title compound (10 mg, 25%). ¹H NMR (DMSO-d₆) δ: 13.79 (br. s., 1H), 9.68 (s, 1H), 8.62 (s, 1H), 8.08-8.14 (m, 1H), 8.03 (d, J=5.8 Hz, 1H), 8.00 (dd, J=8.6, 2.8 Hz, 1H), 7.61 (s, 1H), 6.96 (s, 1H), 6.08 (d, J=6.1 Hz, 1H), 5.77 (s, 1H), 3.78 (s, 3H), 3.58 (s, 3H), 3.18 (s, 3H); MS (m/z) 444 (M+H⁺).

### Example 7

### 2-(ethyloxy)-5-({4-[(5-fluoro-1H-indazol-3-yl)amino]-2-pyrimidinyl}amino)-N,N-dimethylbenzenesulfonamide

### Coupling Method G

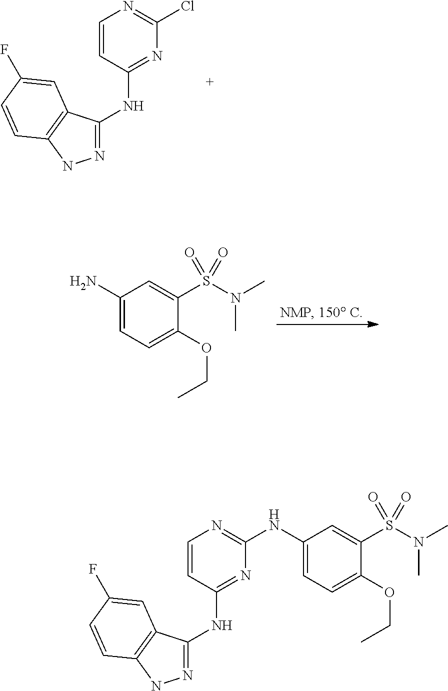 [SMILES: Fc1ccc2[nH]nc(Nc3ccnc(Cl)n3)c2c1 ; CCOc1ccc(N)cc1S(=O)(=O)N(C)C ; CCOc1ccc(Nc2nccc(Nc3n[nH]c4ccc(F)cc34)n2)cc1S(=O)(=O)N(C)C]

A solution of N-(2-chloro-4-pyrimidinyl)-5-fluoro-1H-indazol-3-amine (30.0 mg, 0.114 mmol) and 5-amino-2-(ethyloxy)-N,N-dimethylbenzenesulfonamide (27.8 mg, 0.114 mmol) in N-Methyl-2-pyrrolidone (NMP) (569 μl) was heated to 150° C. overnight. The reaction mixture was filtered through a 0.2 μm PTFE frit and purified via prep HPLC using a 0.5 μm, 30×150 mm, C18 column eluting with 17-51% MeCN/water (with 0.1% TFA) to give the title compound as the TFA salt. ¹H NMR (METHANOL-d₄) δ: 8.08-8.12 (m, 1H), 8.05-8.08 (m, 1H), 8.01-8.05 (m, 1H), 7.96-8.01 (m, 1H), 7.44-7.54 (m, 2H), 7.39-7.44 (m, 1H), 7.27-7.38 (m, 2H), 7.20-7.27 (m, 1H), 6.68-6.75 (m, 1H), 3.59-3.68 (m, 1H), 3.06 (d, J=7.3 Hz, 3H), 2.73-2.83 (m, 3H), 1.83-1.93 (m, 2H), 1.51-1.64 (m, 2H).; MS (m/z) 484 (M+H⁺).

### Example 8

### N2-(1,1-dioxido-1-benzothien-4-yl)-N4-(5-fluoro-1H-indazol-3-yl)-2,4-pyrimidinediamine

### Coupling Method H

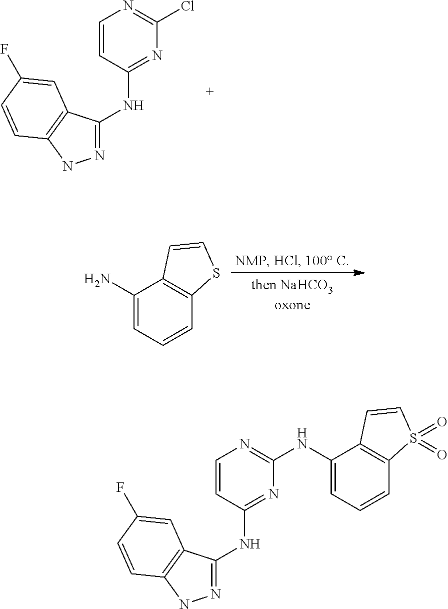 [SMILES: Fc1ccc2[nH]nc(Nc3ccnc(Cl)n3)c2c1 ; Nc1cccc2sccc12 ; O=S1(=O)C=Cc2c(Nc3nccc(Nc4n[nH]c5ccc(F)cc45)n3)cccc21]

A solution of N-(2-chloro-4-pyrimidinyl)-5-fluoro-1H-indazol-3-amine (25.00 mg, 0.095 mmol) and 1-benzothien-4-ylamine (14.15 mg, 0.095 mmol) in N-Methyl-2-pyrrolidone (NMP) (474 μl) was treated with 2 drops of 2N HCl in Et2O and stirred at 100° C. for 20 hours. Solid NaHCO3 was added followed by oxone (58.3 mg, 0.095 mmol), and a few drops of water. The reaction was stirred at rt for 20 hours. The reaction mixture was filtered through a 0.2 μm ptfe frit and diluted with MeOH then purified via prep HPLC using a Sunfire 5 μm, 30×150 mm, C18 column eluting with 25-65% MeCN/water (with 0.1% TFA) to give the title compound as the TFA salt. ¹H NMR (DMSO-d₆) δ: 12.97 (s, 1H), 9.32 (s, 1H), 9.18 (s, 1H), 8.18 (s, 1H), 7.68 (d, J=5.6 Hz, 1H), 7.59 (dd, J=9.0, 4.2 Hz, 1H), 7.55 (d, J=5.6 Hz, 1H), 7.42 (d, J=7.8 Hz, 1H), 7.25-7.37 (m, 3H), 6.57 (t, J=8.0 Hz, 1H).; MS (m/z) 409 (M+H⁺).

### Example 9

### 3-((4-((5-fluoro-6-methyl-1H-indazol-3-yl)(methyl)amino)pyrimidin-2-yl)amino)-5-methylbenzenesulfonamide

### Coupling Method I

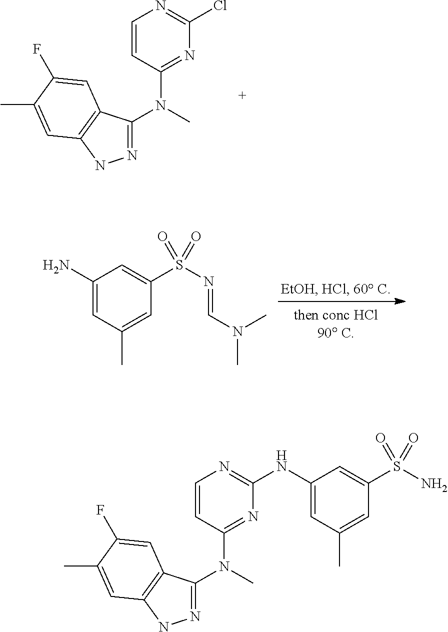 [SMILES: Cc1cc2[nH]nc(N(C)c3ccnc(Cl)n3)c2cc1F ; Cc1cc(N)cc(S(=O)(=O)/N=C/N(C)C)c1 ; Cc1cc(Nc2nccc(N(C)c3n[nH]c4cc(C)c(F)cc34)n2)cc(S(N)(=O)=O)c1]

A mixture of N-(2-chloropyrimidin-4-yl)-5-fluoro-N,6-dimethyl-1H-indazol-3-amine (60.4 mg, 0.207 mmol) abd (E)-N′-((3-amino-5-methylphenyl)sulfonyl)-N,N-dimethylformimidamide (50.0 mg, 0.207 mmol) in Ethanol (950 μl) was treated with 2 drops of 4 N HCl in dioxane and the reaction was heated to 60° C. for one hour. Hydrochloric acid (86 μl, 1.036 mmol) was added and the reaction was heated to 90° C. for one hour. The reaction was complete and was cooled to rt then filtered washing the solid with EtOH. The crude solid was purified via prep HPLC using a Sunfire 5 μm, 30×150 mm, C18 column eluting with 15-60% MeCN/water (with 0.1% TFA). The fractions containing product were combined and neutralized with sat aq NaHCO3 then extracted with DCM. The combined extracts were passed through a hydrophobic frit and concentrated to give the title compound. ¹H NMR (DMSO-d₆) δ: 13.05 (br. s., 1H), 9.55 (s, 1H), 8.29-8.35 (m, 1H), 7.94 (d, J=6.1 Hz, 1H), 7.67 (s, 1H), 7.48 (d, J=6.3 Hz, 2H), 7.11-7.29 (m, 4H), 5.86 (d, J=5.8 Hz, 1H), 3.54 (s, 3H), 2.37 (d, J=1.3 Hz, 3H), 2.28 (s, 3H).; MS (m/z) 409 (M+H⁺).

### Example 10

### N2-[2,3-dimethyl-5-(methylsulfonyl)phenyl]-N4-(5-fluoro-1H-indazol-3-yl)-2,4-pyrimidinediamine

### Coupling Method J

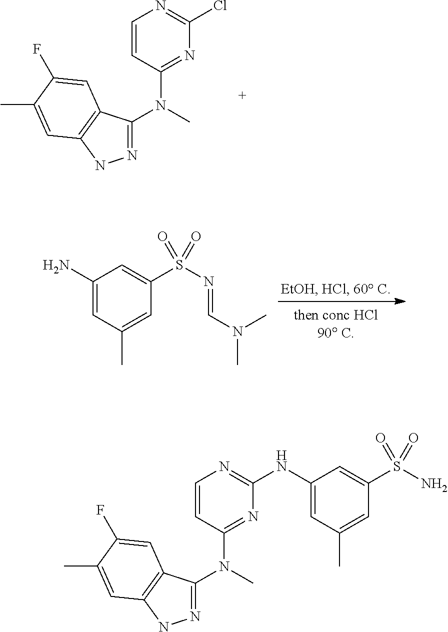 [SMILES: Cc1cc2[nH]nc(N(C)c3ccnc(Cl)n3)c2cc1F ; Cc1cc(N)cc(S(=O)(=O)/N=C/N(C)C)c1 ; Cc1cc(Nc2nccc(N(C)c3n[nH]c4cc(C)c(F)cc34)n2)cc(S(N)(=O)=O)c1]

A solution of N-(2-chloro-4-pyrimidinyl)-5-fluoro-1H-indazol-3-amine (25.00 mg, 0.095 mmol) and [2,3-dimethyl-5-(methylsulfonyl)phenyl]amine (18.89 mg, 0.095 mmol) in N-Methyl-2-pyrrolidone (NMP) (474 μl) was treated with 2 drops of 2 N HCl in Et2O then heated to 100° C. for 16 hours. The crude mixture was filtered through a 0.2 μm ptfe frit and was purified via prep HPLC using a Sunfire 30×150 mm, 5 μm, C18 column eluting with 10-50% MeCN/water (with 0.1% TFA). The fractions containing product were combined and concentrated to give the title compound as the TFA salt. ¹H NMR (DMSO-d₆) δ: 12.92-13.05 (m, 1H), 7.96-8.06 (m, 1H), 7.43-7.75 (m, 3H), 7.16-7.30 (m, 1H), 6.64-6.94 (m, 1H), 2.94-3.12 (m, 3H), 2.30 (br. s., 3H), 2.13 (br. s., 3H).; MS (m/z) 427 (M+H⁺).

### Example 11

### 2-{[3-({4-[(5-fluoro-1H-indazol-3-yl)amino]-2-pyrimidinyl}amino)phenyl]sulfinyl}ethanol

### Coupling Method K

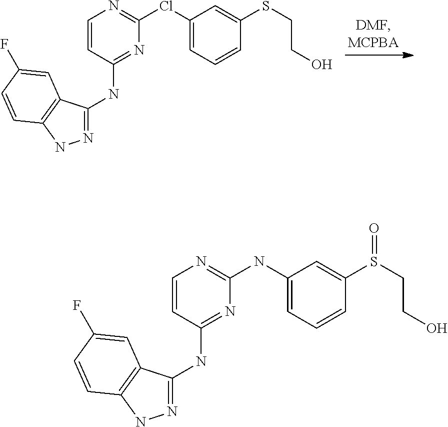

A solution of 2-{[3-({4-[(5-fluoro-1H-indazol-3-yl)amino]-2-pyrimidinyl}amino)phenyl]thio}ethanol (50.0 mg, 0.126 mmol) in N,N-Dimethylformamide (DMF) (631 μl) was treated with MCPBA (21.76 mg, 0.126 mmol. The reaction was stirred for 16 hours then the crude mixture was passed through a 0.2 μm ptfe frit and purified via prep HPLC using a Sunfire 5 μm, 30×150 mm, C18 column eluting with 10-50% MeCN/water (with 0.1% TFA). The fractions containing product were combined and neutralized to afford the title compound. ¹H NMR (DMSO-d₆) δ: 12.76 (s, 1H), 9.85 (br. s., 1H), 9.51 (br. s., 1H), 8.15 (d, J=5.8 Hz, 1H), 7.91-7.99 (m, 2H), 7.56-7.61 (m, 1H), 7.53 (dd, J=9.2, 4.1 Hz, 1H), 7.24-7.36 (m, 2H), 7.16 (d, J=7.8 Hz, 1H), 6.81 (br. s., 1H), 5.03-5.18 (m, 1H), 3.76-3.85 (m, 1H), 3.65 (dt, J=11.0, 5.2 Hz, 1H), 2.86-2.96 (m, 1H), 2.73-2.84 (m, 1H).; MS (m/z) 413 (M+H*).

The following compounds were prepared using procedures analogous to those described above (Methods A-K).

| | | | | MS | |
|---|---|---|---|---|---|
| | | | | (M + | |
| Ex. | Name | Structure | 1H NMR | H)⁺ | Method |
| 12 | N⁴-[5-(methyloxy)- 1H-indazol-3-yl]- N²-[3,4,5- tris(methyloxy) phenyl]-2,4- pyrimidinediamine | 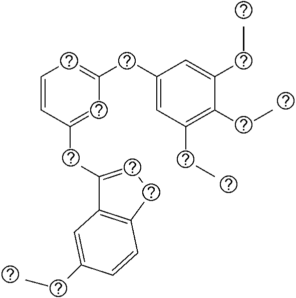 [SMILES: CCc1ccc2c(c1)C(Cc1cccc(Cc3cc(CC)c(CC)c(CC)c3)c1)=CC2] | ¹H NMR (DMSO-d₆, 500 MHz): δ 12.70- 12.85 (m, 1 H), 10.56- 10.93 (m, 1 H), 9.73- 10.19 (m, 1H), 7.99 (d, J = 6.6 Hz, 1 H), 7.41 (d, J = 9.0 Hz, 1 H), 7.02 (br. s., 2 H), 6.73-6.88 (m, 2 H), 6.65 (br. s., 1 H), 3.60 (s, 6 H), 3.48-3.57 ppm (m, 6 H) | 423 | A |
| 13 | N⁴-[5-(methyloxy)- 1H-indazol-3-yl]- N²-[3,4,5- tris(methyloxy) phenyl]-2,4- pyrimidinediamine trifluoroacetate | 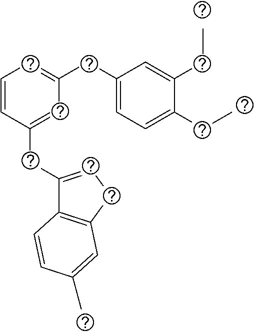 [SMILES: CCc1ccc(Cc2cccc(CC3=CCc4cc(C)ccc43)c2)cc1CC] | ¹H NMR (400 MHz, DMSO-d₆) δ 13.08 (br. s., 1H), 10.84-11.13 (m, 1H), 9.94-10.18 (m, 1H), 7.95-8.08 (m, 1H), 7.71-7.87 (m, 1H), 7.61 (s, 1H), 6.61-7.14 (m, 5H), 3.73 (s, 3H), 3.59 (br. s., 3H) | 397 | A |
| 14 | N²-[3,4- bis(methyloxy) phenyl]-N⁴-(5- chloro-1H-indazol- 3-yl)-2,4- pyrimidinediamine trifluoroacetate | 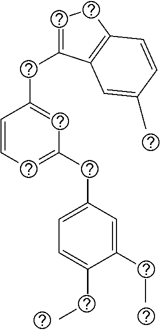 [SMILES: CCc1ccc(Cc2cccc(CC3=CCc4ccc(C)cc43)c2)cc1CC] | ¹H NMR (DMS0-d₆) δ 10.85 (br. s., 1H), 9.96 (br. s., 1H), 8.00 (d, J = 6.6 Hz, 1H), 7.84 (br. s., 1H), 7.55 (d, J = 8.8 Hz, 1H), 7.37 (d, J = 8.8 Hz, 1H), 6.94-7.10 (m, 2H), 6.79 (br. s., 1H), 3.72 (s, 3H), 3.53-3.59 (m, 3H) | 397 | A |
| 15 | N²-[3,4- bis(methyloxy) phenyl]-N⁴-[5- (methyloxy)-1H- indazol-3-yl]-2,4- pyrimidinediamine | 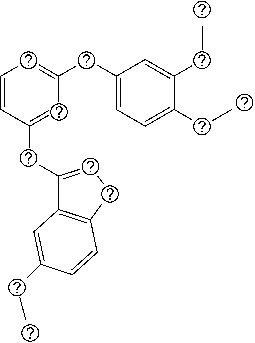 [SMILES: CCc1ccc2c(c1)C(Cc1cccc(Cc3ccc(CC)c(CC)c3)c1)=CC2] | ¹H NMR (DMSO-d₆) δ: 12.44 (br. s., 1H), 9.50 (br. s., 1H), 8.85 (br. s., 1H), 8.05 (d, J = 5.0 Hz, 1H), 7.17-7.45 (m, 4H), 7.01 (d, J = 8.6 Hz, 1H), 6.72 (d, J = 8.1 Hz, 2H), 3.68 (br. s., 6H), 3.58 (br. s., 3H) | 393 | A |
| 16 | N²-[3,4- bis(methyloxy) phenyl]-N⁴-(7- fluoro-1H-indazol- 3-yl)-2,4- pyrimidinediamine trifluoroacetate | 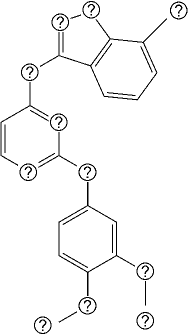 [SMILES: CCc1ccc(Cc2cccc(CC3=CCc4c(C)cccc43)c2)cc1CC] | ¹H NMR (DMSO-d₆) δ 10.79 (br. s., 1H), 9.65- 10.01 (m, 1H), 8.00 (br. s., 1H), 7.60 (br. s., 1H), 7.24 (dd, J = 11.1, 7.4 Hz, 1H), 7.03 (br. s., 3H), 6.68-6.91 (m, 2H), 3.72 (s, 3H), 3.60 (br. s., 3H) | 381 | A |
| 17 | N⁴-1H-indazoI-3- yl-N²-[4-(4-methyl- 1- piperazinyl)phenyl]- 2,4- pyrimidinediamine trifluoroacetate | 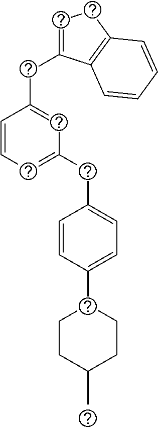 [SMILES: CC1CCC(c2ccc(Cc3cccc(CC4=CCc5ccccc54)c3)cc2)CC1] | ¹H NMR (DMSO-d₆, 400 MHz): d = 9.69- 9.90 (m, 2 H), 8.53- 8.76 (m, 1 H), 8.26 (d, J = 6.0 Hz, 1 H), 7.88- 7.97 (m, 1 H), 7.52- 7.61 (m, 2 H), 7.46 (t, J = 7.5 Hz, 1 H), 7.18- 7.36 (m, 1 H), 6.98- 7.15 (m, 3 H), 3.84 (d, 2 H), 3.51-3.63 (m, 2 H), 3.16-3.29 (m, 2 H), 2.82-3.03 ppm (m, 5 H) | 401 | A |
| 18 | N²-[3,4- bis(methyloxy) phenyl]-N⁴-(4- fluoro-1H-indazol- 3-yl)-2,4- pyrimidinediamine trifluoroacetate | 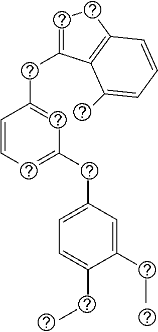 [SMILES: CCc1ccc(Cc2cccc(CC3=CCc4cccc(C)c43)c2)cc1CC] | ¹H NMR (DMSO-d₆) δ 10.24-10.60 (m, 1H), 9.59-9.94 (m, 1H), 7.97 (br. s., 1H), 7.37 (br. s., 2H), 7.04 (br. s., 1H), 6.78-6.94 (m, 2H), 6.64 (br. s., 1H). 6.39 (br. s., 1H), 3.65-3.71 (m, 3H), 3.55 (br. s., 3H) | 381 | A |
| 19 | N²-[3,4- bis(methyloxy) phenyl]-N⁴-[6- (methyloxy)-1H- indazol-3-yl]-2,4- pyrimidinediamine trifluoroacetate | 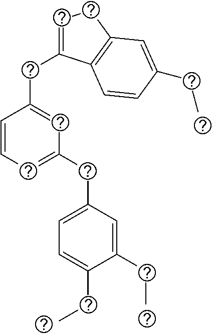 [SMILES: CCc1ccc2c(c1)CC=C2Cc1cccc(Cc2ccc(CC)c(CC)c2)c1] | ¹H NMR (DMSO-d₆) δ 12.66 (br. s., 1H), 10.71- 10.90 (m, 1H), 9.83- 9.98 (m, 1H), 7.97 (br. s., 1H), 7.62 (br. s., 1H), 7.09 (br. s., 1H), 6.97- 7.04 (m, 1H), 6.87 (d, J = 1.7 Hz, 1H), 6.80-6.86 (m, 1H), 6.69 (br. s., 1H), 3.82 (s, 3H), 3.73 (s, 3H), 3.61 (br. s., 3H) | 393 | A |
| 20 | N²-[3,4- bis(methyloxy) phenyl]-N⁴-(4,5- dichloro-1H- indazol-3-yl)-2,4- pyrimidinediamine trifluoroacetate | 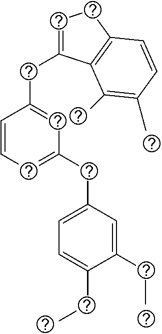 [SMILES: CCc1ccc(Cc2cccc(CC3=CCc4ccc(C)c(C)c43)c2)cc1CC] | ¹H NMR (DMSO-d₆) δ 10.50 (br. s., 1H), 9.88 (br. s., 1H), 7.94 (br. s., 1H), 7.54-7.64 (m, 2H), 6.94 (br. s., 1H), 6.78 (br. s., 1H), 6.54-6.71 (m, 1H), 6.34 (br. s., 1H), 3.69 (s, 3H), 3.58 (br. s., 3H) | 431 | A |
| 21 | N²-[3,4- bis(methyloxy) phenyl]-N⁴-(6- chloro-1-methyl- 1H-indazol-3-yl)- 2,4- pyrimidinediamine | 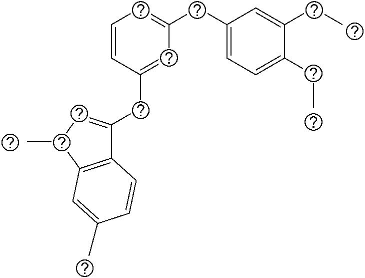 [SMILES: CCc1ccc(Cc2cccc(CC3=CC(C)c4cc(C)ccc43)c2)cc1CC] | ¹H NMR (DMSO-d₆) δ 9.78-9.88 (m, 1H), 8.95 (s, 1H), 8.10 (d, J = 5.8 Hz, 1H), 7.87 (d, J = 8.8 Hz, 1H), 7.78 (d, J = 1.3 Hz, 1H), 7.22-7.35 (m, 2H), 7.06 (dd, J = 8.6, 1.5 Hz, 1H), 6.83 (d, J = 4.5 Hz, 1H), 6.73 (d, J = 8.6 Hz, 1H), 3.98 (s, 3H), 3.69 (s, 3H). 3.62 (s, 3H) | 411 | B |
| 22 | 3-[(4-{[5- (methyloxy)-1H- indazol-3- yl]amino}-2- pyrimidinyl)amino] benzene sulfonamide trifluoroacetate | 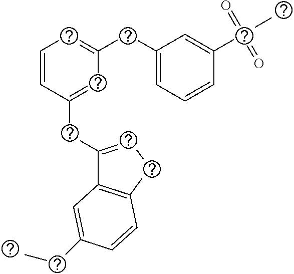 | ¹H NMR (DMSO-d₆, 400 MHz): d = 12.71- 12.87 (m, 1 H), 10.44- 10.72 (m, 1 H), 10.17- 10.36 (m, 1 H), 8.11 (d, J = 6.8 Hz, 1 H), 8.00 (br. s., 2 H), 7.46 (d, J = 9.0 Hz, 2 H), 7.39 (s, 2 H), 7.19 (br. s., 2 H), 7.07 (dd, J = 9.0, 2.3 Hz, 1 H), 6.80 (br. s., 1 H), 3.70 ppm (s, 3 H) | 412 | A |
| 23 | N²-[3,4- bis(methyloxy) phenyl]-N⁴-(5- fluoro-1H-indazol- 3-yl)-2,4- pyrimidinediamine trifluoroacetate | 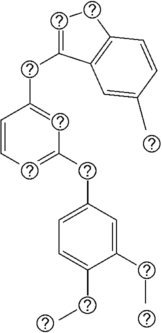 [SMILES: CCc1ccc(Cc2cccc(CC3=CCc4ccc(C)cc43)c2)cc1CC] | ¹H NMR (DMSO-d₆) δ 10.81 (br. s., 1H), 9.93 (br. s., 1H), 7.98 (br. s., 1H), 7.55 (dd, J = 9.2, 4.3 Hz, 1H), 7.43-7.52 (m, 1H), 7.27 (s, 1H), 7.04 (br. s., 2H), 6.79 (br. s., 2H), 3.72 (s, 3H), 3.51-3.61 (m, 3H) | 381 | A |
| 24 | N²-[3,4- bis(methyloxy) phenyl]-N⁴-(5,7- difluoro-1H- indazol-3-yl)-2,4- pyrimidinediamine trifluoroacetate | 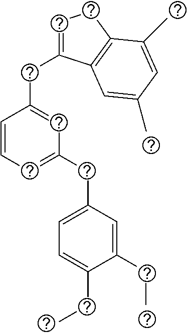 [SMILES: CCc1ccc(Cc2cccc(CC3=CCc4c(C)cc(C)cc43)c2)cc1CC] | ¹H NMR (DMSO-d₆) δ 10.81 (br. s., 1H), 9.87 (br. s., 1H), 8.01 (br. s., 1H), 7.35 (br. s., 2H), 7.02 (br. s., 2H), 6.80 (br. s., 2H), 3.72 (s, 3H), 3.56 (br. s., 3H) | 399 | A |
| 25 | N²-[3,4- bis(methyloxy) phenyl]-N⁴-(6,7- difluoro-1H- indazol-3-yl)-2,4- pyrimidinediamine trifluoroacetate | 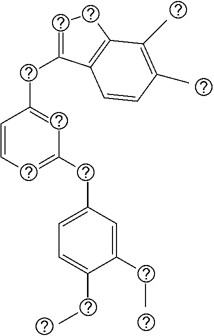 [SMILES: CCc1ccc(Cc2cccc(CC3=CCc4c3ccc(C)c4C)c2)cc1CC] | ¹H NMR (DMSO-d₆) δ 10.84 (br. s., 1H), 9.81 (br. s., 1H), 8.03 (br. s., 1H), 7.52-7.68 (m, 1H), 7.05 (d, J = 6.6 Hz, 3H), 6.84 (br. s., 2H), 3.72 (s, 3H), 3.60 (br. s., 3H) | 399 | A |
| 26 | N⁴-(5-fluoro-1H- indazol-3-yl)-N²-[3- (1-pyrrolidinyl sulfonyl)phenyl]- 2,4- pyrimidinediamine trifluoroacetate | 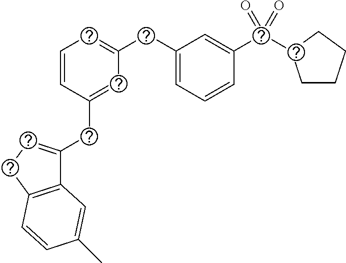 | ¹H NMR (DMSO-d₆) δ 12.84-13.01 (m, 1H), 10.42 (br. s., 1H), 9.97 (br. s., 1H), 8.12 (d, J = 6.3 Hz, 2H), 7.85 (br. s., 1H), 7.47-7.64 (m, 2H), 7.21-7.42 (m, 3H), 6.80 (br. s., 1H), 3.12 (t, J = 6.3 Hz, 4H), 1.65 (t, 4H) | 454 | C |
| 27 | N²-[4-fluoro-3- (methylsulfonyl) phenyl]-N⁴-[5- (methyloxy)-1H- indazol-3-yl]-2,4- pyrimidinediamine trifluoroacetate | 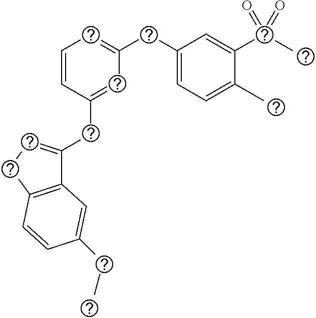 | ¹H NMR (DMSO-d₆) δ 12.54 (br. s., 1H), 9.81 (br. s., 1H), 9.61 (br. s., 1H), 8.34 (br. s., 1H), 8.11 (d, J = 6.0 Hz, 2H), 7.41 (d, J = 9.0 Hz, 1H), 7.21 (br. s., 2H), 7.03 (dd, J = 9.0, 2.5 Hz, 1H), 6.77 (br. s., 1H), 3.71 (s, 3H), 3.30 (s, 3H) | 429 | C |
| 28 | N⁴-(5-fluoro-1H- indazol-3-yl)-N²-[4- fluoro-3- (methylsulfonyl) phenyl]-2,4- pyrimidinediamine trifluoroacetate | 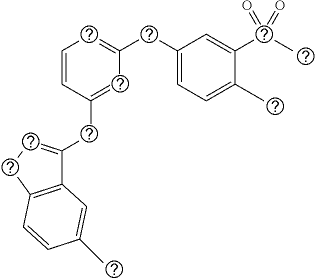 | ¹H NMR (DMSO-d₆) δ 12.71 (br. s., 1H), 9.70 (br. s., 1H), 9.46 (br. s., 1H), 8.33 (br. s., 1H), 8.14 (d, J = 5.6 Hz, 2H), 7.52 (dd, J = 9.0, 4.2 Hz, 2H), 7.14-7.30 (m, 2H), 6.67-6.88 (m, 1H), 3.22 (s., 3H) | 417 | C |
| 29 | N⁴-(5-fluoro-1H- indazol-3-yl)-N⁴- methyl-N²-[3,4,5- tris(methyloxy) phenyl]-2,4- pyrimidinediamine trifluoroacetate | 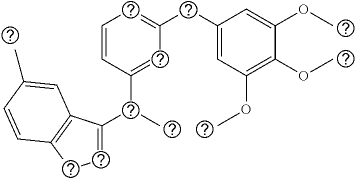 [SMILES: COc1cc(Cc2cccc(C(C)C3=CCc4ccc(C)cc43)c2)cc(OC)c1OC] | ¹H NMR (500 MHz, DMSO-d₆) δ = 9.09 (s, 1 H), 7.90 (d, J = 5.9 Hz, 1 H), 7.61 (dd, J = 3.8, 8.7 Hz, 1 H), 7.37-7.25 (m, 2 H), 7.25-7.14 (m, 2 H), 5.80 (d, J = 5.9 Hz, 1 H), 3.72 (s, 6 H), 3.61 (s, 3 H), 3.56 (s, 3 H) | 425 | A |
| 30 | N⁴-(5-fluoro-1H- indazol-3-yl)-N⁴- methyl-N²-[3- (methylsulfonyl) phenyl]-2,4- pyrimidinediamine trifluoroacetate | 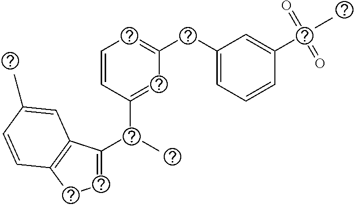 | ¹H NMR (400 MHz, DMSO-d₆) δ = 13.22 (s, 1 H), 9.74 (s, 1 H), 8.66 (br. s., 1 H), 7.98 (d, J = 5.8 Hz, 1 H), 7.87 (d, J = 3.0 Hz, 1 H), 7.65 (dd, J = 4.0, 8.6 Hz, 1 H), 7.43 (br. s., 2 H), 7.38-7.26 (m, 2 H), 5.90 (d, J = 5.8 Hz, 1 H), 3.39-3.29 (m, 3 H), 3.18 (s, 3 H) | 413 | A |
| 31 | N2-[3,4- bis(methyloxy) phenyl]-N4-[1- methyl-5- (methyloxy)-1H- indazol-3-yl]-2,4- pyrimidinediamine trifluoroacetate | 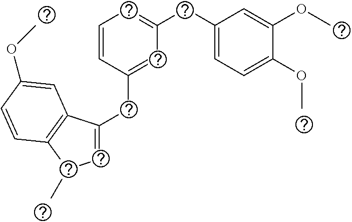 [SMILES: COc1ccc2c(c1)C(Cc1cccc(Cc3ccc(OC)c(OC)c3)c1)=CC2C] | ¹H NMR (METHANOL- d₄) δ 7.76-7.89 (m, 1H), 7.43-7.53 (m, 1H), 6.69- 7.22 (m, 5H), 6.42- 6.63 (m, 1H), 4.04 (s, 3H), 3.72-3.97 (m, 6H) | 407 | A |
| 32 | N4-[5-(methyloxy)- 1H-indazol-3-yl]- N2-[3,4,5- tris(methyloxy) phenyl]-2,4- pyrimidinediamine | 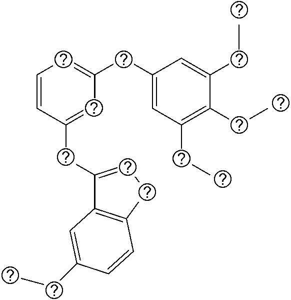 [SMILES: CCc1ccc2c(c1)C(Cc1cccc(Cc3cc(CC)c(CC)c(CC)c3)c1)=CC2] | ¹H NMR (DMSO-d₆) δ 12.49 (s, 1H), 9.53 (s, 1H), 8.93 (s, 1H), 8.06 (d, J = 5.8 Hz, 1H), 7.38 (d, J = 8.8 Hz, 1H), 7.14- 7.21 (m, 1H), 7.08 (s, 2H), 7.00 (dd, J = 9.0, 2.3 Hz, 1H), 6.56-6.64 (m, 1H), 3.64 (br. s., 3H), 3.57 (s, 6H) | 423 | F |
| 33 | N²-[4-fluoro-3- (methyloxy)phenyl]- N⁴-[5-(methyloxy)- 1H-indazol-3-yl]- 2,4- pyrimidinediamine | 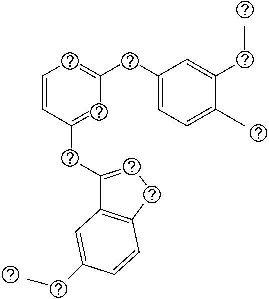 [SMILES: CCc1ccc2c(c1)C(Cc1cccc(Cc3ccc(C)c(CC)c3)c1)=CC2] | ¹H NMR (DMSO-d₆, 500 MHz): d = 12.76 (br. s., 1 H), 10.51-10.87 (m, 1 H), 9.91-10.14 (m, 1 H), 8.03 (d, J = 6.8 Hz. 1 H), 7.43 (d, J = 9.0 Hz, 1 H), 7.26-7.37 (m, 1 H), 6.95-7.15 (m, 5 H), 6.70 (br. s., 1 H), 3.63 (br. s., 3 H), 3.56 ppm (none, 3 H) | 381 | A |
| 34 | N-[2- (dimethylamino) ethyl]-N-methyl-3- [(4-{[5- (methyloxy)-1H- indazol-3- yl]amino}-2- pyrimidinyl)amino] benzene sulfonamide trifluoroacetate | 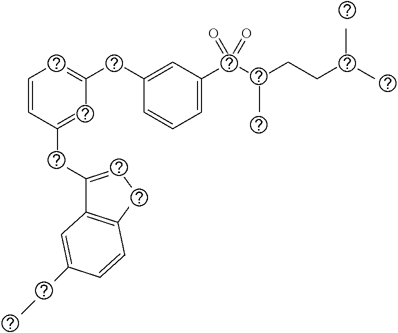 | ¹H NMR(DMSO-d₆) δ 12.75 (br. s., 1H), 9.70 (br. s., 1H), 8.12 (d, J = 6.6 Hz, 2H), 7.87 (br. s., 1H), 7.44 (m, 3H), 7.17 (br. s., 1H), 7.05 (dd, J = 9.0, 2.0 Hz, 1H), 6.78 (br. s., 1H), 3.68 (s, 3H), 2.83 (s, 3H), 2.69 (s, 3H), 2.64 (s, 3H), 2.16 (t, J = 8.1 Hz, 2H), 1.90 (s, 2H) | 497 | D |
| 35 | N,N-dimethyl-3- [(4-{[5- (methyloxy)-1H- indazol-3- yl]amino}-2- pyrimidinyl)amino] benzene sulfonamide trifluoroacetate | 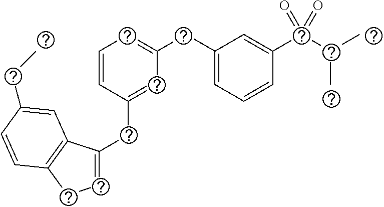 | ¹H NMR (DMSO-d₆) δ 12.71 (br. s., 1H), 10.44 (br. s., 1H), 10.06 (br. s., 1H), 8.10 (d, J = 6.3 Hz, 3H), 7.79 (br. s., 1H), 7.43 (d, J = 9.0 Hz, 1H), 7.31 (br. s., 1H), 7.16 (br. s., 1H), 7.04 (dd, J = 9.0, 2.2 Hz, 1H), 6.77 (br. s., 1H), 3.68 (s, 3H), 2.59 (s, 6H) | 440 | C |
| 36 | N4-[5-(methyloxy)- 1H-indazol-3-yl]- N2-[3-(1- pyrrolidinylsulfonyl) phenyl]-2,4- pyrimidinediamine trifluoroacetate | 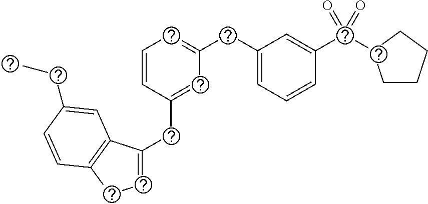 | ¹H NMR (DMSO-d₆) δ 9.91 (br. s., 1H), 9.52 (br. s., 1H), 8.15 (s, 1H), 8.05 (d, J = 6.1 Hz, 1H), 7.76-7.92 (m, 3H), 7.55 (t, J = 8.1 Hz, 2H), 7.38 (m, 2H), 3.52 (s, 3H), 3.07-3.22 (m, 4H), 1.66 (m, 4H) | 466 | C |
| 37 | N2-{3-[1- (dimethylamino)- 2,2,2-trifluoroethyl] phenyl}-N4-(5- fluoro-1H-indazol- 3-yl)-2,4- pyrimidinediamine trifluoroacetate | 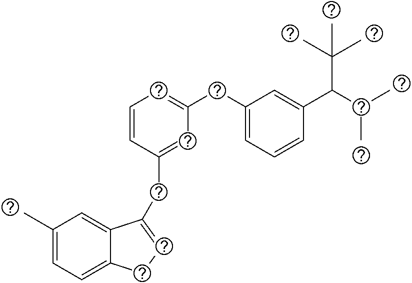 [SMILES: Cc1ccc2c(c1)C(Cc1cccc(Cc3cccc(C(C(C)C)C(C)(C)C)c3)c1)=CC2] | ¹H NMR (DMSO-d₆) δ 11.00 (br. s., 1H), 10.35 (br. s., 1H), 9.85 (s, 1H), 8.25 (s, 1H), 8.08 (d, J = 7.1 Hz, 1H), 7.87-8.02 (m, 2H), 7.45-7.63 (m, 2H), 7.22-7.42 (m, 2H), 7.07 (d, J = 5.9 Hz, 1H), 5.35 (s, 1H), 2.13 (s., 6H) | 446 | C |
| 38 | 2-[3-({4-[(5-fluoro- 1H-indazol-3- yl)amino]-2- pyrimidinyl}amino) phenyl]-2- methylpropane nitrile | 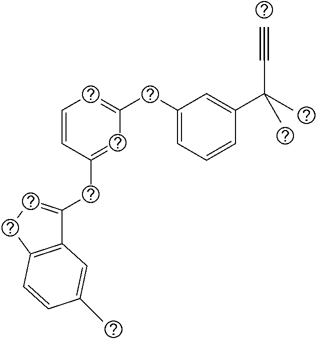 [SMILES: C#CC(C)(C)c1cccc(Cc2cccc(CC3=CCc4ccc(C)cc43)c2)c1] | ¹H NMR (DMSO-d₆) δ 12.70 (s, 1H), 9.60 (br. s., 1H), 9.17 (br. s., 1H), 8.12 (d, J = 5.9 Hz, 1H), 7.85 (d, J = 7.8 Hz, 1H), 7.74 (br. s., 1H), 7.48- 7.61 (m, 2H), 7.21-7.33 (m, 1H), 7.16 (br. s., 1H), 6.99 (d, J = 7.6 Hz, 1H), 6.79 (br. s., 1H), 1.60 (s, 6H) | 388 | C |
| 39 | N4-(5-fluoro-1H- indazol-3-yl)-N2- [3-(methylsulfonyl) phenyl]-2,4- pyrimidinediamine | 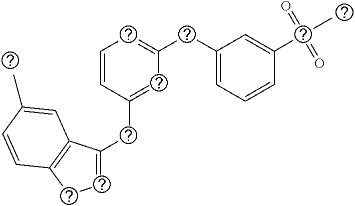 | ¹H NMR (DMSO-d₆) δ 12.69-12.80 (m, 1H), 9.75 (s, 1H), 9.52 (s, 1H), 8.21-8.29 (m, 1H), 8.13-8.21 (m, 2H), 7.56- 7.64 (m, 1H), 7.53 (dd, J = 9.1, 4.3 Hz, 1H), 7.39 (br. s., 2H), 7.27 (td, J = 9.1, 2.5 Hz, 1H), 6.80- 6.90 (m, 1H), 3.15 (s, 3H) | 398 (M⁺) | A |
| 40 | N2-(2,3-dihydro- 1,4-benzodioxin-6- yl)-N4-(5-fluoro- 1H-indazol-3-yl)- 2,4- pyrimidinediamine | 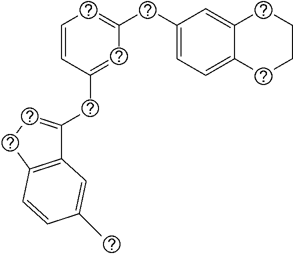 [SMILES: Cc1ccc2c(c1)C(Cc1cccc(Cc3ccc4c(c3)CCCC4)c1)=CC2] | ¹H NMR (DMSO-d₆) δ 12.67 (s, 1H), 9.61 (s, 1H), 8.88 (s, 1H), 8.07 (d, J = 5.8 Hz, 1H), 7.58- 7.64 (m, 1H), 7.50 (dd, J = 9.1. 4.0 Hz, 1H), 7.31 (br. s., 1H), 7.26 (td, J = 9.0, 2.4 Hz, 1H), 7.13- 7.19 (m, 1H), 6.75-6.83 (m, 1H), 6.58-6.65 (m, 1H), 4.17 (d, 4H) | 378 (M⁺) | A |
| 41 | N4-(5-fluoro-1H- indazol-3-yl)-N2- [4-methyl-3- (methylsulfonyl) phenyl]-2,4- pyrimidinediamine trifluoroacetate | 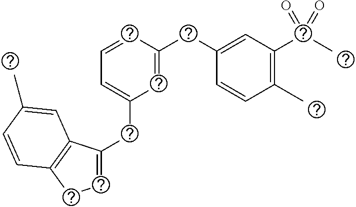 | ¹H NMR (DMSO-d₆) δ 12.96 (br. s., 1H), 10.52 (br. s., 1H), 10.03 (br. s., 1H), 8.10 (d, J = 6.5 Hz, 1H), 7.96 (d, J = 1.5 Hz, 2H), 7.56 (dd, J = 9.2, 4.1 Hz, 2H), 7.22-7.35 (m, 1H), 7.15 (br. s., 1H), 6.80 (br. s., 1H), 3.15 (s, 3H), 2.55 (s, 3H) | 413 | D |
| 42 | 2-{[3-({4-[(5-fluoro- 1H-indazol-3- yl)amino]-2- pyrimidinyl}amino) phenyl]sulfonyl}-2- methyl-1-propanol | 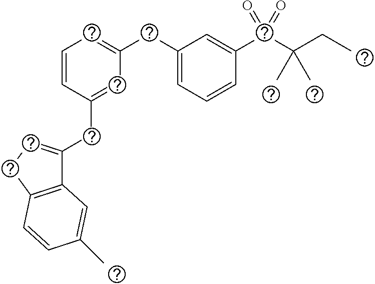 | ¹H NMR (DMSO-d₆) δ 12.75 (s, 1H), 9.72 (s, 1H), 9.48 (s, 1H), 8.32- 8.40 (m, 1H), 8.15 (d, J = 5.8 Hz, 1H), 8.03 (s, 1H), 7.56-7.64 (m, 1H), 7.52 (dd, J = 9.1, 4.3 Hz, 1H), 7.32-7.41 (m, 1H), 7.23- 7.32 (m, 2H), 6.81- 6.91 (m, 1H), 5.04 (s, 1H), 3.50 (d, J = 6.1 Hz, 2H), 1.19 (s, 6H) | 456 (M⁺) | D |
| 43 | 4-({4-[(5- FLUORO-1H- INDAZOL-3- YL)AMINO]-2- PYRIMIDINYL} AMINO)-2,6- BIS(METHYLOXY) PHENOL | 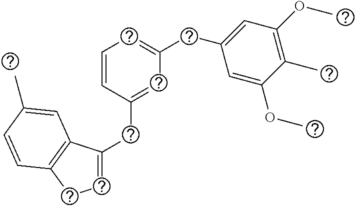 [SMILES: COc1cc(Cc2cccc(CC3=CCc4ccc(C)cc43)c2)cc(OC)c1C] | ¹H NMR (500 MHz, DMSO-d₆) δ ppm 3.51- 3.77 (m, 6 H) 6.72 (br. s., 3 H) 7.22-7.33 (m, 1 H) 7.43-7.53 (m, 1 H) 7.55 (dd, J = 9.03, 4.15 Hz, 1 H) 7.94 (br. s., 1 H) 8.29 (br. s., 1 H) | 397 | D |
| 44 | N4-(5-FLUORO- 1H-INDAZOL-3- YL)-N2-1H- INDOL-6-YL-2,4- PYRIMIDINE- DIAMINE | 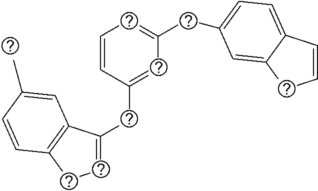 [SMILES: Cc1ccc2c(c1)C(Cc1cccc(Cc3ccc4c(c3)CC=C4)c1)=CC2] | ¹H NMR (500 MHz, DMSO-d₆) δ ppm 6.42- 6.58 (m, 1 H) 6.70-6.99 (m, 2 H) 7.19-7.28 (m, 2 H) 7.31 (td, J = 9.09, 2.08 Hz, 2 H) 7.36 (br. s., 1 H) 7.58 (dd, J = 8.91, 4.27 Hz, 2 H) 8.00 (br. s., 1 H) 9.92-10.21 (m, 1 H) 10.90-11.09 (m, 1 H) 11.23-11.36 (m, 1 H) | 360 | D |
| 45 | N4-(5-FLUORO- 1H-INDAZOL-3- YL)-N2-1H- INDOL-4-YL-2,4- PYRIMIDINE- DIAMINE | 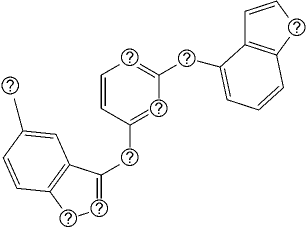 [SMILES: Cc1ccc2c(c1)C(Cc1cccc(Cc3cccc4c3C=CC4)c1)=CC2] | ¹H NMR (500 MHz, DMSO-d₆) δ ppm 6.42 (br. s., 1 H) 7.00-7.12 (m, 2 H) 7.25-7.33 (m, 2 H) 7.35 (br. s., 2 H) 7.47 (s, 1 H) 7.56 (dd, J = 9.03, 4.15 Hz, 2 H) 7.93 (br. s., 1 H) 9.94- 10.24 (m, 1 H) 10.81- 10.98 (m, 1 H) 10.99- 11.17 (m, 1 H) | 360 | D |
| 46 | N4-(5-FLUORO- 1H-INDAZOL-3- YL)-N2-[1- (METHYL- SULFONYL)-1H- INDOL-6-YL]-2,4- PYRIMIDINE- DIAMINE | 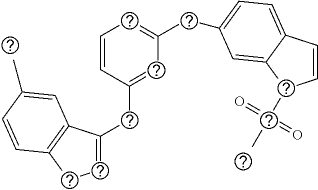 | ¹H NMR (500 MHz, DMSO-d₆) δ ppm 6.78 (d, J = 2.93 Hz, 2 H) 7.25 (br. s., 1 H) 7.36-7.67 (m, 5 H) 7.82-7.97 (m, 1 H) 8.03 (d, J = 6.35 Hz, 1 H) 10.11 (br. s., 1 H) 10.68 (br. s., 1 H) 12.97 (none, 1 H) | 438 | D |
| 47 | N4-(5-fluoro-1H- indazol-3-yl)-N2- (2-methyl-3- (methylsulfonyl) phenyl]-2,4- pyrimidinediamine | 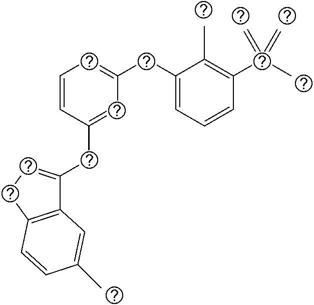 | ¹H NMR (DMSO-d₆) δ 12.64 (s, 1H), 9.66 (s, 1H), 8.49 (s, 1H), 8.06 (d, J = 5.8 Hz, 1H), 7.83 (d, J = 7.6 Hz, 1H), 7.69 (d, J = 7.3 Hz, 1H), 7.55- 7.64 (m, 1H), 7.47 (dd, J = 9.1, 4.3 Hz, 1H), 7.33 (t, J = 8.0 Hz, 1H), 7.24 (td, J = 9.1, 2.5 Hz, 1H), 6.84-6.94 (m, 1H), 3.17- 3.21 (m, 3H), 2.53 (s, 3H) | 412 (M⁺) | E |
| 48 | N4-(5-FLUORO- 1H-INDAZOL-3- YL)-N2-[1- (METHYL- SULFONYL)-1H- INDOL-5-YL]-2,4- PYRIMIDINE- DIAMINE | 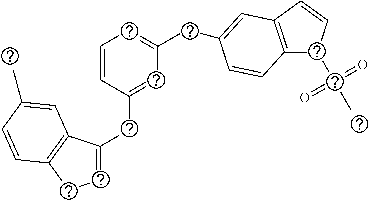 | ¹H NMR (500 MHz, DMSO-d₆) δ ppm 3.17 (s, 3 H) 6.52 (br. s., 1 H) 6.63-6.81 (m, 1 H) 7.28 (td, J = 9.03, 2.20 Hz, 1 H) 7.32-7.44 (m, 1 H) 7.49-7.56 (m, 2 H) 7.59 (dd, J = 9.03, 4.15 Hz, 2 H) 7.62-7.72 (m, 1 H) 7.72-7.91 (m, 1 H) 8.04 (d, J = 6.59 Hz, 1 H) 10.21-10.41 (m, 1 H) 10.92 (br. s., 1 H) | 438 | D |
| 49 | N4-(7-CHLORO- 1H-INDAZOL-3- YL)-N2-[4- METHYL-3- (METHYL- SULFONYL) PHENYL]-2,4- PYRIMIDINE- DIAMINE | 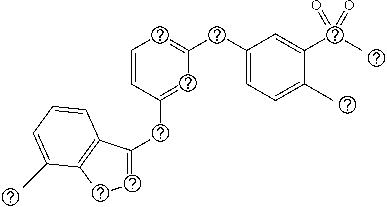 | ¹H NMR (400 MHz, DMSO-d₆) δ ppm 2.54 (s, 3 H) 3.16 (s, 3 H) 6.87 (br. s., 1 H) 7.07 (t, 1 H) 7.15-7.25 (m, 1 H) 7.44-7.52 (m, 1 H) 7.84 (d, J = 8.03 Hz, 1 H) 8.12 (br. s., 1 H) 8.16 (d, J = 5.77 Hz, 2 H) 9.49 (br. s., 1 H) 9.95 (br. s., 1 H) 13.16 (s, 1 H) | 429 | D |
| 50 | N2-[4-methyl-3- (methylsulfonyl) phenyl]-N4-[7- (trifluoromethyl)- 1H-indazol-3-yl]- 2,4- pyrimidinediamine | 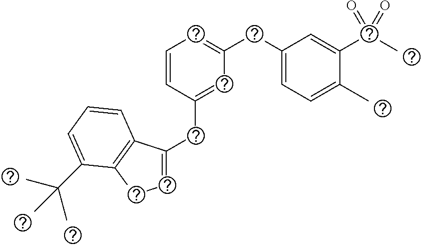 | ¹H NMR (400 MHz, DMSO-d₆) δ ppm 2.52- 2.56 (m, 3 H) 3.11-3.23 (m, 3 H) 6.87 (br. s., 1 H) 7.16 (d, J = 8.03 Hz, 1 H) 7.23 (t, J = 7.65 Hz, 1 H) 7.77 (d, J = 7.28 Hz, 1 H) 8.05-8.24 (m, 4 H) 9.41 (br. s., 1 H) 9.91 (br. s., 1 H) 12.46-13.70 (m, 1 H) | 463 | D |
| 51 | N4-(5-FLUORO- 1H-INDAZOL-3- YL)-N2-[3- METHYL-5- (METHYL- SULFONYL) PHENYL]-2,4- PYRIMIDINE- DIAMINE | 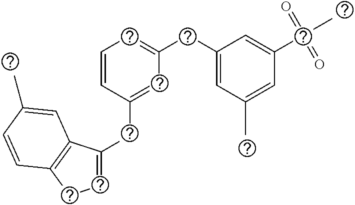 | ¹H NMR (400 MHz, DMSO-d₆) δ 12.76 (s, 1H), 9.77 (s, 1H), 9.46 (s, 1H), 8.15 (d, J = 5.81 Hz, 1H), 7.99 (s, 1H), 7.96 (s, 1H), 7.53 (dd, J = 4.29, 8.84 Hz, 2H), 7.23-7.32 (m, 1H), 7.20 (s, 1H), 6.76 (br. s., 1H), 3.13 (s, 3H), 2.17 (br. s., 3H) | 413 | E |
| 52 | N4-(5-fluoro-1H- indazol-3-yl)-N2- [4-(methylsulfonyl) phenyl]-2,4- pyrimidinediamine | 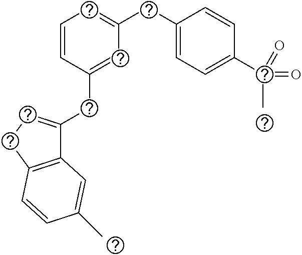 | ¹H NMR (DMSO-d₆) δ 12.77-12.82 (m, 1H), 9.79-9.84 (m, 1H), 9.72- 9.77 (m, 1H), 8.19 (d, J = 5.8 Hz, 0H), 8.00 (d, J = 8.6 Hz, 0H), 7.65 (d, J = 8.6 Hz, 2H), 7.55 (dd, J = 9.1, 4.3 Hz, 1H), 7.29 (td, J = 9.0, 2.5 Hz, 1H), 3.12 (s, 3H) | 399 | E |
| 53 | N4-(5-fluoro-1H- indazol-3-yl)-N2- [3-(4-morpholinyl sulfonyl)phenyl]- 2,4- pyrimidinediamine | 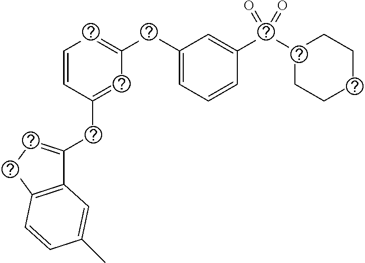 | ¹H NMR (DMSO-d₆) δ 12.69-12.81 (m, 1H), 9.73 (s, 1H), 9.46-9.53 (m, 1H), 8.29-8.35 (m, 1H), 8.16 (d, J = 5.8 Hz, 1H), 7.97-8.00 (m, 1H), 7.56-7.62 (m, 1H), 7.53 (dd, J = 9.1, 4.0 Hz, 1H), 7.34-7.41 (m, 1H), 7.28 (td, J = 9.1, 2.3 Hz, 1H), 7.20 (s, 1H), 6.80-6.88 (m, 1H), 3.60-3.68 (m, 4H), 2.82-2.91 (m, 4H) | 469 (M⁺) | E |
| 54 | 3-({4-[(5- FLUORO-1H- INDAZOL-3- YL)AMINO]-2- PYRIMIDINYL} AMINO)-N- (METHYLOXY) BENZENE SULFONAMIDE | 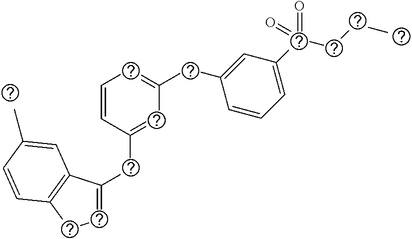 | ¹H NMR (400 MHz, DMSO-d₆) δ ppm 3.66 (s, 3 H) 6.84 (br. s., 1 H) 7.30 (td, J = 9.10, 2.38 Hz, 2 H) 7.43 (d, J = 7.78 Hz, 1 H) 7.57 (dd, J = 9.03, 4.27 Hz, 2 H) 7.93 (br. s., 1 H) 8.14 (d, J = 6.27 Hz, 2 H) 10.11 (br. s., 1 H) 10.52 (s, 2 H) 12.97 (br. s., 1 H) | 430 | D |
| 55 | 3-({4-[(5- FLUORO-1H- INDAZOL-3- YL)AMINO]-2- PYRIMIDINYL} AMINO)-N- METHYL-N- (METHYLOXY) BENZENE SULFONAMIDE | 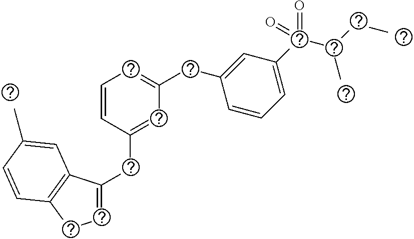 | ¹H NMR (400 MHz, DMSO-d₆) δ ppm 2.73 (s, 3 H) 3.71 (s, 3 H) 6.82 (br. s., 1 H) 7.28 (td, J = 9.03, 2.51 Hz, 1 H) 7.37 (br. s., 2 H) 7.55 (dd, J = 9.03, 4.27 Hz, 2 H) 7.91 (br. s., 1 H) 8.14 (d, J = 6.27 Hz, 1 H) 8.25 (br. s., 1 H) 9.99 (br. s., 1 H) 10.37 (br. s., 1 H) 12.93 (br. s., 1 H) | 444 | D |
| 56 | 5-({4-[(5- FLUORO-1H- INDAZOL-3- YL)AMINO]-2- PYRIMIDINYL} AMINO)-N,2- DIMETHYL-N- (METHYLOXY) BENZENE SULFONAMIDE | 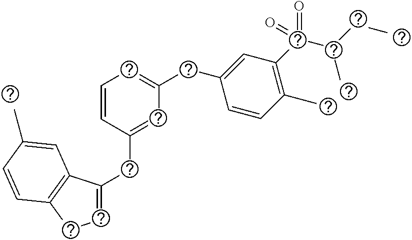 | ¹H NMR (400 MHz, DMSO-d₆) δ ppm 2.53 (s, 3 H) 2.78 (s, 3 H) 3.58 (s, 3 H) 6.80 (br. s., 1 H) 7.10-7.23 (m, 1 H) 7.28 (td, J = 9.10, 2.38 Hz, 1 H) 7.55 (dd, J = 9.03, 4.27 Hz, 2 H) 7.87 (br. s., 1 H) 8.10 (d, J = 6.52 Hz, 2 H) 9.81- 10.26 (m, 1 H) 10.26- 10.84 (m, 1 H) 12.81- 13.17 (m, 1 H) | 458 | D |
| 57 | N2-[3- (ETHYLOXY)-5- (METHYL- SULFONYL) PHENYL]-N4-(5- FLUORO-1H- INDAZOL-3-YL)- 2,4-PYRIMIDINE- DIAMINE | 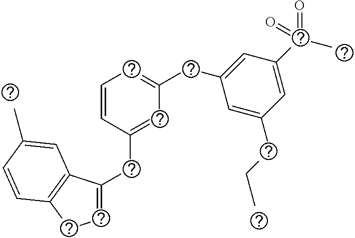 | ¹H NMR (400 MHz, DMSO-d₆) δ ppm 1.28 (t, J = 6.90 Hz, 3 H) 3.10- 3.23 (m, 2 H) 3.87 (s, 3 H) 6.77 (br. s., 1 H) 6.99 (s, 1 H) 7.26 (td, J = 9.03, 2.26 Hz, 1 H) 7.42-7.50 (m, 1 H) 7.54 (dd, J = 9.03, 4.27 Hz, 1 H) 7.56-7.66 (m, 2 H) 8.13 (d, J = 6.27 Hz, 1 H) 9.97 (m, 1 H) 10.37-10.79 (m, 1 H) 12.77-13.09 (m, 1 H) | 443 | D |
| 58 | N4-(5-FLUORO- 1H-INDAZOL-3- YL)-N2-{3-[(1- METHYLETHYL) SULFONYL] PHENYL}-2,4- PYRIMIDINE- DIAMINE | 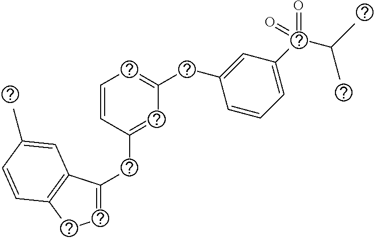 | ¹H NMR (400 MHz, DMSO-d₆) δ ppm 1.11- 1.21 (m, 6 H) 3.29 (m, 1 H) 6.84 (br. s., 1 H) 7.23- 7.34 (m, 2 H) 7.38 (br. s., 1 H) 7.53 (dd, J = 9.03, 4.27 Hz, 1 H) 7.59 (d, J = 9.29 Hz, 1 H) 8.08 (s, 1 H) 8.16 (d, J = 5.77 Hz, 1 H) 8.32 (br. s., 1 H) 9.51 (s, 1 H) 9.75 (s, 1 H) 12.75 (s, 1 H) | 427 | D |
| 59 | N2-(3,5- dimethylphenyl)- N4-(5-fluoro-1H- indazol-3-yl)-2,4- pyrimidinediamine | 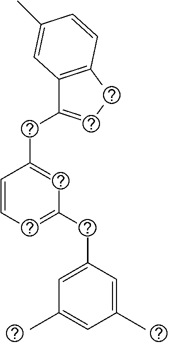 [SMILES: Cc1cc(C)cc(Cc2cccc(CC3=CCc4ccc(C)cc43)c2)c1] | ¹H NMR (DMSO-d₆) δ 12.72 (s, 1H), 9.61 (s, 1H), 8.91 (s, 1H), 8.09 (d, J = 5.8 Hz, 1H), 7.52 (dd, J = 9.0, 4.4 Hz, 2H), 7.24-7.32 (m, 3H), 6.69 (br. s., 1H), 6.46 (s, 1H), 2.06 (s, 6H) | 349 | E |
| 60 | N4-(5-fluoro-1H- indazol-3-yl)-N2- {4-methyl-3-[(1- methylethyl) sulfonyl]phenyl}- 2,4- pyrimidinediamine trifluoroacetate | 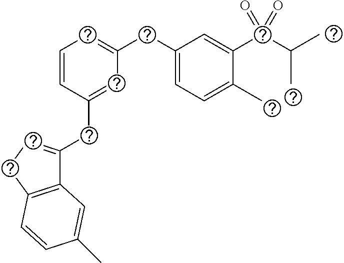 | ¹H NMR (DMSO-d₆) δ 12.95 (br. s., 1H), 10.53 (s, 1H), 9.98 (s, 1H), 8.10 (d, J = 6.3 Hz, 2H), 7.88 (br. s., 1H), 7.56 (dd, J = 9.0, 4.3 Hz, 2H), 7.28 (td, J = 9.1, 2.4 Hz, 1H), 7.14 (br. s., 1H), 6.79 (br. s., 1H), 3.38 (s., 1H), 3.17 (s., 3H) 1.15 (d, J = 6.8 Hz, 6H) | 441 | E |
| 61 | N4-(5-fluoro-1H- indazol-3-yl)-N2- [4-methyl-3- (methylsulfonyl) phenyl]-2,4- pyrimidinediamine | 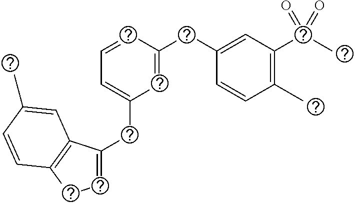 | ¹H NMR (DMSO-d₆) δ 12.76 (br. s., 1H), 9.83 (br. s., 1H), 9.48 (br. s., 1H), 8.14 (d, J = 5.8 Hz, 3H), 7.53 (dd, J = 9.0, 4.2 Hz, 2H), 7.28 (td, J = 9.0, 2.4 Hz, 2H), 6.83 (br. s., 1H), 3.17 (s, 3H), 2.17 (s, 3H) | 413 | E |
| 62 | 2-(ethyloxy)-5-({4- [(5-fluoro-1H- indazol-3- yl)amino]-2- pyrimidinyl}amino)- N,N-dimethyl benzene sulfonamide trifluoroacetate | 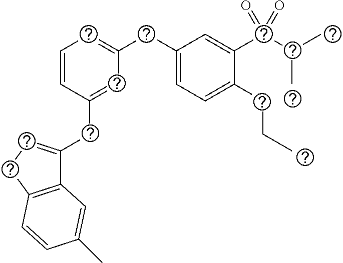 | ¹H NMR (DMSO-d₆) δ 12.97-13.05 (m, 1H), 10.69-10.88 (m, 1H), 9.94-10.12 (m, 1H), 8.01-8.07 (m, 2H), 7.78-7.91 (m, 1H), 7.65-7.73 (m, 1H), 7.56 (dd, J = 9.1, 4.3 Hz, 1H), 7.43-7.52 (m, 1H), 7.23-7.31 (m, 1H), 6.86-7.03 (m, 1H), 6.67-6.82 (m, 1H), 4.05-4.16 (m, 2H), 2.70 (s, 6H), 1.37 (s, 3H) | 472 | G |
| 63 | N4-(5-FLUORO- 1H-INDAZOL-3- YL)-N2-[3- (METHYLOXY)-5- (METHYL- SULFONYL) PHENYL]-2,4- PYRIMIDINE- DIAMINE | 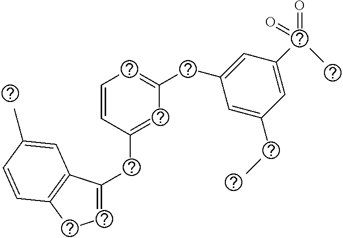 | ¹H NMR (500 MHz, DMSO-d₆) δ ppm 3.14 (s, 3 H) 3.68 (br. s., 3 H) 6.79 (br. s., 1 H) 6.99 (s, 1 H) 7.26 (td, J = 9.09, 2.32 Hz, 1 H) 7.44-7.50 (m, 1 H) 7.53 (dd, J = 9.03, 4.15 Hz, 1 H) 7.65 (m, 2 H) 8.12 (d, J = 6.10 Hz, 1 H) 9.86 (br. s., 1 H) 10.31 (br. s., 1 H) 12.86 (br. s., 1 H) | 429 | D |
| 64 | N2-[3-(ETHYL SULFONYL)-5- (METHYLOXY) PHENYL]-N4-(5- FLUORO-1H- INDAZOL-3-YL)- 2,4-PYRIMIDINE- DIAMINE | 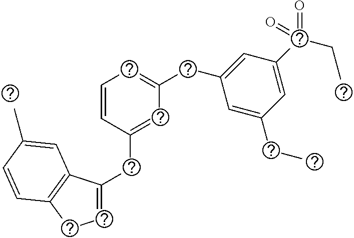 | ¹H NMR (500 MHz, DMSO-d₆) δ ppm 1.10 (t, J = 7.32 Hz, 3 H) 3.20 (m, 2 H) 3.66 (br. s., 3 H) 6.77 (br. s., 1 H) 6.94 (s, 1 H) 7.25 (td, J = 9.03, 2.20 Hz, 1 H) 7.42-7.50 (m, 1 H) 7.53 (dd, 2 H) 7.68 (br. s., 1 H) 8.12 (d, J = 6.35 Hz. 1 H) 9.81-10.09 (m, 1 H) 10.24-10.61 (m, 1 H) 12.75-13.01 (m, 1 H) | 443 | D |
| 65 | N2-{4-(ethyloxy)-3- [(1-methylethyl) sulfonyl]phenyl}- N4-(5-fluoro-1H- indazol-3-yl)-2,4- pyrimidinediamine | 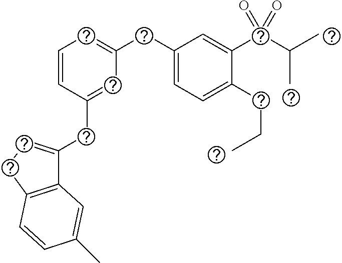 | ¹H NMR (DMSO-d₆) δ 12.76-12.81 (m, 1H), 9.88-10.01 (m, 1H), 9.34-9.46 (m, 1H), 8.12-8.21 (m, 1H), 8.09 (d, J = 6.1 Hz, 1H), 7.89-7.93 (m, 1H), 7.55-7.61 (m, 1H), 7.52 (dd, J = 9.0, 4.2 Hz, 1H), 7.27 (s, 1H), 6.98-7.05 (m, 1H), 6.76-6.85 (m, 1H), 4.14 (d, J = 6.8 Hz, 2H), 3.64-3.74 (m, 1H), 1.35 (t, J = 6.9 Hz, 3H), 1.16 (d, 6H) | 471 | E |
| 66 | N4-(5-FLUORO- 1H-INDAZOL-3- YL)-N4-METHYL- N2-[4-METHYL-3- (METHYL- SULFONYL) PHENYL]-2,4- PYRIMIDINE- DIAMINE | 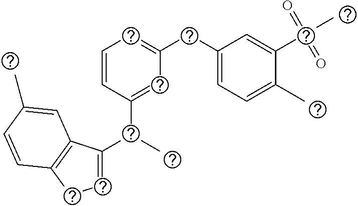 | ¹H NMR (400 MHz, DMSO-d₆) δ 13.20 (s, 1H), 9.60 (s, 1H), 8.65 (br. s., 1H), 7.94 (d, J = 6.06 Hz, 1H), 7.78 (d, J = 8.34 Hz, 1H), 7.64 (dd, J = 4.29, 9.85 Hz, 1H), 7.28-7.38 (m, 2H), 7.24 (d, J = 8.08 Hz, 1H), 5.85 (d, J = 6.06 Hz, 1H), 3.56 (s, 3H), 3.18 (s, 3H), 2.56 (s, 3H) | 427 | A |
| 67 | N4-(5-FLUORO- 1H-INDAZOL-3- YL)-N4-METHYL- N2-[3-METHYL-5- (METHYL- SULFONYL) PHENYL]-2,4- PYRIMIDINE- DIAMINE | 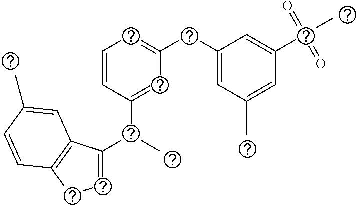 | ¹H NMR (400 MHz, DMSO-d₆) δ 13.21 (s, 1H), 9.65 (s, 1H), 8.43 (br. s., 1H), 7.98 (d, J = 5.81 Hz, 1H), 7.75 (s, 1H), 7.63 (dd, J = 4.29, 9.60 Hz, 1H), 7.28- 7.36 (m, 2H), 7.26 (s, 1H), 5.90 (d, J = 5.81 Hz, 1H), 3.57 (s, 3H), 3.16 (s, 3H), 2.31 (s, 3H) | 427 | A |
| 68 | N4-ethyl-N4-(5- fluoro-1H-indazol- 3-yl)-N2-[3-methyl- 5-(methylsulfonyl) phenyl]-2,4- pyrimidinediamine | 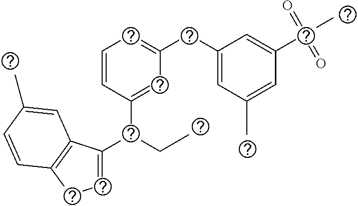 | ¹H NMR (CHLOROFORM-d) δ 8.27 (t, J = 7.7 Hz, 2H), 7.56 (d, J = 8.3 Hz, 2H), 7.43 (d, J = 11.6 Hz, 2H), 7.28 (s, 2H), 3.50 (q, J = 6.9 Hz, 1H), 2.98- 3.15 (m, 3H), 2.49 (q, J = 7.2 Hz, 2H), 1.54- 1.67 (m, 3H), 0.98- 1.08 (m, 3H) | 441 | E |
| 69 | N⁴-(5-fluoro-1H- indazol-3-yl)-N²-[3- (methylsulfonyl)-5- (4-morpholinyl) phenyl]-2,4- pyrimidinediamine | 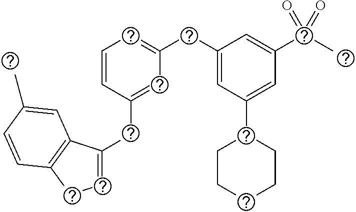 | ¹H NMR (400 MHz, DMSO-d₆) δ ppm 3.02 (br. s., 4 H) 3.14 (s, 3 H) 3.68-3.75 (m, 4 H) 6.74 (br: s., 1 H) 6.91 (s, 1 H) 7.26 (td, J = 9.03, 2.40 Hz, 1 H) 7.45-7.59 (m, 2 H) 7.73 (d, J = 9.35 Hz, 2 H) 8.15 (d, J = 5.56 Hz, 1 H) 9.28 (s, 1 H) 9.71 (s, 1 H) 12.73 (br. s., 1 H) | 484 | D |
| 70 | N4-(5-fluoro-1H- indazol-3-yl)-N2- (1-methyl-1H- imidazol-2-yl)-2,4- pyrimidinediamine | 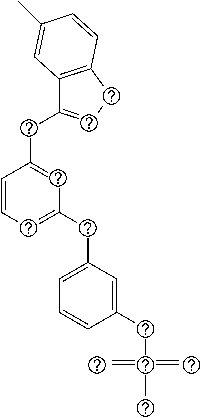 | ¹H NMR (DMSO-d₆) δ 12.69 (br. s., 1H), 9.57 (br. s., 1H), 9.10 (br. s., 1H), 8.12 (d, J = 5.8 Hz, 1H), 7.58-7.67 (m, 2H), 7.48-7.55 (m, 2H), 7.27 (td, J = 9.1, 2.3 Hz, 1H), 7.06 (t, J = 8.2 Hz, 1H), 6.86 (br. s., 1H), 6.73 (d, J = 7.6 Hz, 1H), 5.76 (s, 1H), 2.97 (s, 3H). | 414 | E |
| 71 | N4-(5-fluoro-1H- indazol-3-yl)-N2- [3-(methyloxy)-5- (methylsulfonyl) phenyl]-2,4- pyrimidinediamine | 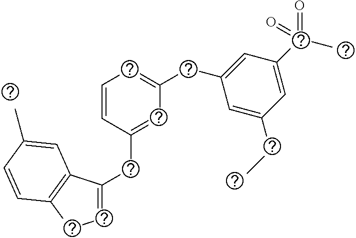 | ¹H NMR (DMSO-d₆) δ 12.72 (s, 1H), 9.75 (s, 1H), 9.46 (s, 1H), 8.16 (d, J = 5.8 Hz, 1H), 7.87 (s, 1H), 7.80 (s, 1H), 7.46-7.60 (m, 2H), 7.26 (td, J = 9.0, 2.4 Hz, 1H), 6.92 (s, 1H), 6.79-6.86 (m, 1H), 3.73 (s, 3H), 3.16 (s, 3H). | 429 | E |
| 72 | N⁴-(5-fluoro-1H- indazol-3-yl)-N²-[3- fluoro-5- (methylsulfonyl) phenyl]-2,4- pyrimidinediamine | 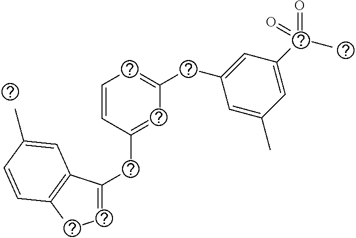 | ¹H NMR (DMSO-d₆) δ 12.85 (br. s., 1H), 10.20 (br. s., 1H), 10.04 (br. s., 1H), 8.11-8.31 (m, 2H), 7.84 (br. s., 1H), 7.55 (dd, J = 9.0, 4.3 Hz, 2H), 7.20-7.34 (m, 2H), 6.83 (br. s., 1H) | 417 | D |
| 73 | 2-{[5-({4-[(5-fluoro- 1H-indazol-3- yl)amino]-2- pyrimidinyl}amino)- 2-methylphenyl] sulfonyl}-2-methyl- 1-propanol | 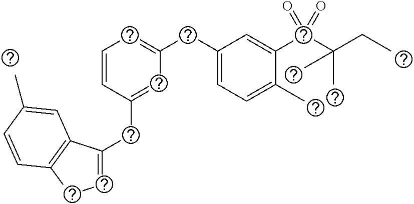 | ¹H NMR (methanol-d₄) δ 8.08 (d, J = 5.8 Hz, 2H), 7.89 (dd, J = 8.5, 1.9 Hz, 1H), 7.38-7.58 (m, 2H), 7.10-7.32 (m, 2H), 6.73 (br. s., 1H), 3.75 (s, 3H), 3.14 (s, 2H), 1.34 (s, 6H) | 471 | E |
| 74 | N4-(5-fluoro-1H- indazol-3-yl)-N2- {4-fluoro-3-[(1- methylethyl) sulfonyl]phenyl}- 2,4- pyrimidinediamine | 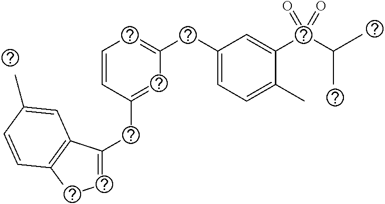 | ¹H NMR (METHANOL- d₄) δ 8.02-8.16 (m, 3H), 7.51 (dd, J = 9.1, 4.0 Hz, 1H), 7.40 (dd, J = 8.8, 2.3 Hz, 1H), 7.23 (td, J = 9.0, 2.4 Hz, 1H), 7.09 (t, J = 9.2 Hz, 1H), 6.71 (d, J = 5.6 Hz, 1H), 3.43-3.55 (m, 1H), 1.31 (d, 6H) | 445 | E |
| 75 | N4-(5-fluoro-1H- indazol-3-yl)-N4- methyl-N2-[3- (methyloxy)-5- (methylsulfonyl) phenyl]-2,4- pyrimidinediamine | 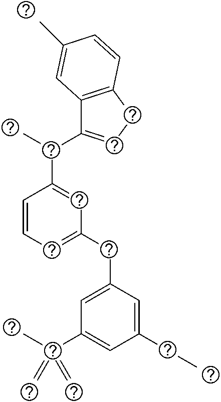 | ¹H NMR (DMSO-d₆) δ 13.21 (br. s., 1H), 9.70 (s, 1H), 8.20 (s, 1H), 7.96 (d, J = 6.1 Hz, 1H), 7.59-7.70 (m, 2H), 7.27-7.37 (m, 2H), 6.94-6.99 (m, 1H), 3.82 (s, 3H), 3.57 (s, 3H), 3.19 (s, 3H) | 443 | A |
| 76 | N⁴-(5-fluoro-1H- indazol-3-yl)-N²-[3- fluoro-4-methyl-5- (methylsulfonyl) phenyl]-2,4- pyrimidinediamine | 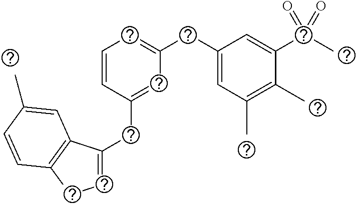 | ¹H NMR(400 MHz, METHANOL-d₄) d ppm 2.49 (d, 3 H) 3.15 (s, 3 H) 6.71 (d, J = 5.31 Hz, 1 H) 7.23 (td, J = 9.03, 2.40 Hz, 1 H) 7.39 (dd, J = 8.97, 2.15 Hz, 1 H) 7.51 (dd, J = 9.09, 3.79 Hz, 1 H) 7.88 (s, 1 H) 8.03-8.17 (m, 2 H) | 431 | D |
| 77 | N-[3-({4-[(5-fluoro- 1H-indazol-3- yl)amino]-2- pyrimidinyl}amino) phenyl]-N- methylmethane sulfonamide | 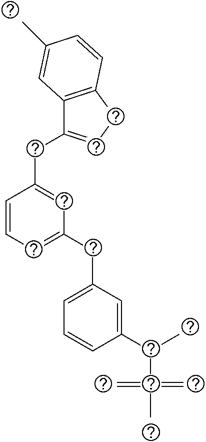 | ¹H NMR (DMSO-d₆) δ 12.73 (s, 1H), 9.65 (s, 1H), 9.19 (s, 1H), 8.13 (d, J = 5.8 Hz, 1H), 7.79- 7.92 (m, 1H), 7.49- 7.71 (m, 3H), 7.24- 7.34 (m, 1H), 7.10- 7.19 (m, 1H), 6.92 (d, J = 7.6 Hz, 1H), 6.81 (br. s., 1H), 3.16 (s, 3H), 2.94 (s, 3H). | 428 | D |
| 78 | N²-[3- (dimethylamino)-5- (methylsulfonyl) phenyl]-N⁴-(5- fluoro-1H-indazol- 3-yl)-2,4- pyrimidinediamine | 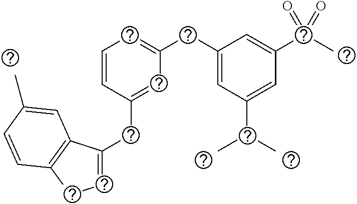 | ¹H NMR (DMSO-d₆) □: 12.72 (br. s., 1H), 9.66 (br. s., 1H), 9.18 (br. s., 1H), 8.14 (d, J = 5.8 Hz, 1H), 7.61 (br. s., 1H), 7.44-7.58 (m, 3H), 7.25 (t, J = 8.8 Hz, 1H), 6.79 (br. s., 1H), 6.68 (br. s., 1H), 3.11 (s, 3H), 2.87 (br. s., 3H). | 442 | D |
| 79 | 3-({4-[(5-fluoro-1H- indazol-3- yl)amino]-2- pyrimidinyl}amino) benzamide | 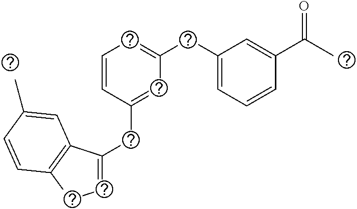 [SMILES: CC(=O)c1cccc(Cc2cccc(CC3=CCc4ccc(C)cc43)c2)c1] | ¹H NMR (DMSO-d₆) δ 12.82 (br. s., 1H), 10.02- 10.19 (m, 1H), 9.43- 9.60 (m, 1H), 8.12 (d, J = 6.3 Hz, 1H), 7.96 (br. s., 2H), 7.84 (br. s., 1H), 7.57-7.63 (m, 1H), 7.54 (dd, J = 9.0, 4.3 Hz, 1H), 7.42 (d, J = 7.8 Hz, 1H), 7.24-7.34 (m, 2H), 7.12-7.22 (m, 1H), 6.86 (none, 1H) | 364 | E |
| 80 | N4-(5-fluoro-1H- indazol-3-yl)-N2- [2-fluoro-4- (methylsulfonyl) phenyl]-2,4- pyrimidinediamine | 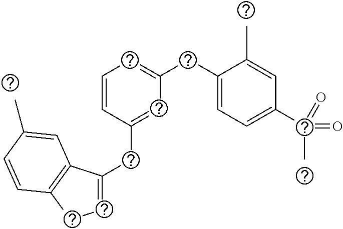 | ¹H NMR (DMSO-d₆) δ 12.82-12.90 (m, 1H), 10.03-10.21 (m, 1H), 9.06-9.20 (m, 1H), 8.40-8.50 (m, 1H), 8.20 (d, J = 6.0 Hz, 1H), 7.74 (dd, J = 10.7, 1.9 Hz, 1H), 7.57-7.63 (m, 1H), 7.54 (dd, J = 9.3, 4.3 Hz, 2H), 7.28 (td, J = 9.1, 2.4 Hz, 1H), 6.92 (none, 1H) | 417 | E |
| 81 | N4-(5-fluoro-1H- indazol-3-yl)-N2- [3-(methylsulfonyl)- 5-(1-pyrrolidinyl) phenyl]-2,4- pyrimidinediamine | 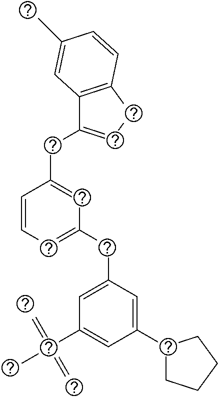 | ¹H NMR (DMSO-d₆) δ 12.72 (s, 1H), 9.66 (s, 1H), 9.18 (s, 1H), 8.09- 8.19 (m, 1H), 7.43- 7.57 (m, 3H), 7.19- 7.34 (m, 2H), 6.74 (br. s., 1H), 6.49 (s, 1H), 3.03-3.14 (m, 7H), 1.87-1.95 (m, 4H) | 468 | D |
| 82 | N2-[3- (ETHYLOXY)-5- (ETHYL SULFONYL) PHENYL]-N4-(5- FLUORO-1H- INDAZOL-3-YL)- 2,4-PYRIMIDINE- DIAMINE | 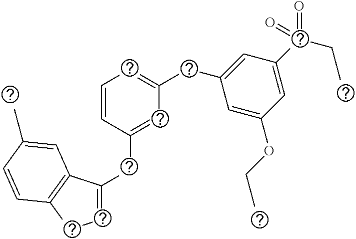 | ¹H NMR (400 MHz, DMSO-d₆) δ ppm 1.11 (m, 6 H) 3.21 (m, 4 H) 6.79 (br. s., 1 H) 6.96 (s, 1 H) 7.26 (td, J = 9.03, 2.15 Hz, 1 H) 7.47 (br. s., 1 H) 7.52- 7.61 (m, 2 H) 7.69 (br. s., 1 H) 8.13 (d, J = 6.57 Hz, 1 H) 9.80-10.20 (m, 1 H) 10.49 (m, 1 H) 12.71-13.08 (m, 1 H) | 457 | D |
| 83 | N4-(5-fluoro-1H- indazol-3-yl)-N2- [4- (methylsulfonyl)- 2,3-dihydro-1- benzofuran-6-yl]- 2,4- pyrimidinediamine | 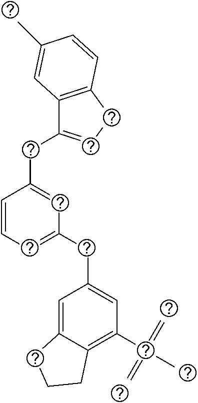 | ¹H NMR (DMSO-d₆) δ 12.71 (br. s., 1H), 9.63 (br. s., 1H), 9.14 (d, J = 1.3 Hz, 1H), 8.09 (d, J = 6.1 Hz, 2H), 7.45-7.85 (m, 3H), 7.26 (br. s., 1H), 6.73 (br. s., 1H), 4.67 (br. s., 2H), 3.01- 3.33 (m, 5H). | 441 | D |
| 84 | N4-(5-FLUORO- 1H-INDAZOL-3- YL)-N2-[4- FLUORO-3- (METHYL- SULFONYL) PHENYL]-N4- METHYL-2,4- PYRIMIDINE- DIAMINE | 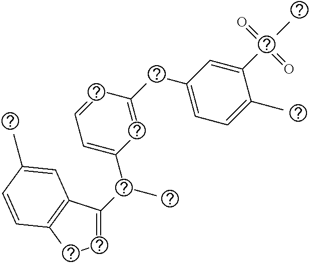 | ¹H NMR (400 MHz, DMSO-d₆) δ 13.21 (s, 1H), 9.72 (s, 1H), 8.62 (br. s., 1H), 7.96 (d, J = 5.81 Hz, 1H), 7.91 (dd, J = 4.04, 7.83 Hz, 1H), 7.64 (dd, J = 4.29, 9.85 Hz, 1H), 7.26-7.39 (m, 3H), 5.88 (d, J = 6.06 Hz, 1H), 3.56 (s, 3H), 3.32 (s, 3H) | 431 | A |
| 85 | N4-(5-fluoro-1H- indazol-3-yl)-N4- methyl-N2-[2- methyl-3- (methylsulfonyl) phenyl]-2,4- pyrimidinediamine | 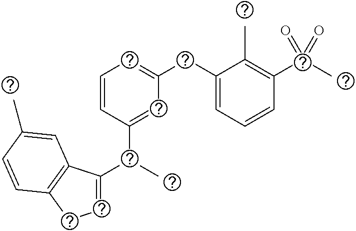 | ¹H NMR (DMSO-d₆) δ 13.12-13.28 (m, 1H), 8.61 (s, 1H), 8.52 (s, 1H), 7.89 (d, J = 6.1 Hz, 1H), 7.61 (dd, J = 9.6, 4.3 Hz, 1H), 7.41-7.52 (m, 2H), 7.27-7.34 (m, J = 6.7, 4.6, 2.5, 2.5 Hz, 2H), 5.80 (d, J = 5.8 Hz, 1H), 3.48 (s, 3H), 3.16 (s, 3H), 2.38 (s, 3H) | 427 | E |
| 86 | N2-[3,4- DIMETHYL-5- (METHYL- SULFONYL) PHENYL]-N4-(5- FLUORO-1H- INDAZOL-3-YL)- N4-METHYL-2,4- PYRIMIDINE- DIAMINE | 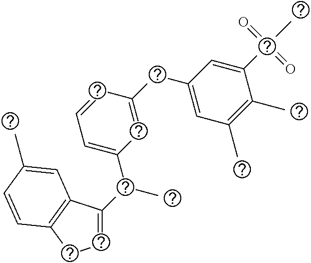 | ¹H NMR (400 MHz, DMSO-d₆) δ 13.19 (s, 1H), 9.51 (s, 1H), 8.51 (br. s., 1H), 7.94 (d, J = 6.06 Hz, 1H), 7.77 (s, 1H), 7.63 (dd, J = 4.29, 9.60 Hz, 1H), 7.26- 7.36 (m, 2H), 5.85 (d, J = 6.06 Hz, 1H), (3.56 (s, 3H), 3.18 (s, 3H), 2.48 (s, 3H), 2.23 (s, 3H) | 441 | A |
| 87 | N4-(5-FLUORO- 1H-INDAZOL-3- YL)-N2-[2- (METHYLOXY)-5- (METHYL- SULFONYL) PHENYL]-2,4- PYRIMIDINE- DIAMINE | 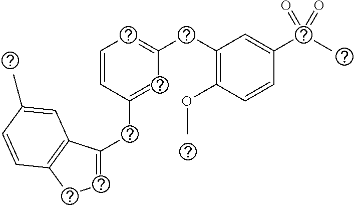 | ¹H NMR (400 MHz, DMSO-d₆) δ ppm 3.07 (s, 3 H) 3.95 (s, 3 H) 6.87 (m, 1 H) 6.96- 7.01 (m, 1 H) 7.05- 7.10 (m, 1 H) 7.12 (d, J = 2.51 Hz, 1 H) 7.31 (d, J = 10.29 Hz, 2 H) 7.54 (dd, J = 9.03, 4.02 Hz, 1 H) 7.67 (br. s., 1 H) 8.14 (br. s., 1 H) 10.48- 11.24 (m, 1 H) 12.96 (br. s., 1 H) | 429 | D |
| 88 | 3-({4-[(5- FLUORO-1H- INDAZOL-3- YL)AMINO]-2- PYRIMIDINYL} AMINO)-4- (METHYLOXY) BENZENE SULFONAMIDE | 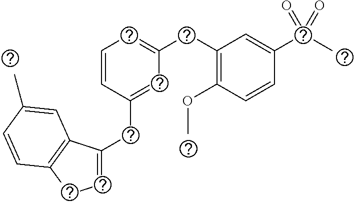 | ¹H NMR (400 MHz, DMSO-d₆) δ ppm 3.92 (br. s., 3 H) 6.96 (br. s., 1 H) 7.22-7.40 (m, 3 H) 7.56 (dd, J = 9.03, 4.02 Hz, 2 H) 7.61- 7.73 (m, 2 H) 8.10 (d, J = 8.28 Hz, 2 H) 8.43 (m, 1 H) 10.79 (s, 1 H) 13.02 (br. s., 1 H) | 430 | D |
| 89 | N4-(5-fluoro-1H- indazolL-3-yl)-N2- {3- [(trifluoromethyl) sulfonyl]phenyl}- 2,4- pyrimidineamine | 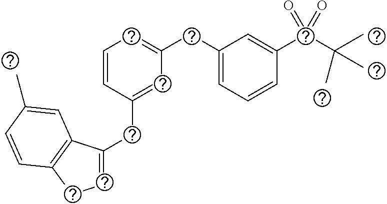 | ¹H NMR (400 MHz, DMSO-d₆) δ ppm 6.83 (br. s., 1 H) 7.26 (td, J = 9.10, 2.38 Hz, 1 H) 7.54 (dd, 3 H) 7.61 (s, 1 H) 8.17 (d, J = 6.27 Hz, 1 H) 8.21-8.36 (m, 1 H) 8.44 (br. s., 1 H) 10.06 (br. s., 1 H) 10.28 (br. s., 1 H) 12.90 (br. s., 1 H) | 453 | D |
| 90 | N2-1-benzothien- 4-yl-N4-(5-fluoro- 1H-indazol-3-yl)- 2,4- pyrimidinediamine trifluoroacetate | 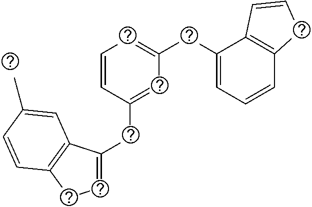 [SMILES: Cc1ccc2c(c1)C(Cc1cccc(Cc3cccc4c3C=CC4)c1)=CC2] | ¹H NMR (DMSO-d₆) δ 12.58-12.85 (m, 1H), 9.65-9.76 (m, 1H), 9.01-9.08 (m, 1H), 8.13 (d, J = 5.5 Hz, 1H), 7.95-8.01 (m, 1H), 7.74 (d, J = 5.8 Hz, 1H), 7.64 (d, J = 5.5 Hz, 3H), 7.47-7.55 (m, 1H), 7.26 (td, J = 9.0, 2.3 Hz, 1H), 7.11-7.20 (m, 1H), 6.92 (none, 1H) | 377 | J |
| 91 | N2-[2,4-dimethyl- 5-(methylsulfonyl) phenyl]-N4-(5- fluoro-1H-indazol- 3-yl)-2,4- pyrimidinediamine trifluoroacetate | 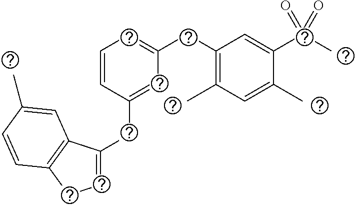 | ¹H NMR (DMSO-d₆) δ 12.56-12.73 (m, 1H), 9.64-9.70 (m, 1H), 8.43-8.49 (m, 1H), 8.06 (d, J = 5.8 Hz, 1H), 7.94-7.97 (m, 1H), 7.54-7.61 (m, 1H), 7.43-7.50 (m, 1H), 7.23-7.26 (m, 2H), 7.18-7.23 (m, 1H), 6.87-6.95 (m, 1H), 3.12-3.15 (m, 3H), 2.56 (s, 3H), 2.24 (none, 3H) | 427 | J |
| 92 | 7-({4-[(5- FLUORO-1H- INDAZOL-3- YL)AMINO]-2- PYRIMIDINYL} AMINO)-2,3- DIHYDRO-1- BENZOFURAN-5- SULFONAMIDE | 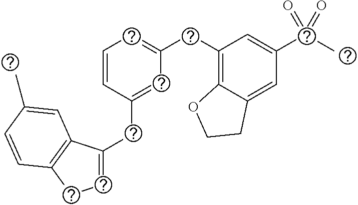 | ¹H NMR (400 MHz, DMSO-d₆) δ ppm 3.17 (s, 2 H) 4.57 (m, 2 H) 6.92-7.03 (m, 1 H) 7.21-7.40 (m, 3 H) 7.50-7.65 (m, 3 H) 8.04 (br. s., 1 H) 10.72- 11.11 (m, 1 H) 12.93- 13.21 (m, 1 H) | 442 | D |
| 93 | N4-(5-fluoro-1H- indazol-3-yl)-N2- [2-fluoro-3- (methylsulfonyl) phenyl]-2,4- pyrimidinediamine trifluoroacetate | 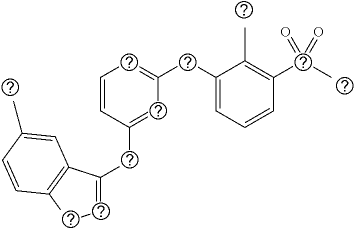 | ¹H NMR (DMSO-d₆) δ 12.89-13.02 (m, 1H), 10.31-10.78 (m, 1H), 9.34-9.83 (m, 1H), 8.14 (d, J = 6.5 Hz, 2H), 7.52 (d, J = 4.0 Hz, 4H), 7.23-7.31 (m, 1H), 6.78-6.96 (m, 1H), 3.20-3.26 (m, 3H) | 417 | J |
| 94 | N2-[4-methyl-3- (methylsulfonyl) phenyl]-N4-[6- (trifluoromethyl)- 1H-indazol-3-yl]- 2,4- pyrimidinediamine | 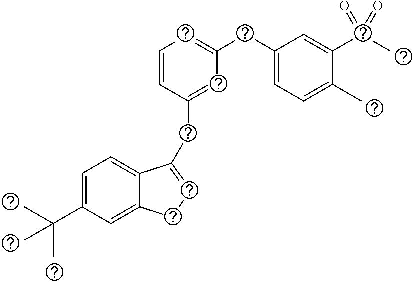 | ¹H NMR (400 MHz, DMSO-d₆) δ ppm 2.51 (s, 3 H) 3.14 (s, 3 H) 6.83 (br. s., 1 H) 7.11 (br. s., 1 H) 7.33 (d, J = 8.53 Hz, 1 H) 7.87 (s, 1 H) 8.06 (d, J = 8.53 Hz, 1 H) 8.11 (br. s., 2 H) 8.16 (d, J = 5.77 Hz, 1 H) 9.42 (br. s., 1 H) 9.94 (br. s., 1 H) 13.09 (s, 1 H) | 463 | C |
| 95 | N4-(6,7- DIFLUORO-1H- INDAZOL-3-YL)- N2-[4-METHYL-3- (METHYL- SULFONYL) PHENYL]-2,4- PYRIMIDINE- DIAMINE | 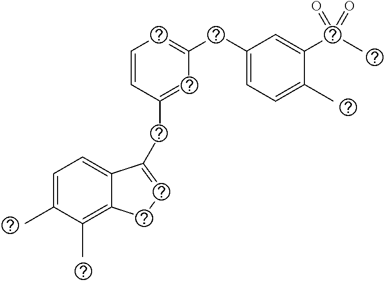 | ¹H NMR (400 MHz, DMSO-d₆) δ ppm 2.54 (s, 3 H) 3.16 (s, 3 H) 6.92 (br. s., 1 H) 7.10 (d, J = 1.52 Hz, 1 H) 7.23 (d, J = 8.59 Hz, 1 H) 7.71 (br. s., 1 H) 8.06-8.30 (m, 3 H) 9.41 (s, 1 H) 9.88 (s, 5 H) 13.28 (br. s., 1 H) | 431 | C |
| 96 | N4-(6-METHYL- 1H-INDAZOL-3- YL)-N2-[4- METHYL-3- (METHYL- SULFONYL) PHENYL]-2,4- PYRIMIDINE- DIAMINE | 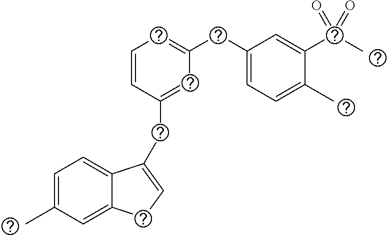 | ¹H NMR (400 MHz, DMSO-d₆) δ ppm 2.41- 2.46 (m, 3 H) 2.53 (s, 3 H) 3.16 (s, 3 H) 6.77- 6.87 (m, 1 H) 6.90 (d, J = 8.28 Hz, 1 H) 7.19 (d, J = 8.03 Hz, 1 H) 7.25 (s, 1 H) 7.70 (d, J = 8.28 Hz, 1 H) 8.12 (d, J = 5.77 Hz, 1 H) 8.14 (d, J = 1.76 Hz, 1 H) 8.17-8.28 (m, 1 H) 9.37 (s, 1 H) 9.68 (br. s., 1 H) | 409 | C |
| 97 | N4-(5-CHLORO-? 1H-INDAZOL-3- YL)-N2-[4- METHYL-3- (METHYL- SULFONYL) PHENYL]-2,4- PYRIMIDINE- DIAMINE | 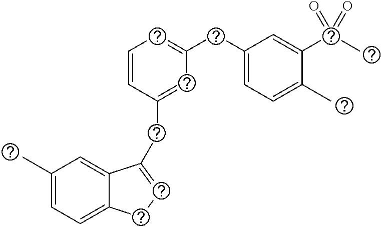 | ¹H NMR (400 MHz, DMSO-d₆) δ ppm 2.55 (none, 3 H) 3.16 (s, 3 H) 6.91 (br. s., 1 H) 7.19 (d, J = 8.28 Hz, 1 H) 7.37 (dd, J = 8.78, 2.01 Hz, 1 H) 7.48-7.58 (m, 1 H) 7.96 (s, 1 H) 8.13 (d, J = 2.01 Hz, 1 H) 8.16 (d, J = 5.77 Hz, 1 H) 8.21 (d, J = 8.28 Hz, 1 H) 9.42 (s, 1 H) 9.78 (br. s., 1 H) | 429 | C |
| 98 | N4-(4-CHLORO- 1H-INDAZOL-3- YL)-N2-[4- METHYL-3- (METHYL- SULFONYL) PHENYL]-2,4- PYRIMIDINE- DIAMINE | 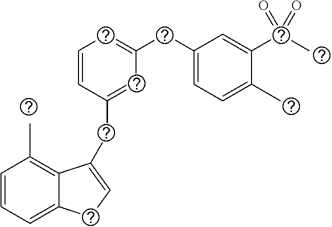 | ¹H NMR (400 MHz, DMSO-d₆) δ ppm 2.48 (s, 3 H) 3.09-3.16 (m, 3 H) 6.27-6.37 (m, 1 H) 6.94 (br. s., 1 H) 7.12 (d, J = 7.28 Hz, 1 H) 7.30-7.40 (m, 1 H) 7.48-7.58 (m, 1 H) 7.75-7.90 (m, 1 H) 8.06 (d, J = 5.77 Hz, 1 H) 8.17 (br. s., 1 H) 9.11 (br. s., 1 H) 9.38 (s, 1 H) 13.29 (m, 1 H) | 429 | C |
| 99 | N4-(7-FLUORO- 1H-INDAZOL-3- YL)-N2-[4- METHYL-3- (METHYL- SULFONYL) PHENYL]-2,4- PYRIMIDINE- DIAMINE | 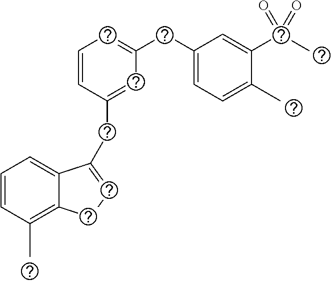 | ¹H NMR (400 MHz, DMSO-d₆) δ ppm 2.54 (s, 3 H) 3.16 (s, 3 H) 6.86 (br. s., 1 H) 7.04 (td, J = 7.83, 4.29 Hz, 1 H) 7.12-7.31 (m, 2 H) 7.68 (d, J = 8.08 Hz, 1 H) 8.11-8.18 (m, 2 H) 8.20 (d, J = 8.08 Hz, 1 H) 9.40 (s, 1 H) 9.80 (s, 5 H) 13.06-13.24 (s, 1 H) | 413 | C |
| 100 | N4-(5,7- DIFLUORO-1H- INDAZOL-3-YL)- N2-[4-METHYL-3- (METHYL- SULFONYL) PHENYL]-2,4- PYRIMIDINE- DIAMINE | 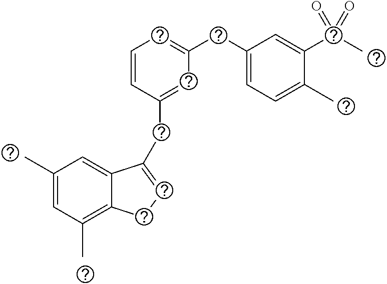 | ¹H NMR (400 MHz, DMSO-d₆) δppm 2.53 (s, 3 H) 3.13-3.19 (m, 3 H) 6.83 (br. s., 1 H) 7.20 (d, J = 7.03 Hz, 1 H) 7.35 (t, J = 9.91 Hz, 1 H) 7.47 (d, J = 8.28 Hz, 1 H) 8.04-8.23 (m, 3 H) 9.40 (br. s., 1 H) 9.81 (br. s., 1 H) 13.32 (br. s., 1 H | 431 | C |
| 101 | N4-(5,6-difluoro- 1H-indazol-3-yl)- N2-[4-methyl-3- (methylsulfonyl) phenyl]-2,4- pyrimidinediamine | 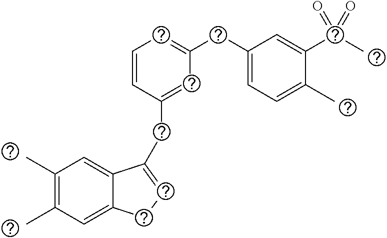 | ¹H NMR (DMSO-d₆) δ 12.79 (s, 1H), 9.77 (s, 1H), 9.38 (s, 1H), 8.12- 8.20 (m, 3H), 7.86 (d, J = 1.5 Hz, 1H), 7.54 (dd, J = 10.6, 6.8 Hz, 1H), 7.20 (d, J = 8.3 Hz, 1H), 6.84-6.92 (m, 1H), 3.16 (s, 3H), 2.54 (s, 3H) | 431 | E |
| 102 | N4-(5-fluoro-1H- indazol-3-yl)-N2- [2-methyl-5- (methylsulfonyl) phenyl]-2,4- pyrimidinediamine | 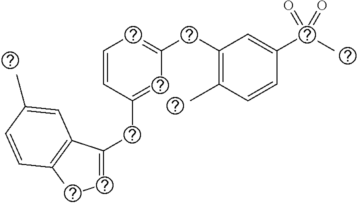 | ¹H NMR (DMSO-d₆) δ 12.64 (s, 1H), 9.72 (s, 1H), 8.45 (s, 1H), 8.08- 8.12 (m, 2H), 7.60 (d, J = 9.3 Hz, 1H), 7.42- 7.55 (m, 3H), 7.19- 7.28 (m, 1H), 6.95 (br. s., 1H), 3.13 (s, 3H), 2.32 (s, 3H) | 413 | J |
| 103 | N4-(5-FLUORO- 1H-INDAZOL-3- YL)-N2-[3,4,5- TRIS(METHYL- OXY)PHENYL]- 2,4-PYRIMIDINE- DIAMINE | 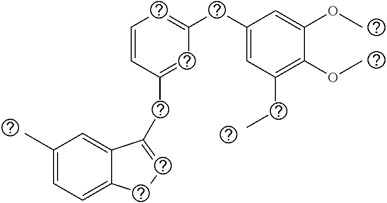 | ¹H NMR (500 MHz, DMSO-d₆) δ ppm 3.58 (s, 3 H) 3.59 (br. s., 6 H) 6.70 (br. s., 1 H) 7.08 (br. s., 2 H) 7.24 (td, J = 8.97, 2.32 Hz, 1 H) 7.47-7.57 (m, 2 H) 8.09 (br. s., 1 H) 8.89 (br. s., 1 H) 9.57 (br. s., 1 H) 12.70 (br. s., 1 H) | 411 | D |
| 104 | N2-[4-METHYL-3- (METHYL- SULFONYL) PHENYL]-N4-[5- (METHYLOXY)- 1H-INDAZOL-3- YLJ-2,4- PYRIMIDINE- DIAMINE | 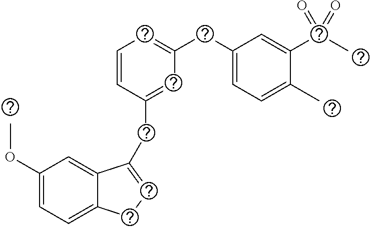 | ¹H NMR (500 MHz, DMSO-d₆) δ ppm 2.53 (s, 3 H) 3.16 (s, 3 H) 3.71 (s, 3 H) 6.76 (br. s., 1 H) 7.02 (dd, J = 9.03, 1.95 Hz, 1 H) 7.12-7.20 (m, 1 H) 7.23 (br. s., 1 H) 7.39 (d, J = 8.79 Hz, 1 H) 8.10 (d, J = 5.86 Hz, 1 H) 8.14 (br. s., 1 H) 8.19 (d, J = 8.06 Hz, 1 H) 9.34 (s, 1 H) 9.56 (s, 1 H) 12.46 (s, 1 H) | 425 | C |
| 105 | N′-[3-({4-[(5-fluoro- 1H-indazol-3- yl)amino]-2- pyrimidinyl}amino) phenyl]-N,N- dimethylsulfamide | 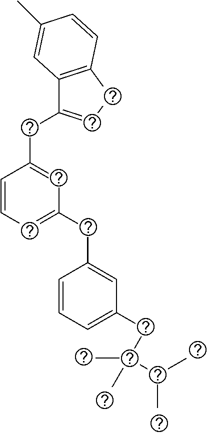 [SMILES: Cc1ccc2c(c1)C(Cc1cccc(Cc3cccc(CC(C)(C)C(C)C)c3)c1)=CC2] | ¹H NMR (DMSO-d₆) δ 12.70 (br. s., 1H), 9.65 (br. s., 1H), 9.55 (s, 1H), 9.12 (s, 1H), 8.11 (d, J = 5.8 Hz, 1H), 7.48- 7.68 (m, 4H), 7.27 (td, J = 9.1, 2.3 Hz, 1H), 7.02 (t, J = 8.1 Hz, 1H), 6.85 (br. s., 1H), 6.73 (d, J = 7.8 Hz, 1H), 2.72 (s, 6H). | 443 | D |
| 106 | 3-({4-[(5-fluoro-1H- indazol-3- yl)amino]-2- pyrimidinyl}amino) benzene sulfonamide hydrochloride | 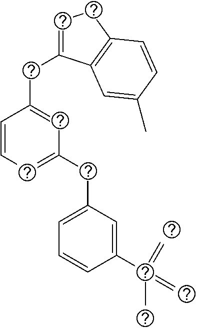 | ¹H NMR (DMSO-d₆) δ 13.12-13.36 (m, 1H), 11.33 (br. s., 1H), 10.94 (br. s., 1H), 8.12-8.21 (m, 1H), 7.71-8.07 (m, 2H), 7.56-7.68 (m, 2H), 7.37-7.54 (m, 3H), 7.32 (td, J = 9.1, 2.3 Hz, 1H), 6.84 (br. s., 1H) | 400 | E |
| 107 | 5-({4-[(5-fluoro-1H- indazol-3- yl)amino]-2- pyrimidinyl}amino)- 2-methylbenzene sulfonamide | 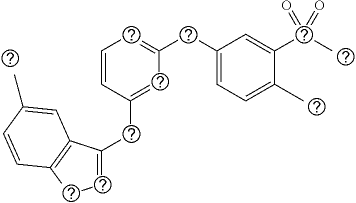 | ¹H NMR (DMSO-d₆) δ 12.71 (s, 1H), 9.62 (s, 1H), 9.34 (s, 1H), 8.25- 8.29 (m, 1H), 8.13 (d, J = 5.8 Hz, 1H), 7.95 (dd, J = 8.3, 2.3 Hz, 1H), 7.58-7.64 (m, 1H), 7.52 (dd, J = 9.0, 4.2 Hz, 1H), 7.33 (s, 2H), 7.28 (td, J = 9.1, 2.3 Hz, 1H), 7.07-7.14 (m, 1H), 6.85-6.91 (m, 1H) | 414 (M⁺) | E |
| 108 | N4-(6,7-difluoro- 1H-indazol-3-yl)- N4-methyl-N2-[4- methyl-3- (methylsulfonyl) phenyl]-2,4- pyrimidinediamine | 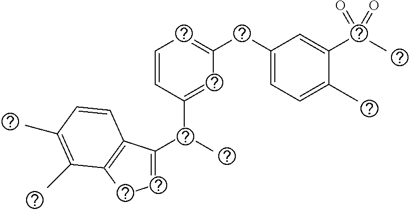 | ¹H NMR (DMSO-d₆) δ 13.80 (s, 1H), 9.61 (s, 1H), 8.62 (s, 1H), 7.97 (d, J = 6.1 Hz, 1H), 7.75 (dd, J = 8.3, 2.0 Hz, 1H), 7.37 (dd, J = 8.7, 3.9 Hz, 1H), 7.24 (d, J = 8.3 Hz, 1H), 7.14-7.22 (m, 1H), 5.94 (d, J = 5.8 Hz, 1H), 3.57 (s, 3H), 3.17 (s, 3H), 2.55 (s, 3H) | 445 | E |
| 109 | N4-(6,7-difluoro- 1H-indazol-3-yl)- N4-methyl-N2-[3- methyl-5- (methylsulfonyl) phenyl]-2,4- pyrimidinediamine | 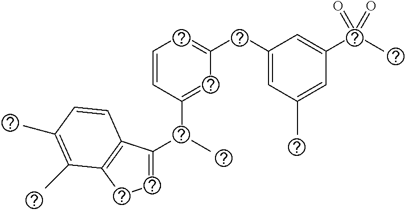 | ¹H NMR (DMSO-d₆) δ 13.80 (s, 1H), 9.66 (s, 1H), 8.36 (br. s., 1H), 8.01 (d, J = 5.8 Hz, 1H), 7.70 (s, 1H), 7.37 (dd, J = 8.7, 3.9 Hz, 1H), 7.25 (s, 1H), 7.18 (ddd, J = 10.7, 8.8, 6.7 Hz, 1H), 6.00 (d, J = 6.1 Hz, 1H), 3.57 (s, 3H), 3.14 (s, 3H), 2.28 (s, 3H) | 445 | E |
| 110 | N4-(6,7-difluoro- 1H-indazol-3-yl)- N4-methyl-N2- [3,4,5- tris(methyloxy) phenyl]-2,4- pyrimidinediamine | 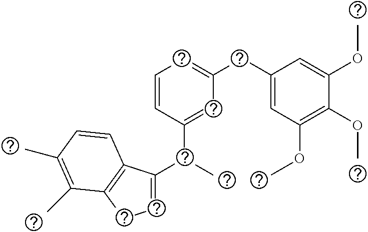 | ¹H NMR (DMSO-d₆) δ 13.77 (s, 1H), 9.14 (s, 1H), 7.94 (d, J = 5.8 Hz, 1H), 7.37 (dd, J = 8.7, 3.9 Hz, 1H), 7.18 (s, 3H), 5.90 (d, J = 6.1 Hz, 1H), 3.71 (s, 6H), 3.60 (s, 3H), 3.57 (s, 3H) | 443 | E |
| 111 | N4-(7-fluoro-1H- indazol-3-yl)-N4- methyl-N2-[4- methyl-3- (methylsulfonyl) phenyl]-2,4- pyrimidinediamine | 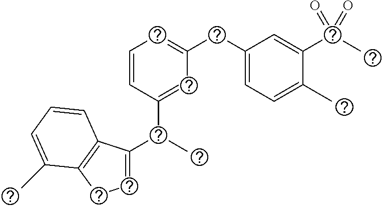 | ¹H NMR (DMSO-d₆) δ 13.65 (s, 1H), 9.61 (s, 1H), 8.65 (s, 1H), 7.95 (d, J = 6.1 Hz, 1H), 7.76 (dd, J = 8.5, 2.1 Hz, 1H), 7.36 (d, J = 8.1 Hz, 1H), 7.21-7.31 (m, 2H), 7.10 (td, J = 7.9, 4.4 Hz, 1H), 5.90 (d, J = 6.1 Hz, 1H), 3.58 (s, 3H), 3.17 (s, 3H), 2.55 (s, 3H) | 427 | E |
| 112 | N4-(7-fluoro-1H- indazol-3-yl)-N4- methyl-N2-[3- methyl-5- (methylsulfonyl) phenyl]-2,4- pyrimidinediamine | 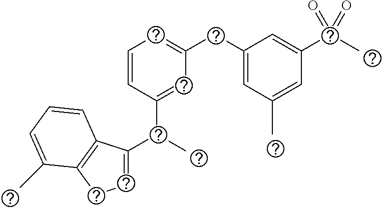 | ¹H NMR (DMSO-d₆) δ 13.62-13.69 (m, 1H), 9.63-9.68 (m, 1H), 8.36-8.42 (m, 1H), 7.99 (d, J = 6.1 Hz, 1H), 7.69-7.73 (m, 1H), 7.36 (d, J = 8.1 Hz, 1H), 7.25 (s, 2H), 7.10 (td, J = 7.8, 4.3 Hz, 1H), 5.96 (d, J = 6.1 Hz, 1H), 3.58 (s, 3H), 3.14 (s, 3H), 2.28 (s, 3H) | 427 | E |
| 113 | N4-(7-fluoro-1H- indazol-3-yl)-N4- methyl-N2-[3- (methyloxy)-5- (methylsulfonyl) phenyl]-2,4- pyrimidinediamine | 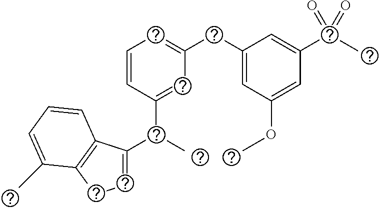 | ¹H NMR (DMSO-d₆) δ 13.66 (s, 1H), 9.70 (s, 1H), 8.17 (s, 1H), 7.98 (d, J = 6.1 Hz, 1H), 7.63 (s, 1H), 7.36 (d, J = 8.1 Hz, 1H), 7.27 (dd, J = 11.5, 7.5 Hz, 1H), 7.10 (td, J = 7.9, 4.4 Hz, 1H), 6.94-6.99 (m, 1H), 5.94 (d, J = 6.1 Hz, 1H), 3.81 (s, 3H), 3.59 (s, 3H), 3.18 (s, 3H) | 443 | E |
| 114 | N4-(7-fluoro-1H- indazol-3-yl)-N4- methyl-N2-[3,4,5- tris(methyloxy) phenyl]-2,4- pyrimidinediamine | 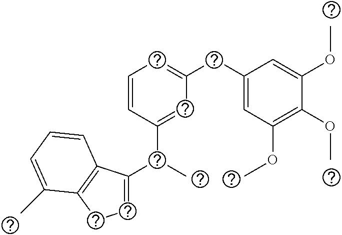 | ¹H NMR (DMSO-d₆) δ 13.58-13.67 (m, 1H), 9.14 (s, 1H), 7.92 (d, J = 5.8 Hz, 1H), 7.35 (d, J = 8.1 Hz, 1H), 7.26 (dd, J = 11.4, 7.6 Hz, 1H), 7.21 (s, 2H), 7.09 (td, J = 7.9, 4.4 Hz, 1H), 5.85 (d, J = 5.8 Hz, 1H), 3.72 (s, 6H), 3.61 (s, 3H), 3.58 (s, 3H) | 425 | E |
| 115 | N4-(5,6-difluoro- 1H-indazol-3-yl)- N4-methyl-N2- [3,4,5- tris(methyloxy) phenyl]-2,4- pyrimidinediamine | 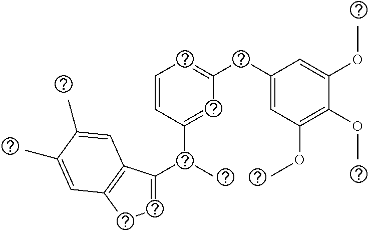 | ¹H NMR (DMSO-d₆) δ 13.22-13.31 (m, 1H), 9.12 (s, 1H), 7.92 (d, J = 5.8 Hz, 1H), 7.57- 7.70 (m, 2H), 7.19 (s, 2H), 5.84 (d, J = 5.8 Hz, 1H), 3.71 (s, 6H), 3.61 (s, 3H), 3.55 (s, 3H) | 443 | E |
| 116 | N4-(5,6-difluoro- 1H-indazol-3-yl)- N4-methyl-N2-[3- (methyloxy)-5- (methylsulfonyl) phenyl]-2,4- pyrimidinediamine | 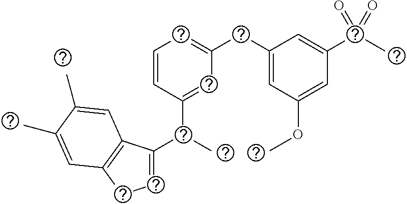 | ¹H NMR (DMSO-d₆) δ 13.25-13.31 (m, 1H), 9.68 (s, 1H), 8.16 (s, 1H), 7.98 (d, J = 5.8 Hz, 1H), 7.59-7.71 (m, 3H), 6.93-6.99 (m, 1H), 5.92 (d, J = 5.8 Hz, 1H), 3.80 (s, 3H), 3.56 (s, 3H), 3.18 (s, 3H) | 461 | E |
| 117 | N4-(5,6-difluoro- 1H-indazol-3-yl)- N4-methyl-N2-[4- methyl-3- (methylsulfonyl) phenyl]-2,4- pyrimidinediamine | 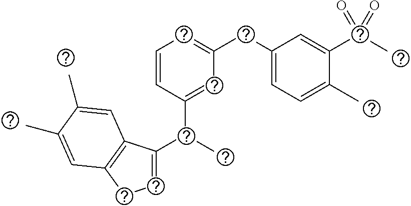 | ¹H NMR (DMSO-d₆) δ 13.08-13.59 (m, 1H), 9.62 (s, 1H), 8.64 (br. s., 1H), 7.95 (d, J = 6.0 Hz, 1H), 7.73-7.80 (m, 1H), 7.69 (dd, J = 10.4, 6.7 Hz, 1H), 7.62 (dd, J = 9.9, 7.9 Hz, 1H), 7.24 (d, J = 8.3 Hz, 1H), 5.88 (d, J = 5.8 Hz, 1H), 3.55 (s, 3H), 3.18 (s, 3H), 2.55 (s, 3H) | 445 | E |
| 118 | N4-(5,6-difluoro- 1H-indazol-3-yl)- N4-methyl-N2-[3- methyl-5- (methylsulfonyl) phenyl]-2,4- pyrimidinediamine | 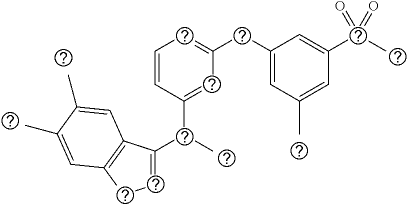 | ¹H NMR (DMSO-d₆) δ 13.38 (s, 1H), 9.96 (br. s., 1H), 8.40 (br. s., 1H), 7.99 (d, J = 6.3 Hz, 1H), 7.63-7.74 (m, 3H), 7.33 (s, 1H), 5.99 (d, J = 5.8 Hz, 1H), 3.57 (s, 3H), 3.17 (s, 3H), 2.32 (s, 3H) | 445 | E |
| 119 | N4-(6,7-difluoro- 1H-indazol-3-yl)- N4-methyl-N2-[3- (methyloxy)-5- (methylsulfonyl) phenyl]-2,4- pyrimidinediamine | 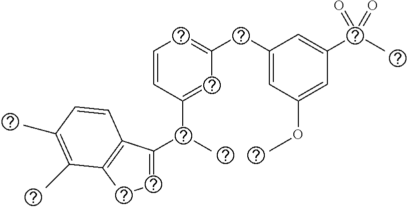 | ¹H NMR (DMSO-d₆) δ 13.78 (br. s., 2H), 9.69 (s, 3H), 8.09 (br. s., 1H), 8.02 (d, J = 5.8 Hz, 1H), 7.56 (br. s., 1H), 7.37 (dd, J = 8.7, 3.9 Hz, 1H), 7.12-7.22 (m, 1H), 6.93-6.97 (m, 1H), 6.01 (d, J = 5.8 Hz, 1H), 3.79 (s, 3H), 3.57 (s, 3H), 3.17 (s, 3H) | 461 | E |
| 120 | N4-(1H-INDAZOL- 3-YL)-N2-(3- METHYL-5- (METHYL- SULFONYL) PHENYL) PYRIMIDINE-2,4- DIAMINE | 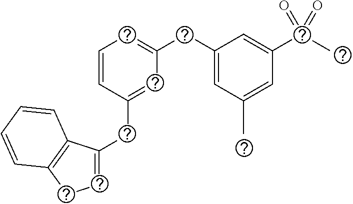 | ¹H NMR (400 MHz, DMSO-d₆) δ ppm 2.16 (br. s., 3 H) 3.12 (s, 3 H) 6.77 (br. s., 1 H) 7.06 (t, J = 7.33 Hz, 1 H) 7.18 (s, 1 H) 7.37 (t, J = 7.20 Hz, 1 H) 7.49 (d, J = 8.59 Hz, 1 H) 7.78 (d, J = 8.34 Hz, 1 H) 7.96 (br. s., 1 H) 8.02 (s, 1 H) 8.14 (d, J = 5.81 Hz, 1 H) 9.42 (s, 1 H) 9.73 (s, 1 H) 12.63 (s, 1 H) | 395 | E |
| 121 | N4-(1H-INDAZOL- 3-YL)-N2-(4- METHYL-3- (METHYL- SULFONYL) PHENYL) PYRIMIDINE-2,4- DIAMINE | 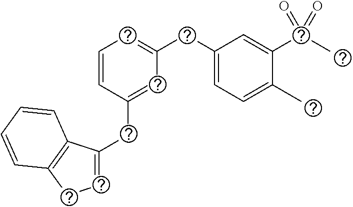 | ¹H NMR (400 MHz, DMSO-d₆) δ ppm 2.53 (s, 3 H) 3.16 (s, 3 H) 6.85 (d, J = 5.31 Hz, 1 H) 7.07 (t, J = 7.20 Hz, 1 H) 7.17 (d, J = 8.08 Hz, 1 H) 7.37 (t, J = 7.20 Hz, 1 H) 7.48 (d, J = 8.59 Hz, 1 H) 7.84 (d, J = 8.08 Hz, 1 H) 8.10-8.18 (m, 2 H) 8.18-8.27 (m, 1 H) 9.38 (s, 1 H) 9.69 (s, 1 H) 12.60 (s, 1 H) | 395 | E |
| 122 | N4-(7-FLUORO- 1H-INDAZOL-3- YL)-N2-(3- METHYL-5- (METHYL- SULFONYL) PHENYL) PYRIMIDINE-2,4- DIAMINE | 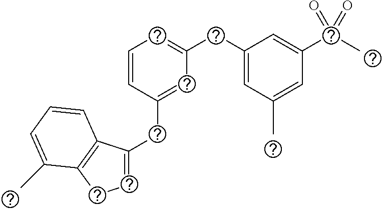 | ¹H NMR (400 MHz, DMSO-d₆) δ ppm 2.11- 2.21 (m, 3 H) 3.12 (s, 3 H) 6.67-6.87 (m, 1 H) 7.02 (td, J = 7.89, 4.42 Hz, 1 H) 7.13-7.30 (m, 2 H) 7.61 (d, J = 8.08 Hz, 1 H) 7.96 (d, J = 16.93 Hz, 2 H) 8.17 (d, J = 5.56 Hz, 1 H) 9.44 (s, 1 H) 9.83 (s, 1 H) 13.20 (s, 1 H) | 413 | E |
| 123 | N4-(4-FLUORO- 1H-INDAZOL-3- YL)-N4-METHYL- N2-(4-METHYL-3- (METHYL- SULFONYL) PHENYL) PYRIMIDINE-2,4- DIAMINE | 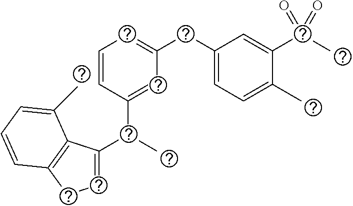 | ¹H NMR (400 MHz, DMSO-d₆) δ ppm 2.54 (s, 3 H) 3.17 (s, 3 H) 3.52 (s, 3 H) 5.73-5.93 (m, 1 H) 6.82-6.99 (m, 1 H) 7.21 (br. s., 1 H) 7.33-7.52 (m, 2 H) 7.73 (d, J = 7.58 Hz, 1 H) 7.88-8.02 (m, 1 H) 8.62 (br. s., 1 H) 9.59 (s, 1 H) 13.38 (s, 1 H) | 427 | E |
| 124 | N4-(6,7- DIFLUORO-1H- INDAZOL-3-YL)- N2-(3-METHYL-5- (METHYL- SULFONYL) PHENYL) PYRIMIDINE-2,4- DIAMINE | 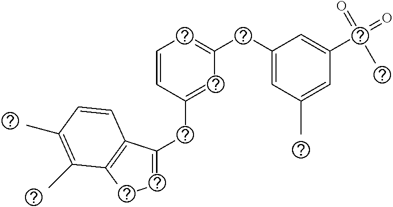 | ¹H NMR (400 MHz, DMSO-d₆) δ ppm 2.20 (br. s., 3 H) 3.12 (s, 3 H) 6.85 (br. s., 1 H) 7.02-7.17 (m, 1 H) 7.21 (s, 1 H) 7.65 (d, J = 5.05 Hz, 1 H) 7.96 (d, J = 8.59 Hz, 2 H) 8.19 (d, J = 5.81 Hz, 1 H) 9.44 (s, 1 H) 9.91 (s, 1 H) 13.33 (br. s., 1 H) | 431 | E |
| 125 | N4-(4-FLUORO- 1H-INDAZOL-3- YL)-N4-METHYL- N2-(3-METHYL-5- (METHYL- SULFONYL) PHENYL) PYRIMIDINE-2,4- DIAMINE | 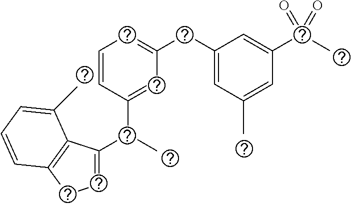 | ¹H NMR (400 MHz, DMSO-d₆) δ ppm 2.28 (br. s., 3 H) 3.14 (s, 3 H) 3.51 (s, 3 H) 5.90 (br. s., 1 H) 6.79-6.97 (m, 1 H) 7.24 (s, 1 H) 7.32-7.49 (m, 2 H) 7.71 (br. s., 1 H) 7.98 (d, J = 5.81 Hz, 1 H) 8.38 (br. s., 1 H) 9.64 (s, 1 H) 13.39 (br. s., 1 H) | 427 | E |
| 126 | N4-(5-fluoro-6- methyl-1H-indazol- 3-yl)-N4-methyl- N2-[3-methyl-5- (methylsulfonyl) phenyl]-2,4- pyrimidinediamine | 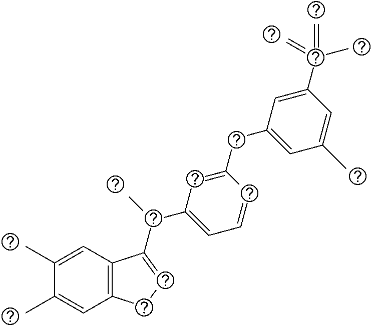 | ¹H NMR (DMSO-d₆) δ 9.64 (s, 1H), 8.42 (s., 1H), 7.96 (br. m., 2H), 7.73 (m, 1H), 7.25 (s, 2H), 6.95 (s, 1H), 5.93 (br. s., 1H), 3.56 (s, 3H), 3.15 (s, 3H), 2.54 (s, 3H), 1.24 (s, 3H) | 441 | E |
| 127 | N4-(7-fluoro-6- methyl-1H-indazol- 3-yl)-N4-methyl- N2-[3-methyl-5- (methylsulfonyl) phenyl]-2,4- pyrimidinediamine | 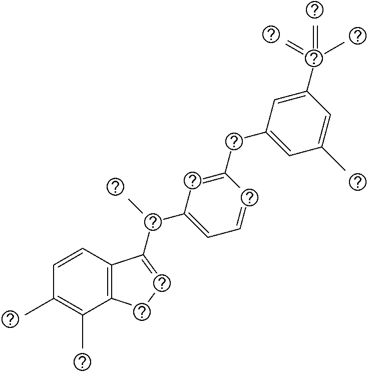 | ¹H NMR (METHANOL- d₄) δ 8.48 (s., 1H), 7.93 (s, 1H), 7.63 (br. s., 1H), 7.40-7.47 (m, 1H), 7.34 (s, 1H), 7.12 (d, J = 1.3 Hz, 1H), 5.94 (br. s., 1H), 3.64 (s, 3H), 3.11 (s, 3H), 2.40- 2.46 (m, 3H), 2.38 (s, 3H) | 441 | E |
| 128 | N4-(5,6-difluoro- 1H-indazol-3-yl)- N2-[3-methyl-5- (methylsulfonyl) phenyl]-2,4- pyrimidinediamine | 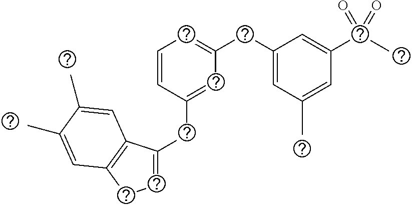 | ¹H NMR (DMSO-d₆) δ 12.81 (s, 1H), 9.80 (s, 1H), 9.42 (s, 1H), 8.17 (d, J = 5.8 Hz, 1H), 7.98 (s, 2H), 7.76-7.84 (m, 1H), 7.56 (dd, J = 10.6, 6.8 Hz, 1H), 7.21 (s, 1H), 6.74-6.85 (m, 1H), 3.13 (s, 3H), 2.21 (br. s., 3H) | 431 | E |
| 129 | N4-(5,6-difluoro- 1H-indazol-3-yl)- N2-[3-(methyloxy)- 5-(methylsulfonyl) phenyl]-2,4- pyrimidinediamine | 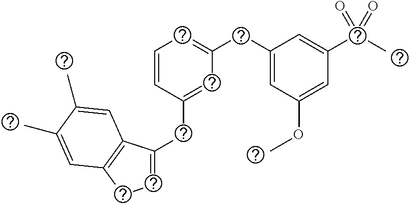 | ¹H NMR (DMSO-d₆) δ 12.79 (br. s., 1H), 9.81 (s, 1H), 9.46 (s, 1H), 8.17 (d, J = 5.6 Hz, 1H), 7.73-7.85 (m, 3H), 7.55 (dd, J = 10.6, 6.8 Hz, 1H), 6.92 (s, 1H), 6.83 (br. s., 1H), 3.74 (s, 3H), 3.16 (s, 3H) | 447 | E |
| 130 | N4-(5,6-difluoro- 1H-indazol-3-yl)- N2-[3,4,5- tris(methyloxy) phenyl]-2,4- pyrimidinediamine | 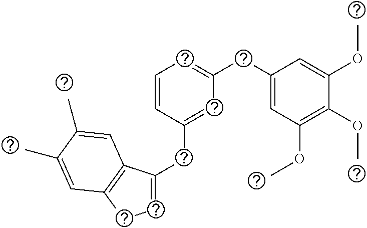 | ¹H NMR (DMSO-d₆) δ 12.77 (br. s., 1H), 9.69 (s, 1H), 8.93 (s, 1H), 8.11 (d, J = 5.6 Hz, 1H), 7.73-7.85 (m, 1H), 7.53 (dd, J = 10.6, 6.6 Hz, 1H), 7.06 (s, 2H), 6.76 (br. s., 1H), 3.61 (br. s., 6H), 3.58 (s, 3H) | 429 | E |
| 131 | N4-(6-fluoro-5- methyl-1H-indazol- 3-yl)-N4-methyl- N2-[3-methyl-5- (methylsulfonyl) phenyl]-2,4- pyrimidinediamine trifluoroacetate | 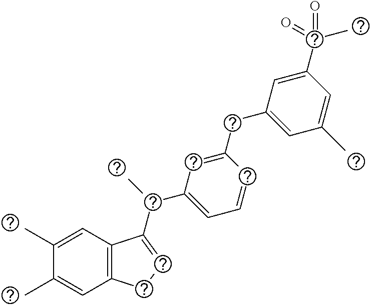 | ¹H NMR (DMSO-d₆) δ 13.05 (br. s., 1H), 9.66 (s, 1H), 8.45 (br. s., 1H), 7.95 (d, J = 5.8 Hz, 1H), 7.75 (s, 1H), 7.44 (d, J = 7.6 Hz, 1H), 7.35 (d, J = 10.1 Hz, 1H), 7.26 (s, 1H), 5.87 (d, J = 5.8 Hz, 1H), 3.56 (s, 3H), 3.15 (s, 3H), 2.32 (s, 3H), 2.27 (d, 3H) | 441 | E |
| 132 | 2-chloro-5-({4-[(5- fluoro-1H-indazol- 3-yl)amino]-2- pyrimidinyl}amino) benzene sulfonamide | 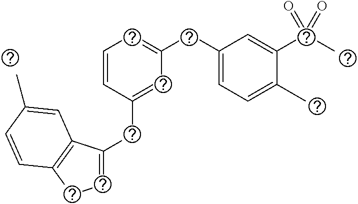 | ¹H NMR (DMSO-d₆) δ 12.75 (s, 1H), 9.70 (s, 1H), 9.59 (s, 1H), 8.37 (br. s., 1H), 8.16 (d, J = 5.8 Hz, 2H), 7.57-7.64 (m, 1H), 7.49-7.56 (m, 3H), 7.25-7.38 (m, 2H), 6.88 (br. s., 1H) | 434 | E |
| 133 | N4-(5-fluoro-6- methyl-1H-indazol- 3-yl)-N4-methyl- N2-[3-methyl-5- (methylsulfonyl) phenyl]-2,4- pyrimidinediamine hydrochloride | 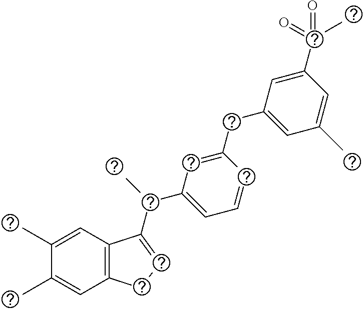 | ¹H NMR (DMSO-d₆) δ 9.64 (s, 1H), 8.44 (br. s., 1H), 7.95 (d, J = 5.8 Hz, 1H), 7.75 (s, 1H), 7.49 (d, J = 6.1 Hz, 1H), 7.16-7.31 (m, 2H), 5.88 (d, J = 6.1 Hz, 1H), 3.17 (s, 3H), 3.15 (s, 3H), 2.37 (d, J = 1.3 Hz, 3H), 2.31 (s, 3H) | 441 | E |
| 134 | 3-({4-[(7-fluoro-1H- indazol-3- yl)(methyl)amino]- 2-pyrimidinyl} amino)-5- methylbenzene sulfonamide | 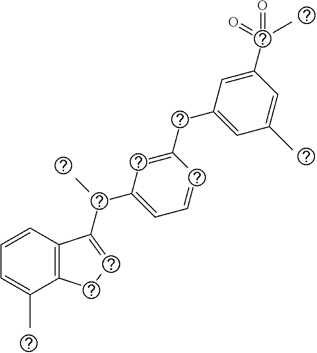 | ¹H NMR (DMSO-d₆) δ 13.82 (br. s., 1H), 10.10 (br. s., 1H), 8.15 (br. s., 1H), 7.97 (d, J = 6.6 Hz, 1H), 7.57 (br. s., 1H), 7.41 (d, J = 8.1 Hz, 1H), 7.25-7.35 (m, 4H), 7.08-7.17 (m, 1H), 6.07 (br. s., 1H), 2.28 (br. s., 3H), 2.08 (s, 3H) | 428 | E |
| 135 | N4-(7-fluoro-1H- indazol-3-yl)-N4- methyl-N2-(3- (methylsulfonyl)-5- (pyrrolidin-1- yl)phenyl) pyrimidine-2,4- diamine | 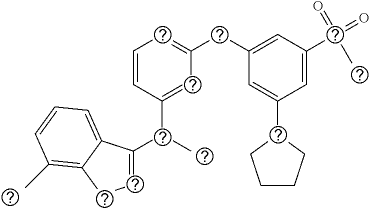 | ¹H NMR (400 MHz, DMSO-d₆) δ ppm 1.97 (m, 4H) 3.12 (s, 3H) 3.24 (s, 3H) 3.44 (m, 4H) 3.59 (s, 3H) 5.97 (br.s., 1H) 6.55 (s, 1H) 7.13 (br. S., 1H) 7.24 (d, J = 8.69 Hz, 2H) 7.35 (d, J = 6.04 Hz, 1H) 7.85 (br, s., 1H) 7.94 (br. S., 1H) 9.44 (br. S., 1H) 13.71 (br. S., 1H) | 482 | E |
| 136 | 3-({4-[(6,7-difluoro- 1H-indazol-3- yl)(methyl)amino]- 2-pyrimidinyl} amino)-5- methylbenzene sulfonamide | 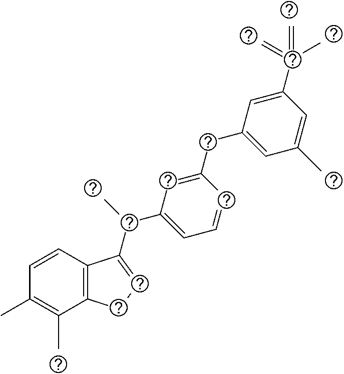 | ¹H NMR (DMSO-d₆) δ 13.91 (br. s., 1H), 9.92 (br. s., 1H), 8.15 (br. s., 1H), 7.99 (d, J = 6.3 Hz, 1H), 7.57 (br. s., 1H), 7.40 (d, J = 4.3 Hz, 1H), 7.27 (s, 4H), 6.08 (br. s., 1H), 2.27 (s, 3H), 2.08 (s, 3H) | 446 | E |
| 137 | N4-(7-fluoro-1H- indazol-3-yl)-N4- methyl-N2-(3- methyl-5- (pyrrolidin-1- yl)phenyl) pyrimidine-2,4- diamine | 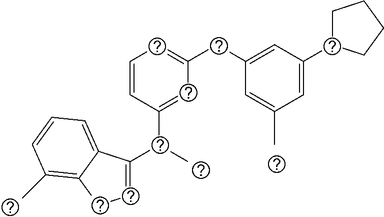 [SMILES: Cc1cc(Cc2cccc(C(C)C3=CCc4c(C)cccc43)c2)cc(C2CCCC2)c1] | ¹HNMR(400 MHz, DMSO-d₆) δ ppm 1.95 (m, 4H) 2.31 (m, 3H) 3.32 (m, 4H) 3.69 (s, 3H) 3.72 (s, 1H) 5.96 (d, J = 6.4 Hz, 1H) 6.16 (s, 1H) 6.76 (s, 1H) 6.81 (s, 1H) 7.16 (br. S., 1H) 7.32 (d, J = 8.69 Hz, 1H) 7.35 (d, J = 6.04 Hz, 1H) 7.78 (br, s., 1H) 7.94 (br. S., 1H) 10.42 (br. S., 1H) | 418 | E |
| 138 | N4-(7-fluoro-1H- indazol-3-yl)-N4- methyl-N2-(3- methyl-5- morpholinophenyl) pyrimidine-2,4- diamine | 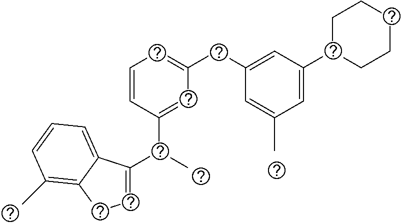 [SMILES: Cc1cc(Cc2cccc(C(C)C3=CCc4c(C)cccc43)c2)cc(C2CCCCC2)c1] | ¹HNMR(400 MHz, DMSO-d₆) δ ppm 2.11 (s, 3H) 3.02 (t, 4H) 3.54 (s, 3H) 3.72 (t, 4H) 5.91 (d, J = 6.1 Hz, 1H) 6.31 (s, 1H) 6.76 (s, 1H) 7.01(s, 1H) 7.10 (m, 1H) 7.25 (d, J = 8.69 Hz, 1H) 7.35 (d, J = 6.04 Hz, 1H) 7.94 (d, J = 5.6 Hz, 1H) 9.01 (S., 1H) 13.42 (br. S., 1H) | 434 | E |
| 139 | N⁴-(7-fluoro-6- methyl-1H-indazol- 3-yl)-N⁴-methyl-N²- [3-(methyloxy)-5- (methylsulfonyl) phenyl]-2,4- pyrimidinediamine | 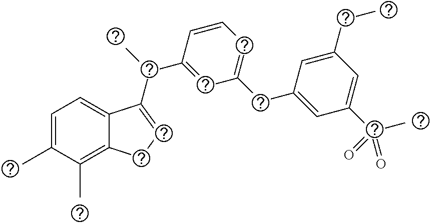 | ¹H NMR (DMSO-d₆) δ 13.47 (s, 1H), 9.69 (s, 1H), 8.18 (s, 1H), 7.97 (d, J = 5.8 Hz, 1H), 7.64 (s, 1H), 7.23 (d, J = 8.3 Hz, 1H), 6.93-7.03 (m, 2H), 5.94 (d, J = 6.1 Hz, 1H), 3.81 (s, 3H), 3.58 (s, 3H), 3.18 (s, 3H), 2.37 (d, J = 2.0 Hz, 3H) | 457 | E |
| 140 | N⁴-(5-fluoro-6- methyl-1H-indazol- 3-yl)-N⁴-methyl-N²- [3-(methyloxy)-5- (methylsulfonyl) phenyl]-2,4- pyrimidinediamine | 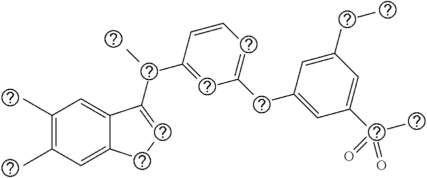 | ¹H NMR (DMSO-d₆) δ 13.05 (s, 1H), 9.69 (s, 1H), 8.21 (s, 1H), 7.95 (d, J = 6.1 Hz, 1H), 7.67 (t, J = 1.9 Hz, 1H), 7.49 (d, J = 6.1 Hz, 1H), 7.27 (d, J = 9.6 Hz, 1H), 6.95- 7.02 (m, 1H), 5.87 (d, J = 5.8 Hz, 1H), 3.75- 3.86 (m, 3H), 3.56 (s, 3H), 3.19 (s, 3H), 2.33- 2.41 (m, 3H) | 457 | E |
| 141 | N4-(6,7-difluoro- 1H-indazol-3-yl)- N2-(1,1-dioxido-1- benzothien-6-yl)- N4-methyl-2,4- pyrimidinediamine | 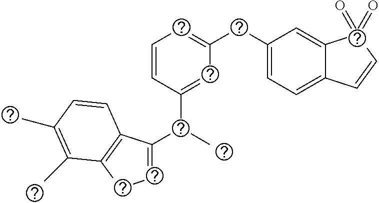 | ¹H NMR (DMSO-d₆) δ 13.78 (br. s., 1H), 9.90 (s, 1H), 8.24 (br. s., 1H), 8.08 (d, J = 5.8 Hz, 1H), 7.77-7.83 (m, 1H), 7.52 (d, J = 7.6 Hz, 1H), 7.36 (s, 2H), 7.14 (d, J = 6.8 Hz, 2H), 6.10 (d, J = 6.1 Hz, 1H), 3.55 (s, 3H) | 441 | E |
| 142 | N4-methyl-N4-(5- methyl-1H-indazol- 3-yl)-N2-[3- (methyloxy)-5- (methylsulfonyl) phenyl]-2,4- pyrimidinediamine | 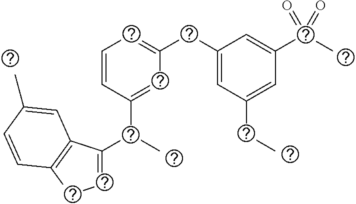 | ¹H NMR (DMSO-d₆) δ 11.42 (s, 2H), 7.00- 7.10 (m, 4H), 6.59 (d, J = 6.3 Hz, 2H), 5.28 (d, J = 5.1 Hz, 2H), 2.82 (s, 3H), 2.80 (s, 3H), 2.58 (s, 6H) | 439 | E |
| 143 | N4-methyl-N4-(5- methyl-1H-indazol- 3-yl)-N2-[3-methyl- 5-(methylsulfonyl) phenyl]-2,4- pyrimidinediamine | 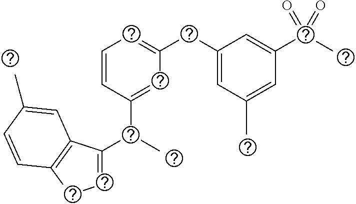 | ¹H NMR (DMSO-d₆) δ 12.97 (br. s., 1H), 9.63- 9.67 (m, 1H), 8.45- 8.50 (m, 1H), 7.94 (d, J = 5.8 Hz, 1H), 7.73- 7.79 (m, 1H), 7.48 (d, J = 8.6 Hz, 1H), 7.22- 7.32 (m, 3H), 5.83 (d, J = 5.8 Hz, 1H), 3.56 (s, 3H), 3.16 (s, 3H), 2.37 (s, 3H), 2.33 (s, 3H) | 423 | E |
| 144 | 3-({4-[(7-fluoro-6- methyl-1H-indazol- 3-yl)(methyl) amino]-2- pyrimidinyl} amino)-5- methylbenzene sulfonamide hydrochloride | 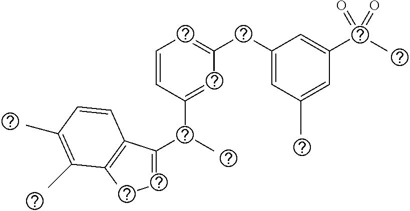 | ¹H NMR (DMSO-d₆) δ 13.46 (br. s., 1H), 9.56 (s, 1H), 8.29 (br. s., 1H), 7.96 (d, J = 6.1 Hz, 1H), 7.63 (br. s., 1H), 7.21 (s, 6H), 6.96-7.02 (m, 1H), 5.93 (d, J = 6.1 Hz, 1H), 3.56 (s, 3H), 2.37 (d, J = 1.8 Hz, 3H), 2.25 (s, 3H) | 442 | I |
| 145 | N4-(7-fluoro-1H- indazol-3-yl)-N2- (3-methoxy-5- (methylsulfonyl) phenyl)pyrimidine- 2,4-diamine | 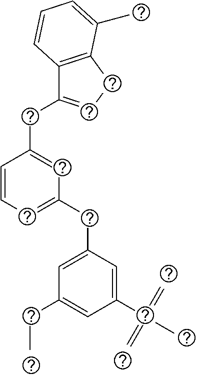 | ¹H NMR (DMSO-d₆) δ 13.17 (br. s., 1H), 9.84 (s, 1H), 9.47 (s, 1H), 8.18 (d, J = 5.8 Hz, 1H), 7.84 (s, 1H), 7.78 (s, 1H), 7.63 (d, J = 8.3 Hz, 1H), 7.21 (dd, J = 11.2, 7.7 Hz, 1H), 6.98-7.06 (m, 1H), 6.91 (s, 1H), 6.85 (d, J = 5.1 Hz, 1H), 3.73 (s, 3H), 3.16 (s, 3H) | 429 | D |
| 146 | 5-((4-((7-fluoro- 1H-indazol-3- yl)(methyl)amino) pyrimidin-2- yl)amino)-2- methoxybenzene sulfonamide | 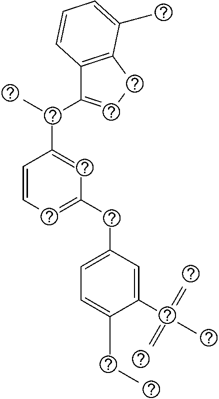 | ¹H NMR (DMSO-d₆) δ 13.63 (br. s., 1H), 9.36 (s, 1H), 8.39 (d, J = 2.5 Hz, 1H), 7.91 (d, J = 5.8 Hz, 1H), 7.75 (dd, J = 9.0, 2.7 Hz, 1H), 7.35 (d, J = 8.1 Hz, 1H), 7.27 (dd, J = 11.5, 7.5 Hz, 1H), 7.07-7.13 (m, 1H), 7.03 (d, J = 9.1 Hz, 1H), 6.98 (s, 2H), 5.84 (d, J = 5.8 Hz, 1H), 3.85 (s, 3H), 3.56 (s, 3H) | 444 | D |
| 147 | N4-(6-methyl-1H- indazol-3-yl)-N2- [3-methyl-5- (methylsulfonyl) phenyl]-2,4- pyrimidinediamine | 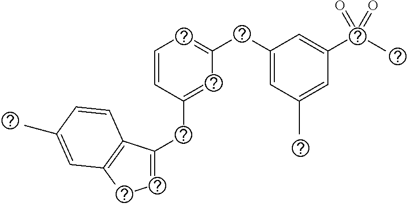 | ¹H NMR (DMSO-d₆) δ 12.45 (s, 1H), 9.68 (s, 1H), 9.41 (br. s., 1H), 8.13 (d, J = 5.8 Hz, 1H), 8.02-8.06 (m, 1H), 7.93-7.99 (m, 1H), 7.62-7.68 (m, 1H), 7.25 (s, 1H), 7.19 (s, 1H), 6.89 (d, J = 8.3 Hz, 1H), 6.77 (br. s., 1H), 3.12 (s, 3H), 2.43 (s, 3H), 2.12-2.22 (m, 3H) | 409 | E |
| 148 | N4-(6,7-difluoro- 1H-indazol-3-yl)- N2-[3-(methyloxy)- 5-(methylsulfonyl) phenyl]-2,4- pyrimidinediamine | 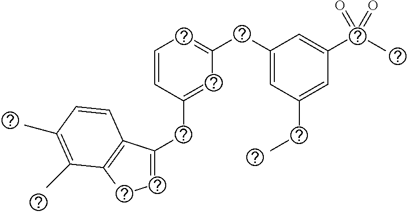 | ¹H NMR (DMSO-d₆) δ 13.30 (s, 1H), 9.92 (s, 1H), 9.48 (s, 1H), 8.19 (d, J = 5.8 Hz, 2H), 7.77- 7.81 (m, 1H), 7.73- 7.77 (m, 1H), 7.60- 7.69 (m, 1H), 7.04- 7.14 (m, 1H), 6.90- 6.93 (m, 1H), 6.85- 6.90 (m, 1H), 3.75 (s, 3H), 3.16 (s, 3H) | 447 | E |
| 149 | 3-((4-((5,6- difluoro-1H- indazol-3- yl)(methyl)amino) pyrimidin-2- yl)amino)-5- methylbenzene sulfonamide | 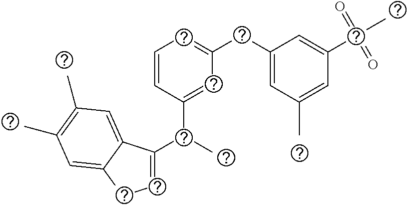 | ¹HNMR(400 MHz, DMSO-d₆) δ ppm 2.0 (s, 2H) 2.34 (s, 3H) 3.56 (s, 3H) 6.0 (br.s., 1H) 7.30 (br. S., 2H) 7.60 (br. S., 1H) 7.75 (br. s., 1H) 7.85 (d, J = 8.69 Hz, 1H) 8.28 (br, s, 1H) 9.51 (br. S., 1H) 13.28 (br.s., 1H) | 446 | E |
| 150 | N4-(5,6-difluoro- 1H-indazol-3-yl)- N2-(4-methoxy-3- (methylsulfonyl) phenyl)-N4- methylpyrimidine- 2,4-diamine | 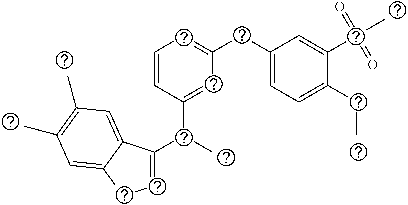 | ¹HNMR(400 MHz, CDCl₃) δ ppm 3.0 (s, 3H) 3.32 (s, 3H) 3.83 (s, 3H) 6.0 (br.s., 1H) 7.30 (br. S., 2H) 7.60 (br. S., 1H) 7.75 (br. s., 1H) 7.85 (d, J = 8.69 Hz, 1H) 8.28 (br, s, 1H) 9.51 (br. S., 1H) 13.28 (br.s., 1H) | 461 | E |
| 151 | N4-(7-fluoro-1H- indazol-3-yl)-N2- (4-methoxy-3- (methylsulfonyl) phenyl)-N4- methylpyrimidine- 2,4-diamine | 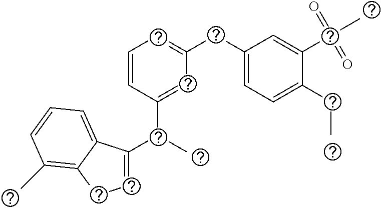 | ¹HNMR(400 MHz, CDCl₃) δ ppm 3.25 (s, 3H) 3.72 (s, 3H) 4.02 (s, 3H) 6.02 (d, J = 6 Hz, 1H) 7.02 (d, J = 8.8 Hz, 1H) 7.23 (m, 2H) 7.35 (m, 1H) 7.65 (d, J = 8.69 Hz, 1H) 7.85 (br, s, 1H) 8.55 (br. S., 1H) 10.75 (br.s., 1H) | 443 | E |
| 152 | N4-(7-fluoro-1H- indazol-3-yl)-N4- methyl-N2-(3- methyl-5- ((tetrahydro-2H- pyran-4- yl)sulfonyl)phenyl) pyrimidine-2,4- diamine | 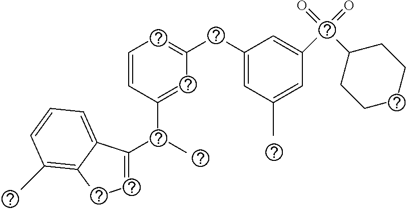 | ¹HNMR(400 MHz, CDCl₃) δ ppm 1.85 (m, 4H) 2.34 (s, 3H) 3.56 (s, 3H) 3.65 (m, 4H) 5.32 (s, 1H) 6.10 (d, J = 6.8 Hz, 1H) 7.12 (d, J = 8.8 Hz, 1H) 7.22 (m, 1H) 7.35 (m, 1H) 7.50 (d, J = 8.69 Hz, 2H) 7.82 (br, s, 1H) 8.50 (br. S., 1H) 10.35 (br.s., 1H) | 497 | E |
| 153 | 3-({4-[(6,7-difluoro- 1H-indazol-3- yl)(methyl)amino]- 2-pyrimidinyl} amino)-5- (methyloxy) benzene sulfonamide | 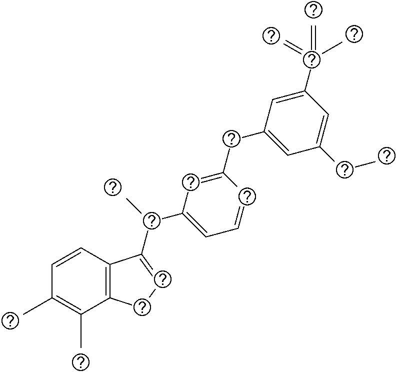 | ¹H NMR (DMSO-d₆) δ 13.75 (br. s., 1H), 9.60 (s, 1H), 7.99 (d, J = 6.1 Hz, 2H), 7.50 (s, 1H), 7.35 (dd, J = 8.6, 4.0 Hz, 1H), 7.26 (s, 2H), 7.10-7.22 (m, 1H), 6.87-6.96 (m, 1H), 5.98 (d, J = 5.8 Hz, 1H), 3.76 (s, 3H), 3.56 (s, 3H) | 462 | E |
| 154 | 3-((4-((5,6- difluoro-1H- indazol-3- yl)(methyl)amino) pyrimidin-2- yl)amino)-5- methoxybenzene sulfonamide | 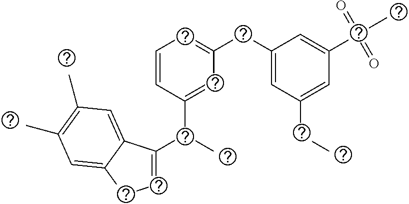 | ¹HNMR(400 MHz, DMSO-d₆) δ ppm 3.50 (s, 2H) 3.70 (s, 3H) 5.90 (d, J = 5.6 Hz, 1H) 6.94 (m, 1H) 7.26 (br. S., 1H) 7.57 (br. S., 1H) 7.61-7.68 (m, 1H) 7.95 (d, J = 8.69 Hz, 1H) 8.02 (br, s, 1H) 9.62 (br. S., 1H) 13.28 (br.s., 1H) | 462 | E |
| 155 | 3-({4-[(5-fluoro-6- methyl-1H-indazol- 3-yl)(methyl) amino]-2- pyrimidinyl} amino)-5- (methyloxy) benzene sulfonamide | 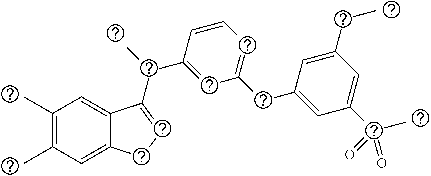 | ¹H NMR (DMSO-d₆) δ 13.04 (br. s., 1H), 9.60 (s, 1H), 8.08 (s, 1H), 7.93 (d, J = 5.8 Hz, 1H), 7.61 (t, J = 2.0 Hz, 1H), 7.49 (d, J = 6.1 Hz, 1H), 7.22-7.35 (m, 3H), 6.89-7.01 (m, 1H), 5.84 (d, J = 5.8 Hz, 1H), 3.78 (s, 3H), 3.55 (s, 3H), 2.32-2.43 (m, 3H) | 458 | E |
| 156 | 3-({4-[(7-fluoro-6- methyl-1H-indazol- 3-yl)(methyl) amino]-2- pyrimidinyl} amino)-5- (methyloxy) benzene sulfonamide | 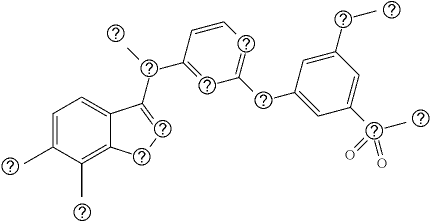 | ¹H NMR (DMSO-d₆) δ 9.61 (s, 1H), 8.06 (s, 1H), 7.92-7.98 (m, 1H), 7.57 (t, J = 1.9 Hz, 1H), 7.36 (br. s., 1H), 7.22 (d, J = 8.3 Hz, 1H), 6.98 (dd, J = 8.1, 6.3 Hz, 1H), 6.92-6.96 (m, 1H), 5.91 (d, J = 5.8 Hz, 1H), 3.77 (s, 3H), 3.57 (s, 3H), 2.37 (d, J = 2.0 Hz, 3H) | 458 | E |
| 157 | 3-({4-[(6,7-difluoro- 1H-indazol-3- yl)amino]-2- pyrimidinyl}amino)- 5-methylbenzene sulfonamide | 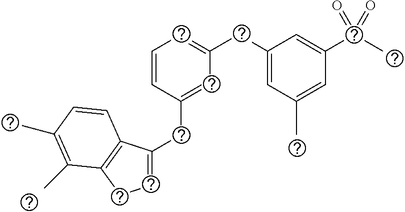 | ¹H NMR (DMSO-d₆) δ 13.32 (s, 1H), 9.86 (s, 1H), 9.40 (s, 1H), 8.18 (d, J = 5.8 Hz, 1H), 7.95- 7.99 (m, 1H), 7.78- 7.83 (m, 1H), 7.64- 7.70 (m, 1H), 7.25 (s, 2H), 7.07-7.17 (m, 2H), 6.85-6.92 (m, 1H), 2.18 (br. s., 3H) | 432 | I |
| 158 | 3-((4-((7-fluoro- 1H-indazol-3- yl)amino)pyrimidin- 2-yl)amino)-5- methoxybenzene sulfonamide | 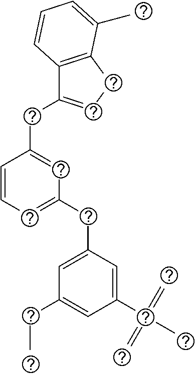 | ¹H NMR (DMSO-d₆) δ 13.17 (s, 1H), 9.79 (s, 1H), 9.43 (s, 1H), 8.17 (d, J = 5.8 Hz, 1H), 7.81 (s, 1H), 7.69 (s, 1H), 7.65 (d, J = 8.1 Hz, 1H), 7.30 (s, 2H), 7.22 (dd, J = 11.5, 7.7 Hz, 1H), 7.04 (td, J = 7.8, 4.5 Hz, 1H), 6.86-6.92 (m, 2H), 3.70 (s, 3H) | 430 | D |
| 159 | 3-((4-((7-fluoro- 1H-indazol-3- yl)(methyl)amino) pyrimidin-2- yl)amino)-5- methoxybenzene sulfonamide | 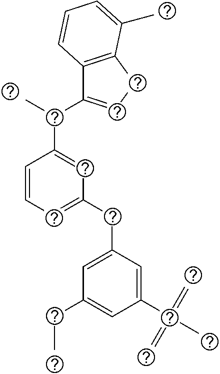 | ¹H NMR (DMSO-d₆) δ 13.64 (br. s., 1H), 9.61 (s, 1H), 8.05 (s, 1H), 7.96 (d, J = 6.1 Hz, 1H), 7.56 (t, J = 2.0 Hz, 1H), 7.35 (d, J = 8.1 Hz, 1H), 7.23-7.30 (m, 3H), 7.10 (td, J = 7.9, 4.4 Hz, 1H), 6.92-6.96 (m, 1H), 5.91 (d, J = 6.1 Hz, 1H), 3.77 (s, 3H), 3.58 (s, 3H) | 444 | D |
| 160 | N²-[4-(ethyloxy)-3- (methylsulfonyl) phenyl]-N⁴-(7- fluoro-1H-indazol- 3-yl)-N⁴-methyl- 2,4- pyrimidinediamine | 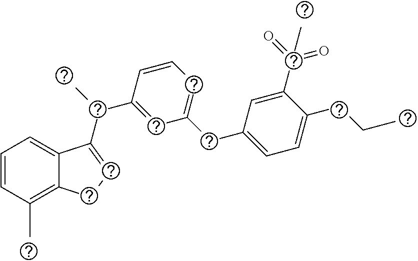 | ¹H NMR (DMSO-d₆) δ 13.64 (br. s, 1H), 9.45 (s, 1H), 8.50 (d, J = 2.3 Hz, 1H), 7.92 (d, J = 5.8 Hz, 1H), 7.81 (dd, J = 9.1, 2.8 Hz, 1H), 7.35 (d, J = 8.1 Hz, 1H), 7.26 (dd, J = 11.4, 7.6 Hz, 1H), 7.04-7.19 (m, 2H), 5.86 (d, J = 6.1 Hz, 1H), 4.17 (q, J = 7.0 Hz, 2H), 3.56 (s, 3H), 3.25 (s, 3H), 1.38 (t, J = 7.0 Hz, 3H) | 457 | E |
| 161 | 3-methoxy-5-((4- (methyl(7- (trifluoromethyl)- 1H-indazol-3- yl)amino)pyrimidin- 2-yl)amino) benzene sulfonamide | 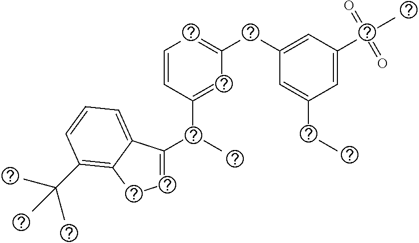 | ¹HNMR(400 MHz, DMSO-d₆) δ ppm 2.0 (s, 2H), 2.40 (s, 3H) 3.50 (s, 3H) 6.01 (d, J = 5.6 Hz, 1H) 7.15 (s, 1H) 7.21 (m, 1H) 7.30 (br. S, 1H) 7.60 (br. S., 1H) 7.80-7.90 (m, H) 7.99 (d, J = 8.69 Hz, 1H) 8.21 (br, s, 1H) 9.61 (br. S., 1H) 13.60 (br.s., 1H) | 494 | E |
| 162 | 3-methyl-5-[(4- {methyl[6- (trifluoromethyl)- 1H-indazol-3- yl]amino}-2- pyrimidinyl)amino] benzene sulfonamide hydrochloride | 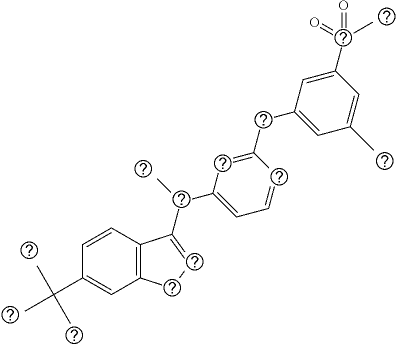 | ¹H NMR (DMSO-d₆) δ 13.77 (br. s., 1H), 10.41 (br. s., 1H), 8.06-8.18 (m, 1H), 8.02 (s, 2H), 7.86 (d, J = 8.6 Hz, 1H), 7.51-7.61 (m, 1H), 7.45 (d, J = 8.8 Hz, 1H), 7.33 (br. s., 3H), 6.14 (br. s., 1H), 3.64 (s, 3H), 2.18-2.36 (m, 3H), | 478 | E |
| 163 | 3-(methyloxy)-5- [(4-{methyl[6- (trifluoromethyl)- 1H-indazol-3- yl]amino}-2- pyrimidinyl)amino] benzene sulfonamide hydrochloride | 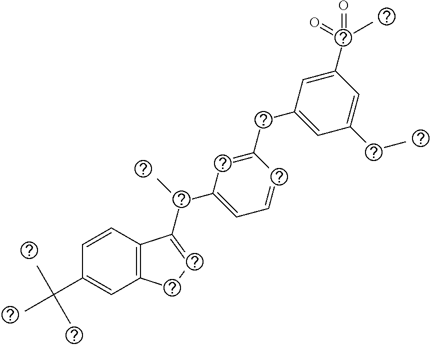 | ¹H NMR (DMSO-d₆) δ 13.79 (br. s., 1H), 10.54 (br. s., 1H), 7.93-8.14 (m, 2H), 7.86 (d, J = 8.3 Hz, 2H), 7.30-7.54 (m, 4H), 7.10 (br. s., 1H), 6.14 (d, J = 4.8 Hz, 1H), 3.79 (s, 3H), 3.65 (s, 3H) | 494 | E |
| 164 | 3-methyl-5-((4- (methyl(7- (trifluoromethyl)- 1H-indazol-3- yl)amino)pyrimidin- 2-yl)amino) benzene sulfonamide | 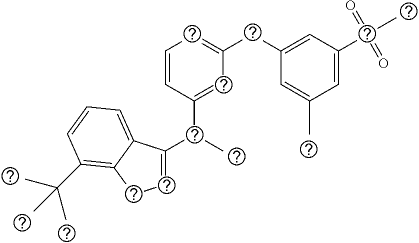 | ¹HNMR(400 MHz, DMSO-d₆) δ ppm 2.2 (s, 2H) 2.5 (s, 3H) 3.50 (s, 3H) 6.01 (d, J = 5.6 Hz, 1H) 7.15 (s, 1H) 7.36 (br. S., 2H) 7.60 (br. S., 1H) 7.61- 7.68 (m, 1H) 7.95 (d, J = 8.69 Hz, 1H) 8.04 (br, s, 1H) 9.65 (br. S., 1H) 13.30 (br.s., 1H) | 478 | E |
| 165 | N,N-dimethyl-3- {[4-(1H- pyrazolo[3,4- b]pyridin-3- ylamino)-2- pyrimidinyl]amino} benzene sulfonamide trifluoroacetate | 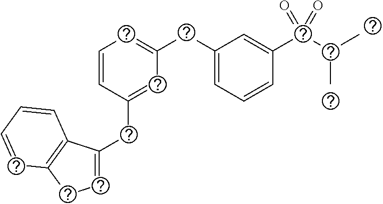 | ¹H NMR (DMSO-d₆) δ 10.50 (s, 1H), 8.72 (d, J = 5.6 Hz, 3H), 7.99- 8.23 (m, 2H), 7.65 (t, J = 8.1 Hz, 1H), 7.39 (dd, J = 13.4, 6.6 Hz, 2H), 6.77 (br. s., 1H), 2.67 (s, 6H) | 411 | C |
| 166 | N4-(5-methyl-1H- pyrazolo[3,4- b]pyridin-3-yl)-N2- [3- (methylsulfonyl) phenyl]-2,4- pyrimidinediamine trifluoroacetate | 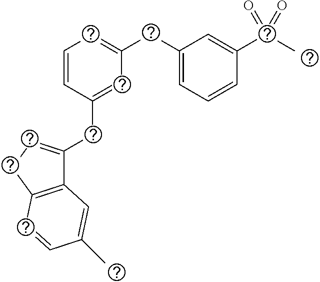 | ¹H NMR (DMSO-d₆) δ 10.50 (s, 1H), 8.72 (d, J = 5.6 Hz, 3H), 7.99- 8.23 (m, 2H), 7.65 (t, J = 8.1 Hz, 1H), 7.39 (dd, J = 13.4, 6.6 Hz, 2H), 6.77 (br. s., 1H), 2.78 (s, 3H), 2.59 (s, 3H) | 396 | C |
| 167 | N2-[4-methyl-3- (methylsulfonyl) phenyl]-N4-(5- methyl-1H- pyrazolo[3,4- b]pyridin-3-yl)-2,4- pyrimidinediamine trifluoroacetate | 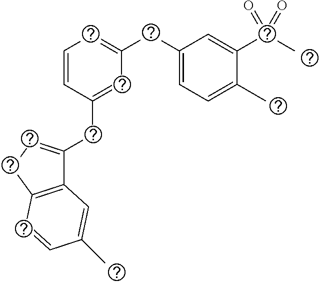 | ¹H NMR (DMSO-d₆) δ 13.25 (br. s., 1H), 10.41- 11.02 (m, 1H), 10.02 (br. s., 1H), 8.38 (d, J = 2.0 Hz, 1H), 8.11 (d, J = 6.5 Hz, 1H), 7.96 (br. s., 3H), 7.11 (br. s., 1H), 6.78 (br. s., 1H), 3.10 (s, 3H), 2.54 (s, 3H), 2.25 (br. s., 3H) | 410 | C |
| 168 | N2-[4-fluoro-3- (methylsulfonyl) phenyl]-N4-(5- methyl-1H- pyrazolo[3,4- b]pyridin-3-yl)-2,4- pyrimidinediamine trifluoroacetate | 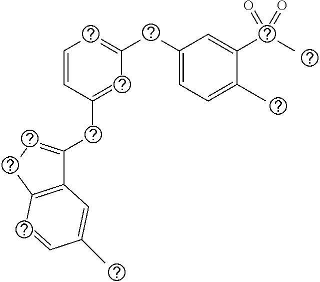 | ¹H NMR (METHANOL- d₄) δ 8.38 (s, 1H), 8.01 (d, J = 7.1 Hz, 1H), 7.87 (br. s., 3H), 7.16 (d, J = 6.3 Hz, 1H), 6.84 (br. s., 1H), 3.15 (br. s., 3H), (br. s., 3H) | 414 | C |
| 169 | N4-(5-fluoro-1H- pyrazolo[3,4- b]pyridin-3-yl)-N2- [3-(methylsulfonyl) phenyl]-2,4- pyrimidinediamine trifluoroacetate | 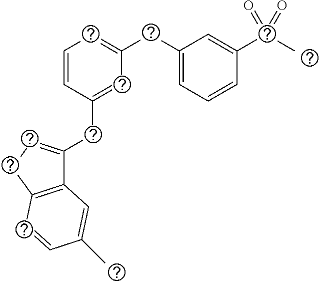 | ¹H NMR (DMSO-d₆) δ 10.54 (br. s., 1H), 9.96 (br. s., 1H), 8.54 (s, 1H), 7.86-8.27 (m, 5H), 7.23-7.59 (m, 2H), 6.79 (br. s., 1H), 3.08 (s, 3H) | 400 | C |
| 170 | N2-[4-fluoro-3- (methylsulfonyl) phenyl]-N4-(5- fluoro-1H- pyrazolo[3,4- b]pyridin-3-yl)-2,4- pyrimidinediamine trifluoroacetate | 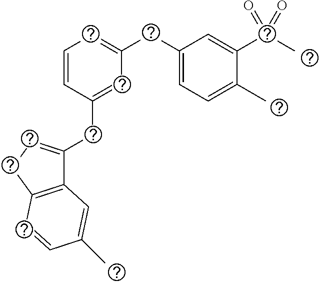 | ¹H NMR (DMSO-d₆) δ 10.51 (br. s., 1H), 9.89 (br. s., 1H), 8.54 (s, 1H), 7.81-8.29 (m, 5H), 7.27 (br. s., 1H), 6.77 (br. s., 1H), 3.23 (s, 3H) | 418 | C |
| 171 | N4-(5-fluoro-1H- pyrazolo[3,4- b]pyridin-3-yl)-N2- [4-methyl-3- (methylsulfonyl) phenyl]-2,4- pyrimidinediamine trifluoroacetate | 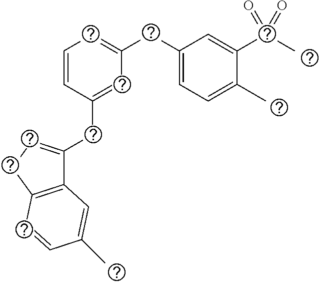 | ¹H NMR (DMSO-d₆) δ 10.66 (br. s., 1H), 9.97 (br. s., 1H), 8.54 (s, 1H), 8.11 (d, J = 6.3 Hz, 2H), 7.67-8.06 (m, 3H), 7.15 (br. s., 1H), 6.76 (br. s., 1H), 3.09 (s, 3H), 2.54 (s, 3H) | 414 | C |
| 172 | N4-(5-fluoro-1H- pyrazolo[3,4- b]pyridin-3-yl)-N2- {4-methyl-3-[(1- methylethyl) sulfonyl]phenyl}- 2,4- pyrimidinediamine trifluoroacetate | 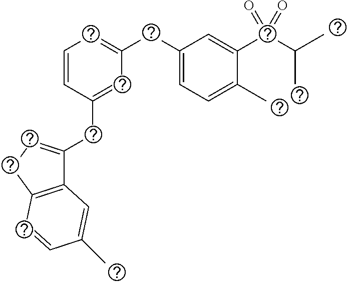 | ¹H NMR (DMSO-d₆) δ 13.56 (br. s., 1H), 10.80 (br. s., 1H), 10.08 (br. s., 1H). 8.54 (s, 1H), 7.66-8.17 (m, 4H), 7.12 (br. s., 1H), 6.74 (br. s., 1H), , 3.34 (m, 1H), 2.34-2.60 (s, 3H), 1.11 (d, J = 6.8 Hz, 6H) | 442 | C |
| 173 | N4-(5-fluoro-1H- pyrazolo[3,4- b]pyridin-3-yl)-N4- methyl-N2-[3- (methylsulfonyl) phenyl]-2,4- pyrimidinediamine | 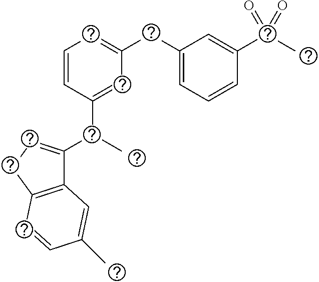 | ¹H NMR (DMSO-d₆) δ 13.85 (br. s., 1H), 9.87 (br. s., 1H), 8.63 (br. s., 1H), 8.56 (br. s., 1H), 7.99-8.06 (m, 3H), 7.79-7.84 (m, 1H), 7.44 (br. s., 2H), 6.07-6.15 (m, 1H), 5.76 (s, 1H), 3.58 (s, 3H), 3.16 (s, 3H) | 413 (M⁺) | A |
| 174 | N4-(5-fluoro-1H- pyrazolo[3,4- b]pyridin-3-yl)-N2- {4-methyl-3-[(1- methylethyl) sulfonyl]phenyl}- 2,4- pyrimidinediamine | 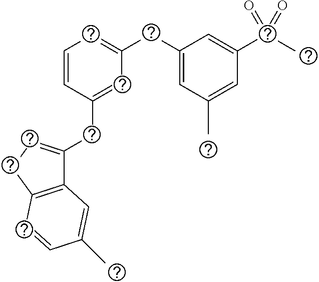 | ¹H NMR (DMSO-d₆) δ 9.96 (s, 1H), 9.43 (s, 1H), 8.46-8.65 (m, 1H), 8.18 (d, J = 5.6 Hz, 1H), 8.07 (br. s., 1H), 7.90 (s, 2H), 7.20 (s, 1H), 6.73 (br. s., 1H), 3.11 (s, 3H), 2.13 (br. s., 3H) | 414 | C |
| 175 | N4-(5-fluoro-1H- pyrazolo[3,4- b]pyridin-3-yl)-N2- [3-(methyloxy)-5- (methylsulfonyl) phenyl]-2,4- pyrimidinediamine | 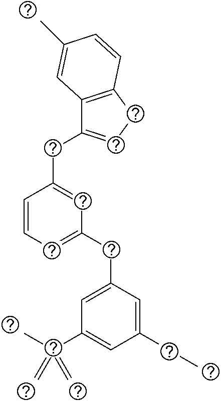 | ¹H NMR (DMSO-d₆) δ 13.33 (br. s., 1H), 9.99 (br. s., 1H), 9.49 (s, 1H), 8.54-8.57 (m, 1H), 8.18 (d, J = 5.8 Hz, 1H), 8.07 (d, J = 9.1 Hz, 1H), 7.72-7.80 (m, 2H), 6.90-6.94 (m, 1H), 6.78 (br. s., 1H), 3.70 (s, 3H), 3.14 (s, 3H) | 430 | A |
| 176 | N4-(5-fluoro-1H- pyrazolo[3,4- b]pyridin-3-yl)-N4- methyl-N2-[3- methyl-5- (methylsulfonyl) phenyl]-2,4- pyrimidinediamine | 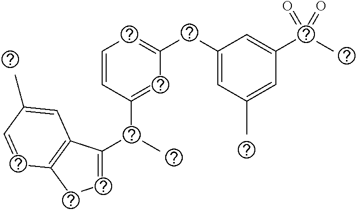 | ¹H NMR (DMSO-d₆) δ 13.81 (br. s., 1H), 9.64 (s, 1H), 8.59-8.64 (m, 1H), 8.33 (br. s., 1H), 8.03 (d, J = 5.8 Hz, 1H), 7.99 (dd, J = 8.6, 2.8 Hz, 1H), 7.70 (s, 1H), 7.24 (s, 1H), 6.08 (d, J = 5.8 Hz, 1H), 3.57 (s, 3H), 3.14 (s, 3H), 2.24 (s, 3H) | 427 (M⁺) | E |
| 177 | N4-(5-fluoro-1H- pyrazolo[3,4- b]pyridin-3-yl)-N4- methyl-N2-[4- methyl-3- (methylsulfonyl) phenyl]-2,4- pyrimidinediamine | 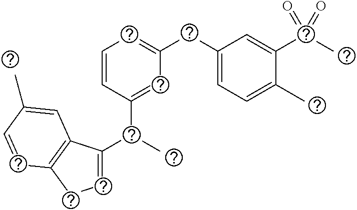 | ¹H NMR (DMSO-d₆) δ 13.78 (br. s., 1H), 9.59 (s, 1H), 8.63 (s, 1H), 8.58 (s, 1H), 7.98-8.03 (m, 2H), 7.71-7.78 (m, 1H), 7.20 (d, J = 8.3 Hz, 1H), 6.03 (d, J = 6.1 Hz, 1H), 3.58 (s, 3H), 3.17 (s, 3H), 2.55 (s, 3H) | 428 | A |
| 178 | N4-(5-fluoro-1H- pyrazolo[3,4- b]pyridin-3-yl)-N2- [3- (methylsulfonyl)-5- (4-morpholinyl) phenyl]-2,4- pyrimidinediamine trifluoroacetate | 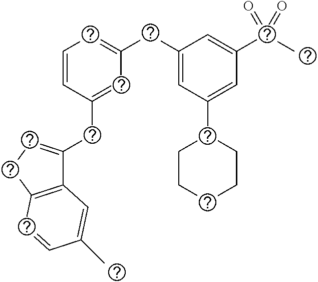 | ¹H NMR (METHANOL- d₄) δ 8.40 (br. s., 1H), 7.99 (d, J = 7.1 Hz, 1H), 7.60 (br. s., 1H), 7.16 (br. s., 3H), 6.63 (br. s., 1H), 3.71 (br. s., 4H), 3.02 (br. s., 3H), 2.82 (br. s., 4H) | 485 | E |
| 179 | N4-(5-fluoro-1H- pyrazolo[3,4- b]pyridin-3-yl)-N2- [4-methyl-3- (methylsulfonyl) phenyl]-2,4- pyrimidinediamine | 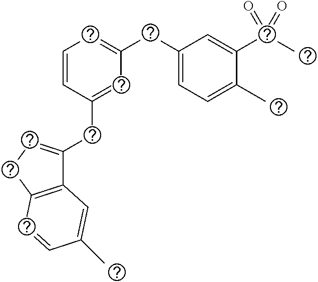 | ¹H NMR (METHANOL- d₄) δ 8.40 (s, 1H), 8.21 (s, 1H), 8.10-8.19 (m, 1H), 7.91-8.05 (m, 1H), 7.82 (d, J = 8.3 Hz, 1H), 7.55-7.65 (m, 1H), 7.29 (d, J = 8.3 Hz, 1H), 3.26 (s, 3H), 2.61 (s, 3H) | 414 | E |
| 180 | N2-[3,4- DIMETHYL-5- (METHYL- SULFONYL) PHENYL]-N4-(5- FLUORO-1H- PYRAZOLO[3,4- B]PYRIDIN-3-YL)- 2,4-PYRIMIDINE- DIAMINE | 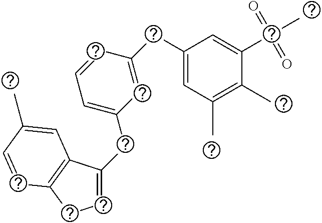 | ¹H NMR (400 MHz, DMSO-d₆) δ ppm 2.05 (s, 3 H) 2.43 (s, 3 H) 3.13 (s, 3 H) 6.71 (br. s., 1 H) 7.87-7.95 (m, 1 H) 7.99 (br. s., 1 H) 8.01-8.08 (m, 1 H) 8.14 (d, J = 5.56 Hz, 1 H) 8.55 (d, J = 1.01 Hz, 1 H) 9.33 (s, 1 H) 9.94 (s, 1 H) 13.33 (br. s., 1 H) | 427 | E |
| 181 | N4-(5-fluoro-1H- pyrazolo[3,4- b]pyridin-3-yl)-N4- methyl-N2-[4- methyl-3- (methylsulfonyl)-5- (1-pyrrolidinyl) phenyl]-2,4- pyrimidinediamine | 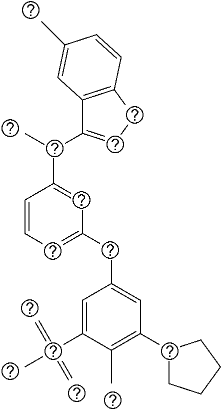 | ¹H NMR (DMSO-d₆) δ 13.76 (br. s., 1H), 9.44 (s, 1H), 8.60 (dd, J = 2.7, 1.6 Hz, 1H), 8.26- 8.30 (m, 1H), 7.95- 8.00 (m, 2H), 7.60- 7.63 (m, 1H), 5.99 (d, J = 6.1 Hz, 1H), 3.58 (s, 3H), 3.18 (s, 3H), 2.97- 3.04 (m, 4H), 2.46 (s, 3H), 1.85-1.92 (m, 4H) | 497 | D |
| 182 | N4-(6-methyl-1H- pyrazolo[3,4- b]pyridin-3-yl)-N2- [3-(methylsulfonyl) phenyl]-2,4- pyrimidinediamine trifluoroacetate | 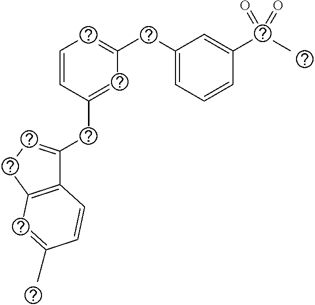 | ¹H NMR (DMSO-d₆) δ 10.58 (br. s., 1H), 10.00 (br. s., 1H), 7.89-8.36 (m, 5H), 7.28-7.67 (m, 2H), 6.94 (d, J = 7.6 Hz, 2H), 3.10 (br. s., 3H), 2.57 (s, 3H) | 396 | C |
| 183 | N2-[3- [(difluoromethyl) oxy]-5- (methylsulfonyl) phenyl]-N4-(5- fluoro-1H- pyrazolo[3,4- b]pyridin-3-yl)-N4- methyl-2,4- pyrimidinediamine | 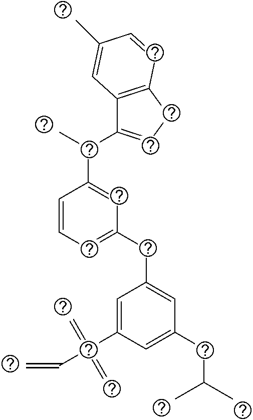 | ¹H NMR (DMSO-d₆) δ 13.81 (br. s., 1H), 9.92 (s, 1H), 8.62 (dd, J = 2.8, 1.8 Hz, 1H), 8.39 (s, 1H), 8.00-8.06 (m, 2H), 7.87-7.90 (m, 1H), 7.11-7.50 (m, 2H), 6.10 (d, J = 6.1 Hz, 1H), 3.58 (s, 3H), 3.23 (s, 3H) | 480 | D |
| 184 | N2-[3-(3,3- difluoro-1- pyrrolidinyl)-5- (methylsulfonyl) phenyl]-N4-(5- fluoro-1H- pyrazolo[3,4- b]pyridin-3-yl)-N4- methyl-2,4- pyrimidinediamine | 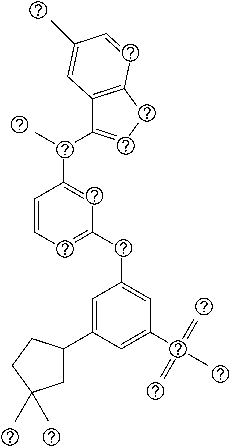 | ¹H NMR (DMSO-d₆) δ 13.78 (br. s., 1H), 9.50 (s, 1H), 8.61 (dd, J = 2.7, 1.6 Hz, 1H), 7.95- 8.03 (m, 2H), 7.92 (s, 1H), 7.22-7.26 (m, 1H), 6.59-6.63 (m, 1H), 6.03 (d, J = 6.1 Hz, 1H), 3.69 (t, J = 13.1 Hz, 2H), 3.58 (s, 3H), 3.44 (t, J = 7.2 Hz, 2H), 3.15 (s, 3H), 2.53-2.63 (m, 2H) | 519 | D |
| 185 | N4-(5-fluoro-1H- pyrazolo[3,4- b]pyridin-3-yl)-N4- methyl-N2-[4- (methylsulfonyl)- 2,3-dihydro-1- benzofuran-6-yl]- 2,4- pyrimidinediamine | 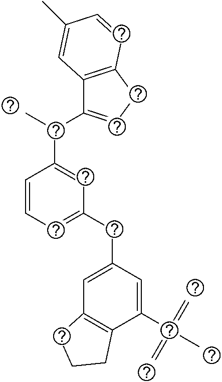 | ¹H NMR (DMSO-d₆) δ 13.76 (br. s., 1H), 9.37 (s, 1H), 8.59-8.64 (m, 1H), 7.91-8.11 (m, 3H), 7.66 (br. s., 1H), 6.02 (d, J = 5.8 Hz, 1H), 4.68 (t, J = 8.6 Hz, 2H), 3.54 (s, 3H), 3.07-3.23 (m, 5H) | 456 | D |
| 186 | N4-(5-FLUORO- 1H- PYRAZOLO[3,4- B]PYRIDIN-3-YL)- N2-[3,4,5- TRIS(METHYL- OXY)PHENYL]- 2,4-PYRIMIDINE- DIAMINE | 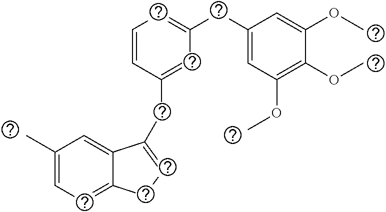 | ¹H NMR (400 MHz, DMSO-d₆) δ ppm 2.05- 2.11 (m, 3 H) 3.60 (s, 6 H) 6.71 (br. s., 3 H) 7.91-8.00 (m, 1 H) 8.04 (d, J = 6.78 Hz, 1 H) 8.54 (br. s., 1 H) 9.85- 10.21 (m, 1 H) 10.82- 11.20 (m, 1 H) 13.62 (br. s., 1 H) | 412 | C |
| 187 | N4-(5-fluoro-1H- pyrazolo[3,4- b]pyridin-3-YL)- N4-methyl-N2- [3,4,5- tris(methyloxy) phenyl]-2,4- pyrimidinediamine | 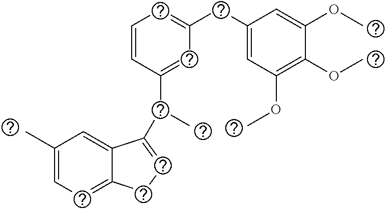 | ¹H NMR (400 MHz, DMSO-d₆) δ ppm 3.61- 3.67 (m, 6 H) 3.70 (br. s., 6 H) 6.29 (br. s., 1 H) 6.86 (br. s., 2 H) 8.01 (br. s., 1 H) 8.05- 8.21 (m, 1 H) 8.63 (br. s., 1 H) 10.58-10.95 (m, 1 H) 14.11 (br. s., 1 H) | 462 | C |
| 188 | N2-[4-methyl-3- (methylsulfonyl) phenyl]-N4-(6- methyl-1H- pyrazolo[3,4- b]pyridin-3-yl)-2,4- pyrimidinediamine | 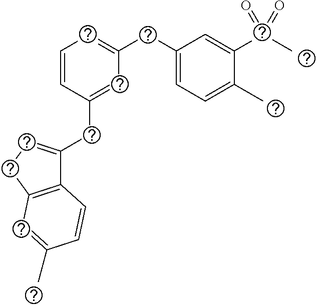 | ¹H NMR (METHANOL- d₄) δ 8.22 (d, J = 2.0 Hz, 1H), 8.06-8.14 (m, 1H), 8.00 (s, 1H), 7.83 (dd, J = 8.3, 2.3 Hz, 1H), 7.18 (d, J = 8.3 Hz, 1H), 6.99 (d, J = 8.1 Hz, 1H), 6.81 (br. s., 1H), 3.09 (s, 3H), 2.88 (s, 3H), 2.65 (s, 3H) | 410 | C |
| 189 | N2-{3-[(1,1- dimethylethyl) sulfonyl]-5- methylphenyl}-N4- (5-fluoro-1H- pyrazolo [3,4- b]pyridin-3-yl)-2,4- pyrimidinediamine | 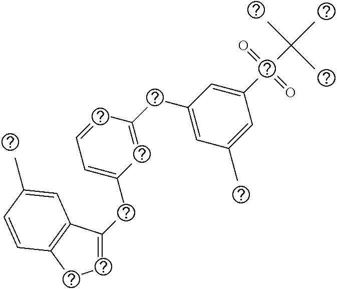 | ¹H NMR (400 MHz, DMSO-d₆) δ ppm 1.22 (s, 9 H) 2.09 (br. s., 3 H) 6.69 (br. s., 1 H) 7.07 (s, 1 H) 7.81 (br. s., 1 H) 7.96 (s, 1 H) 7.99-8.10 (m, 1 H) 8.15 (d, J = 5.81 Hz, 1 H) 8.55 (dd, J = 2.65, 1.64 Hz, 1 H) 9.42 (s, 1 H) 9.95 (br. s., 1 H) 13.35 (br. s., 1 H) | 456 | E |
| 190 | N2-[3-[(1,1- dimethylethyl) sulfonyl]-5- (trifluoromethyl) phenyl]-N4-(5- fluoro-1H-pyrazolo [3,4-b]pyridin-3-yl)- 2,4- pyrimidinediamine | 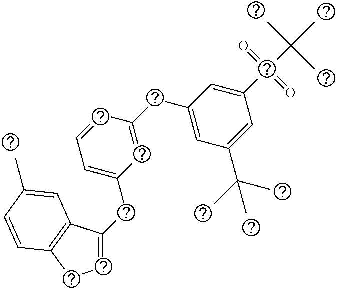 | ¹H NMR (400 MHz, DMSO-d₆) δ ppm 1.19- 1.33 (m, 9 H) 6.73 (br. s., 1 H) 7.41 (s, 1 H) 8.01 (dd, J = 8.72, 2.15 Hz, 1 H) 8.20 (d, J = 5.81 Hz, 1 H) 8.36 (br. s., 1 H) 8.53 (d, J = 1.52 Hz, 2 H) 9.84 (s, 1 H) 10.07 (s, 1 H) 13.36 (br. s., 1 H) | 510 | E |
| 191 | N4-(5-fluoro-6- methyl-1H- pyrazolo[3,4- b]pyridin-3-yl)-N4- methyl-N2-[3- (methyloxy)-5- (methylsulfonyl) phenyl]-2,4- pyrimidinediamine | 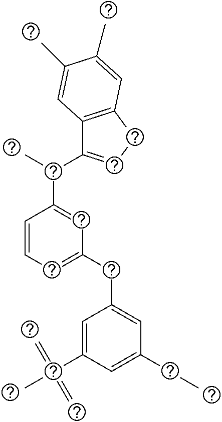 | ¹H NMR (DMSO-d₆) δ 13.57 (br. s., 1H), 9.68 (s, 1H), 8.11 (s, 1H), 8.02 (d, J = 6.1 Hz, 1H), 7.87 (d, J = 9.6 Hz, 1H), 7.62 (s, 1H), 6.94-6.99 (m, 1H), 6.06 (d, J = 6.1 Hz, 1H), 3.78 (s, 3H), 3.57 (s, 3H), 3.17 (s, 3H), 2.57 (d, J = 3.3 Hz, 3H) | 458 | A |
| 192 | N4-(5-fluoro-6- methyl-1H- pyrazolo[3,4- b]pyridin-3-yl)-N4- methyl-N2-[3- methyl-5- (methylsulfonyl) phenyl]pyrimidine- 2,4-diamine | 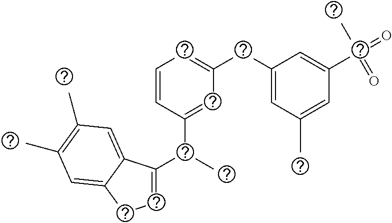 | ¹H NMR(600 MHz, DMSO-d₆) δ ppm 2.25 (br.s,. 3H) 2.53-2.61 (m, 3H) 3.16 (s, 3H) 3.59 (s, 3H) 6.19 (br.s., 1H) 7.35 (br. S., 1H) 7.62 (br. S., 1H) 7.90 (d, J = 8.69 Hz, 1H) 8.03 (d, J = 6.04 Hz, 1H) 8.23 (br, s., 1H) 10.14 (br. S., 1H) 13.71 (br.s., 1H) | 442 | E |
| 193 | N4-(5-fluoro-6- methyl-1H- pyrazolo[3,4- b]pyridin-3-yl)-N2- [3-(methyloxy)-5- (methylsulfonyl) phenyl]-2,4- pyrimidinediamine | 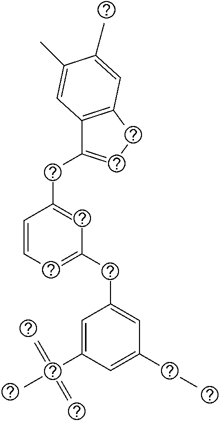 | ¹H NMR (DMSO-d₆) δ 13.11 (br. s., 1H), 9.94 (s, 1H), 9.47 (s, 1H), 8.13-8.21 (m, 1H), 7.91-7.98 (m, 1H), 7.77 (br. s., 1H), 7.74 (s, 1H), 6.89-6.93 (m, 1H), 6.77 (br. s., 1H), 3.70 (s, 3H), 3.14 (s, 3H), 2.54 (d, J = 3.3 Hz, 3H) | 444 | A |
| 194 | N4-(5-fluoro-6- methyl-1H- pyrazolo[3,4- b]pyridin-3-yl)-N4- methyl-N2-[3- (methylsulfonyl)-5- (1-pyrrolidinyl) phenyl]-2,4- pyrimidinediamine | 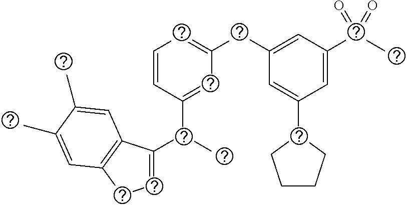 | ¹HNMR(400 MHz, DMSO-d₆) δ ppm 1.97 (m, 4H), 2.53-2.61 (m, 3H) 3.10 (s, 3H) 3.16 (m, 4H) 3.56 (s, 3H) 6.02 (br.s., 1H) 6.52 (br. S, 1H) 7.20 (s., 1H) 7.70 (br. S., 1H) 7.80 (d, J = 8.69 Hz, 1H) 8.00 (d, J = 6.04 Hz, 1H) 9.41 (br. S., 1H) 13.70 (br.s., 1H) | 497 | E |
| 195 | N2-[3- [(difluoromethyl) oxy]-5- (methylsulfonyl) phenyl]-N4-(5- fluoro-6-methyl- 1H-pyrazolo[3,4- b]pyridin-3-yl)-N4- methyl-2,4- pyrimidinediamine | 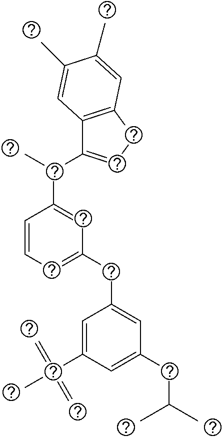 | ¹H NMR (DMSO-d₆) δ 13.59 (br. s., 1H), 9.91 (s, 1H), 8.39 (s, 1H), 8.03 (d, J = 6.1 Hz, 1H), 7.85-7.93 (m, 2H), 7.10-7.50 (m, 2H), 6.08 (d, J = 6.1 Hz, 1H), 3.57 (s, 3H), 3.22 (s, 3H), 2.57 (d, J = 3.3 Hz, 3H) | 494 | E |
| 196 | N4-(5-fluoro-6- methyl-1H- pyrazolo[3,4- b]pyridin-3-yl)-N4- methyl-N2-[4- methyl-3- (methylsulfonyl) phenyl]-2,4- pyrimidinediamine | 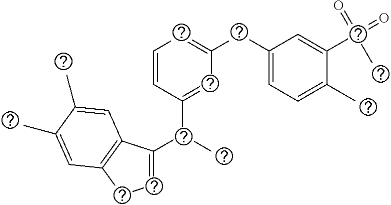 | ¹HNMR(400 MHz, DMSO-d₆) δ ppm 2.50 (br.s,. 3H) 2.57 (br.s; 3H) 3.17 (s, 3H) 3.60 (s, 3H) 6.16 (br.s., 1H) 7.35 (br. S., 1H) 7.68 (br. S., 1H) 7.92 (d, J = 8.69 Hz, 1H) 8.01 (d, J = 6.04 Hz, 1H) 8.50 (br, s., 1H) 10.27 (br. S., 1H) 13.75 (br.s., 1H) | 442 | E |
| 197 | N4-(5-fluoro-6- methyl-1H- pyrazolo[3,4- b]pyridin-3-yl)-N4- methyl-N2-[3- (methylsulfonyl) phenyl]-2,4- pyrimidinediamine | 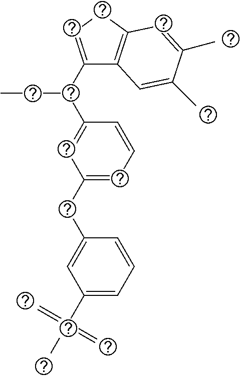 | ¹HNMR(600 MHz, DMSO-d₆) δ ppm 2.50- 2.52 (m, 3H) 3.17 (s, 3H) 3.59 (s, 3H) 6.15 (br.s., 1H)7.45 (br. S., 1H) 7.50 (m, 3H) 7.62 (br. S., 1H) 7.80 (d, J = 8.69 Hz, 1H) 8.03 (d, J = 6.04 Hz, 1H) 8.53 (br, s., 1H) 10.12 (br. S., 1H) 13.70 (br.s., 1H) | 428 | E |
| 198 | N4-(5-fluoro-6- methyl-1H- pyrazolo[3,4- b]pyridin-3-yl)-N4- methyl-N2-[3,4,5- tris(methyloxy) phenyl]-2,4- pyrimidinediamine | 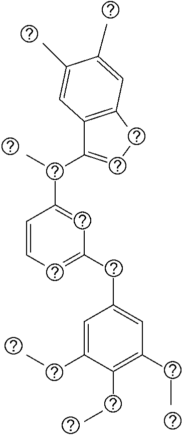 [SMILES: CCc1cc(Cc2cccc(C(C)C3=CCc4cc(C)c(C)cc43)c2)cc(CC)c1CC] | ¹H NMR (DMSO-d₆) δ 13.54 (br. s., 1H), 9.11 (s, 1H), 7.96 (d, J = 6.1 Hz, 1H), 7.85 (d, J = 9.3 Hz, 1H), 7.16 (s, 2H), 5.98 (d, J = 5.8 Hz, 1H), 3.68 (s, 6H), 3.60 (s, 3H), 3.56 (s, 3H), 2.56 (d, J = 3.0 Hz, 3H) | 440 | D |
| 199 | N2-{3-[(1,1- dimethylethyl) sulfonyl]-5- methylphenyl}-N4- (5-fluoro-6-methyl- 1H-pyrazolo[3,4- b]pyridin-3-yl)-N4- methyl-2,4- pyrimidinediamine | 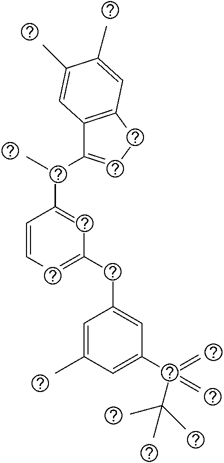 | ¹H NMR (DMSO-d₆) δ 13.56 (br. s., 1H), 9.62 (s, 1H), 8.24 (br. s., 1H), 8.01 (d, J = 6.1 Hz, 1H), 7.86 (d, J = 9.6 Hz, 1H), 7.76 (s, 1H), 7.11 (s, 1H), 6.06 (d, J = 5.8 Hz, 1H), 3.56 (s, 3H), 2.56 (d, J = 3.3 Hz, 3H), 2.23 (s, 3H), 1.25 (s, 9H) | 484 | E |
| 200 | N4-(5-fluoro-1,6- dimethyl-1H- pyrazolo[3,4- b]pyridin-3-yl)-N4- methyl-N2-[3- methyl-5- (methylsulfonyl) phenyl]-2,4- pyrimidinediamine | 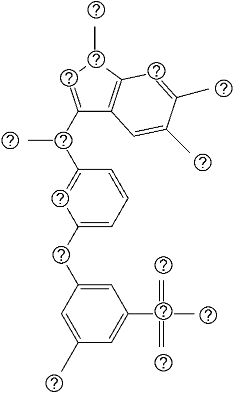 | ¹HNMR(400 MHz, DMSO-d₆) δ ppm 2.25 (br.s,. 3H) 2.53-2.61 (m, 3H) 3.16 (s, 3H) 3.59 (s, 3H) 3.66 (s, 3H) 6.19 (br.s., 1H) 7.35 (br. S., 1H) 7.47 (s, 1H) 7.62 (br. S., 1H) 7.90 (d, J = 8.69 Hz, 1H) 8.03 (d, J = 6.04 Hz, 1H) 8.33 (br, s., 1H) | 456 | E |
| 201 | 3-({4-[(5-fluoro-6- methyl-1H- pyrazolo[3,4- b]pyridin-3- yl)(methyl)amino]- 2-pyrimidinyl} amino)benzene sulfonamide | 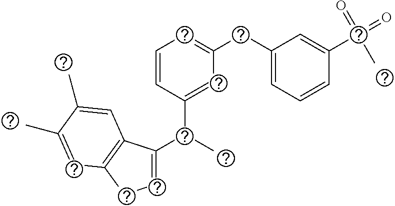 | ¹HNMR(400 MHz, DMSO-d₆) δ ppm 2.0 (s, 2H) 2.53-2.61 (m, 3H) 3.56 (s, 3H) 6.0 (br.s., 1H) 7.30 (br. S., 1H) 7.34 (br. S., 1H) 7.80 (br. s., 1H) 7.95 (d, J = 8.69 Hz, 1H) 8.05 (d, J = 6.04 Hz, 1H) 8.6 (br, s., 1H) 10.12 (br. S., 1H) 13.68 (br.s., 1H) | 429 | E |
| 202 | 3-({4-[(5-fluoro-1H- pyrazolo[3,4- b]pyridin-3- yl)amino]-2- pyrimidinyl}amino) benzene sulfonamide | 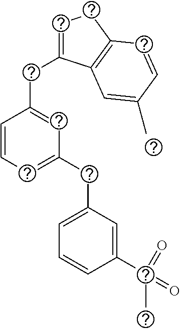 | ¹H NMR (DMSO-d₆) δ 9.45 (s, 1H), 8.53 (d, J = 1.5 Hz, 1H), 8.18 (s, 1H), 8.15 (d, J = 5.8 Hz, 1H), 8.07-8.13 (m, 1H), 8.04 (d, J = 7.1 Hz, 1H), 7.26-7.37 (m, 2H), 6.80 (br. s., 1H) | 401 | E |
| 203 | N4-(5-fluoro-6- methyl-1H- pyrazolo[3,4- b]pyridin-3-yl)-N2- (3-fluoro-5- (methylsulfonyl) phenyl]-N4- methyl-2,4- pyrimidinediamine | 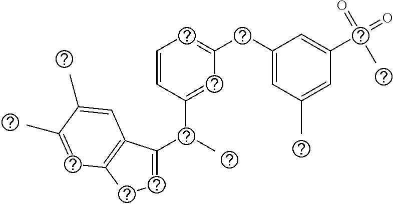 | ¹HNMR(400 MHz, DMSO-d₆) δ ppm 2.49- 2.52 (m, 3H) 3.20 (s, 3H) 3.56 (s, 3H) 6.15 (br.s., 1H) 7.25 (br. S., 1H) 7.85 (d, J = 8.69 Hz, 2H) 8.05 (d, J = 6.04 Hz, 1H) 8.25 (br, s., 1H) 9.95 (br. S., 1H) 13.60 (br.s., 1H) | 446 | E |
| 204 | 3-({4-[(5-fluoro-1H- pyrazolo[3,4- b]pyridin-3- yl)(methyl)amino]- 2-pyrimidinyl} amino)benzene sulfonamide | 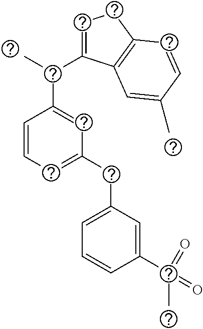 | ¹H NMR (DMSO-d₆) δ 14.01 (s, 1H), 10.48 (br. s., 1H), 8.62-8.69 (m, 1H), 8.33 (br. s., 1H), 8.10 (dd, J = 8.5, 2.7 Hz, 1H), 8.03 (d, J = 6.6 Hz, 1H), 7.70 (d, J = 7.3 Hz, 1H), 7.49 (d, J = 7.6 Hz, 1H), 7.37 (s, 3H), 6.21 (br. s., 1H), 3.61 (s, 3H) | 415 | E |
| 205 | 2-chloro-5-({4-[(5- fluoro-1H- pyrazolo[3,4- b]pyridin-3- yl)amino]-2- pyrimidinyl]amino) benzene sulfonamide | 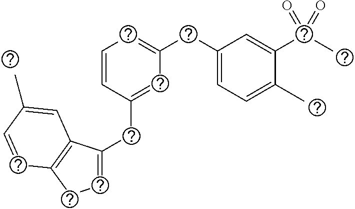 | ¹H NMR (METHANOL- d₄) δ 8.45-8.48 (m, 1H), 8.29-8.32 (m, 1H), 8.13 (d, J = 5.8 Hz, 1H), 7.90-8.00 (m, J = 2.8 Hz, 2H), 7.30 (d, J = 8.6 Hz, 1H), 6.79-6.89 (m, 1H) | 435 | E |
| 206 | 2-chloro-5-({4-[(5- fluoro-1H- pyrazolo[3,4- b]pyridin-3- yl)(methyl)amino]- 2-pyrimidinyl} amino)benzene sulfonamide | 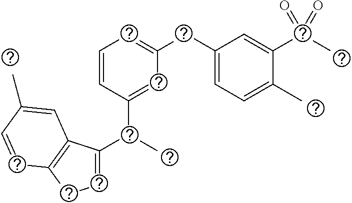 | ¹H NMR (DMSO-d₆) δ 13.79 (br. s., 1H), 9.73 (s, 1H), 8.64-8.68 (m, 1H), 8.63 (dd, J = 2.5, 1.8 Hz, 1H), 7.99-8.03 (m, 2H), 7.80 (dd, J = 8.8, 2.5 Hz, 1H), 7.48 (br. s., 2H), 7.35 (d, J = 8.8 Hz, 1H), 6.03-6.08 (m, 1H), 3.57 (s, 3H) | 449 | E |
| 207 | 5-({4-[(5-fluoro-1H- pyrazolo[3,4- b]pyridin-3- yl)(methyl)amino]- 3- pyrimidinyl}amino)- 2- methylbenzene- sulfonamide | 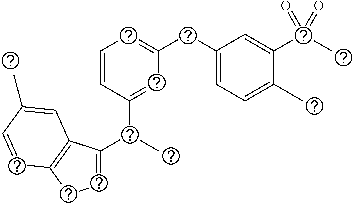 | ¹H NMR (DMSO-d₆) δ 13.79 (br. s., 1H), 9.49 (s, 1H), 8.63 (dd, J = 2.7, 1.6 Hz, 1H), 8.47- 8.50 (m, 1H), 7.95- 8.02 (m, 2H), 7.64- 7.69 (m, 1H), 7.24 (s, 2H), 7.06-7.12 (m, 1H), 6.00 (d, J = 5.8 Hz, 1H), 3.56 (s, 3H) | 429 | E |
| 208 | N4-(5-fluoro-7- oxido-1H- pyrazolo[3,4- b]pyridin-3-yl)-N4- methyl-N2-[3- methyl-5- (methylsulfonyl) phenyl]-2,4- pyrimidinediamine | 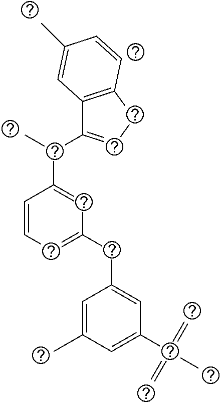 | ¹H NMR (DMSO-d₆) δ 9.55 (s, 1H), 8.54 (br. s., 1H), 8.12 (dd, J = 5.2, 1.9 Hz, 1H), 7.77- 7.86 (m, 2H), 7.16- 7.27 (m, 2H), 5.73 (br. s., 1H), 3.52 (s, 3H), 3.16 (s, 3H), 2.36 (br. s., 3H) | 444 | D |
| 209 | 5-fluoro-6-methyl- 3-(methyl(2-((3- methyl-5- (methylsulfonyl) phenyl)amino) pyrimidin-4- yl)amino)-1H- pyrazolo[3,4-b] pyridine 7-oxide | 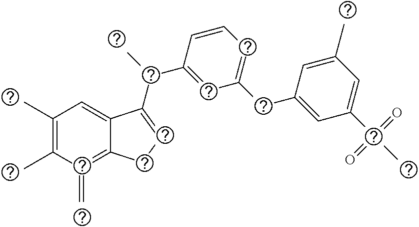 | ¹H NMR (600 MHz, DMSO-d₆) δ ppm 2.34 (br. s., 3 H) 2.43-2.48 (m, 3 H) 3.38 (br. s., 3 H) 3.53 (s, 3 H) 5.76 (br. s., 1 H) 7.25 (br. s., 1 H) 7.29 (d, J = 7.93 Hz, 1 H) 7.77 (br. s., 1 H) 7.83 (d, J = 5.29 Hz, 1 H) 8.54 (br. s., 1 H) 9.57 (br. s., 1 H) | 458 | E |
| 210 | N4-(5-fluoro-6- methyl-1H- pyrazolo[3,4- b]pyridin-3-yl)-N4- methyl-N2-(3- methyl-5- ((tetrahydro-2H- pyran-4- yl)sulfonyl)phenyl) pyrimidine-2,4- diamine | 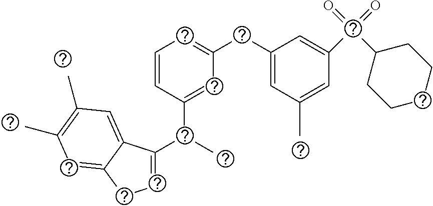 | ¹HNMR(400 MHz, DMSO-d₆) δ ppm 2.25 (m, 4H) 2.40 (br. s., 3H) 2.55-2.56 (m, 3H) 2.95 (s, 3H) 3.10 (m, 1H) 3.55 (m, 4H) 6.04 (br.s., 1H) 6.93 (s, 1H) 7.15 (br. S., 1H) 7.47 (br. S., 1H) 7.84 (d, J = 8.69 Hz, 1H) 7.99 (d, J = 6.04 Hz, 1H) 9.67 (br. S., 1H) 13.75 (br.s., 1H) | 512 | E |

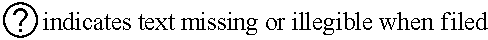

### Pharmaceutical Compositions

### Example A

Tablets are prepared using conventional methods and are formulated as follows:

| Ingredient | Amount per tablet |
|---|---|
| Compound of Example 1 | 5 mg |
| Microcrystalline cellulose | 100 mg |
| Lactose | 100 mg |
| Sodium starch glycollate | 30 mg |
| Magnesium stearate | 2 mg |
| Total | 237 mg |

### Example B

Capsules are prepared using conventional methods and are formulated as follows:

| Ingredient | Amount per tablet |
|---|---|
| Compound of Example 3 | 15 mg |
| Dried starch | 178 mg |
| Magnesium stearate | 2 mg |
| Total | 195 mg |

### Biological In Vitro Assay:

A fluorescent polarization based binding assay was developed to quantitate interaction of novel test compounds at the ATP binding pocket of RIPK2, by competition with a fluorescently labeled ATP competitive ligand. Full length FLAG His tagged RIPK2 was purified from a Baculovirus expression system and was used at a final assay concentration of twice the KDapparent. A fluorescent labeled ligand (5-({[2-({[3-({4-[(5-hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid, prepared as described below) was used at a final assay concentration of 5 nM. Both the enzyme and ligand were prepared in solutions in 50 mM HEPES pH7.5, 150 mM NaCl, 10 mM MgCl2, 1 mM DTT, and 1 mM CHAPS. Test compounds were prepared in 100% DMSO and 100 nL was dispensed to individual wells of a multiwell plate. Next, 5 ul RIPK2 was added to the test compounds at twice the final assay concentration, and incubated at room temperature for 10 minutes. Following the incubation, 5 ul of the fluorescent labeled ligand solution, was added to each reaction, at twice the final assay concentration, and incubated at room temperature for at least 10 minutes. Finally, samples were read on an instrument capable of measuring fluorescent polarization. Test compound inhibition was expressed as percent (%) inhibition of internal assay controls.

For concentration response experiments, normalized data were fit and pIC₅₀ s determined using conventional techniques. For example, the following four parameter logistic equation may be used: y=A+((B−C))/(1+(10^{x})/(10^{C})^{D}), where: y is the % activity (% inhibition) at a specified compound concentration; A is the minimum % activity; B is the maximum % activity; C=log₁₀(IC₅₀); D=Hill slope; x=log₁₀ (compound concentration [M]); and pIC₅₀=(—C).

The pIC₅₀s are averaged to determine a mean value, for a minimum of 2 experiments. As determined using the above method, the compounds of Examples 1-210 exhibited a pIC₅₀ greater than or equal to 6.0. For instance, the compounds of Example 12 and Example 27 inhibited RIP2 kinase in the above method with a mean pIC₅₀ of 8.1 and 7.3 respectively.

### FLAG His Tagged RIPK2 Preparation:

Full-length human RIPK2 (receptor-interacting serine-threonine kinase 2) cDNA was purchased from Invitrogen (Carlsbad, Calif., USA, Clone ID:IOH6368, RIPK2-pENTR 221). Gateway® LR cloning was used to site-specifically recombine RIPK2 downstream to an N-terminal FLAG-6His contained within the destination vector pDEST8-FLAG-His6 according to the protocol described by Invitrogen. Transfection into Spodoptera frugiperda(Sf9) insect cells was performed using Cellfectin® (Invitrogen), according to the manufacturer's protocol.

Sf9 cells were grown in Excell 420 (SAFC Biosciences, Lenexa, Kans., US; Andover, Hampshire UK) growth media at 27° C., 80 rpm in shake flask until of a sufficient volume to inoculate a bioreactor. The cells were grown in a 50 litre working volume bioreactor (Applikon, Foster City, Calif., US; Schiedam, Netherlands) at 27° C., 30% dissolved oxygen and an agitation rate of 60-140 rpm until the required volume was achieved with a cell concentration of approximately 3.7xe6 cells/ml. The insect cells were infected with Baculovirus at a multiplicity of infection (MOI) of 12.7. The cultivation was continued for a 43 hour expression phase. The infected cells were removed from the growth media by centrifugation at 2500 g using a Viafuge (Carr) continuous centrifuge at a flow rate of 80 litres/hour. The cell pellet was immediately frozen and subsequently supplied for purification.

9.83×10¹⁰ Insect cells were re-suspended in 1.4 L lysis buffer (50 mM Tris (pH 8.0), 150 mM NaCl, 0.5 mM NaF, 0.1% Triton X-100, 1 mL/litre Protease Inhibitor Cocktail Set III (available from EMD Group; CalBiochem/Merck Biosciences, Gibbstown, N.J., US; Damstadt, Germany) and processed by dounce homogenization on ice. The suspension was then clarified by centrifugation at 47,900 g for 2 hours, at 4° C. The lysate was decanted from the insoluble pellet and loaded at a linear flow rate of 16 cm/h onto a 55 mL FLAG-M2 affinity column (2.6×10.4 cm) that had been pre-equilibrated with 10 column volumes buffer A (50 mM Tris (pH 8.0), 150 mM NaCl, 0.5 mM NaF, 1 mL/litre Protease Inhibitor Cocktail Set III). The column was then washed with 15 column volumes buffer A, and eluted with 6 column volumes buffer B (buffer A+150 μg/mL 3× FLAG peptide) at a linear flow rate of 57 cm/h. Fractions identified by SDS-PAGE as containing protein of interest were dialyzed to remove the 3×FLAG peptide from the preparation against 5 L of Buffer A (not containing the Protease Inhibitor Cocktail) overnight, using 10 kDa MWCO SnakeSkin Pleated Dialysis Tubing. The purification process yielded 11.3 mg of total protein, with the RIPK2 present at 40% purity by gel densitometry scanning, and identity confirmed by peptide mass fingerprinting. The main contaminating proteins in the preparation were identified as lower molecular weight degraded species of RIPK2.

### Fluorescent Ligand Preparation

### 2-Methyl-5-(2-propen-1-yloxy)aniline

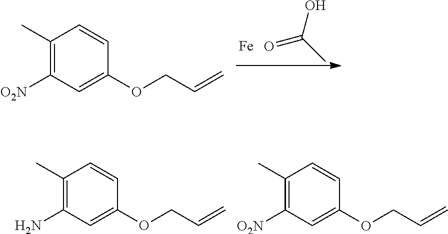

1-Methyl-2-nitro-4-(2-propen-1-yloxy)benzene (25.2 g, 130 mmol) was dissolved in ethanol (280 ml), water (28 ml), and acetic acid (5.6 ml, 98 mmol). Iron (29.1 g, 522 mmol) was added in six portions. The reaction was stirred for 72 hours, and then additional acetic acid (5.6 ml, 98 mmol) and 4 eq. of iron were added. The mixture was filtered through celite rinsing with EtOH and water and the filtrates were concentrated to remove EtOH. Diethylether (300 mL) was added along with 100 mL of 2 N HCl. The layers were separated and the ether layer was extracted with 2×100 mL of 2 N HCl. The acidic aqueous layer was slowly made pH 9 with NaOH pellets, and then dichloromethane (DCM, 300 mL) was added. The resulting emulsion was filtered using a Buchner funnel. The layers were separated and the aqueous layer extracted with DCM (2×100 mL). The combined extracts were dried over MgSO₄), filtered, and concentrated to a dark red oil (15.2 g). The crude material was purified via flash chromatography using a 120 g silica cartridge eluting with 5-15% EtOAc/hexanes for 30 min then 15-30% EtOAc/hexanes for 10 min. to give the titled compound as a red oil. MS (m/z) ¹H NMR (400 MHz, CHLOROFORM-d) 8 ppm 2.23 (s, 3H) 4.51 (dt, J=5.29, 1.51 Hz, 2H) 5.29 (dd, J=10.45, 1.38 Hz, 1H) 5.38-5.46 (m, 1H) 5.99-6.12 (m, 1H) 6.01-6.10 (m, 1H) 6.46 (dd, J=8.31, 2.52 Hz, 1H) 6.56 (d, J=2.52 Hz, 1H) 7.01 (d, J=8.56 Hz, 1H); 164 (M+H+).

### 2-Chloro-N-[2-methyl-5-(2-propen-1-yloxy)phenyl]-4-pyrimidinamine

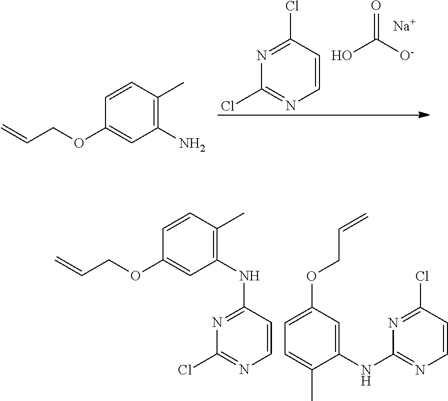

2-Methyl-5-(2-propen-1-yloxy)aniline (11.8 g, 72.3 mmol) was dissolved in tert-butanol (103 ml) and 2,4-dichloropyrimidine (10.77 g, 72.3 mmol) was added followed by sodium bicarbonate (18.22 g, 217 mmol). The reaction was heated at 80° C. for 17 hrs then additional 1,4-dichloropyrimidine (5.38 g, 36.6 mmol) was added and the reaction was stirred for 6 days. Additional 2,4-dichloropyrimidine (2.69 g, 17.8 mmol) was added and the reaction stirred for 2 days. The reaction was cooled to room temp diluting with EtOAc (200 mL) and water (200 mL). The layers were separated and the aqueous layer extracted with EtOAc (2×100 mL). The combined extracts were washed with brine (100 mL), dried over Na₂SO₄, filtered, and concentrated. The crude material was purified via flash chromatography using a 330 g silica cartridge eluting with 1-20% EtOAc/hexanes for min then 20% EtOAc/hexanes for 50 min to give the titled compound (15.1 g). ¹H NMR (400 MHz, CHLOROFORM-d) 8 ppm 2.20 (s, 3H) 4.54 (d, J=5.29 Hz, 2H) 5.32 (dd, J=10.45, 1.38 Hz, 1H) 5.42 (dd, J=17.37, 1.51 Hz, 1H) 5.99-6.12 (m, 1H) 6.35 (d, J=5.79 Hz, 1H) 6.83 (dd, J=8.44, 2.64 Hz, 1H) 6.89 (d, J=2.52 Hz, 6H) 7.14 (br. s., 6H) 7.21 (d, J=8.56 Hz, 7H) 8.10 (d, J=5.79 Hz, 6H); MS (m/z) 276 (M+H⁺).

### 3-[(4-{[2-Methyl-5-(2-propen-1-yloxy)phenyl]amino}-2-pyrimidinyl)amino]benzoic acid

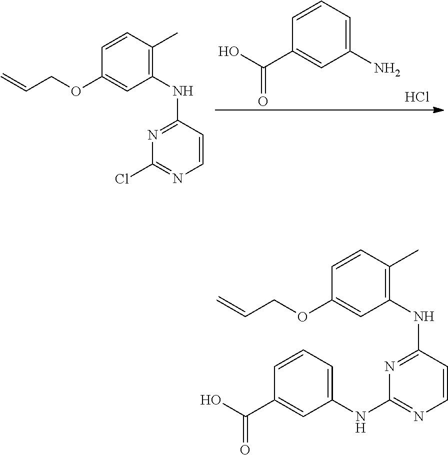 [SMILES: C=CCOc1ccc(C)c(Nc2ccnc(Cl)n2)c1 ; Nc1cccc(C(=O)O)c1 ; Cl ; C=CCOc1ccc(C)c(Nc2ccnc(Nc3cccc(C(=O)O)c3)n2)c1]

2-Chloro-N-[2-methyl-5-(2-propen-1-yloxy)phenyl]-4-pyrimidinamine (8 g, 29.0 mmol), 3-aminobenzoic acid (3.98 g, 29.0 mmol), and HCl (14.51 ml, 29.0 mmol) were dissolved in acetone (58.0 ml) and water (58.0 ml). The reaction was heated to 60° C. for 48 hrs. The reaction was cooled to room temperature passing air over it and a solid crashed out. Water (150 mL) was added and the solid was filtered washing with 3×50 mL water. The solid was dried in the vacuum funnel overnight affording the desired compound (10.9 g). ¹H NMR (400 MHz, METHANOL-d₄) □ ppm 2.21 (s, 3H) 4.47 (d, J=5.04 Hz, 2H) 5.24 (dd, J=10.58, 1.51 Hz, 1H) 5.37 (dd, J=17.25, 1.64 Hz, 1H) 5.97-6.09 (m, 4H) 6.29-6.39 (m, 1H) 6.89 (dd, J=8.44, 2.64 Hz, 4H) 6.96 (d, J=2.77 Hz, 1H) 7.04 (none, 0H) 7.23 (d, J=8.56 Hz, 1H) 7.34-7.41 (m, 1H) 7.75-7.79 (m, 1H) 7.81 (s, 1H) 7.85 (d, J=7.30 Hz, 3H) 7.98-8.09 (m, 3H); MS (m/z) 377 (M+H⁺).

### 1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate

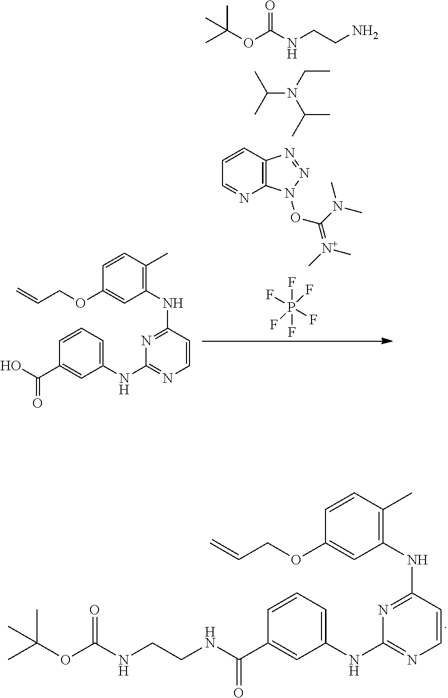

A solution of 3-[(4-{[2-methyl-5-(2-propen-1-yloxy)phenyl]amino}-2-pyrimidinyl)amino]benzoic acid (6.83 g, 18.15 mmol) and DIEA (9.51 ml, 54.4 mmol) in N,N-Dimethylformamide (DMF) (51.8 ml). was treated with N-(2-aminoethyl) carbamic acid tert-butyl ester (3.20 g, 19.96 mmol) and HATU (8.28 g, 21.77 mmol). EtOAc/Et₂O (400 mL, 1:1) was added and the layers separated. The organic layer was washed with water (3×300 mL) and brine (100 mL), dried over Na₂SO₄, filtered, and concentrated to give the titled compound (8.3 g). ¹H NMR (400 MHz, DMSO-d₆) 8 ppm 1.38 (s, 9H) 2.15 (s, 3H) 3.09 (q, J=6.19 Hz, 2H) 3.27 (q, J=6.19 Hz, 2H) 4.51 (d, J=5.27 Hz, 2H) 5.24 (dd, J=10.54, 1.51 Hz, 1H) 5.37 (dd, J=17.32, 1.76 Hz, 1H) 6.02 (m, J=17.29, 10.51, 5.18, 5.18 Hz, 1H) 6.13 (d, J=5.77 Hz, 1H) 6.73 (dd, J=8.41, 2.63 Hz, 1H) 6.90 (t, J=5.65 Hz, 1H) 7.09 (d, J=2.51 Hz, 1H) 7.15 (d, J=8.28 Hz, 1H) 7.17-7.22 (m, 1H) 7.28 (d, J=7.78 Hz, 1H) 7.94-7.99 (m, 2H) 7.99-8.05 (m, 2H) 8.26 (t, J=5.65 Hz, 1H) 8.66 (s, 1H) 9.17 (s, 1H); MS (m/z) 519 (M+H+).

### 1,1-Dimethylethyl {2-({[3-({4-[(5-hydroxy-2-methylphenyl)amino]-2-pyrimidinyl}amino)phenyl]carbonyl}amino)ethyl]carbamate

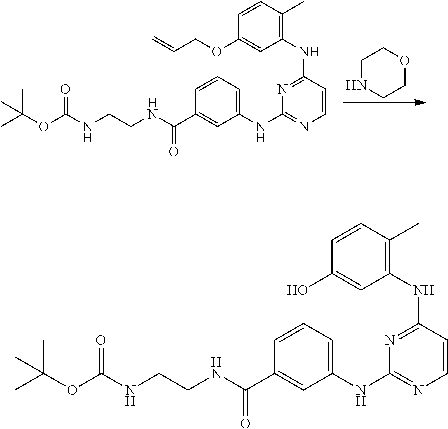 [SMILES: C=CCOc1ccc(C)c(Nc2ccnc(Nc3cccc(C(=O)NCCNC(=O)OC(C)(C)C)c3)n2)c1 ; C1COCCN1 ; Cc1ccc(O)cc1Nc1ccnc(Nc2cccc(C(=O)NCCNC(=O)OC(C)(C)C)c2)n1]

1,1-Dimethylethyl {2-[({3-[(4-{[2-methyl-5-(2-propen-1-yloxy)phenyl]amino}-2-pyrimidinyl)amino]phenyl}carbonyl)amino]ethyl}carbamate (5.5 g, 10.61 mmol) and morpholine (1.016 ml, 11.67 mmol) were dissolved in N,N-dimethylformamide (DMF) (42.4 ml) The atmosphere was exchanged for nitrogen and then it was treated with Tetrakis (1.226 g, 1.061 mmol). The reaction was heated to 80° C. for 3 hrs. The reaction was diluted with EtOAc (250 mL) and washed with water (3×200 mL) then brine (100 mL). The organic layer was dried over Na2SO4, filtered, and concentrated to about 50 mL and let stand overnight. A solid formed and to the suspension was added 50 mL ether. The solid was filtered washing with ether to give the desired product as an orange solid (4.75 g). ¹H NMR (400 MHz, METHANOL-d₄) δ ppm 1.42 (s, 9H) 2.17 (s, 3H) 3.29 (t, J=6.04 Hz, 2H) 3.46 (t, J=6.17 Hz, 2H) 6.04 (d, J=6.04 Hz, 1H) 6.65 (dd, J=8.31, 2.52 Hz, 1H) 6.87 (d, J=2.52 Hz, 1H) 7.09 (d, J=8.31 Hz, 1H) 7.27-7.33 (m, 1H) 7.35-7.41 (m, 1H) 7.53-7.61 (m, 1H) 7.62-7.70 (m, 2H) 7.75 (d, J=8.06 Hz, 1H) 7.91 (d, J=6.04 Hz, 1H) 8.11 (s, 1H); MS (m/z) 479 (M+H⁺).

### N-(2-Aminoethyl)-3-({4-[(5-hydroxy-2-methylphenyl)amino]-2-pyrimidinyl}amino)benzamide

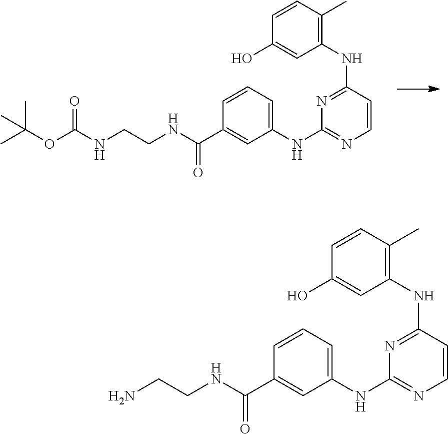 [SMILES: Cc1ccc(O)cc1Nc1ccnc(Nc2cccc(C(=O)NCCNC(=O)OC(C)(C)C)c2)n1 ; Cc1ccc(O)cc1Nc1ccnc(Nc2cccc(C(=O)NCCN)c2)n1]

1,1-Dimethylethyl [2-({[3-({4-[(5-hydroxy-2-methylphenyl)amino]-2-pyrimidinyl}amino)phenyl]carbonyl}amino)ethyl]carbamate (4.75 g, 8.93 mmol) (contaminated with tetrakis or related entities) was dissolved in dichloromethane (DCM) (28.6 ml) and trifluoroacetic acid (TFA) (7.15 ml). The reaction concentrated to give the desired product as the TFA salt containing the same impurities going into the reaction (6.5 g) MS (m/z) 379 (M+H+).

### 5-({[2-({[3-({4-[(5-Hydroxy-2-methylphenyl)amino]-2-pyrimidinyl}amino)phenyl]carbonyl}amino)ethyl]amino}carbonyl)-2-(6-hydroxy-3-oxo-3H-xanthen-9-yl)benzoic acid

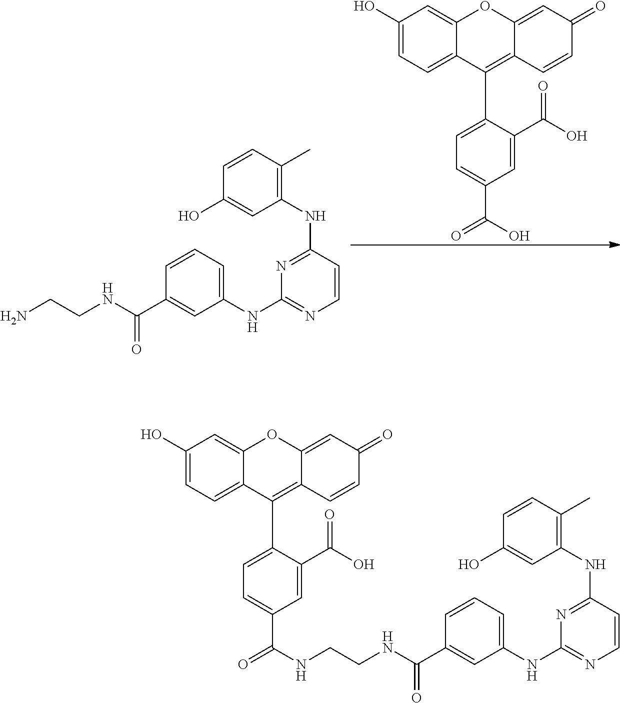 [SMILES: Cc1ccc(O)cc1Nc1ccnc(Nc2cccc(C(=O)NCCN)c2)n1 ; O=C(O)c1ccc(-c2c3ccc(=O)cc-3oc3cc(O)ccc23)c(C(=O)O)c1 ; Cc1ccc(O)cc1Nc1ccnc(Nc2cccc(C(=O)NCCNC(=O)c3ccc(-c4c5ccc(=O)cc-5oc5cc(O)ccc45)c(C(=O)O)c3)c2)n1]

To a suspension of N-(2-aminoethyl)-3-({4-[(5-hydroxy-2-methylphenyl)amino]-2-pyrimidinyl}amino)benzamide (1 g, 1.319 mmol) in N,N-dimethylformamide (DMF) (13.19 ml) was added 5-FAM (5-carboxyfluorescein single isomer) (0.397 g, 1.055 mmol), triethylamine (0.919 ml, 6.60 mmol), EDC (0.506 g, 2.64 mmol), and HOBT (0.202 g, 1.319 mmol). The reaction was stirred overnight then the pH was adjusted to 3 with 2 N HCl. The solution was extracted with EtOAc (100 mL) and the organic layer washed with water (1×50 mL), dried over Na₂SO₄, filtered, and concentrated to give the titled compound. MS (m/z) 737 (M+H⁺).

### Biological In Vivo Assay

The efficacy of the RIP2 inhibitors of this invention may also be evaluated in vivo in rodents. Intraperitoneal (i.p.) or intravenous (i.v.) administration of L18-MDP in mice has been shown to induce an inflammatory response through activation of the NOD2 signaling pathway (Rosenweig, H. L., et al. 2008. Journal of Leukocyte Biology 84:529-536). The level of the inflammatory response in the L18-MDP treated mice/rats is monitored using conventional techniques by measuring increases in cytokine levels (IL8, TNFα, IL6 and IL-1β) in serum and/or peritoneal lavage fluid and by measuring neutrophil influx into the peritoneal space (when L18-MDP is dosed i.p.). Inhibition of the L18-MDP induced inflammatory response in treated rodents may be shown by orally pre-dosing with selected compounds of this invention, then measuring and comparing cytokine levels (IL8, TNFα, IL6 and IL-1β) in serum and/or peritoneal lavage fluid and neutrophil influx into the peritoneal space (when L18-MDP is dosed i.p.) using conventional techniques.

1-35. (canceled)

36. A compound according to Formula (I): 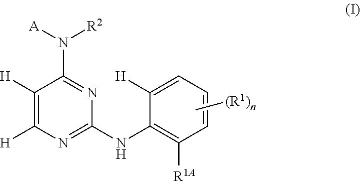
wherein:
R^{1A} is H, fluoro, methyl, methoxy or ethoxy;
n is 1, 2 or 3;
each R¹ is independently selected from halogen, hydroxy, (C₁-C₆)alkyl, cyano(C₁-C₆)alkyl, halo(C₁-C₆)alkyl, (C₁-C₄ alkyl)(C₁-C₄ alkyl)amino-halo(C₂-C₆)alkyl, (C₁-C₄ alkyl)(C₁-C₄ alkyl)amino-, —OR^{x}, —SO₂R^{x}, —NR^{z}SO₂R^{x}, (C₁-C₄ alkyl)(C₁-C₄ alkyl)amino-SO₂NR^{z}—, —CONR^{y}R^{z}, —SO₂NR^{y}R^{z}, —SO₂-heterocycloalkyl, heterocycloalkyl,
and wherein any of said heterocycloalkyl (that is, the heterocycloalkyl group and the heterocycloalkyl moiety of the —SO₂ heterocycloalkyl group) is a 4-7 membered non-aromatic ring containing one heteroatom selected from N, O and S, or containing one nitrogen atom and one additional heteroatom selected from N, O and S; which heterocycloalkyl is optionally substituted by 1-5 substituents independently selected from halogen, (C₁-C₆)alkyl, halo(C₁-C₄)alkyl, —CO(C₁-C₆)alkyl, amino(C₁-C₄)alkyl-, (C₁-C₄ alkyl)amino(C₁-C₄)alkyl-, (C₁-C₄ alkyl)(C₁-C₄ alkyl)amino(C₁-C₄)alkyl- and oxo,
R^{x} is selected from (C₁-C₆)alkyl, halo(C₁-C₆)alkyl, hydroxy(C₂-C₆)alkyl-, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-,
R^{y} is selected from H, (C₁-C₆)alkyl, (C₁-C₆)alkoxy, (C₃-C₇)cycloalkyl, amino(C₂-C₆)alkyl-, ((C₁-C₄)alkyl)amino(C₂-C₆)alkyl-, and ((C₁-C₄)alkyl)((C₁-C₄)alkyl)amino(C₂-C₆)alkyl-, and
R^{z} is H or (C₁-C₆)alkyl;
or one of R^{1A}, taken together with an adjacent R¹ group and the carbon atoms connecting the R^{1A} and R¹ groups form a 5-6 membered, aromatic or non-aromatic heterocyclic ring containing 1 or 2 heteroatom ring moieties independently selected from —NR¹ⁿ—, —O—, —S— and —SO₂—, or two adjacent R¹ groups taken together with the carbon atoms connecting the two groups form a 5-6 membered, aromatic or non-aromatic heterocyclic ring containing 1 or 2 heteroatom ring moieties independently selected from —NR¹ⁿ—, —O—, —S— and —SO₂—, where R¹ⁿ is H or —SO₂(C₁-C₄ alkyl);
R² is H or (C₁-C₄)alkyl;
A is: 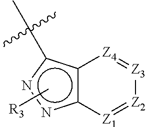
wherein:
R³ is H, (C₁-C₄)alkyl or an optionally substituted phenyl or 5-6 membered heteroaryl, where said phenyl or heteroaryl is optionally substituted by 1-3 groups independently selected from halogen, hydroxy, amino, C₁-C₃ alkyl, haloC₁-C₃ alkyl, C₁-C₃ alkoxy, haloC₁-C₃ alkoxy, (C₁-C₃)alkylamino- and ((C₁-C₃)alkyl)((C₁-C₃)alkyl)amino;
each Z₁, Z₂, Z₃, and Z₄ is independently selected from CH and CR⁴;
any one or two of Z₁, Z₂, Z₃, and Z₄ is N, and each of the remaining two or three of Z₁, Z₂, Z₃, and Z₄ is independently selected from CH and CR⁴, or
any one of Z₁, Z₂, Z₃, and Z₄ is NO, and each of the remaining three of Z₁, Z₂, Z₃, and Z₄ is independently selected from CH and CR⁴,
where each R⁴ is independently selected from halogen, (C₁-C₄)alkyl, halo(C₁-C₄)alkyl, (C₁-C₄)alkoxy, phenyl-oxy, and phenyl(C₁-C₄)alkoxy, wherein the phenyl moiety of said phenyl-oxy or phenyl(C₁-C₄)alkoxy is optionally substituted by 1-3 independently selected (C₁-C₃)alkyl groups;
provided that the compound is not:
N²-(3-{[2-(diethylamino)ethyl]oxy}phenyl)-N-(4-fluoro-2H-indazol-3-yl)-2,4-pyrimidinediamine; or
N²-[3-{[2-(diethylamino)ethyl]oxy}-4-(methyloxy)phenyl]-N⁴-(4-fluoro-2H-indazol-3-yl)-2,4-pyrimidinediamine;
or a salt thereof.

37. A compound according to Formula (I-B):
wherein: 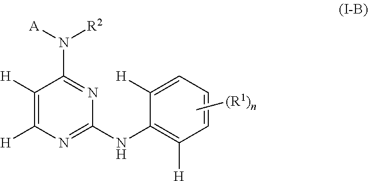
n is 1, 2 or 3;
R¹ is halogen, (C₁-C₆)haloalkoxy, —OR^{x}—SO₂R^{x}, —SO₂NR^{x}R^{y} or heterocycloalkyl,
wherein said heterocycloalkyl is a 5-6 membered non-aromatic ring containing one heteroatom selected from N, O and S, or containing one nitrogen atom and optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, aminoC₁-C₄ alkyl-, (C₁-C₄ alkyl)aminoC₁-C₄ alkyl-, (C₁-C₄ alkyl)(C₁-C₄ alkyl)aminoC₁-C₄ alkyl-, and oxo; and
wherein each R^{x} and R^{y} are independently selected from H, (C₁-C₆ alkyl), (C₃-C₇)cycloalkyl, amino(C₂-C₆ alkyl)-, (C₁-C₄ alkyl)amino(C₂-C₆ alkyl)-, and (C₁-C₄ alkyl)(C₁-C₄ alkyl)amino(C₂-C₆ alkyl)-, or
R^{x} and R^{y} taken together with the nitrogen atom to which they are attached form a 4-7 membered non-aromatic heterocyclic ring optionally containing 1 additional heteroatom selected from N, O and S; which is optionally substituted by 1-5 substituents independently selected from (C₁-C₆)alkyl, (C₁-C₄)haloalkyl, —CO(C₁-C₆)alkyl, amino(C₁-C₄ alkyl)-, (C₁-C₄ alkyl)amino(C₁-C₄ alkyl)-, (C₁-C₄ alkyl)(C₁-C₄ alkyl)amino(C₁-C₄ alkyl)-, and oxo;
R² is H or (C₁-C₄)alkyl;
A is: 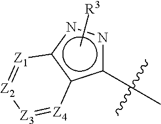
wherein:
R³ is H, (C₁-C₄)alkyl or an optionally substituted phenyl or 5-6 membered heteroaryl, where said phenyl or heteroaryl is optionally substituted by 1-3 groups independently selected from halogen, hydroxy, amino, C₁-C₃ alkyl, haloC₁-C₃ alkyl, C₁-C₃ alkoxy, haloC₁-C₃ alkoxy, (C₁-C₃)alkylamino- and ((C₁-C₃)alkyl)((C₁-C₃)alkyl)amino;
each Z₁, Z₂, Z₃, and Z₄ is independently selected from CH and CR⁴;
or any one or two of Z₁, Z₂, Z₃, and Z₄ is N, and each of the remaining two or three of Z₁, Z₂, Z₃, and Z₄ is independently selected from CH and CR⁴,
where each R⁴ is independently selected from halogen, C₁-C₄ alkyl, C₁-C₄ alkoxy, and phenylC₁-C₄ alkoxy;
provided that the compound is not:
N²-(3-{[2-(diethylamino)ethyl]oxy}phenyl)-N′-(4-fluoro-2H-indazol-3-yl)-2,4-pyrimidinediamine; or
N²-[3-{[2-(diethylamino)ethyl]oxy}-4-(methyloxy)phenyl]-N′-(4-fluoro-2H-indazol-3-yl)-2,4-pyrimidinediamine;
or a salt thereof.

38. The compound or salt according to claim 36, wherein R^{1A} is H.

39. The compound or salt according to claim 36, wherein R^{1A} taken together with an adjacent R¹ group form a —CH═CH₂S— or a —CH═CH₂NH— moiety or two adjacent R¹ groups form a —SO₂CH═CH₂—, —OCH₂CH₂O—, —CH₂CH₂O—, —CH═CH₂NH— or a —CH═CH₂N(SO₂CH₃)— moiety.

40. The compound or salt according to claim 36, wherein each R¹ is independently selected from halogen, (C₁-C₄)alkoxy, —SO₂(C₁-C₄)alkyl, —SO₂NR^{y}R^{z}, and an optionally substituted 6-membered non-aromatic heterocyclic ring containing one heteroatom selected from N, O and S, or containing one nitrogen atom and optionally containing 1 additional heteroatom selected from N, O and S, where said heterocyclic ring is optionally substituted one or two times, independently, by (C₁-C₆)alkyl,
wherein R^{y} and R^{z} are each independently selected from H and (C₁-C₄ alkyl) or R^{y} is H, (C₁-C₂ alkyl), or (C₁-C₂ alkyl)(C₁-C₂ alkyl)amino(C₂-C₃ alkyl)- and R^{z} is H or (C₁-C₂ alkyl), or R^{y} and R^{z}, taken together are —CH₂CH₂CH₂CH₂—.

41. The compound or salt according to claim 36, wherein each R¹ is independently selected from hydroxy, chloro, fluoro, —OCH₃, —OCH₂CH₃, —OCHF₂, —CH₃, —CF₃, —CH(CF₃)N(CH₃)₂, —N(CH₃)₂, —C(CN)(CH₃)₂, —CONH₂, —SO₂CH₃, —SO₂CF₃, —SO₂CH₂CH₃, —SO₂CH(CH₃)₂, —SO₂C(CH₃)₃, —SO₂C(CH₃)₂CH₂OH, —SO₂NH₂, —SO₂N(CH₃)₂, —NHSO₂CH₃, —N(CH₃)SO₂CH₃, —NHSO₂N(CH₃)₂, —SO₂NHOCH₃, —SO₂N(CH₃)OCH₃, —SO₂N(CH₃)(CH₂CH₂N(CH₃)₂), —SO₂-pyrrolidin-1-yl, —SO₂-morpholin-4-yl, —SO₂-tetrahydropyran-4-yl, -pyrrolidin-1-yl, 3,3-difluoro-pyrrolidin-1-yl, 1,1-dioxido-isothiazolidin-2-yl, morpholin-4-yl, and 4-methyl-piperazin-1-yl.

42. The compound or salt according to claim 36, wherein each R¹ is independently selected from chloro, fluoro, methoxy, —SO₂(CH₃), —SO₂ pyrrolidin-1-yl, —SO₂NH₂, —SO₂N(CH₃)₂, —SO₂N(CH₃)(CH₂CH₂N(CH₃)₂), and 4-methyl-piperazin-1-yl.

43. The compound or salt according to claim 36, wherein n is 2 or 3 and each R¹ is independently selected from (C₁-C₄)alkoxy.

44. The compound or salt according to claim 36, wherein R² is H or R² is methyl.

45. The compound or salt according to claim 36, wherein R³ is H or R³ is methyl.

46. The compound or salt according to claim 36, wherein:
each of Z₁, Z₂, Z₃, and Z₄ are CH; or
one of Z₁, Z₂, Z₃, and Z₄ is CR⁴ and the remaining three of Z₁, Z₂, Z₃, and Z₄ are CH; or
two of Z₁, Z₂, Z₃, and Z₄ are CR⁴ and the remaining two of Z₁, Z₂, Z₃, and Z₄ are CH.

47. The compound or salt according to claim 36, wherein:
one of Z₁, Z₂, Z₃, and Z₄ is N and each of the remaining three of Z₁, Z₂, Z₃, and Z₄ are CH; or
one of Z₁, Z₂, Z₃, and Z₄ is N, another one of Z₁, Z₂, Z₃, and Z₄ is CR⁴, and the remaining two of Z₁, Z₂, Z₃, and Z₄ are CH; or
one of Z₁, Z₂, Z₃, and Z₄ is N, two of the remaining Z₁, Z₂, Z₃, and Z₄ are CR⁴, and the remaining Z₁, Z₂, Z₃, or Z₄ is CH.

48. The compound or salt according to claim 36, wherein:
one of Z₁, Z₂, Z₃, and Z₄ is NO and each of the remaining three of Z₁, Z₂, Z₃, and Z₄ are CH; or
one of Z₁, Z₂, Z₃, and Z₄ is NO, another one of Z₁, Z₂, Z₃, and Z₄ is CR⁴, and the remaining two of Z₁, Z₂, Z₃, and Z₄ are CH; or
one of Z₁, Z₂, Z₃, and Z₄ is NO, two of the remaining Z₁, Z₂, Z₃, and Z₄ are CR⁴, and the remaining Z₁, Z₂, Z₃, or Z₄ is CH.

49. The compound or salt according to claim 36, wherein each R⁴ is independently selected from halogen, (C₁-C₂)alkyl, halo(C₁-C₂)alkyl, (C₁-C₄)alkoxy, phenyl-oxy, and phenyl(C₁-C₄)alkoxy, wherein the phenyl moiety of said phenyl-oxy or phenyl(C₁-C₄)alkoxy is optionally substituted by 1 or 2 independently selected (C₁-C₂)alkyl groups.

50. The compound or salt according to claim 36, wherein each R⁴ is independently selected from halogen, C₁-C₃ alkyl, C₁-C₃ alkoxy, and phenylC₁-C₂ alkoxy.

51. The compound or salt according to claim 36, wherein each R⁴ is independently selected from chloro, fluoro, methyl, and methoxy.

52. The compound or salt according to claim 36, wherein A is 1H-indazol-3-yl, 4-(methyloxy)-1H-indazol-3-yl, 5-(methyloxy)-1H-indazol-3-yl, 6-(methyloxy)-1H-indazol-3-yl, 6-(n-propyloxy)-1H-indazol-3-yl, 5-methyl-1H-indazol-3-yl, 6-methyl-1H-indazol-3-yl, 5-trifluoromethyl-1H-indazol-3-yl, 6-trifluoromethyl-1H-indazol-3-yl, 7-trifluoromethyl-1H-indazol-3-yl, 4-chloro-1H-indazol-3-yl, 5-chloro-1H-indazol-3-yl, 6-chloro-1H-indazol-3-yl, 7-chloro-1H-indazol-3-yl, 4,5-dichloro-1H-indazol-3-yl, 5-methyl-6-fluoro-indazol-3-yl, 6-methyl-7-fluoro-indazol-3-yl, 5-fluoro-6-methyl-indazol-3-yl, 5-methyloxy-1-methyl-indazol-3-yl, 5-fluoro-1,6-dimethyl-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,6-difluoro-1H-indazol-3-yl, 5,7-difluoro-1H-indazol-3-yl, 6,7-difluoro-1H-indazol-3-yl, 1H-pyrazolo[3,4-b]pyridin-3-yl, 6-benzyloxy-1H-indazol-3-yl, 4-[(3-methylphenyl)oxy]-1H-indazol-3-yl, 1H-pyrazolo[3,4-b]pyridin-3-yl, 5-methyl-1H-pyrazolo[3,4-b]pyridin-3-yl, 6-methyl-1H-pyrazolo[3,4-b]pyridin-3-yl, 5-fluoro-6-methyl-1H-pyrazolo[3,4-b]pyridin-3-yl, 5-fluoro-1H-pyrazolo[3,4-b]pyridin-3-yl), 5-fluoro-7-oxido-1H-pyrazolo[3,4-b]pyridin-3-yl, or 5-fluoro-6-methyl-7-oxido-1H-pyrazolo[3,4-b]pyridin-3-yl.

53. The compound or salt according to claim 36, wherein A is 1H-indazol-3-yl, 4-(methyloxy)-1H-indazol-3-yl, 5-(methyloxy)-1H-indazol-3-yl, 6-(methyloxy)-1H-indazol-3-yl, 5-methyl-1H-indazol-3-yl, 6-methyl-1H-indazol-3-yl, 5-chloro-1H-indazol-3-yl, 6-chloro-1H-indazol-3-yl, 4,5-dichloro-1H-indazol-3-yl, 6-chloro-1-methyl-indazol-3-yl, 4-fluoro-1H-indazol-3-yl, 5-fluoro-1H-indazol-3-yl, 7-fluoro-1H-indazol-3-yl, 5,7-difluoro-1H-indazol-3-yl, 6,7-difluoro-1H-indazol-3-yl, 1H-pyrazolo[3,4-b]pyridin-3-yl, 6-benzyloxy-1H-indazol-3-yl.

54. A compound which is:
N⁴-(5-fluoro-1H-indazol-3-yl)-N⁴-methyl-N²-[3,4,5-tris(methyloxy)phenyl]-2,4-pyrimidinediamine,
N²-[3,4-bis(methyloxy)phenyl]-N⁴-1H-pyrazolo[3,4-b]pyridin-3-yl-2,4-pyrimidinediamine,
3-({4-[(5-fluoro-1H-indazol-3-yl)amino]-2-pyrimidinyl}amino)-N,N-dimethyl-benzenesulfonamide,
N⁴-(5-fluoro-1H-indazol-3-yl)-N²-[3-(methoxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-1H-pyrazolo[3,4-b]pyridin-3-yl)-N⁴-methyl-N²-[3-(methyloxy)-5-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N²-(1,1-dioxido-1-benzothien-4-yl)-N⁴-(5-fluoro-1H-indazol-3-yl)-2,4-pyrimidinediamine,
3-((4-((5-fluoro-6-methyl-1H-indazol-3-yl)(methyl)amino)pyrimidin-2-yl)amino)-5-methylbenzenesulfonamide,
N²-[2,3-dimethyl-5-(methylsulfonyl)phenyl]-N⁴-(5-fluoro-1H-indazol-3-yl)-2,4-pyrimidinediamine,
2-{[3-({4-[(5-fluoro-1H-indazol-3-yl)amino]-2-pyrimidinyl}amino)phenyl]sulfinyl}ethanol,
N²-[3,4-bis(methyloxy)phenyl]-N⁴-[4-(methyloxy)-1H-indazol-3-yl]-2,4-pyrimidinediamine,
N²-[3,4-bis(methyloxy)phenyl]-N⁴-(6-methyl-1H-indazol-3-yl)-2,4-pyrimidinediamine,
N²-[3,4-bis(methyloxy)phenyl]-N⁴-{6-[(phenylmethyl)oxy]-1H-indazol-3-yl}-2,4-pyrimidinediamine,
N²-[3,4-bis(methyloxy)phenyl]-N⁴-1H-indazol-3-yl-2,4-pyrimidinediamine,
N⁴-[5-(methyloxy)-1H-indazol-3-yl]-N²-[3,4,5-tris(methyloxy)phenyl]-2,4-pyrimidinediamine,
N²-[3,4-bis(methyloxy)phenyl]-N⁴-(6-chloro-1H-indazol-3-yl)-2,4-pyrimidinediamine,
N²-[3,4-bis(methyloxy)phenyl]-N⁴-(5-chloro-1H-indazol-3-yl)-2,4-pyrimidinediamine,
N²-[3,4-bis(methyloxy)phenyl]-N⁴-[5-(methyloxy)-1H-indazol-3-yl]-2,4-pyrimidinediamine,
N²-[3,4-bis(methyloxy)phenyl]-N⁴-(7-fluoro-1H-indazol-3-yl)-2,4-pyrimidinediamine,
N⁴-1H-indazol-3-yl-N²-[4-(4-methyl-1-piperazinyl)phenyl]-2,4-pyrimidinediamine,
N²-[3,4-bis(methyloxy)phenyl]-N⁴-(4-fluoro-1H-indazol-3-yl)-2,4-pyrimidinediamine,
N²-[3,4-bis(methyloxy)phenyl]-N⁴-[6-(methyloxy)-1H-indazol-3-yl]-2,4-pyrimidinediamine,
N²-[3,4-bis(methyloxy)phenyl]-N⁴-(4,5-dichloro-1H-indazol-3-yl)-2,4-pyrimidinediamine,
N²-[3,4-bis(methyloxy)phenyl]-N⁴-(6-chloro-1-methyl-1H-indazol-3-yl)-2,4-pyrimidinediamine,
3-[(4-{[5-(methyloxy)-1H-indazol-3-yl]amino}-2-pyrimidinyl)amino]benzenesulfonamide,
N²-[3,4-bis(methyloxy)phenyl]-N⁴-(5-fluoro-1H-indazol-3-yl)-2,4-pyrimidinediamine,
N²-[3,4-bis(methyloxy)phenyl]-N⁴-(5,7-difluoro-1H-indazol-3-yl)-2,4-pyrimidinediamine,
N²-[3,4-bis(methyloxy)phenyl]-N⁴-(6,7-difluoro-1H-indazol-3-yl)-2,4-pyrimidinediamine,
N⁴-(5-fluoro-1H-indazol-3-yl)-N²-[3-(1-pyrrolidinylsulfonyl)phenyl]-2,4-pyrimidinediamine,
N²-[4-fluoro-3-(methylsulfonyl)phenyl]-N⁴-[5-(methyloxy)-1H-indazol-3-yl]-2,4-pyrimidinediamine,
N⁴-(5-fluoro-1H-indazol-3-yl)-N²-[4-fluoro-3-(methylsulfonyl)phenyl]-2,4-pyrimidinediamine,
N⁴-(5-fluoro-1H-indazol-3-yl)-N⁴-methyl-N²-[3,4,5-tris(methyloxy)phenyl]-2,4-pyrimidinediamine,
N⁴-(5-fluoro-1H-indazol-3-yl)-N⁴-methyl-N²-[3-(methylsulfonyl)phenyl]-2,4-pyrimidinediamine,
N²-[3,4-bis(methyloxy)phenyl]-N⁴-[1-methyl-5-(methyloxy)-1H-indazol-3-yl]-2,4-pyrimidinediamine,
N⁴-[5-(methyloxy)-1H-indazol-3-yl]-N²-[3,4,5-tris(methyloxy)phenyl]-2,4-pyrimidinediamine,
N²-[4-fluoro-3-(methyloxy)phenyl]-N⁴-[5-(methyloxy)-1H-indazol-3-yl]-2,4-pyrimidinediamine,
N-[2-(dimethylamino)ethyl]-N-methyl-3-[(4-{[5-(methyloxy)-1H-indazol-3-yl]amino}-2-pyrimidinyl)amino]benzenesulfonamide,
N,N-dimethyl-3-[(4-{[5-(methyloxy)-1H-indazol-3-yl]amino}-2-pyrimidinyl)amino]benzenesulfonamide,
N⁴-[5-(methyloxy)-1H-indazol-3-yl]-N²-[3-(1-pyrrolidinylsulfonyl)phenyl]-2,4-pyrimidinediamine,
N²-{3-[1-(dimethylamino)-2,2,2-trifluoroethyl]phenyl}-N⁴-(5-fluoro-1H-indazol-3-yl)-2,4-pyrimidinediamine,
2-[3-({4-[(5-fluoro-1H-indazol-3-yl)amino]-2-pyrimidinyl}amino)phenyl]-2-methylpropanenitrile,
N⁴-(5-fluoro-1H-indazol-3-yl)-N²-[3-(methylsulfonyl)phenyl]-2,4-pyrimidinediamine,
N²-(2,3-dihydro-1,4-benzodioxin-6-yl)-N⁴-(5-fluoro-1H-indazol-3-yl)-2,4-pyrimidinediamine,
N⁴-(5-fluoro-1H-indazol-3-yl)-N²-[4-methyl-3-(methylsulfonyl)phenyl]-2,4-pyrimidinediamine,
2-{[3-({4-[(5-fluoro-1H-indazol-3-yl)amino]-2-pyrimidinyl}amino)phenyl]sulfonyl}-2-methyl-1-propanol,
4-({4-[(5-fluoro-1H-indazol-3-yl)amino]-2-pyrimidinyl}amino)-2,6-bis(methyloxy)phenol,
N⁴-(5-fluoro-1H-indazol-3-yl)-N²-1H-indol-6-yl-2,4-pyrimidinediamine,
N⁴-(5-fluoro-1H-indazol-3-yl)-N²-1H-indol-4-yl-2,4-pyrimidinediamine,
N⁴-(5-fluoro-1H-indazol-3-yl)-N²-[1-(methylsulfonyl)-1H-indol-6-yl]-2,4-pyrimidinediamine,
N⁴-(5-fluoro-1H-indazol-3-yl)-N²-[2-methyl-3-(methylsulfonyl)phenyl]-2,4-pyrimidinediamine,
N⁴-(5-fluoro-1H-indazol-3-yl)-N²-[1-(methylsulfonyl)-1H-indol-5-yl]-2,4-pyrimidinediamine,
N⁴-(7-chloro-1H-indazol-3-yl)-N²-[4-methyl-3-(methylsulfonyl)phenyl]-2,4-pyrimidinediamine,
N²-[4-methyl-3-(methylsulfonyl)phenyl]-N⁴-[7-(trifluoromethyl)-1H-indazol-3-yl]-2,4-pyrimidinediamine,
N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[4-(methylsulfonyl)phenyl]-2,4-pyrimidinediamine,
N⁴-(5-fluoro-1H-indazol-3-yl)-N²-[3-(4-morpholinylsulfonyl)phenyl]-2,4-pyrimidinediamine,
3-({4-[(5-fluoro-1H-indazol-3-yl)amino]-2-pyrimidinyl}amino)-N-(methyloxy)benzenesulfonamide,
3-({4-[(5-fluoro-1H-indazol-3-yl)amino]-2-pyrimidinyl}amino)-N-methyl-N-(methyloxy)benzenesulfonamide,
5-({4-[(5-fluoro-1H-indazol-3-yl)amino]-2-pyrimidinyl}amino)-N,2-dimethyl-N-(methyloxy)benzenesulfonamide,
N²-[3-(ethyloxy)-5-(methylsulfonyl)phenyl]-N⁴-(5-fluoro-1H-indazol-3-yl)-2,4-pyrimidinediamine,
N⁴-(5-fluoro-1H-indazol-3-yl)-N²-{3-[(1-methylethyl)sulfonyl]phenyl}-2,4-pyrimidinediamine,
N²-(3,5-dimethylphenyl)-N⁴-(5-fluoro-1H-indazol-3-yl)-2,4-pyrimidinediamine,
N⁴-(5-fluoro-1H-indazol-3-yl)-N²-{4-methyl-3-[(1-methylethyl)sulfonyl]phenyl}-2,4-pyrimidinediamine,
N⁴-(5-fluoro-1H-indazol-3-yl)-N²-[4-methyl-3-(methylsulfonyl)phenyl]-2,4-pyrimidinediamine,
2-(ethyloxy)-5-({4-[(5-fluoro-1H-indazol-3-yl)amino]-2-pyrimidinyl}amino)-N,N-dimethylbenzenesulfonamide,
N⁴-(5-fluoro-1H-indazol-3-yl)-N²-[3-(methyloxy)-5-(methylsulfonyl)phenyl]-2,4-pyrimidinediamine,
N²-[3-(ethylsulfonyl)-5-(methyloxy)phenyl]-N⁴-(5-fluoro-1H-indazol-3-yl)-2,4-pyrimidinediamine,
N²-{4-(ethyloxy)-3-[(1-methylethyl)sulfonyl]phenyl}-N⁴-(5-fluoro-1H-indazol-3-yl)-2,4-pyrimidinediamine,
N⁴-(5-fluoro-1H-indazol-3-yl)-N⁴-methyl-N²-[4-methyl-3-(methylsulfonyl)phenyl]-2,4-pyrimidinediamine,
N⁴-(5-fluoro-1H-indazol-3-yl)-N⁴-methyl-N²-[3-methyl-5-(methylsulfonyl)phenyl]-2,4-pyrimidinediamine,
N⁴-ethyl-N⁴-(5-fluoro-1H-indazol-3-yl)-N²-[3-methyl-5-(methylsulfonyl)phenyl]-2,4-pyrimidinediamine,
N⁴-(5-fluoro-1H-indazol-3-yl)-N²-[3-(methylsulfonyl)-5-(4-morpholinyl)phenyl]-2,4-pyrimidinediamine,
N⁴-(5-fluoro-1H-indazol-3-yl)-N²-(1-methyl-1H-imidazol-2-yl)-2,4-pyrimidinediamine,
N⁴-(5-fluoro-1H-indazol-3-yl)-N²-[3-(methyloxy)-5-(methylsulfonyl)phenyl]-2,4-pyrimidinediamine,
N⁴-(5-fluoro-1H-indazol-3-yl)-N²-[3-fluoro-5-(methylsulfonyl)phenyl]-2,4-pyrimidinediamine,
2-{[5-({4-[(5-fluoro-1H-indazol-3-yl)amino]-2-pyrimidinyl}amino)-2-methylphenyl]sulfonyl}-2-methyl-1-propanol,
N⁴-(5-fluoro-1H-indazol-3-yl)-N²-{4-fluoro-3-[(1-methylethyl)sulfonyl]phenyl}-2,4-pyrimidinediamine,
N⁴-(5-fluoro-1H-indazol-3-yl)-N⁴-methyl-N²-[3-(methyloxy)-5-(methylsulfonyl)phenyl]-2,4-pyrimidinediamine,
N⁴-(5-fluoro-1H-indazol-3-yl)-N²-[3-fluoro-4-methyl-5-(methylsulfonyl)phenyl]-2,4-pyrimidinediamine,
N-[3-({4-[(5-fluoro-1H-indazol-3-yl)amino]-2-pyrimidinyl}amino)phenyl]-N-methylmethanesulfonamide,
N²-[3-(dimethylamino)-5-(methylsulfonyl)phenyl]-N⁴-(5-fluoro-1H-indazol-3-yl)-2,4-pyrimidinediamine,
3-({4-[(5-fluoro-1H-indazol-3-yl)amino]-2-pyrimidinyl}amino)benzamide,
N⁴-(5-fluoro-1H-indazol-3-yl)-N²-[2-fluoro-4-(methylsulfonyl)phenyl]-2,4-pyrimidinediamine,
N⁴-(5-fluoro-1H-indazol-3-yl)-N²-[3-(methylsulfonyl)-5-(1-pyrrolidinyl)phenyl]-2,4-pyrimidinediamine,
N²-[3-(ethyloxy)-5-(ethylsulfonyl)phenyl]-N⁴-(5-fluoro-1H-indazol-3-yl)-2,4-pyrimidinediamine,
N⁴-(5-fluoro-1H-indazol-3-yl)-N²-[4-(methylsulfonyl)-2,3-dihydro-1-benzofuran-6-yl]-2,4-pyrimidinediamine,
N⁴-(5-fluoro-1H-indazol-3-yl)-N²-[4-fluoro-3-(methylsulfonyl)phenyl]-N⁴-methyl-2,4-pyrimidinediamine,
N⁴-(5-fluoro-1H-indazol-3-yl)-N⁴-methyl-N²-[2-methyl-3-(methylsulfonyl)phenyl]-2,4-pyrimidinediamine,
N²-[3,4-dimethyl-5-(methylsulfonyl)phenyl]-N⁴-(5-fluoro-1H-indazol-3-yl)-N⁴-methyl-2,4-pyrimidinediamine,
N⁴-(5-fluoro-1H-indazol-3-yl)-N²-[2-(methyloxy)-5-(methylsulfonyl)phenyl]-2,4-pyrimidinediamine,
3-({4-[(5-fluoro-1H-indazol-3-yl)amino]-2-pyrimidinyl}amino)-4-(methyloxy)benzenesulfonamide,
N⁴-(5-fluoro-1H-indazol-3-yl)-N²-{3-[(trifluoromethyl)sulfonyl]phenyl}-2,4-pyrimidinediamine,
N²-1-benzothien-4-yl-N⁴-(5-fluoro-1H-indazol-3-yl)-2,4-pyrimidinediamine,
N²-[2,4-dimethyl-5-(methylsulfonyl)phenyl]-N⁴-(5-fluoro-1H-indazol-3-yl)-2,4-pyrimidinediamine,
7-({4-[(5-fluoro-1H-indazol-3-yl)amino]-2-pyrimidinyl}amino)-2,3-dihydro-1-benzofuran-5-sulfonamide,
N⁴-(5-fluoro-1H-indazol-3-yl)-N²-[2-fluoro-3-(methylsulfonyl)phenyl]-2,4-pyrimidinediamine,
N²-[4-methyl-3-(methylsulfonyl)phenyl]-N⁴-[6-(trifluoromethyl)-1H-indazol-3-yl]-2,4-pyrimidinediamine,
N⁴-(6,7-difluoro-1H-indazol-3-yl)-N²-[4-methyl-3-(methylsulfonyl)phenyl]-2,4-pyrimidinediamine,
N⁴-(6-methyl-1H-indazol-3-yl)-N²-[4-methyl-3-(methylsulfonyl)phenyl]-2,4-pyrimidinediamine,
N⁴-(5-chloro-1H-indazol-3-yl)-N²-[4-methyl-3-(methylsulfonyl)phenyl]-2,4-pyrimidinediamine,
N⁴-(4-chloro-1H-indazol-3-yl)-N²-[4-methyl-3-(methylsulfonyl)phenyl]-2,4-pyrimidinediamine,
N⁴-(7-fluoro-1H-indazol-3-yl)-N²-[4-methyl-3-(methylsulfonyl)phenyl]-2,4-pyrimidinediamine,
N⁴-(5,7-difluoro-1H-indazol-3-yl)-N²-[4-methyl-3-(methylsulfonyl)phenyl]-2,4-pyrimidinediamine,
N⁴-(5,6-difluoro-1H-indazol-3-yl)-N²-[4-methyl-3-(methylsulfonyl)phenyl]-2,4-pyrimidinediamine,
N⁴-(5-fluoro-1H-indazol-3-yl)-N²-[2-methyl-5-(methylsulfonyl)phenyl]-2,4-pyrimidinediamine,
N⁴-(5-fluoro-1H-indazol-3-yl)-N²-[3,4,5-tris(methyloxy)phenyl]-2,4-pyrimidinediamine,
N²-[4-methyl-3-(methylsulfonyl)phenyl]-N⁴-[5-(methyloxy)-1H-indazol-3-yl]-2,4-pyrimidinediamine,
N′-[3-({4-[(5-fluoro-1H-indazol-3-yl)amino]-2-pyrimidinyl}amino)phenyl]-N,N-dimethylsulfamide,
3-({4-[(5-fluoro-1H-indazol-3-yl)amino]-2-pyrimidinyl}amino)benzenesulfonamide,
5-({4-[(5-fluoro-1H-indazol-3-yl)amino]-2-pyrimidinyl}amino)-2-methylbenzenesulfonamide,
N⁴-(6,7-difluoro-1H-indazol-3-yl)-N⁴-methyl-N²-[4-methyl-3-(methylsulfonyl)phenyl]-2,4-pyrimidinediamine,
N⁴-(6,7-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3,4,5-tris(methyloxy)phenyl]-2,4-pyrimidinediamine,
N⁴-(7-fluoro-1H-indazol-3-yl)-N⁴-methyl-N²-[4-methyl-3-(methylsulfonyl)phenyl]-2,4-pyrimidinediamine,
N⁴-(7-fluoro-1H-indazol-3-yl)-N⁴-methyl-N²-[3-methyl-5-(methylsulfonyl)phenyl]-2,4-pyrimidinediamine,
N⁴-(7-fluoro-1H-indazol-3-yl)-N⁴-methyl-N²-[3-(methyloxy)-5-(methylsulfonyl)phenyl]-2,4-pyrimidinediamine,
N⁴-(7-fluoro-1H-indazol-3-yl)-N⁴-methyl-N²-[3,4,5-tris(methyloxy)phenyl]-2,4-pyrimidinediamine,
N⁴-(5,6-difluoro-1H-indazol-3-yl)-N⁴-methyl-N²-[3,4,5-tris(methyloxy)phenyl]-2,4-pyrimidinediamine,
N⁴-(5,6-difluoro-1H-indazol-3-yl)-N⁴-methyl-N²-[3-(methyloxy)-5-(methylsulfonyl)phenyl]-2,4-pyrimidinediamine,
N⁴-(5,6-difluoro-1H-indazol-3-yl)-N⁴-methyl-N²-[4-methyl-3-(methylsulfonyl)phenyl]-2,4-pyrimidinediamine,
N⁴-(5,6-difluoro-1H-indazol-3-yl)-N⁴-methyl-N²-[3-methyl-5-(methylsulfonyl)phenyl]-2,4-pyrimidinediamine,
N⁴-(6,7-difluoro-1H-indazol-3-yl)-N⁴-methyl-N²-[3-(methyloxy)-5-(methylsulfonyl)phenyl]-2,4-pyrimidinediamine,
N⁴-(1H-indazol-3-yl)-N-(3-methyl-5-(methylsulfonyl)phenyl)pyrimidine-2,4-diamine,
N⁴-(1H-indazolol-3-yl)-N-(4-methyl-3-(methylsulfonyl)phenyl)pyrimidine-2,4-diamine,
N⁴-(7-fluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(4-methyl-3-(methylsulfonyl)phenyl)pyrimidine-2,4-diamine,
N⁴-(6,7-difluoro-1H-indazol-3-yl)-N²-(3-methyl-5-(methylsulfonyl)phenyl)pyrimidine-2,4-diamine,
N⁴-(4-fluoro-1H-indazol-3-yl)-N⁴-methyl-N²-(3-methyl-5-(methylsulfonyl)phenyl)pyrimidine-2,4-diamine,
N⁴-(5-fluoro-6-methyl-1H-indazol-3-yl)-N⁴-methyl-N²-[3-methyl-5-(methylsulfonyl)phenyl]-2,4-pyrimidinediamine,
N⁴-(7-fluoro-6-methyl-1H-indazol-3-yl)-N⁴-methyl-N²-[3-methyl-5-(methylsulfonyl)phenyl]-2,4-pyrimidinediamine,
N⁴-(5,6-difluoro-1H-indazol-3-yl)-N²-[3-methyl-5-(methylsulfonyl)phenyl]-2,4-pyrimidinediamine,
N⁴-(5,6-difluoro-1H-indazol-3-yl)-N²-[3-(methyloxy)-5-(methylsulfonyl)phenyl]-2,4-pyrimidinediamine,
N⁴-(5,6-difluoro-1H-indazol-3-yl)-N²-[3,4,5-tris(methyloxy)phenyl]-2,4-pyrimidinediamine,
N⁴-(6-fluoro-5-methyl-1H-indazol-3-yl)-N⁴-methyl-N²-[3-methyl-5-(methylsulfonyl)phenyl]-2,4-pyrimidinediamine,
2-chloro-5-({4-[(5-fluoro-1H-indazol-3-yl)amino]-2-pyrimidinyl}amino)benzenesulfonamide,
N⁴-(5-fluoro-6-methyl-1H-indazol-3-yl)-N⁴-methyl-N²-[3-methyl-5-(methylsulfonyl)phenyl]-2,4-pyrimidinediamine,
3-({4-[(7-fluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-(methylsulfonyl)-5-(pyrrolidin-1-yl)phenyl)pyrimidine-2,4-diamine,
3-({4-[(6,7-difluoro-1H-indazol-3-yl)(methyl)amino]-2-pyrimidinyl}amino)-5-methylbenzenesulfonamide,
N⁴-(7-fluoro-1H-indazol-3-yl)-N⁴-methyl-N²-(3-methyl-5-(pyrrolidin-1-yl)phenyl)pyrimidine-2,4-diamine,
N⁴-(7-fluoro-1H-indazol-3-yl)-N⁴-methyl-N²-(3-methyl-5-morpholinophenyl)pyrimidine-2,4-diamine,
N⁴-(7-fluoro-6-methyl-1H-indazol-3-yl)-N⁴-methyl-N²-[3-(methyloxy)-5-(methylsulfonyl)phenyl]-2,4-pyrimidinediamine,
N⁴-(5-fluoro-6-methyl-1H-indazol-3-yl)-N⁴-methyl-N²-[3-(methyloxy)-5-(methylsulfonyl)phenyl]-2,4-pyrimidinediamine,
N⁴-(6,7-difluoro-1H-indazol-3-yl)-N²-(1,1-dioxido-1-benzothien-6-yl)-N⁴-methyl-2,4-pyrimidinediamine,
N⁴-methyl-N⁴-(5-methyl-1H-indazol-3-yl)-N²-[3-(methyloxy)-5-(methylsulfonyl)phenyl]-2-pyrimidinediamine,
N⁴-methyl-N⁴-(5-methyl-1H-indazol-3-yl)-N²-[3-methyl-5-(methylsulfonyl)phenyl]-2,4-pyrimidinediamine,
3-({4-[(7-fluoro-6-methyl-1H-indazol-3-yl)(methyl)amino]-2-pyrimidinyl}amino)-5-methylbenzenesulfonamide,
N⁴-(7-fluoro-1H-indazol-3-yl)-N²-(3-methoxy-5-(methylsulfonyl)phenyl)pyrimidine-2,4-diamine,
5-((4-((7-fluoro-1H-indazol-3-yl)(methyl)amino)pyrimidin-2-yl)amino)-2-methoxybenzenesulfonamide,
N⁴-(6-methyl-1H-indazol-3-yl)-N²-[3-methyl-5-(methylsulfonyl)phenyl]-2,4-pyrimidinediamine,
N⁴-(6,7-difluoro-1H-indazol-3-yl)-N²-[3-(methyloxy)-5-(methylsulfonyl)phenyl]-2,4-pyrimidinediamine,
3-((4-((5,6-difluoro-1H-indazol-3-yl)(methyl)amino)pyrimidin-2-yl)amino)-5-methylbenzenesulfonamide,
N⁴-(5,6-difluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N²-(4-methoxy-3-(methylsulfonyl)phenyl)-N⁴-methylpyrimidine-2,4-diamine,
N⁴-(7-fluoro-1H-indazol-3-yl)-N⁴-methyl-N²-(3-methyl-5-((tetrahydro-2H-pyran-4-yl)sulfonyl)phenyl)pyrimidine-2,4-diamine,
3-({4-[(6,7-difluoro-1H-indazol-3-yl)(methyl)amino]-2-pyrimidinyl}amino)-5-(methyloxy)benzenesulfonamide,
3-((4-((5,6-difluoro-1H-indazol-3-yl)(methyl)amino)pyrimidin-2-yl)amino)-5-methoxybenzenesulfonamide,
3-({4-[(5-fluoro-6-methyl-1H-indazol-3-yl)(methyl)amino]-2-pyrimidinyl}amino)-5-(methyloxy)benzenesulfonamide,
3-({4-[(7-fluoro-6-methyl-1H-indazol-3-yl)(methyl)amino]-2-pyrimidinyl}amino)-5-(methyloxy)benzenesulfonamide,
3-({4-[(6,7-difluoro-1H-indazol-3-yl)amino]-2-pyrimidinyl}amino)-5-methylbenzenesulfonamide,
3-((4-((7-fluoro-1H-indazol-3-yl)amino)pyrimidin-2-yl)amino)-5-methoxybenzenesulfonamide,
3-((4-((7-fluoro-1H-indazol-3-yl)(methyl)amino)pyrimidin-2-yl)amino)-5-methoxybenzenesulfonamide,
N²-[4-(ethyloxy)-3-(methylsulfonyl)phenyl]-N⁴-(7-fluoro-1H-indazol-3-yl)-N⁴-methyl-2,4-pyrimidinediamine,
3-methoxy-5-((4-(methyl(7-(trifluoromethyl)-1H-indazol-3-yl)amino)pyrimidin-2-yl)amino)benzenesulfonamide,
3-methyl-5-[(4-{methyl[6-(trifluoromethyl)-1H-indazol-3-yl]amino}-2-pyrimidinyl)amino]benzenesulfonamide,
3-(methyloxy)-5-[(4-{methyl[6-(trifluoromethyl)-1H-indazol-3-yl]amino}-2-pyrimidinyl)amino]benzenesulfonamide,
3-methyl-5-((4-(methyl(7-(trifluoromethyl)-1H-indazol-3-yl)amino)pyrimidin-2-yl)amino)benzenesulfonamide,
N,N-dimethyl-3-{[4-(1H-pyrazolo[3,4-b]pyridin-3-ylamino)-2-pyrimidinyl]amino}benzenesulfonamide,
N⁴-(5-methyl-1H-pyrazolo[3,4-b]pyridin-3-yl)-N²-[3-(methylsulfonyl)phenyl]-2,4-pyrimidinediamine,
N²-[4-methyl-3-(methylsulfonyl)phenyl]-N⁴-(5-methyl-1H-pyrazolo[3,4-b]pyridin-3-yl)-2,4-pyrimidinediamine,
N²-[4-fluoro-3-(methylsulfonyl)phenyl]-N⁴-(5-methyl-1H-pyrazolo[3,4-b]pyridin-3-yl)-2,4-pyrimidinediamine,
N⁴-(5-fluoro-1H-pyrazolo[3,4-b]pyridin-3-yl)-N²-[3-(methylsulfonyl)phenyl]-2,4-pyrimidinediamine,
N²-[4-fluoro-3-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N⁴-methyl-N²-[3-(methylsulfonyl)phenyl]-2,4-pyrimidinediamine,
N⁴-(5-fluoro-1H-pyrazolo[3,4-b]pyridin-3-yl)-N²-{4-methyl-3-[(1-methylethyl)sulfonyl]phenyl}-2,4-pyrimidinediamine,
N⁴-(5-fluoro-1H-pyrazolo[3,4-b]pyridin-3-yl)-N²-[3-(methyloxy)-5-(methylsulfonyl)phenyl]-2,4-pyrimidinediamine,
N⁴-(5-fluoro-1H-pyrazolo[3,4-b]pyridin-3-yl)-N⁴-methyl-N²-[3-methyl-5-(methylsulfonyl)phenyl]-2,4-pyrimidinediamine,
N⁴-(5-fluoro-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-1H-pyrazolo[3,4-b]pyridin-3-yl)-N²-[3-(methylsulfonyl)-5-(4-morpholinyl)phenyl]-2,4-pyrimidinediamine,
N⁴-(5-fluoro-1H-pyrazolo[3,4-b]pyridin-3-yl)-N²-[4-methyl-3-(methylsulfonyl)phenyl]-2,4-pyrimidinediamine,
N²-[3,4-dimethyl-5-(methylsulfonyl)phenyl]-N⁴-(5-fluoro-1H-pyrazolo[3,4-b]pyridin-3-yl)-2,4-pyrimidinediamine,
N⁴-(5-fluoro-1H-pyrazolo[3,4-b]pyridin-3-yl)-N⁴-methyl-N²-[4-methyl-3-(methylsulfonyl)-5-(1-pyrrolidinyl)phenyl]-2,4-pyrimidinediamine,
N⁴-(6-methyl-1H-pyrazolo[3,4-b]pyridin-3-yl)-N²-[3-(methylsulfonyl)phenyl]-2,4-pyrimidinediamine,
N²-[3-[(difluoromethyl)oxy]-5-(methylsulfonyl)phenyl]-N⁴-(5-fluoro-1H-pyrazolo[3,4-b]pyridin-3-yl)-N⁴-methyl-2,4-pyrimidinediamine,
N²-[3-(3,3-difluoro-1-pyrrolidinyl)-5-(methylsulfonyl)phenyl]-N⁴-(5-fluoro-1H-pyrazolo[3,4-b]pyridin-3-yl)-N⁴-methyl-2,4-pyrimidinediamine,
N⁴-(5-fluoro-1H-pyrazolo[3,4-b]pyridin-3-yl)-N⁴-methyl-N²-[4-(methylsulfonyl)-2,3-dihydro-1-benzofuran-6-yl]-2,4-pyrimidinediamine,
N⁴-(5-fluoro-1H-pyrazolo[3,4-b]pyridin-3-yl)-N²-[3,4,5-tris(methyloxy)phenyl]-2,4-pyrimidinediamine,
N⁴-(5-fluoro-1H-pyrazolo[3,4-b]pyridin-3-yl)-N⁴-methyl-N²-[3,4,5-tris(methyloxy)phenyl]-2,4-pyrimidinediamine,
N²-[4-methyl-3-(methylsulfonyl)phenyl]-N⁴-(6-methyl-1H-pyrazolo[3,4-b]pyridin-3-yl)-2,4-pyrimidinediamine,
N²-{3-[(1,1-dimethylethyl)sulfonyl]-5-methylphenyl}-N⁴-(5-fluoro-1H-pyrazolo[3,4-b]pyridin-3-yl)-2,4-pyrimidinediamine,
N^{2-[}3-[(1,1-dimethylethyl)sulfonyl]-5-(trifluoromethyl)phenyl]-N⁴-(5-fluoro-1H-pyrazolo[3,4-b]pyridin-3-yl)-2,4-pyrimidinediamine,
N⁴-(5-fluoro-6-methyl-1H-pyrazolo[3,4-b]pyridin-3-yl)-N⁴-methyl-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-methyl-5-(methylsulfonyl)phenyl]pyrimidine-2,4-diamine,
N⁴-(5-fluoro-6-methyl-1H-pyrazolo[3,4-b]pyridin-3-yl)-N²-[3-(methyloxy)-5-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)-5-(1-pyrrolidinyl)phenyl]-2,4-pyrimidinediamine,
N²-[3-[(difluoromethyl)oxy]-5-(methylsulfonyl)phenyl]-N⁴-(5-fluoro-6-methyl-1H-pyrazolo[3,4-b]pyridin-3-yl)-N⁴-methyl-2,4-pyrimidinediamine,
N⁴-(5-fluoro-6-methyl-1H-pyrazolo[3,4-b]pyridin-3-yl)-N⁴-methyl-N²-[4-methyl-3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3-(methylsulfonyl)phenyl]-2,4-pyrimidinediamine,
N⁴-(5-fluoro-6-methyl-1H-pyrazolo[3,4-b]pyridin-3-yl)-N⁴-methyl-N²-[3,4,5-tris(methyloxy)phenyl]-2,4-pyrimidinediamine,
N²-{3-[(1,1-dimethylethyl)sulfonyl]-5-methylphenyl}-N⁴-(5-fluoro-6-methyl-1H-pyrazolo[3,4-b]pyridin-3-yl)-N⁴-methyl-2,4-pyrimidinediamine,
N⁴-(5-fluoro-1,6-dimethyl-1H-pyrazolo[3,4-b]pyridin-3-yl)-N⁴-methyl-N²-[3-methyl-5-(methylsulfonyl)phenyl]-2,4-pyrimidinediamine,
3-({4-[(5-fluoro-6-methyl-1H-pyrazolo[3,4-b]pyridin-3-yl)(methyl)amino]-2-pyrimidinyl}amino)benzenesulfonamide,
3-({4-[(5-fluoro-1H-pyrazolo[3,4-b]pyridin-3-yl)amino]-2-pyrimidinyl}amino)benzenesulfonamide,
N⁴-(5-fluoro-6-methyl-1H-pyrazolo[3,4-b]pyridin-3-yl)-N²-[3-fluoro-5-(methylsulfonyl)phenyl]-N⁴-methyl-2,4-pyrimidinediamine,
3-({4-[(5-fluoro-1H-pyrazolo[3,4-b]pyridin-3-yl)(methyl)amino]-2-pyrimidinyl}amino)benzenesulfonamide,
2-chloro-5-({4-[(5-fluoro-1H-pyrazolo[3,4-b]pyridin-3-yl)amino]-2-pyrimidinyl}amino)benzenesulfonamide,
2-chloro-5-({4-[(5-fluoro-1H-pyrazolo[3,4-b]pyridin-3-yl)(methyl)amino]-2-pyrimidinyl}amino)benzenesulfonamide,
5-({4-[(5-fluoro-1H-pyrazolo[3,4-b]pyridin-3-yl)(methyl)amino]-2-pyrimidinyl}amino)-2-methylbenzenesulfonamide,
N⁴-(5-fluoro-7-oxido-1H-pyrazolo[3,4-b]pyridin-3-yl)-N⁴-methyl-N²-[3-methyl-5-(methylsulfonyl)phenyl]-2,4-pyrimidinediamine,
5-fluoro-6-methyl-3-(methyl(2-((3-methyl-5-(methylsulfonyl)phenyl)amino)pyrimidin-4-yl)amino)-1H-pyrazolo[3,4-b]pyridine 7-oxide, or
N⁴-(5-fluoro-6-methyl-1H-pyrazolo[3,4-b]pyridin-3-yl)-N⁴-methyl-N²-(3-methyl-5-((tetrahydro-2H-pyran-4-yl)sulfonyl)phenyl)pyrimidine-2,4-diamine,
or a pharmaceutically acceptable salt thereof.

55. A pharmaceutical composition comprising the compound according to claim 36, or a pharmaceutically acceptable salt thereof, and a pharmaceutically acceptable excipient.
